(19) United States
(12) Patent Application Publication
(10) Pub. No.: US 2020/0268762 A1
(43) Pub. Date: Aug. 27, 2020

(54) 7-SUBSTITUTED SULFONIMIDOYLPURINONE COMPOUNDS AND DERIVATIVES FOR THE TREATMENT AND PROPHYLAXIS OF LIVER CANCER

(71) Applicant: Hoffmann-La Roche Inc., Little Falls, NJ (US)

(72) Inventors: THOMAS POESCHINGER, STARNBERG (DE); CAROLA RIES, PENZBERG (DE); HONG SHEN, SHANGHAI (CN); HONGYING YUN, SHANGHAI (CN); SABINE HOVES, HABACH (DE); CARINA HAGE, PENZBERG (DE)

(73) Assignee: Hoffmann-La Roche Inc., Little Falls, NJ (US)

(21) Appl. No.: 16/286,151

(22) Filed: Feb. 26, 2019

(51) Int. Cl. A61K 31/522; A61K 39/395; A61P 35/00; A61K 31/44

(52) U.S. Cl. CPC A61K 31/522; A61K 39/3955; A61K 2039/505; A61K 31/44; A61P 35/00

(57) ABSTRACT

The present invention relates to compounds of formula (I),

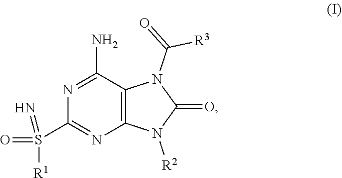

wherein R¹, R² and R³ are as described herein, and their prodrugs or pharmaceutically acceptable salt, enantiomer or diastereomer thereof, for (use in) the treatment and/or prophylaxis of liver cancer.

### CROSS REFERENCE TO RELATED APPLICATION

This application claims the benefit of priority under 35 U.S.C. § 119 to PCT/CN2018/077501, filed Feb. 28, 2018, which application is hereby incorporated by reference in its entirety.

### SEQUENCE LISTING

The instant application contains a Sequence Listing submitted via EFS-Web and hereby incorporated by reference in its entirety. Said ASCII copy, created on Feb. 22, 2019, is named P34694USSeqList.txt, and is 25,347 bytes in size.

### FIELD OF THE INVENTION

This invention related to methods of treating of liver cancer novel sulfonimidoylpurinones derivatives of formula (I) that have in vivo Toll-like receptor agonism activity

### BACKGROUND OF THE INVENTION

Liver cancer is the fifth most common form of cancer. Each year, approximately 750,000 cases are diagnosed and about 700,000 people die from the disease each year, making it the third most common cause of cancer death in the world (Ferlay et al., Int. J. Cancer 127:2893-2917 (2010)). In the United States, the incidence of primary liver cancer has been rising, and while some progress has been made in detecting and treating localized disease, the five year survival rate for late stage liver cancer is still well below 10% (American-Cancer-Society. 2012. Cancer Facts &amp; FIGS. 2012. Atlanta: American Cancer Society).

Established treatments for liver cancer include surgical removal of the part of the liver containing the tumor (partial hepatectomy), liver transplantation, transcatheter arterial chemoembolization (TACE), in situ tumor destruction by various methods such as radiofrequency ablation (RFA) or cryosurgery and administration of Sorafenib. Treatment options for late stage liver patients are limited. Thus, effective treatments of liver cancer remain a significant unmet medical need.

The present invention relates to compounds of formula (I),

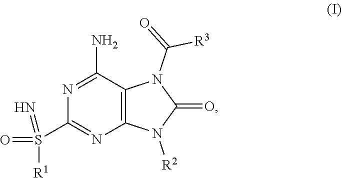

wherein R¹ to R³ are described below, or pharmaceutically acceptable salt, enantiomer or diastereomer thereof.

Toll-like receptors (TLRs) detect a wide range of conserved pathogen-associated molecular patterns (PAMPs). They play an important role of sensing invading pathogens and subsequent initiation of innate immune responses. There are 10 known members of the TLR family in human, which are type I transmembrane proteins featuring an extracellular leucine-rich domain and a cytoplasmic tail that contains a conserved Toll/interleukin (IL)-1 receptor (TIR) domain. Within this family, TLR3, TLR7, TLR8 and TLR9 are located within endosomes.

TLR7 can be activated by binding to a specific small molecule ligand (i.e., TLR7 agonist) or its native ligand (i.e., single-stranded RNA, ssRNA). Following binding of ssRNA to TLR7, the receptor in its dimerized form is believed to undergo a structural change leading to the subsequent recruitment of adapter proteins at its cytoplasmic domain, including the myeloid differentiation primary response gene 88 (MyD88). Following the initiation of the receptor signalling cascade via the MyD88 pathway, cytoplasmic transcription factors such as interferon regulatory factor 7 (IRF-7) and nuclear factor kappa B (NF-κB) are activated. These transcription factors then translocate to the nucleus and initiate the transcription of various genes, e.g., IFN-α and other antiviral cytokine genes.

WO201772662 relates to TLR7 agonist-anti HER2 conjugates for the treatment of HER2 positive cancers. Hotz et al, Oncoimmunology 2012, 227-228 relates to cancer treatment with TLR7 agonists. However so far no TLR7 agonist is used systemically for the treatment of cancer. Only topical TLR7 agonist imiquimod is known to induce immune-mediated rejection of skin metastases in patients with breast cancer (Adams S., Kozhaya L., Martiniuk F., Meng T. C., Chiriboga L., Liebes L., Hochman T., Shuman N., Axelrod D., Speyer J., et al. Clin. Cancer Res. 2012; 18:6748-6757.

### SUMMARY OF THE INVENTION

The present invention relates to a series of novel 6-amino-2-sulfonimidoyl-9-substituted-7-substituted-purin-8-one compounds with Toll-like receptor agonistic activity and their prodrugs for use in the treatment or prophylaxis (prevention) of liver cancer.

It was found out that the potent and safe TLR7 agonist prodrugs described herein are effective in the treatment of liver cancer either alone or in combination with other agents.

The present invention provides a series of novel 6-amino-2-sulfonimidoyl-9-substituted-7-substituted-purin-8-one compounds that have Toll-like receptor agonistic activity and their prodrugs. The invention also provides the bio-activity of such compounds to induce cytokine/chemokine release, SEAP level increase by activating Toll-like receptors, such as TLR7 receptor, the metabolic conversion of prodrugs to parent compounds in the presence of human hepatocytes, and the therapeutic or prophylactic use of such compounds and their pharmaceutical compositions comprising these compounds and their prodrugs to treat or prevent liver cancer. The present invention also provides compounds with superior activity. In addition, the compounds of formula (I) also show good solubility and PK profiles.

The present invention relates to novel compounds of formula (I),

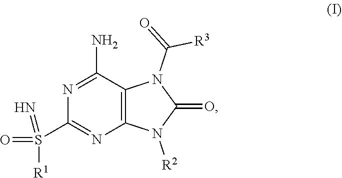

wherein
R¹ is C₁₋₆alkyl;
R² is benzyl, said benzyl being unsubstituted or substituted by one, two or three substituents independently selected from halogen and C₁₋₆alkyl;
R³ is —NR⁴R⁵, wherein
   R⁴ is C₁₋₆-alkyl or C₁₋₆alkoxyC₁₋₆alkyl;
   R⁵ is (C₁₋₆alkyl)₂NCOOC₁₋₆alkyl, C₁₋₆alkoxyC₁₋₆alkyl, C₁₋₆alkoxycarbonyl(C₁₋₆alkyl)aminoC₁₋₆ alkyl, C₁₋₆alkoxycarbonyl(phenyl)C₁₋₆alkyl, C₁₋₆alkoxycarbonylC₁₋₆alkyl, C₁₋₆ alkoxycarbonyloxyC₁₋₆alkyl, C₁₋₆alkyl, C₁₋₆alkylcarbonyl(C₁₋₆alkyl)aminoC₁₋₆alkyl or pyrrolidinylcarbamoyloxyC₁₋₆alkyl; or
   R⁴ and R⁵ together with the nitrogen they are attached to form a heterocyclyl;
      or pharmaceutically acceptable salt, enantiomer or diastereomer thereof,
      for use in the treatment or prophylaxis of liver cancer;
      with the proviso that
6-amino-9-benzyl-2-(propylsulfonimidoyl)-7-(pyrrolidine-1-carbonyl)purin-8-one;
6-amino-9-benzyl-7-(piperidine-1-carbonyl)-2-(propylsulfonimidoyl)purin-8-one;
6-amino-9-benzyl-7-(morpholine-4-carbonyl)-2-(propylsulfonimidoyl)purin-8-one;
6-amino-9-benzyl-7-(3,3-dimethylpyrrolidine-1-carbonyl)-2-(propylsulfonimidoyl)purin-8-one;
ethyl 1-[6-amino-9-benzyl-8-oxo-2-(propylsulfonimidoyl)purine-7-carbonyl]pyrrolidine-2-carboxylate;
6-amino-7-(2-azaspiro[3.3]heptane-2-carbonyl)-9-benzyl-2-(propylsulfonimidoyl)purin-8-one;
6-amino-9-benzyl-7-(2-oxa-6-azaspiro[3.3]heptane-6-carbonyl)-2-(propylsulfonimidoyl)purin-8-one;
6-amino-9-benzyl-7-(3,3-difluoropyrrolidine-1-carbonyl)-2-(propylsulfonimidoyl)purin-8-one;
6-amino-9-benzyl-7-(3-fluoro-3-methyl-pyrrolidine-1-carbonyl)-2-(propylsulfonimidoyl)purin-8-one;
   and their enantiomers or diastereomers are excluded.

These prodrug compounds are especially useful for the treatment of liver cancer as they are activated (converted into their active form) in the liver. They show valuable anti-tumor efficacy in vivo in liver cancer cell models (either alone or in combination with anti-PD1/PD1 antibodies or with anti-angiogenic agents) and in vitro against liver cancer cells (by activation of peripheral blood cells and/or factors).

The invention also relates to their use for the manufacture of a medicament for the treatment or prophylaxis of liver cancer, medicaments based on a compound in accordance with the invention for the treatment or prophylaxis of liver cancer. Accordingly, the compounds of formula (I) are useful for the treatment or prophylaxis of liver cancer, especially for the treatment or prophylaxis of hepatocellular carcinoma, hepatoma, cholangiocarcinoma, hepatoblastoma, hepatic carcinoma, hepatic angiosarcoma, or metastatic liver cancer.

### DETAILED DESCRIPTION OF THE INVENTION

Unless otherwise defined, all technical and scientific terms used herein have the same meaning as commonly understood by one of ordinary skill in the art to which this invention belongs. Furthermore, the following definitions are set forth to illustrate and define the meaning and scope of the various terms used to describe the invention.

The term “C₁₋₆alkyl” denotes a saturated, linear or branched chain alkyl group containing 1 to 6, particularly 1 to 4 carbon atoms, for example methyl, ethyl, n-propyl, isopropyl, n-butyl, isobutyl, tert-butyl and the like. Particular “C₁₋₆alkyl” groups are methyl, ethyl and n-propyl.

The term “C₁₋₆alkoxy” denotes a group of the formula C₁₋₆alkyl-O—. Examples of C₁₋₆alkoxy group include, but not limited to, methoxy, ethoxy, n-propoxy, isopropoxy, n-butoxy, isobutoxy and tert-butoxy.

Particular “C₁₋₆alkoxy” groups are methoxy, ethoxy and isopropoxy. A more particular C₁₋₆alkoxy group is ethoxy.

The term “halogen” and “halo” are used interchangeably herein and denote fluoro, chloro, bromo, or iodo.

The term “heterocyclyl” denotes a monovalent saturated or partly unsaturated mono or bicyclic ring system of 3 to 10 ring atoms, comprising 1 to 5 ring heteroatoms selected from N, O and S, the remaining ring atoms being carbon. In particular embodiments, heterocyclyl is a monovalent saturated monocyclic ring system of 4 to 7 ring atoms, comprising 1, 2, or 3 ring heteroatoms selected from N, O and S, the remaining ring atoms being carbon. Examples for monocyclic saturated heterocyclyl are aziridinyl, oxiranyl, azetidinyl, oxetanyl, pyrrolidinyl, dimethylpyrrolidinyl, ethoxycarbonylpyrrolidinyl, tetrahydrofuranyl, tetrahydro-thienyl, pyrazolidinyl, imidazolidinyl, oxazolidinyl, isoxazolidinyl, thiazolidinyl, piperidinyl, tetrahydropyranyl, tetrahydrothiopyranyl, piperazinyl, morpholinyl, thiomorpholinyl, dioxothiomorpholinyl, azepanyl, diazepanyl, homopiperazinyl, or oxazepanyl. Monocyclic saturated heterocyclyl can be further substituted by one to three substituents independently selected from halogen, C₁₋₆alkyl and C₁₋₆ alkoxycarbonyl. Examples for substituted monocyclic saturated heterocyclyl are 4-methylpiperazinyl, dimethylpyrrolidinyl, ethoxycarbonylpyrrolidinyl, difluoropyrrolidinyl, fluoro(methyl)pyrrolidinyl. Examples for bicyclic saturated heterocyclyl are azabicyclo[3.2.1]octyl, quinuclidinyl, oxaazabicyclo[3.2.1]octyl, azabicyclo[3.3.1]nonyl, oxaazabicyclo[3.3.1]nonyl, thiaazabicyclo[3.3.1]nonyl, azaspiro[3.3]heptanyl and oxaazaspiro[3.3]heptanyl. Examples for partly unsaturated heterocyclyl are dihydrofuryl, imidazolinyl, dihydrooxazolyl, tetrahydropyridinyl and dihydropyranyl.

The term “carbonyl” alone or in combination refers to the group —C(O)—.

The term “C₁₋₆alkylcarbonyl” refers to a group C₁₋₆alkyl-C(O)—, wherein the “C₁₋₆alkyl” is as defined above. Particular “C₁₋₆alkylcarbonyl” group is acetyl.

The term “enantiomer” denotes two stereoisomers of a compound which are non-superimposable mirror images of one another.

The term “diastereomer” denotes a stereoisomer with two or more centers of chirality and whose molecules are not mirror images of one another. Diastereomers have different physical properties, e.g. melting points, boiling points, spectral properties, and reactivities.

The term “pharmaceutically acceptable salts” denotes salts which are not biologically or otherwise undesirable. Pharmaceutically acceptable salts include both acid and base addition salts.

The term “pharmaceutically acceptable acid addition salt” denotes those pharmaceutically acceptable salts formed with inorganic acids such as hydrochloric acid, hydrobromic acid, sulfuric acid, nitric acid, carbonic acid, phosphoric acid, and organic acids selected from aliphatic, cycloaliphatic, aromatic, araliphatic, heterocyclic, carboxylic, and sulfonic classes of organic acids such as formic acid, acetic acid, propionic acid, glycolic acid, gluconic acid, lactic acid, pyruvic acid, oxalic acid, malic acid, maleic acid, maloneic acid, succinic acid, fumaric acid, tartaric acid, citric acid, aspartic acid, ascorbic acid, glutamic acid, anthranilic acid, benzoic acid, cinnamic acid, mandelic acid, embonic acid, phenylacetic acid, methanesulfonic acid, ethanesulfonic acid, p-toluenesulfonic acid, and salicyclic acid.

The term “pharmaceutically acceptable base addition salt” denotes those pharmaceutically acceptable salts formed with an organic or inorganic base. Examples of acceptable inorganic bases include sodium, potassium, ammonium, calcium, magnesium, iron, zinc, copper, manganese, and aluminum salts. Salts derived from pharmaceutically acceptable organic nontoxic bases includes salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-diethylaminoethanol, trimethamine, dicyclohexylamine, lysine, arginine, histidine, caffeine, procaine, hydrabamine, choline, betaine, ethylenediamine, glucosamine, methylglucamine, theobromine, purines, piperizine, piperidine, N-ethylpiperidine, and polyamine resins.

Compounds of the general formula (I) and their prodrugs which contain one or several chiral centers can either be present as racemates, diastereomeric mixtures, or optically active single isomers. The racemates can be separated according to known methods into the enantiomers. Particularly, diastereomeric salts which can be separated by crystallization are formed from the racemic mixtures by reaction with an optically active acid such as e.g. D- or L-tartaric acid, mandelic acid, malic acid, lactic acid or camphorsulfonic acid.

The term “prodrug” denotes a form or derivative of a compound which is metabolized in vivo, e.g., by biological fluids or enzymes by a subject after administration, into a pharmacologically active form of the compound in order to produce the desired pharmacological effect. Prodrugs are described e.g. in “The Organic Chemistry of Drug Design and Drug Action”, by Richard B. Silverman, Academic Press, San Diego, 2004, Chapter 8 Prodrugs and Drug Delivery Systems, pp. 497-558.

“A pharmaceutically active metabolite” is intended to mean a pharmacologically active product produced through metabolism in the body of a specified compound or salt thereof. After entry into the body, most drugs are substrates for chemical reactions that may change their physical properties and biologic effects. These metabolic conversions, which usually affect the polarity of the compounds of the invention, alter the way in which drugs are distributed in and excreted from the body. However, in some cases, metabolism of a drug is required for therapeutic effect.

The term “therapeutically effective amount” denotes an amount of a compound or molecule of the present invention that, when administered to a subject, (i) treats or prevents the particular disease, condition or disorder, (ii) attenuates, ameliorates or eliminates one or more symptoms of the particular disease, condition, or disorder, or (iii) prevents or delays the onset of one or more symptoms of the particular disease, condition or disorder described herein. The therapeutically effective amount will vary depending on the compound, the disease state being treated, the severity of the disease treated, the age and relative health of the subject, the route and form of administration, the judgement of the attending medical or veterinary practitioner, and other factors.

The term “pharmaceutical composition” denotes a mixture or solution comprising a therapeutically effective amount of an active pharmaceutical ingredient together with pharmaceutically acceptable excipients to be administered to a mammal, e.g., a human in need thereof.

### TLR7 Agonist and Prodrug

The present invention relates to a compound of formula (I),

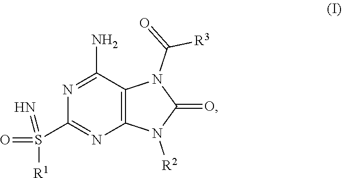

wherein
R¹ is C₁₋₆alkyl;
R² is benzyl, said benzyl being unsubstituted or substituted by one, two or three substituents independently selected from halogen and C₁₋₆alkyl;
R³ is —NR⁴R⁵, wherein
   R⁴ is C₁₋₆alkyl or C₁₋₆alkoxyC₁₋₆alkyl;
   R⁵ is (C₁₋₆alkyl)₂NCOOC₁₋₆alkyl, C₁₋₆alkoxyC₁₋₆alkyl, C₁₋₆alkoxycarbonyl(C₁₋₆alkyl)aminoC₁₋₆ alkyl, C₁₋₆alkoxycarbonyl(phenyl)C₁₋₆alkyl, C₁₋₆alkoxycarbonylC₁₋₆alkyl, C₁₋₆ alkoxycarbonyloxyC₁₋₆alkyl, C₁₋₆alkyl, C₁₋₆alkylcarbonyl(C₁₋₆alkyl)aminoC₁₋₆alkyl or pyrrolidinylcarbamoyloxyC₁₋₆alkyl; or
   R⁴ and R⁵ together with the nitrogen they are attached to form a heterocyclyl;
      or pharmaceutically acceptable salt, enantiomer or diastereomer thereof;
      for use in the treatment or prophylaxis of liver cancer;
      with the proviso that
6-amino-9-benzyl-2-(propylsulfonimidoyl)-7-(pyrrolidine-1-carbonyl)purin-8-one;
6-amino-9-benzyl-7-(piperidine-1-carbonyl)-2-(propylsulfonimidoyl)purin-8-one;
6-amino-9-benzyl-7-(morpholine-4-carbonyl)-2-(propylsulfonimidoyl)purin-8-one;
6-amino-9-benzyl-7-(3,3-dimethylpyrrolidine-1-carbonyl)-2-(propylsulfonimidoyl)purin-8-one;
ethyl 1-[6-amino-9-benzyl-8-oxo-2-(propylsulfonimidoyl)purine-7-carbonyl]pyrrolidine-2-carboxylate;
6-amino-7-(2-azaspiro[3.3]heptane-2-carbonyl)-9-benzyl-2-(propylsulfonimidoyl)purin-8-one;
6-amino-9-benzyl-7-(2-oxa-6-azaspiro[3.3]heptane-6-carbonyl)-2-(propylsulfonimidoyl)purin-8-one;
6-amino-9-benzyl-7-(3,3-difluoropyrrolidine-1-carbonyl)-2-(propylsulfonimidoyl)purin-8-one;
6-amino-9-benzyl-7-(3-fluoro-3-methyl-pyrrolidine-1-carbonyl)-2-(propylsulfonimidoyl)purin-8-one;
   and their enantiomers or diastereomers are excluded.

A further embodiment of present invention is (ii) a compound of formula (I), wherein
R¹ is C₁₋₆alkyl;
R² is benzyl, said benzyl being unsubstituted or substituted by halogen or C₁₋₆alkyl;
R³ is azetidinyl;
   piperazinyl substituted by C₁₋₆alkyl;
   piperidinyl substituted by piperidinyl;
   pyrrolidinyl; or
   —NR⁴R⁵, wherein
      R⁴ is C₁₋₆alkyl or C₁₋₆alkoxyC₁₋₆alkyl;
      R⁵ is (C₁₋₆alkyl)₂NCOOC₁₋₆alkyl, C₁₋₆alkoxyC₁₋₆alkyl, C₁₋₆alkoxycarbonyl(C₁₋₆alkyl)aminoC₁₋₆ alkyl, C₁₋₆alkoxycarbonyl(phenyl)C₁₋₆alkyl, C₁₋₆alkoxycarbonylC₁₋₆alkyl, C₁₋₆ alkoxycarbonyloxyC₁₋₆alkyl, C₁₋₆alkyl, C₁₋₆alkylcarbonyl(C₁₋₆alkyl)aminoC₁₋₆alkyl or pyrrolidinylcarbamoyloxyC₁₋₆alkyl;
         or pharmaceutically acceptable salt, enantiomer or diastereomer thereof, for use in the treatment or prophylaxis of liver cancer.

A further embodiment of present invention is (iii) a compound of formula (I), wherein
R¹ is ethyl or propyl;
R² is benzyl, bromobenzyl, chlorobenzyl, fluorobenzyl or methylbenzyl;
R³ is azetidinyl;
   4-methylpiperazinyl;
   piperidinylpiperidinyl;
   pyrrolidinyl; or
   —NR⁴R⁵, wherein
      R⁴ is methyl, ethyl, propyl or methoxyethyl;
      R⁵ is acetyl(methyl)aminoethyl, butyl, butyl(methyl)carbamoyloxyethyl, diethylcarbamoyloxyethyl, ethoxycarbonyl(methyl)aminoethyl, ethoxycarbonylethyl, ethoxycarbonylisobutyl, ethoxycarbonylisopentyl, ethoxycarbonylmethyl, ethoxycarbonyloxyethyl, ethoxycarbonyl(phenyl)ethyl, ethyl, isobutyl, isopropoxycarbonylisopentyl, isopropoxycarbonyl(phenyl)ethyl, isopropyl, methoxycarbonyl(methyl)aminoethyl, methoxyethyl, methoxypropyl, propyl, propyl(methyl)carbamoyloxyethyl, pyrrolidinylcarbamoyloxyethyl, tert-butoxycarbonyl(methyl)aminoethyl, tert-butoxycarbonylethyl, tert-butoxycarbonylisopentyl or tert-butoxycarbonyl(phenyl)ethyl;
or pharmaceutically acceptable salt, enantiomer or diastereomer thereof, for use in the treatment or prophylaxis of liver cancer.

A further embodiment of present invention is (iii-1) a compound of formula (I), wherein
R¹ is ethyl or propyl;
R² is benzyl, chlorobenzyl, fluorobenzyl or methylbenzyl;
R³ is azetidinyl;
   4-methylpiperazinyl;
   piperidinylpiperidinyl;
   pyrrolidinyl; or
   —NR⁴R⁵, wherein
      R⁴ is methyl, ethyl, propyl or methoxyethyl;
      R⁵ is acetyl(methyl)aminoethyl, butyl, butyl(methyl)carbamoyloxyethyl, diethylcarbamoyloxyethyl, ethoxycarbonyl(methyl)aminoethyl, ethoxycarbonylethyl, ethoxycarbonylisobutyl, ethoxycarbonylisopentyl, ethoxycarbonylmethyl, ethoxycarbonyloxyethyl, ethoxycarbonyl(phenyl)ethyl, ethyl, isobutyl, isopropoxycarbonylisopentyl, isopropoxycarbonyl(phenyl)ethyl, isopropyl, methoxycarbonyl(methyl)aminoethyl, methoxyethyl, methoxypropyl, propyl, propyl(methyl)carbamoyloxyethyl, pyrrolidinylcarbamoyloxyethyl, tert-butoxycarbonyl(methyl)aminoethyl, tert-butoxycarbonylethyl, tert-butoxycarbonylisopentyl or tert-butoxycarbonyl(phenyl)ethyl;
or pharmaceutically acceptable salt, enantiomer or diastereomer thereof, for use in the treatment or prophylaxis of liver cancer.

A further embodiment of present invention is (iv) a compound of formula (I), wherein R³ is azetidinyl, 4-methylpiperazinyl, piperidinylpiperidinyl, pyrrolidinyl, acetyl(methyl)aminoethyl(methyl)amino, bis(methoxyethyl)amino, butyl(ethyl)amino, butyl(methyl)amino, butyl(methyl)carbamoyloxyethyl(methyl)amino, diethylcarbamoyloxyethyl(methyl)amino, ethoxycarbonyl(methyl)aminoethyl(methyl)amino, ethoxycarbonylethyl(methyl)amino, ethoxycarbonylisobutyl(methyl)amino, ethoxycarbonylisopentyl(methyl)amino, ethoxycarbonylmethyl(methyl)amino, ethoxycarbonyloxyethyl(methyl)amino, ethoxycarbonyl(phenyl)ethyl(methyl)amino, ethyl(methyl)amino, isobutyl(methyl)amino, isopropoxycarbonylisopentyl(methyl)amino, isopropoxycarbonyl(phenyl)ethyl(methyl)amino, isopropyl(methyl)amino, methoxycarbonyl(methyl)aminoethyl(methyl)amino, methoxyethyl(ethyl)amino, methoxyethyl(methyl)amino, methoxyethyl(propyl)amino, methoxypropyl(methyl)amino, propyl(ethyl)amino, propyl(methyl)amino, propyl(methyl)carbamoyloxyethyl(methyl)amino, pyrrolidinylcarbamoyloxyethyl(methyl)amino, tert-butoxycarbonyl(methyl)aminoethyl(methyl)amino, tert-butoxycarbonylethyl(methyl)amino, tert-butoxycarbonylisopentyl(methyl)amino or tert-butoxycarbonyl(phenyl)ethyl(methyl)amino; or pharmaceutically acceptable salt, enantiomer or diastereomer thereof, for use in the treatment or prophylaxis of liver cancer.

A further embodiment of present invention is (v) a compound of formula (I), wherein R¹ is ethyl, for use in the treatment or prophylaxis of liver cancer.

A further embodiment of present invention is (vi) a compound of formula (I), wherein R² is benzyl substituted by halogen or C₁₋₆alkyl, for use in the treatment or prophylaxis of liver cancer.

A further embodiment of present invention is (vii) a compound of formula (I), wherein R² is bromobenzyl, chlorobenzyl, fluorobenzyl or methylbenzyl, for use in the treatment or prophylaxis of liver cancer.

A further embodiment of present invention is (vii-1) a compound of formula (I), wherein R² is chlorobenzyl, fluorobenzyl or methylbenzyl, for use in the treatment or prophylaxis of liver cancer.

A further embodiment of present invention is (viii) a compound of formula (I), wherein R² is bromobenzyl, chlorobenzyl or fluorobenzyl, for use in the treatment or prophylaxis of liver cancer.

A further embodiment of present invention is (viii-1) a compound of formula (I), wherein R² is chlorobenzyl or fluorobenzyl, for use in the treatment or prophylaxis of liver cancer.

A further embodiment of present invention is (ix) a compound of formula (I), wherein R³ is —NR⁴R⁵, wherein R⁴ is C₁₋₆-alkyl, R⁵ is C₁₋₆alkyl, for use in the treatment or prophylaxis of liver cancer.

A further embodiment of present invention is (x) a compound of formula (I), wherein R³ is propyl(methyl)amino or ethyl(methyl)amino, for use in the treatment or prophylaxis of liver cancer.

A further embodiment of present invention is (xi) a compound of formula (I), wherein
R¹ is C₁₋₆alkyl;
R² is benzyl, said benzyl being substituted by halogen or C₁₋₆alkyl;
R³ is —NR⁴R⁵, wherein R⁴ is C₁₋₆alkyl, R⁵ is C₁₋₆alkyl;
or pharmaceutically acceptable salt, enantiomer or diastereomer thereof, for use in the treatment or prophylaxis of liver cancer.

A further embodiment of present invention is (xii) a compound of formula (I), wherein
R¹ is ethyl;
R² is methylbenzyl, bromobenzyl, chlorobenzyl or fluorobenzyl;
R³ is propyl(methyl)amino or ethyl(methyl)amino;
or pharmaceutically acceptable salt, enantiomer or diastereomer thereof, for use in the treatment or prophylaxis of liver cancer.

A further embodiment of present invention is (xii-1) a compound of formula (I), wherein
R¹ is ethyl;
R² is methylbenzyl, chlorobenzyl or fluorobenzyl;
R³ is propyl(methyl)amino or ethyl(methyl)amino;
   or pharmaceutically acceptable salt, enantiomer or diastereomer thereof, for use in the treatment or prophylaxis of liver cancer.

Another embodiment of present invention is that (xiii) particular compounds of formula (I) are the following:
6-Amino-9-benzyl-N-methyl-8-oxo-N-propyl-2-(propylsulfonimidoyl)purine-7-carboxamide;
6-Amino-9-benzyl-N-(2-methoxyethyl)-N-methyl-8-oxo-2-(propylsulfonimidoyl)purine-7-carboxamide;
6-Amino-9-benzyl-N-ethyl-8-oxo-N-propyl-2-(propylsulfonimidoyl)purine-7-carboxamide;
6-Amino-9-benzyl-7-[4-(1-piperidyl)piperidine-1-carbonyl]-2-(propylsulfonimidoyl)purin-8-one;
6-Amino-9-benzyl-N-ethyl-N-(2-methoxyethyl)-8-oxo-2-(propylsulfonimidoyl)purine-7-carboxamide;
6-Amino-9-benzyl-N-butyl-N-ethyl-8-oxo-2-(propylsulfonimidoyl)purine-7-carboxamide;
6-Amino-9-benzyl-N-(2-methoxyethyl)-8-oxo-N-propyl-2-(propylsulfonimidoyl)purine-7-carboxamide;
6-Amino-9-benzyl-N,N-bis(2-methoxyethyl)-8-oxo-2-(propylsulfonimidoyl)purine-7-carboxamide;
6-Amino-7-(azetidine-1-carbonyl)-9-benzyl-2-(propylsulfonimidoyl)purin-8-one;
6-Amino-9-benzyl-N-isopropyl-N-methyl-8-oxo-2-(propylsulfonimidoyl)purine-7-carboxamide;
6-Amino-9-benzyl-7-(4-methylpiperazine-1-carbonyl)-2-(propylsulfonimidoyl)purin-8-one;
6-Amino-9-benzyl-N-(3-methoxypropyl)-N-methyl-8-oxo-2-(propylsulfonimidoyl)purine-7-carboxamide;
6-Amino-9-benzyl-N-isobutyl-N-methyl-8-oxo-2-(propylsulfonimidoyl)purine-7-carboxamide;
Ethyl 2-[[6-amino-9-benzyl-8-oxo-2-(propylsulfonimidoyl)purine-7-carbonyl]-methyl-amino]acetate;
Ethyl 3-[[6-amino-9-benzyl-8-oxo-2-(propylsulfonimidoyl)purine-7-carbonyl]-methyl-amino]propanoate;
tert-Butyl 3-[[6-amino-9-benzyl-8-oxo-2-(propylsulfonimidoyl)purine-7-carbonyl]-methyl-amino]propanoate;
Ethyl (2S)-2-[[6-amino-9-benzyl-8-oxo-2-(propsunimidylsulfonimidoyl)purine-7-carbonyl]-methyl-amino]propanoate;
tert-Butyl (2S)-2-[[6-amino-9-benzyl-8-oxo-2-(propylsulfonimidoyl)purine-7-carbonyl]-methyl-amino]-4-methyl-pentanoate;
Isopropyl (2S)-2-[[6-amino-9-benzyl-8-oxo-2-(propylsulfonimidoyl)purine-7-carbonyl]-methyl-amino]-4-methyl-pentanoate;
Ethyl (2S)-2-[[6-amino-9-benzyl-8-oxo-2-(propylsulfonimidoyl)purine-7-carbonyl]-methyl-amino]-3-methyl-butanoate;
Ethyl (2S)-2-[[6-amino-9-benzyl-8-oxo-2-(propylsulfonimidoyl)purine-7-carbonyl]-methyl-amino]-4-methyl-pentanoate;
Ethyl (2S)-2-[[6-amino-9-benzyl-8-oxo-2-(propylsulfonimidoyl)purine-7-carbonyl]-methyl-amino]-3-phenyl-propanoate;
Isopropyl (2S)-2-[[6-amino-9-benzyl-8-oxo-2-(propylsulfonimidoyl)purine-7-carbonyl]-methyl-amino]-3-phenyl-propanoate;
tert-Butyl (2S)-2-[[6-amino-9-benzyl-8-oxo-2-(propylsulfonimidoyl)purine-7-carbonyl]-methyl-amino]-3-phenyl-propanoate;
N-[2-[Acetyl(methyl)amino]ethyl]-6-amino-9-benzyl-N-methyl-8-oxo-2-(propylsulfonimidoyl)purine-7-carboxamide;
Methyl N-[2-[[6-amino-9-benzyl-8-oxo-2-(propylsulfonimidoyl)purine-7-carbonyl]-methyl-amino]ethyl]-N-methyl-carbamate;
tert-Butyl N-[2-[[6-amino-9-benzyl-8-oxo-2-(propylsulfonimidoyl)purine-7-carbonyl]-methyl-amino]ethyl]-N-methyl-carbamate;
Ethyl N-[2-[[6-amino-9-benzyl-8-oxo-2-(propylsulfonimidoyl)purine-7-carbonyl]-methyl-amino]ethyl]-N-methyl-carbamate;
2-[[6-Amino-9-benzyl-8-oxo-2-(propylsulfonimidoyl)purine-7-carbonyl]-methyl-amino]ethyl N-butyl-N-methyl-carbamate;
2-[[6-Amino-9-benzyl-8-oxo-2-(propylsulfonimidoyl)purine-7-carbonyl]-methyl-amino]ethyl pyrrolidine-1-carboxylate;
2-[[6-Amino-9-benzyl-8-oxo-2-(propylsulfonimidoyl)purine-7-carbonyl]-methyl-amino]ethyl N-methyl-N-propyl-carbamate;
2-[[6-Amino-9-benzyl-8-oxo-2-(propylsulfonimidoyl)purine-7-carbonyl]-methyl-amino]ethyl N,N-diethylcarbamate;
2-[[6-Amino-9-benzyl-8-oxo-2-(propylsulfonimidoyl)purine-7-carbonyl]-methyl-amino]ethyl ethyl carbonate;
6-Amino-N-butyl-9-[(4-chlorophenyl)methyl]-N-methyl-8-oxo-2-[S(S)-propylsulfonimidoyl]purine-7-carboxamide;
6-amino-N-butyl-9-[(4-chlorophenyl)methyl]-N-methyl-8-oxo-2-[S(S)-propylsulfonimidoyl]purine-7-carboxamide;
6-Amino-9-[(4-chlorophenyl)methyl]-N-ethyl-N-methyl-8-oxo-2-(propylsulfonimidoyl)purine-7-carboxamide;
6-Amino-N-methyl-8-oxo-N-propyl-2[S(S)-propylsulfonimidoyl]-9-(p-tolylmethyl)purine-7-carboxamide;
6-Amino-N-methyl-8-oxo-N-propyl-2[S(R)-propylsulfonimidoyl]-9-(p-tolylmethyl)purine-7-carboxamide;
6-Amino-2-[S(S)-propylsulfonimidoyl]-9-(p-tolylmethyl)-7-(pyrrolidine-1-carbonyl)purin-8-one;
6-Amino-2-[S(R)-propylsulfonimidoyl]-9-(p-tolylmethyl)-7-(pyrrolidine-1-carbonyl)purin-8-one;
6-Amino-N-(2-methoxyethyl)-N-methyl-8-oxo-2-[S(S)-propylsulfonimidoyl]-9-(p-tolylmethyl)purine-7-carboxamide;
6-Amino-N-(2-methoxyethyl)-N-methyl-8-oxo-2-[S(R)-propylsulfonimidoyl]-9-(p-tolylmethyl)purine-7-carboxamide;
6-Amino-N-ethyl-N-methyl-8-oxo-2-(propylsulfonimidoyl)-9-(p-tolylmethyl)purine-7-carboxamide;
6-Amino-N-butyl-N-methyl-8-oxo-2-(propylsulfonimidoyl)-9-(p-tolylmethyl)purine-7-carboxamide;
6-Amino-9-[(4-chlorophenyl)methyl]-2-[S(R)-ethylsulfonimidoyl]-N-methyl-8-oxo-N-propyl-purine-7-carboxamide;
6-Amino-9-[(4-chlorophenyl)methyl]-2-[S(S)-ethylsulfonimidoyl]-N-methyl-8-oxo-N-propyl-purine-7-carboxamide;
6-Amino-9-[(4-chlorophenyl)methyl]-N-ethyl-2 [S(S)-ethylsulfonimidoyl]-N-methyl-8-oxo-purine-7-carboxamide;
6-Amino-9-[(4-chlorophenyl)methyl]-N-ethyl-2-[S(R)-ethylsulfonimidoyl]-N-methyl-8-oxo-purine-7-carboxamide;
6-Amino-2-[S(S)-ethylsulfonimidoyl]-N-methyl-8-oxo-N-propyl-9-(p-tolylmethyl)purine-7-carboxamide;
6-Amino-2-[S(R)-ethylsulfonimidoyl]-N-methyl-8-oxo-N-propyl-9-(p-tolylmethyl)purine-7-carboxamide;
6-Amino-N-ethyl-2[S(S)-ethylsulfonimidoyl]-N-methyl-8-oxo-9-(p-tolylmethyl)purine-7-carboxamide;
6-Amino-N-ethyl-2-[S(R)-ethylsulfonimidoyl]-N-methyl-8-oxo-9-(p-tolylmethyl)purine-7-carboxamide;
6-Amino-2-[S(S)ethylsulfonimidoyl]-9-[(4-fluorophenyl)methyl]-N-methyl-8-oxo-N-propyl-purine-7-carboxamide;
6-Amino-2-[S(R)ethylsulfonimidoyl]-9-[(4-fluorophenyl)methyl]-N-methyl-8-oxo-N-propyl-purine-7-carboxamide;
6-Amino-N-ethyl-2-(ethylsulfonimidoyl)-9-[(4-fluorophenyl)methyl]-N-methyl-8-oxo-purine-7-carboxamide;
6-Amino-N-ethyl-2-[S(S)-(ethylsulfonimidoyl)]-9-[(4-fluorophenyl)methyl]-N-methyl-8-oxo-purine-7-carboxamide;
6-Amino-N-ethyl-2-[S(R)-(ethylsulfonimidoyl)]-9-[(4-fluorophenyl)methyl]-N-methyl-8-oxo-purine-7-carboxamide;
6-Amino-9-[(4-bromophenyl)methyl]-2-(ethylsulfonimidoyl)-N-methyl-8-oxo-N-propyl-purine-7-carboxamide;
6-Amino-2-[S(R)-ethylsulfonimidoyl]-9-[(4-bromophenyl)methyl]-N-methyl-8-oxo-N-propyl-purine-7-carboxamide;
6-Amino-2-[S(S)-ethylsulfonimidoyl]-9-[(4-bromophenyl)methyl]-N-methyl-8-oxo-N-propyl-purine-7-carboxamide;
6-Amino-9-[(4-bromophenyl)methyl]-N-ethyl-2-(ethylsulfonimidoyl)-N-methyl-8-oxo-purine-7-carboxamide;
6-Amino-9-[(4-bromophenyl)methyl]-N-ethyl-2-[S(S)-(ethylsulfonimidoyl)]-N-methyl-8-oxo-purine-7-carboxamide; and
6-Amino-9-[(4-bromophenyl)methyl]-N-ethyl-2-[S(R)-(ethylsulfonimidoyl)]-N-methyl-8-oxo-purine-7-carboxamide;
   or pharmaceutically acceptable salt, enantiomer or diastereomer thereof, for use in the treatment or prophylaxis of liver cancer.

Another embodiment of present invention is that (xiv) more particular compounds of formula (I) are the following:
6-Amino-9-benzyl-N-methyl-8-oxo-N-propyl-2-(propylsulfonimidoyl)purine-7-carboxamide;
6-Amino-9-[(4-chlorophenyl)methyl]-2-[S(R)-ethylsulfonimidoyl]-N-methyl-8-oxo-N-propyl-purine-7-carboxamide;
6-Amino-9-[(4-chlorophenyl)methyl]-2-[S(S)-ethylsulfonimidoyl]-N-methyl-8-oxo-N-propyl-purine-7-carboxamide;
6-Amino-9-[(4-chlorophenyl)methyl]-N-ethyl-2 [S(S)-ethylsulfonimidoyl]-N-methyl-8-oxo-purine-7-carboxamide;
6-Amino-9-[(4-chlorophenyl)methyl]-N-ethyl-2-[S(R)-ethylsulfonimidoyl]-N-methyl-8-oxo-purine-7-carboxamide;
6-Amino-2-[S(S)-ethylsulfonimidoyl]-N-methyl-8-oxo-N-propyl-9-(p-tolylmethyl)purine-7-carboxamide;
6-Amino-2-[S(R)-ethylsulfonimidoyl]-N-methyl-8-oxo-N-propyl-9-(p-tolylmethyl)purine-7-carboxamide;
6-Amino-N-ethyl-2[S(S)-ethylsulfonimidoyl]-N-methyl-8-oxo-9-(p-tolylmethyl)purine-7-carboxamide;
6-Amino-N-ethyl-2-[S(R)-ethylsulfonimidoyl]-N-methyl-8-oxo-9-(p-tolylmethyl)purine-7-carboxamide;
6-amino-2-(ethylsulfonimidoyl)-9-[(4-fluorophenyl)methyl]-N-methyl-8-oxo-N-propyl-purine-7-carboxamide;
6-Amino-2-[S(S)ethylsulfonimidoyl]-9-[(4-fluorophenyl)methyl]-N-methyl-8-oxo-N-propyl-purine-7-carboxamide;
6-Amino-2-[S(R)ethylsulfonimidoyl]-9-[(4-fluorophenyl)methyl]-N-methyl-8-oxo-N-propyl-purine-7-carboxamide;
6-Amino-N-ethyl-2-(ethylsulfonimidoyl)-9-[(4-fluorophenyl)methyl]-N-methyl-8-oxo-purine-7-carboxamide;
6-Amino-N-ethyl-2-[S(S)-(ethylsulfonimidoyl)]-9-[(4-fluorophenyl)methyl]-N-methyl-8-oxo-purine-7-carboxamide;
6-Amino-N-ethyl-2-[S(R)-(ethylsulfonimidoyl)]-9-[(4-fluorophenyl)methyl]-N-methyl-8-oxo-purine-7-carboxamide;
6-Amino-9-[(4-bromophenyl)methyl]-2-(ethylsulfonimidoyl)-N-methyl-8-oxo-N-propyl-purine-7-carboxamide;
6-Amino-2-[S(R)-ethylsulfonimidoyl]-9-[(4-bromophenyl)methyl]-N-methyl-8-oxo-N-propyl-purine-7-carboxamide;
6-Amino-2-[S(S)-ethylsulfonimidoyl]-9-[(4-bromophenyl)methyl]-N-methyl-8-oxo-N-propyl-purine-7-carboxamide;
6-Amino-9-[(4-bromophenyl)methyl]-N-ethyl-2-(ethylsulfonimidoyl)-N-methyl-8-oxo-purine-7-carboxamide;
6-Amino-9-[(4-bromophenyl)methyl]-N-ethyl-2-[S(S)-(ethylsulfonimidoyl)]-N-methyl-8-oxo-purine-7-carboxamide; and
6-Amino-9-[(4-bromophenyl)methyl]-N-ethyl-2-[S(R)-(ethylsulfonimidoyl)]-N-methyl-8-oxo-purine-7-carboxamide;
   or pharmaceutically acceptable salt, enantiomer or diastereomer thereof, for use in the treatment or prophylaxis of liver cancer.

Another embodiment of present invention is that (xv) more particular compounds of formula (I) are the following:
6-Amino-9-[(4-chlorophenyl)methyl]-N-ethyl-2 [S(S)-ethylsulfonimidoyl]-N-methyl-8-oxo-purine-7-carboxamide;
6-Amino-9-[(4-chlorophenyl)methyl]-N-ethyl-2-[S(R)-ethylsulfonimidoyl]-N-methyl-8-oxo-purine-7-carboxamide;
6-Amino-9-[(4-bromophenyl)methyl]-N-ethyl-2-(ethylsulfonimidoyl)-N-methyl-8-oxo-purine-7-carboxamide;
6-Amino-9-[(4-bromophenyl)methyl]-N-ethyl-2-[S(S)-(ethylsulfonimidoyl)]-N-methyl-8-oxo-purine-7-carboxamide; and
6-Amino-9-[(4-bromophenyl)methyl]-N-ethyl-2-[S(R)-(ethylsulfonimidoyl)]-N-methyl-8-oxo-purine-7-carboxamide;
   or pharmaceutically acceptable salt, enantiomer or diastereomer thereof, for use in the treatment or prophylaxis of liver cancer.

In some embodiments, compounds of present invention were tested and compared with the following reference compounds. As the most successful biopharmaceutical companies focusing on discovery and development of TLR7 agonists for treating liver diseases, Gilead has the most advanced TLR7 agonist pipeline with leading compounds such as GS-9620 which has entered into Phase II studies. Gilead compound GS-9620 disclosed in US20100143301 as example 49, compound S-2 and compound S-3 disclosed in JP1999193282 were all chosen as the reference compounds in this application:

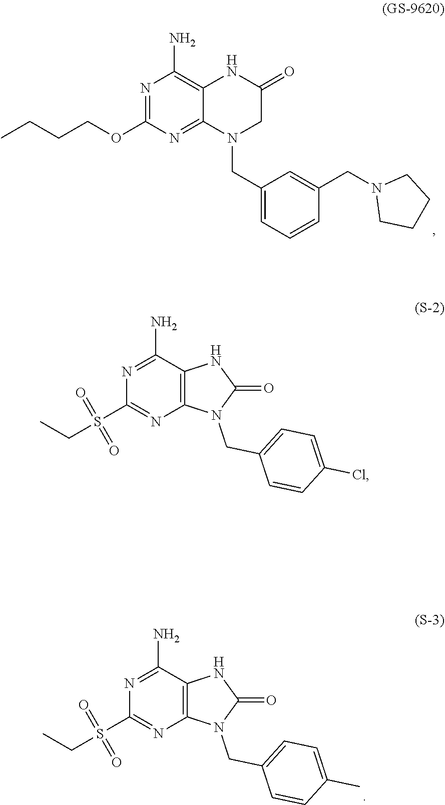 [SMILES: CCCCOc1nc(N)c2c(n1)N(Cc1cccc(CN3CCCC3)c1)CC(=O)N2 ; CCS(=O)(=O)c1nc(N)c2[nH]c(=O)n(Cc3ccc(Cl)cc3)c2n1 ; CCS(=O)(=O)c1nc(N)c2[nH]c(=O)n(Cc3ccc(C)cc3)c2n1]

### Synthesis

The compounds of the present invention can be prepared by any conventional means. Suitable processes for synthesizing these compounds as well as their starting materials are provided in the schemes below and in the examples. All substituents, in particular, R¹ to R¹⁴ are as defined above unless otherwise indicated. Furthermore, and unless explicitly otherwise stated, all reactions, reaction conditions, abbreviations and symbols have the meanings well known to a person of ordinary skill in organic chemistry.

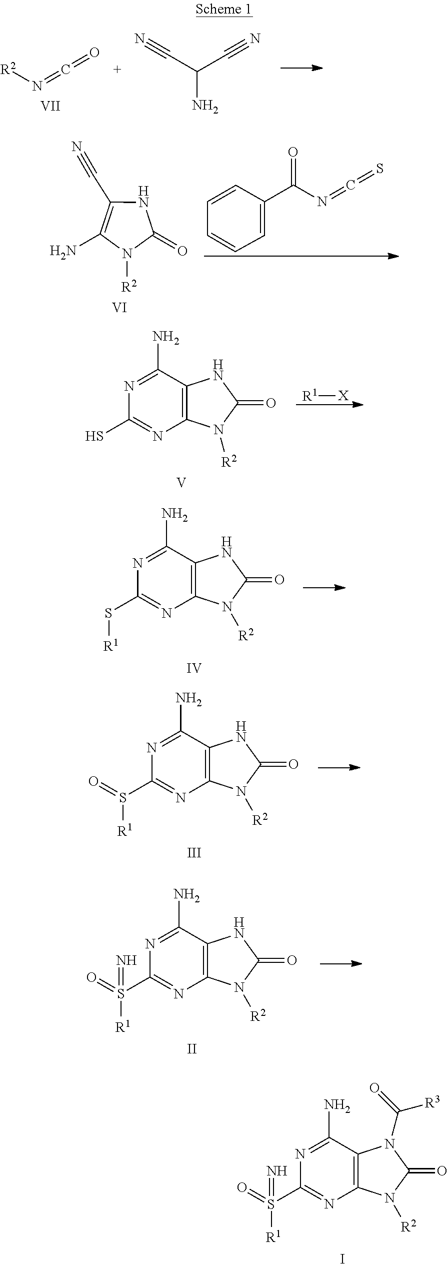

A compound of formula VI is prepared by cyclization of isocyanate VII with aminomalononitrile p-toluenesulfonate. Then bicycle V is synthesized by reaction of compound of formula VI with benzoyl isothiocyanate in the presence of inorganic base, such as NaOH or KOH. Alkylation of bicycle V with alkylhalide in the presence of base, such as K₂CO₃, NaH or Cs₂CO₃, gives compound of formula IV. Compound of formula III is prepared by oxidation of compound of formula IV with an oxidant, such as meta-chloroperoxybenzoic acid, urea-hydrogen peroxide adduct and HIO₄. Compound of formula II is obtained by imination of compound of formula III with imination reagent, such as sodium azide in acid, said acid is, for example, Eaton's reagent or PPA. Compound of formula I is obtained by reaction of compound of formula II with carbamoyl chloride in the presence of a mixed base such as pyridine and triethylamine, pyridine and DIPEA, DMAP and triethylamine, or DMAP and DIPEA.

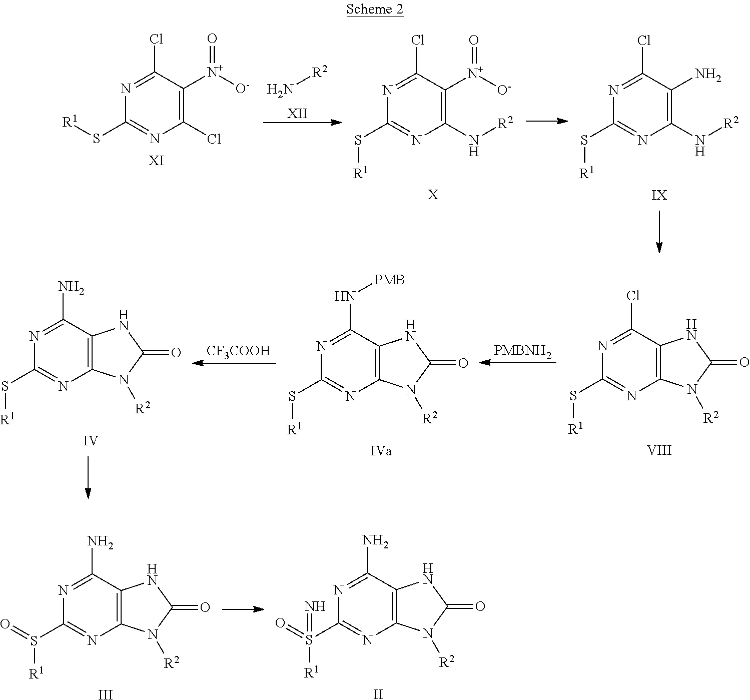

Compound of formula II can also be prepared as Scheme 2.

A compound of formula X is prepared by reaction of compound of formula XI with R²NH₂. Reduction of compound X with reducing reagent, such as Zinc or Iron powder in AcOH, gives the compound of formula IX. Cyclization of compound of formula IX with cyclization reagents, such as phosgene, carbonyl diimidazole, diethyl carbonate and triphosgene, affords compound of formula VIII. A compound of formula IVa is prepared by treating the compound of formula VIII with PMBNH₂. A compound of formula III is prepared by deprotection of compound of formula IVa with acid, such as CF₃COOH, followed by oxidation with an oxidant, such as meta-chloroperoxybenzoic acid, urea-hydrogen peroxide adduct and HIO₄. Compound of formula II is obtained by the imination of compound of formula III with imination reagent, such as sodium azide in acid, said acid is for example Eaton's reagent or PPA.

Also described is a process for the preparation of a compound of formula (I) comprising the reaction of:

the reaction of a compound of formula (II),

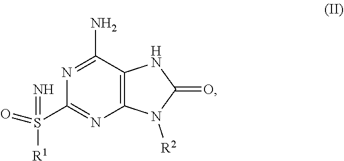

with carbamoyl chloride in the presence of a mixed base;

wherein R¹ and R² are defined above.

In above step, the mixed base can be, for example, pyridine and triethylamine, pyridine and DIPEA, DMAP and triethylamine, or DMAP and DIPEA.

A compound of formula (I) when manufactured according to the above process, for use in the treatment or prophylaxis of liver cancer is also an object of the invention.

### Pharmaceutical Compositions and Administration

Another embodiment provides pharmaceutical compositions or medicaments, for use in the treatment or prophylaxis of liver cancer containing the compounds of the invention and a therapeutically inert carrier, diluent or excipient, as well as methods of using the compounds of the invention to prepare such compositions and medicaments. In one example, compounds of formula (I) may be formulated by mixing at ambient temperature at the appropriate pH, and at the desired degree of purity, with physiologically acceptable carriers, i.e., carriers that are non-toxic to recipients at the dosages and concentrations employed into a galenical administration form. The pH of the formulation depends mainly on the particular use and the concentration of compound, but preferably ranges anywhere from about 3 to about 8. In one example, a compound of formula (I) are formulated in an acetate buffer, at pH 5. In another embodiment, the compounds of formula (I) are sterile. The compound may be stored, for example, as a solid or amorphous composition, as a lyophilized formulation or as an aqueous solution.

Compositions are formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “effective amount” of the compound to be administered will be governed by such considerations, and is the minimum amount necessary to activate TLR7 receptor and lead to produce INF-α and other cytokines, which can be used, but not limited, for the treatment or prevention of hepatitis B and/or C viral infected patients.

In one example, the pharmaceutically effective amount of the compound of the invention administered parenterally per dose will be in the range of about 0.1 to 50 mg/kg, alternatively about 0.1 to 30 mg/kg of patient body weight per day, with the typical initial range of compound used being 0.3 to 15 mg/kg/day. In another embodiment, oral unit dosage forms, such as tablets and capsules, preferably contain from about 20 to about 1000 mg of the compound of the invention.

The compounds of the invention may be administered by any suitable means, including oral, topical (including buccal and sublingual), rectal, vaginal, transdermal, parenteral, subcutaneous, intraperitoneal, intrapulmonary, intradermal, intrathecal and epidural and intranasal, and, if desired for local treatment, intralesional administration. Parenteral infusions include intramuscular, intravenous, intraarterial, intraperitoneal, or subcutaneous administration.

The compounds of the present invention may be administered in any convenient administrative form, e.g., tablets, powders, capsules, solutions, dispersions, suspensions, syrups, sprays, suppositories, gels, emulsions, patches, etc. Such compositions may contain components conventional in pharmaceutical preparations, e.g., diluents, carriers, pH modifiers, sweeteners, bulking agents, and further active agents.

A typical formulation is prepared by mixing a compound of the present invention and a carrier or excipient. Suitable carriers and excipients are well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., a compound of the present invention or pharmaceutical composition thereof) or aid in the manufacturing of the pharmaceutical product (i.e., medicament).

An example of a suitable oral dosage form is a tablet containing about 20 to 1000 mg of the compound of the invention compounded with about 30 to 90 mg anhydrous lactose, about 5 to 40 mg sodium croscarmellose, about 5 to 30 mg polyvinylpyrrolidone (PVP) K30, and about 1 to 10 mg magnesium stearate. The powdered ingredients are first mixed together and then mixed with a solution of the PVP. The resulting composition can be dried, granulated, mixed with the magnesium stearate and compressed to tablet form using conventional equipment. An example of an aerosol formulation can be prepared by dissolving the compound, for example 20 to 1000 mg, of the invention in a suitable buffer solution, e.g. a phosphate buffer, adding a tonicifier, e.g. a salt such sodium chloride, if desired. The solution may be filtered, e.g., using a 0.2 micron filter, to remove impurities and contaminants.

An embodiment, therefore, includes a pharmaceutical composition comprising a compound of formula (I) or pharmaceutically acceptable salts or enantiomers or diastereomers thereof.

In a further embodiment includes a pharmaceutical composition comprising a compound of formula (I) or pharmaceutically acceptable salts or enantiomers or diastereomers thereof, together with a pharmaceutically acceptable carrier or excipient.

Another embodiment includes a pharmaceutical composition comprising a compound of formula (I) or pharmaceutically acceptable salts or enantiomers or diastereomers thereof for use in the treatment of hepatitis B virus infection.

### Indications and Methods of Treatment

The present invention provides methods for treating or preventing liver cancer in a patient in need thereof. In some embodiments, the liver cancer is hepatocellular carcinoma, hepatoma, cholangiocarcinoma, hepatoblastoma, hepatic carcinoma, hepatic angiosarcoma, or metastatic liver cancer. In some embodiments, the liver cancer is a refractory cancer.

The terms “cancer” and “cancerous” refer to or describe the physiological condition in mammals that is typically characterized by unregulated cell growth/proliferation. Examples of liver cancer include, but are not limited to, hepatocellular carcinoma, hepatoma, hepatoblastoma, cholangiocarcinoma, hepatoblastoma, hepatic carcinoma, sarcoma, lymphoma and hepatic angiosarcoma. In various embodiments, the liver cancer (e.g., HCC) can be intermediate, advanced, or terminal stage. The liver cancer (e.g., HCC) can be metastatic or non-metastatic. The liver cancer (e.g., HCC) can be resectable or unresectable. The liver cancer (e.g., HCC) can comprise a single tumor, multiple tumors, or a poorly defined tumor with an infiltrative growth pattern (into portal veins or hepatic veins). The liver cancer (e.g., HCC) can comprise a fibrolamellar, pseudoglandular (adenoid), pleomorphic (giant cell), or clear cell pattern. The liver cancer (e.g., HCC) can comprise a well differentiated form, and tumor cells resemble hepatocytes, form trabeculae, cords, and nests, and/or contain bile pigment in cytoplasm. The liver cancer (e.g., HCC) can comprise a poorly differentiated form, and malignant epithelial cells are discohesive, pleomorphic, anaplastic, and/or giant. In some embodiments, the liver cancer (e.g., HCC) is associated with hepatits B, hepatitis C, cirhhosis, or type 2 diabetes. The terms “cell proliferative disorder” and “proliferative disorder” refer to disorders that are associated with some degree of abnormal cell proliferation. In one embodiment, the cell proliferative disorder is cancer.

In one embodiment of the present invention the compounds (and pharmaceutical compositions and medicaments thereof) described herein are used in the prophylaxis/prevention of liver cancer in patients which have a high risk of developing liver cancer.

In one preferred embodiment of the invention the compounds described herein are especially useful as prodrugs which are converted into the active drug predominantly in the liver. One embodiment of the invention embodiment are the prodrug compounds described herein for use in the treatment of liver cancer wherein the compounds are prodrugs of the formula (I),

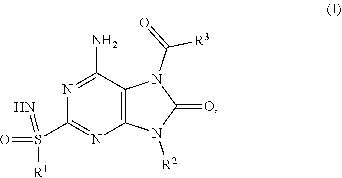

wherein
R¹ is C₁₋₆alkyl;
R² is benzyl, said benzyl being unsubstituted or substituted by one, two or three substituents independently selected from halogen and C₁₋₆alkyl;
R³ is —NR⁴R⁵, wherein
   R⁴ is C₁₋₆-alkyl or C₁₋₆alkoxyC₁₋₆alkyl;
   R⁵ is (C₁₋₆alkyl)₂NCOOC₁₋₆alkyl, C₁₋₆alkoxyC₁₋₆alkyl, C₁₋₆alkoxycarbonyl(C₁₋₆alkyl)aminoC₁₋₆ alkyl, C₁₋₆alkoxycarbonyl(phenyl)C₁₋₆alkyl, C₁₋₆alkoxycarbonylC₁₋₆alkyl, C₁₋₆ alkoxycarbonyloxyC₁₋₆alkyl, C₁₋₆alkyl, C₁₋₆alkylcarbonyl(C₁₋₆alkyl)aminoC₁₋₆alkyl or pyrrolidinylcarbamoyloxyC₁₋₆alkyl; or
   R⁴ and R⁵ together with the nitrogen they are attached to form a heterocyclyl;
      or pharmaceutically acceptable salt, enantiomer or diastereomer thereof,
      for use in the treatment or prophylaxis of liver cancer;
      with the proviso that
6-amino-9-benzyl-2-(propylsulfonimidoyl)-7-(pyrrolidine-1-carbonyl)purin-8-one;
6-amino-9-benzyl-7-(piperidine-1-carbonyl)-2-(propylsulfonimidoyl)purin-8-one;
6-amino-9-benzyl-7-(morpholine-4-carbonyl)-2-(propylsulfonimidoyl)purin-8-one;
6-amino-9-benzyl-7-(3,3-dimethylpyrrolidine-1-carbonyl)-2-(propylsulfonimidoyl)purin-8-one;
ethyl 1-[6-amino-9-benzyl-8-oxo-2-(propylsulfonimidoyl)purine-7-carbonyl]pyrrolidine-2-carboxylate;
6-amino-7-(2-azaspiro[3.3]heptane-2-carbonyl)-9-benzyl-2-(propylsulfonimidoyl)purin-8-one;
6-amino-9-benzyl-7-(2-oxa-6-azaspiro[3.3]heptane-6-carbonyl)-2-(propylsulfonimidoyl)purin-8-one;
6-amino-9-benzyl-7-(3,3-difluoropyrrolidine-1-carbonyl)-2-(propylsulfonimidoyl)purin-8-one;
6-amino-9-benzyl-7-(3-fluoro-3-methyl-pyrrolidine-1-carbonyl)-2-(propylsulfonimidoyl)purin-8-one;
   and their enantiomers or diastereomers are excluded,
   and wherein the prodrug compounds of formula I are converted in the human liver into the active drug of the formula II

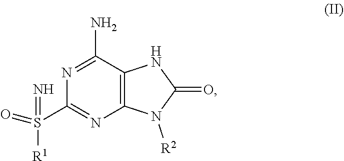

wherein R¹ and R² are defined above.

Exemplary conversion ratios using human liver microsomes are shown in Example 50. Also example 57 demonstrates the liver as the primary site of conversion of the prodrug into its active form.

One preferred embodiment of the invention are the (prodrug) compounds described herein wherein the compounds are susceptible for the conversion into its active form by the liver enzymes CYP2C9 and CYP2C19. One preferred embodiment of the invention are the (prodrug) compounds described herein wherein the compounds show a conversion rate into the active compound of ≥10 nmol/min/mg protein in human hepatocytes and of ≤2 nmol/min/mg protein in human enterocytes (as measured in an appropriate assay using human hepatocytes and human enterocytes.

### Combination Treatment

One aspect of the invention is the combined treatment (combination treatment) of a patient suffering from liver cancer with the compound of formula I with

Surprisingly, we found that that a combination therapy of the compounds of formula I and an anti-PD-L1/PD1 axis treatment is highly effective for liver tumors
Therefore one aspect of the invention is a compound of the formula (I) (or a medicament or a pharmaceutical composition comprising such compound),

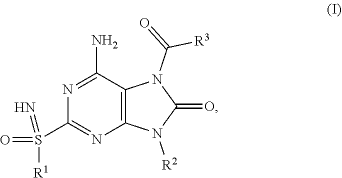

wherein
R¹ is C₁₋₆alkyl;
R² is benzyl, said benzyl being unsubstituted or substituted by one, two or three substituents independently selected from halogen and C₁₋₆alkyl;
R³ is —NR⁴R⁵, wherein
   R⁴ is C₁₋₆alkyl or C₁₋₆alkoxyC₁₋₆alkyl;
   R⁵ is (C₁₋₆alkyl)₂NCOOC₁₋₆alkyl, C₁₋₆alkoxyC₁₋₆alkyl, C₁₋₆alkoxycarbonyl(C₁₋₆alkyl)aminoC₁₋₆ alkyl, C₁₋₆alkoxycarbonyl(phenyl)C₁₋₆alkyl, C₁₋₆alkoxycarbonylC₁₋₆alkyl, C₁₋₆ alkoxycarbonyloxyC₁₋₆alkyl, C₁₋₆alkyl, C₁₋₆alkylcarbonyl(C₁₋₆alkyl)aminoC₁₋₆alkyl or pyrrolidinylcarbamoyloxyC₁₋₆alkyl; or
   R⁴ and R⁵ together with the nitrogen they are attached to form a heterocyclyl;
or pharmaceutically acceptable salt, enantiomer or diastereomer thereof,
with the proviso that
6-amino-9-benzyl-2-(propylsulfonimidoyl)-7-(pyrrolidine-1-carbonyl)purin-8-one;
6-amino-9-benzyl-7-(piperidine-1-carbonyl)-2-(propylsulfonimidoyl)purin-8-one;
6-amino-9-benzyl-7-(morpholine-4-carbonyl)-2-(propylsulfonimidoyl)purin-8-one;
6-amino-9-benzyl-7-(3,3-dimethylpyrrolidine-1-carbonyl)-2-(propylsulfonimidoyl)purin-8-one;
ethyl 1-[6-amino-9-benzyl-8-oxo-2-(propylsulfonimidoyl)purine-7-carbonyl]pyrrolidine-2-carboxylate;
6-amino-7-(2-azaspiro[3.3]heptane-2-carbonyl)-9-benzyl-2-(propylsulfonimidoyl)purin-8-one;
6-amino-9-benzyl-7-(2-oxa-6-azaspiro[3.3]heptane-6-carbonyl)-2-(propylsulfonimidoyl)purin-8-one;
6-amino-9-benzyl-7-(3,3-difluoropyrrolidine-1-carbonyl)-2-(propylsulfonimidoyl)purin-8-one;
6-amino-9-benzyl-7-(3-fluoro-3-methyl-pyrrolidine-1-carbonyl)-2-(propylsulfonimidoyl)purin-8-one;
   and their enantiomers or diastereomers are excluded,
      for use in
   a) the treatment of liver cancer in combination with an antagonistic PD1 or antagonistic PD-L1 antibody,
   or
   b) the treatment of a patient suffering from liver cancer in combination with an antagonistic PD1 or antagonistic PD-L1 antibody.
One embodiment of the invention is a compound of the formula (I) (or a medicament or a pharmaceutical composition comprising such compound),

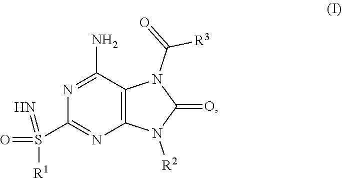

wherein
R¹ is C₁₋₆alkyl;
R² is benzyl, said benzyl being unsubstituted or substituted by one, two or three substituents independently selected from halogen and C₁₋₆alkyl;
R³ is —NR⁴R⁵, wherein
   R⁴ is C₁₋₆alkyl or C₁₋₆alkoxyC₁₋₆alkyl;
   R⁵ is (C₁₋₆alkyl)₂NCOOC₁₋₆alkyl, C₁₋₆alkoxyC₁₋₆alkyl, C₁₋₆alkoxycarbonyl(C₁₋₆alkyl)aminoC₁₋₆ alkyl, C₁₋₆alkoxycarbonyl(phenyl)C₁₋₆alkyl, C₁₋₆alkoxycarbonylC₁₋₆alkyl, C₁₋₆ alkoxycarbonyloxyC₁₋₆alkyl, C₁₋₆alkyl, C₁₋₆alkylcarbonyl(C₁₋₆alkyl)aminoC₁₋₆alkyl or pyrrolidinylcarbamoyloxyC₁₋₆alkyl; or
   R⁴ and R⁵ together with the nitrogen they are attached to form a heterocyclyl;
or pharmaceutically acceptable salt, enantiomer or diastereomer thereof;
with the proviso that
6-amino-9-benzyl-2-(propylsulfonimidoyl)-7-(pyrrolidine-1-carbonyl)purin-8-one;
6-amino-9-benzyl-7-(piperidine-1-carbonyl)-2-(propylsulfonimidoyl)purin-8-one;
6-amino-9-benzyl-7-(morpholine-4-carbonyl)-2-(propylsulfonimidoyl)purin-8-one;
6-amino-9-benzyl-7-(3,3-dimethylpyrrolidine-1-carbonyl)-2-(propylsulfonimidoyl)purin-8-one;
ethyl 1-[6-amino-9-benzyl-8-oxo-2-(propylsulfonimidoyl)purine-7-carbonyl]pyrrolidine-2-carboxylate;
6-amino-7-(2-azaspiro[3.3]heptane-2-carbonyl)-9-benzyl-2-(propylsulfonimidoyl)purin-8-one;
6-amino-9-benzyl-7-(2-oxa-6-azaspiro[3.3]heptane-6-carbonyl)-2-(propylsulfonimidoyl)purin-8-one;
6-amino-9-benzyl-7-(3,3-difluoropyrrolidine-1-carbonyl)-2-(propylsulfonimidoyl)purin-8-one;
6-amino-9-benzyl-7-(3-fluoro-3-methyl-pyrrolidine-1-carbonyl)-2-(propylsulfonimidoyl)purin-8-one;
   and their enantiomers or diastereomers are excluded,
      for use in the treatment or or prophylaxis of liver cancer
   wherein an antagonistic PD1 or antagonistic PD-L1 antibody is co-administered (wherein the treatment is in combination with an antagonistic PD1 or antagonistic PD-L1 antibody).

One embodiment of the invention is the use of a compound of the formula (I),

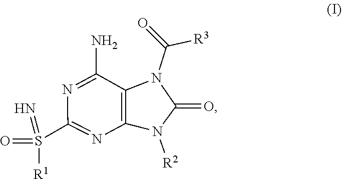

wherein
R¹ is C₁₋₆alkyl;
R² is benzyl, said benzyl being unsubstituted or substituted by one, two or three substituents independently selected from halogen and C₁₋₆alkyl;
R³ is —NR⁴R⁵, wherein
   R⁴ is C₁₋₆alkyl or C₁₋₆alkoxyC₁₋₆alkyl;
   R⁵ is (C₁₋₆alkyl)₂NCOOC₁₋₆alkyl, C₁₋₆alkoxyC₁₋₆alkyl, C₁₋₆alkoxycarbonyl(C₁₋₆alkyl)aminoC₁₋₆ alkyl, C₁₋₆alkoxycarbonyl(phenyl)C₁₋₆alkyl, C₁₋₆alkoxycarbonylC₁₋₆alkyl, C₁₋₆ alkoxycarbonyloxyC₁₋₆alkyl, C₁₋₆alkyl, C₁₋₆alkylcarbonyl(C₁₋₆alkyl)aminoC₁₋₆alkyl or pyrrolidinylcarbamoyloxyC₁₋₆alkyl; or
   R⁴ and R⁵ together with the nitrogen they are attached to form a heterocyclyl;
or pharmaceutically acceptable salt, enantiomer or diastereomer thereof;
with the proviso that
6-amino-9-benzyl-2-(propylsulfonimidoyl)-7-(pyrrolidine-1-carbonyl)purin-8-one;
6-amino-9-benzyl-7-(piperidine-1-carbonyl)-2-(propylsulfonimidoyl)purin-8-one;
6-amino-9-benzyl-7-(morpholine-4-carbonyl)-2-(propylsulfonimidoyl)purin-8-one;
6-amino-9-benzyl-7-(3,3-dimethylpyrrolidine-1-carbonyl)-2-(propylsulfonimidoyl)purin-8-one;
ethyl 1-[6-amino-9-benzyl-8-oxo-2-(propylsulfonimidoyl)purine-7-carbonyl]pyrrolidine-2-carboxylate;
6-amino-7-(2-azaspiro[3.3]heptane-2-carbonyl)-9-benzyl-2-(propylsulfonimidoyl)purin-8-one;
6-amino-9-benzyl-7-(2-oxa-6-azaspiro[3.3]heptane-6-carbonyl)-2-(propylsulfonimidoyl)purin-8-one;
6-amino-9-benzyl-7-(3,3-difluoropyrrolidine-1-carbonyl)-2-(propylsulfonimidoyl)purin-8-one;
6-amino-9-benzyl-7-(3-fluoro-3-methyl-pyrrolidine-1-carbonyl)-2-(propylsulfonimidoyl)purin-8-one;
   and their enantiomers or diastereomers are excluded,
      for the preparation of a medicament for the treatment or prophylaxis of liver cancer
   wherein an antagonistic PD1 or antagonistic PD-L1 antibody is co-administered (wherein the treatment is in combination with an antagonistic PD1 or antagonistic PD-L1 antibody).

In another embodiment of present invention, the particular compounds of formula (I) which are used in the combination therapy with the antagonistic PD1 or antagonistic PD-L1 antibody are selected from the following:
6-Amino-9-benzyl-N-methyl-8-oxo-N-propyl-2-(propylsulfonimidoyl)purine-7-carboxamide;
6-Amino-9-benzyl-N-(2-methoxyethyl)-N-methyl-8-oxo-2-(propylsulfonimidoyl)purine-7-carboxamide;
6-Amino-9-benzyl-N-ethyl-8-oxo-N-propyl-2-(propylsulfonimidoyl)purine-7-carboxamide;
6-Amino-9-benzyl-7-[4-(1-piperidyl)piperidine-1-carbonyl]-2-(propylsulfonimidoyl)purin-8-one;
6-Amino-9-benzyl-N-ethyl-N-(2-methoxyethyl)-8-oxo-2-(propylsulfonimidoyl)purine-7-carboxamide;
6-Amino-9-benzyl-N-butyl-N-ethyl-8-oxo-2-(propylsulfonimidoyl)purine-7-carboxamide;
6-Amino-9-benzyl-N-(2-methoxyethyl)-8-oxo-N-propyl-2-(propylsulfonimidoyl)purine-7-carboxamide;
6-Amino-9-benzyl-N,N-bis(2-methoxyethyl)-8-oxo-2-(propylsulfonimidoyl)purine-7-carboxamide;
6-Amino-7-(azetidine-1-carbonyl)-9-benzyl-2-(propylsulfonimidoyl)purin-8-one;
6-Amino-9-benzyl-N-isopropyl-N-methyl-8-oxo-2-(propylsulfonimidoyl)purine-7-carboxamide;
6-Amino-9-benzyl-7-(4-methylpiperazine-1-carbonyl)-2-(propylsulfonimidoyl)purin-8-one;
6-Amino-9-benzyl-N-(3-methoxypropyl)-N-methyl-8-oxo-2-(propylsulfonimidoyl)purine-7-carboxamide;
6-Amino-9-benzyl-N-isobutyl-N-methyl-8-oxo-2-(propylsulfonimidoyl)purine-7-carboxamide;
Ethyl 2-[[6-amino-9-benzyl-8-oxo-2-(propylsulfonimidoyl)purine-7-carbonyl]-methyl-amino]acetate;
Ethyl 3-[[6-amino-9-benzyl-8-oxo-2-(propylsulfonimidoyl)purine-7-carbonyl]-methyl-amino]propanoate;
tert-Butyl 3-[[6-amino-9-benzyl-8-oxo-2-(propylsulfonimidoyl)purine-7-carbonyl]-methyl-amino]propanoate;
Ethyl (2S)-2-[[6-amino-9-benzyl-8-oxo-2-(propylsulfonimidoyl)purine-7-carbonyl]-methyl-amino]propanoate;
tert-Butyl (2S)-2-[[6-amino-9-benzyl-8-oxo-2-(propylsulfonimidoyl)purine-7-carbonyl]-methyl-amino]-4-methyl-pentanoate;
Isopropyl (2S)-2-[[6-amino-9-benzyl-8-oxo-2-(propylsulfonimidoyl)purine-7-carbonyl]-methyl-amino]-4-methyl-pentanoate;
Ethyl (2S)-2-[[6-amino-9-benzyl-8-oxo-2-(propylsulfonimidoyl)purine-7-carbonyl]-methyl-amino]-3-methyl-butanoate;
Ethyl (2S)-2-[[6-amino-9-benzyl-8-oxo-2-(propylsulfonimidoyl)purine-7-carbonyl]-methyl-amino]-4-methyl-pentanoate;
Ethyl (2S)-2-[[6-amino-9-benzyl-8-oxo-2-(propylsulfonimidoyl)purine-7-carbonyl]-methyl-amino]-3-phenyl-propanoate;
Isopropyl (2S)-2-[[6-amino-9-benzyl-8-oxo-2-(propylsulfonimidoyl)purine-7-carbonyl]-methyl-amino]-3-phenyl-propanoate;
tert-Butyl (2S)-2-[[6-amino-9-benzyl-8-oxo-2-(propylsulfonimidoyl)purine-7-carbonyl]-methyl-amino]-3-phenyl-propanoate;
N-[2-[Acetyl(methyl)amino]ethyl]-6-amino-9-benzyl-N-methyl-8-oxo-2-(propylsulfonimidoyl)purine-7-carboxamide;
Methyl N-[2-[[6-amino-9-benzyl-8-oxo-2-(propylsulfonimidoyl)purine-7-carbonyl]-methyl-amino]ethyl]-N-methyl-carbamate;
tert-Butyl N-[2-[[6-amino-9-benzyl-8-oxo-2-(propylsulfonimidoyl)purine-7-carbonyl]-methyl-amino]ethyl]-N-methyl-carbamate;
Ethyl N-[2-[[6-amino-9-benzyl-8-oxo-2-(propylsulfonimidoyl)purine-7-carbonyl]-methyl-amino]ethyl]-N-methyl-carbamate;
2-[[6-Amino-9-benzyl-8-oxo-2-(propylsulfonimidoyl)purine-7-carbonyl]-methyl-amino]ethyl N-butyl-N-methyl-carbamate;
2-[[6-Amino-9-benzyl-8-oxo-2-(propylsulfonimidoyl)purine-7-carbonyl]-methyl-amino]ethyl pyrrolidine-1-carboxylate;
2-[[6-Amino-9-benzyl-8-oxo-2-(propylsulfonimidoyl)purine-7-carbonyl]-methyl-amino]ethyl N-methyl-N-propyl-carbamate;
2-[[6-Amino-9-benzyl-8-oxo-2-(propylsulfonimidoyl)purine-7-carbonyl]-methyl-amino]ethyl N,N-diethylcarbamate;
2-[[6-Amino-9-benzyl-8-oxo-2-(propylsulfonimidoyl)purine-7-carbonyl]-methyl-amino]ethyl ethyl carbonate;
6-Amino-N-butyl-9-[(4-chlorophenyl)methyl]-N-methyl-8-oxo-2-[S(S)-propylsulfonimidoyl]purine-7-carboxamide;
6-amino-N-butyl-9-[(4-chlorophenyl)methyl]-N-methyl-8-oxo-2-[S(S)-propylsulfonimidoyl]purine-7-carboxamide;
6-Amino-9-[(4-chlorophenyl)methyl]-N-ethyl-N-methyl-8-oxo-2-(propylsulfonimidoyl)purine-7-carboxamide;
6-Amino-N-methyl-8-oxo-N-propyl-2 [S(S)-propylsulfonimidoyl]-9-(p-tolylmethyl)purine-7-carboxamide;
6-Amino-N-methyl-8-oxo-N-propyl-2[S(R)-propylsulfonimidoyl]-9-(p-tolylmethyl)purine-7-carboxamide;
6-Amino-2-[S(S)-propylsulfonimidoyl]-9-(p-tolylmethyl)-7-(pyrrolidine-1-carbonyl)purin-8-one;
6-Amino-2-[S(R)-propylsulfonimidoyl]-9-(p-tolylmethyl)-7-(pyrrolidine-1-carbonyl)purin-8-one;
6-Amino-N-(2-methoxyethyl)-N-methyl-8-oxo-2-[S(S)-propylsulfonimidoyl]-9-(p-tolylmethyl)purine-7-carboxamide;
6-Amino-N-(2-methoxyethyl)-N-methyl-8-oxo-2-[S(R)-propylsulfonimidoyl]-9-(p-tolylmethyl)purine-7-carboxamide;
6-Amino-N-ethyl-N-methyl-8-oxo-2-(propylsulfonimidoyl)-9-(p-tolylmethyl)purine-7-carboxamide;
6-Amino-N-butyl-N-methyl-8-oxo-2-(propylsulfonimidoyl)-9-(p-tolylmethyl)purine-7-carboxamide;
6-Amino-9-[(4-chlorophenyl)methyl]-2-[S(R)-ethylsulfonimidoyl]-N-methyl-8-oxo-N-propyl-purine-7-carboxamide;
6-Amino-9-[(4-chlorophenyl)methyl]-2-[S(S)-ethylsulfonimidoyl]-N-methyl-8-oxo-N-propyl-purine-7-carboxamide;
6-Amino-9-[(4-chlorophenyl)methyl]-N-ethyl-2 [S(S)-ethylsulfonimidoyl]-N-methyl-8-oxo-purine-7-carboxamide;
6-Amino-9-[(4-chlorophenyl)methyl]-N-ethyl-2-[S(R)-ethylsulfonimidoyl]-N-methyl-8-oxo-purine-7-carboxamide;
6-Amino-2-[S(S)-ethylsulfonimidoyl]-N-methyl-8-oxo-N-propyl-9-(p-tolylmethyl)purine-7-carboxamide;
6-Amino-2-[S(R)-ethylsulfonimidoyl]-N-methyl-8-oxo-N-propyl-9-(p-tolylmethyl)purine-7-carboxamide;
6-Amino-N-ethyl-2[S(S)-ethylsulfonimidoyl]-N-methyl-8-oxo-9-(p-tolylmethyl)purine-7-carboxamide;
6-Amino-N-ethyl-2-[S(R)-ethyl sulfonimidoyl]-N-methyl-8-oxo-9-(p-tolylmethyl)purine-7-carboxamide;
6-Amino-2-[S(S)ethylsulfonimidoyl]-9-[(4-fluorophenyl)methyl]-N-methyl-8-oxo-N-propyl-purine-7-carboxamide;
6-Amino-2-[S(R)ethylsulfonimidoyl]-9-[(4-fluorophenyl)methyl]-N-methyl-8-oxo-N-propyl-purine-7-carboxamide;
6-Amino-N-ethyl-2-(ethylsulfonimidoyl)-9-[(4-fluorophenyl)methyl]-N-methyl-8-oxo-purine-7-carboxamide;
6-Amino-N-ethyl-2-[S(S)-(ethylsulfonimidoyl)]-9-[(4-fluorophenyl)methyl]-N-methyl-8-oxo-purine-7-carboxamide;
6-Amino-N-ethyl-2-[S(R)-(ethylsulfonimidoyl)]-9-[(4-fluorophenyl)methyl]-N-methyl-8-oxo-purine-7-carboxamide;
6-Amino-9-[(4-bromophenyl)methyl]-2-(ethylsulfonimidoyl)-N-methyl-8-oxo-N-propyl-purine-7-carboxamide;
6-Amino-2-[S(R)-ethylsulfonimidoyl]-9-[(4-bromophenyl)methyl]-N-methyl-8-oxo-N-propyl-purine-7-carboxamide;
6-Amino-2-[S(S)-ethyl sulfonimidoyl]-9-[(4-bromophenyl)methyl]-N-methyl-8-oxo-N-propyl-purine-7-carboxamide;
6-Amino-9-[(4-bromophenyl)methyl]-N-ethyl-2-(ethylsulfonimidoyl)-N-methyl-8-oxo-purine-7-carboxamide;
6-Amino-9-[(4-bromophenyl)methyl]-N-ethyl-2-[S(S)-(ethylsulfonimidoyl)]-N-methyl-8-oxo-purine-7-carboxamide; and
6-Amino-9-[(4-bromophenyl)methyl]-N-ethyl-2-[S(R)-(ethylsulfonimidoyl)]-N-methyl-8-oxo-purine-7-carboxamide;
   or pharmaceutically acceptable salt, enantiomer or diastereomer thereof.

In another embodiment of present invention, the particular compounds of formula (I) which are used in the combination therapy with the antagonistic PD1 or antagonistic PD-L1 antibody are selected from the following:
6-Amino-9-[(4-chlorophenyl)methyl]-N-ethyl-2[S(S)-ethylsulfonimidoyl]-N-methyl-8-oxo-purine-7-carboxamide;
6-Amino-9-[(4-chlorophenyl)methyl]-N-ethyl-2-[S(R)-ethylsulfonimidoyl]-N-methyl-8-oxo-purine-7-carboxamide;
6-Amino-9-[(4-bromophenyl)methyl]-N-ethyl-2-(ethylsulfonimidoyl)-N-methyl-8-oxo-purine-7-carboxamide;
6-Amino-9-[(4-bromophenyl)methyl]-N-ethyl-2-[S(S)-(ethylsulfonimidoyl)]-N-methyl-8-oxo-purine-7-carboxamide; and
6-Amino-9-[(4-bromophenyl)methyl]-N-ethyl-2-[S(R)-(ethylsulfonimidoyl)]-N-methyl-8-oxo-purine-7-carboxamide;
   or pharmaceutically acceptable salt, enantiomer or diastereomer thereof.

In another embodiment of present invention, the particular compound of formula (I) which is used in the combination therapy with the antagonistic PD1 or antagonistic PD-L1 antibody is: 6-Amino-9-[(4-chlorophenyl)methyl]-N-ethyl-2 [S(S)-ethylsulfonimidoyl]-N-methyl-8-oxo-purine-7-carboxamide In one embodiment the co-administration (or combination therapy or treatment in combination with or combination treatment) of the compound of formula I and the antagonistic PD1 or antagonistic PD-L1 antibody is simultaneously. In one embodiment the co-administration (or combination therapy or treatment in combination with or combination treatment) of the compound of formula I and the antagonistic PD1 or antagonistic PD-L1 antibody is sequentially.

The terms “administered in combination with” or “co-administration”, “co-administering”, “combination therapy”, “treatment in combination with “or “combination treatment” refer to the administration of the Compound of formula I as described herein, and the antagonistic PD1 or PD-L1 antibody, as described herein e.g. as separate formulations/applications (or as one single formulation/application). The co-administration can be simultaneous or sequential in either order, wherein there is a time period while both (or all) active agents simultaneously exert their biological activities. The co-administration is either simultaneously or sequentially (e.g. intravenous (i.v.) through a continuous infusion. In one embodiment the co-administration is simultaneously. In one embodiment the co-administration is sequentially. The co-administration is either simultaneously or sequentially (e.g. intravenous (i.v.) through a continuous infusion.

It is self-evident that the antibodies are administered to the patient in a “therapeutically effective amount” (or simply “effective amount”) which is the amount of the respective compound or combination that will elicit the biological or medical response of a tissue, system, animal or human that is being sought by the researcher, veterinarian, medical doctor or other clinician.

The amount of co-administration and the timing of co-administration will depend on the type (species, gender, age, weight, etc.) and condition of the patient being treated and the severity of the disease or condition being treated. Said compounds of formula I and said antibodies are suitably co-administered to the patient at one time or over a series of treatments e.g. on the same day or on the day after.

### PD-1/PD-L1/PD-L2 Pathway:

An important negative co-stimulatory signal regulating T cell activation is provided by programmed death—1 receptor (PD-1)(CD279), and its ligand binding partners PD-L1 (B7-H1, CD274; SEQ ID NO: 13) and PD-L2 (B7-DC, CD273). The negative regulatory role of PD-1 was revealed by PD-1 knock outs (Pdcd1−/−), which are prone to autoimmunity. Nishimura et al., Immunity 11: 141-51 (1999); Nishimura et al., Science 291: 319-22 (2001). PD-1 is related to CD28 and CTLA-4, but lacks the membrane proximal cysteine that allows homodimerization. The cytoplasmic domain of PD-1 contains an immunoreceptor tyrosine-based inhibition motif (ITIM, V/IxYxxL/V). PD-1 only binds to PD-L1 and PD-L2. Freeman et al., J. Exp. Med. 192: 1-9 (2000); Dong et al., Nature Med. 5: 1365-1369 (1999); Latchman et al., Nature Immunol. 2: 261-268 (2001); Tseng et al., J. Exp. Med. 193: 839-846 (2001).

PD-1 can be expressed on T cells, B cells, natural killer T cells, activated monocytes and dendritic cells (DCs). PD-1 is expressed by activated, but not by unstimulated human CD4+ and CD8+ T cells, B cells and myeloid cells. This stands in contrast to the more restricted expression of CD28 and CTLA-4. Nishimura et al., Int. Immunol. 8: 773-80 (1996); Boettler et al., J. Virol. 80: 3532-40 (2006). There are at least 4 variants of PD-1 that have been cloned from activated human T cells, including transcripts lacking (i) exon 2, (ii) exon 3, (iii) exons 2 and 3 or (iv) exons 2 through 4. Nielsen et al., Cell. Immunol. 235: 109-16 (2005). With the exception of PD-1deltaex3, all variants are expressed at similar levels as full length PD-1 in resting peripheral blood mononuclear cells (PBMCs). Expression of all variants is significantly induced upon activation of human T cells with anti-CD3 and anti-CD28. The PD-1deltaex3 variants lacks a transmembrane domain, and resembles soluble CTLA-4, which plays an important role in autoimmunity. Ueda et al., Nature 423: 506-11 (2003). This variant is enriched in the synovial fluid and sera of patients with rheumatoid arthritis. Wan et al., J. Immunol. 177: 8844-50 (2006).

The two PD-1 ligands differ in their expression patterns. PD-L1 is constitutively expressed on mouse T and B cells, CDs, macrophages, mesenchymal stem cells and bone marrow-derived mast cells. Yamazaki et al., J. Immunol. 169: 5538-45 (2002). PD-L1 is expressed on a wide range of nonhematopoietic cells (e.g., cornea, lung, vascular epithelium, liver nonparenchymal cells, mesenchymal stem cells, pancreatic islets, placental synctiotrophoblasts, keratinocytes, etc.) [Keir et al., Annu. Rev. Immunol. 26: 677-704 (2008)], and is upregulated on a number of cell types after activation. Both type I and type II interferons IFN's) upregulate PD-L1. Eppihimer et al., Microcirculation 9: 133-45 (2002); Schreiner et al., J. Neuroimmunol. 155: 172-82 (2004). PD-L1 expression in cell lines is decreased when MyD88, TRAF6 and MEK are inhibited. Liu et al., Blood 110: 296-304 (2007). JAK2 has also been implicated in PD-L1 induction. Lee et al., FEBS Lett. 580: 755-62 (2006); Liu et al., Blood 110: 296-304 (2007). Loss or inhibition of phosphatase and tensin homolog (PTEN), a cellular phosphatase that modified phosphatidylinositol 3-kinase (PI3K) and Akt signaling, increased post-transcriptional PD-L1 expression in cancers. Parsa et al., Nat. Med. 13: 84-88 (2007).

PD-L2 expression is more restricted than PD-L1. PD-L2 is inducibly expressed on DCs, macrophages, and bone marrow-derived mast cells. PD-L2 is also expressed on about half to two-thirds of resting peritoneal B1 cells, but not on conventional B2 B cells. Zhong et al., Eur. J. Immunol. 37: 2405-10 (2007). PD-L2+B1 cells bind phosphatidylcholine and may be important for innate immune responses against bacterial antigens. Induction of PD-L2 by IFN-gamma is partially dependent upon NF-kappaB. Liang et al., Eur. J. Immunol. 33: 2706-16 (2003). PD-L2 can also be induced on monocytes and macrophages by GM-CF, IL-4 and IFN-gamma. Yamazaki et al., J. Immunol. 169: 5538-45 (2002); Loke et al., PNAS 100:5336-41 (2003).

PD-1 signaling typically has a greater effect on cytokine production than on cellular proliferation, with significant effects on IFN-gamma, TNF-alpha and IL-2 production. PD-1 mediated inhibitory signaling also depends on the strength of the TCR signaling, with greater inhibition delivered at low levels of TCR stimulation. This reduction can be overcome by costimulation through CD28 [Freeman et al., J. Exp. Med. 192: 1027-34 (2000)] or the presence of IL-2 [Carter et al., Eur. J. Immunol. 32: 634-43 (2002)]. Evidence is mounting that signaling through PD-L1 and PD-L2 may be bidirectional. That is, in addition to modifying TCR or BCR signaling, signaling may also be delivered back to the cells expressing PD-L1 and PD-L2. While treatment of dendritic cells with a naturally human anti-PD-L2 antibody isolated from a patient with Waldenstrom's macroglobulinemia was not found to upregulate MHC II or B7 costimulatory molecules, such cells did produce greater amount of proinflammatory cytokines, particularly TNF-alpha and IL-6, and stimulated T cell proliferation. Nguyen et al., J. Exp. Med. 196: 1393-98 (2002). Treatment of mice with this antibody also (1) enhanced resistance to transplanted b16 melanoma and rapidly induced tumor-specific CTL. Radhakrishnan et al., J. Immunol. 170: 1830-38 (2003); Radhakrishnan et al., Cancer Res. 64: 4965-72 (2004); Heckman et al., Eur. J. Immunol. 37: 1827-35 (2007); (2) blocked development of airway inflammatory disease in a mouse model of allergic asthma. Radhakrishnan et al., J. Immunol. 173: 1360-65 (2004); Radhakrishnan et al., J. Allergy Clin. Immunol. 116: 668-74 (2005).

Further evidence of reverse signaling into dendritic cells (“DC's”) results from studies of bone marrow derived DC's cultured with soluble PD-1 (PD-1 EC domain fused to Ig constant region—“s-PD-1”). Kuipers et al., Eur. J. Immunol. 36: 2472-82 (2006). This sPD-1 inhibited DC activation and increased IL-10 production, in a manner reversible through administration of anti-PD-1.

Additionally, several studies show a receptor for PD-L1 or PD-L2 that is independent of PD-1. B7.1 has already been identified as a binding partner for PD-L1. Butte et al., Immunity 27: 111-22 (2007). Chemical crosslinking studies suggest that PD-L1 and B7.1 can interact through their IgV-like domains. B7.1:PD-L1 interactions can induce an inhibitory signal into T cells. Ligation of PD-L1 on CD4+ T cells by B7.1 or ligation of B7.1 on CD4+ T cells by PD-L1 delivers an inhibitory signal. T cells lacking CD28 and CTLA-4 show decreased proliferation and cytokine production when stimulated by anti-CD3 plus B7.1 coated beads. In T cells lacking all the receptors for B7.1 (i.e., CD28, CTLA-4 and PD-L1), T cell proliferation and cytokine production were no longer inhibited by anti-CD3 plus B7.1 coated beads. This indicates that B7.1 acts specifically through PD-L1 on the T-cell in the absence of CD28 and CTLA-4. Similarly, T cells lacking PD-1 showed decreased proliferation and cytokine production when stimulated in the presence of anti-CD3 plus PD-L1 coated beads, demonstrating the inhibitory effect of PD-L1 ligation on B7.1 on T cells. When T cells lacking all known receptors for PD-L1 (i.e., no PD-1 and B7.1), T cell proliferation was no longer impaired by anti-CD3 plus PD-L1 coated beads. Thus, PD-L1 can exert an inhibitory effect on T cells either through B7.1 or PD-1.

The direct interaction between B7.1 and PD-L1 suggests that the current understanding of costimulation is incomplete, and underscores the significance to the expression of these molecules on T cells. Studies of PD-L1−/− T cells indicate that PD-L1 on T cells can downregulate T cell cytokine production. Latchman et al., Proc. Natl. Acad. Sci. USA 101: 10691-96 (2004). Because both PD-L1 and B7.1 are expressed on T cells, B cells, DCs and macrophages, there is the potential for directional interactions between B7.1 and PD-L1 on these cells types. Additionally, PD-L1 on non-hematopoietic cells may interact with B7.1 as well as PD-1 on T cells, raising the question of whether PD-L1 is involved in their regulation. One possible explanation for the inhibitory effect of B7.1:PD-L1 interaction is that T cell PD-L1 may trap or segregate away APC B7.1 from interaction with CD28.

As a result, the antagonism of signaling through PD-L1, including blocking PD-L1 from interacting with either PD-1, B7.1 or both, thereby preventing PD-L1 from sending a negative co-stimulatory signal to T-cells and other antigen presenting cells is likely to enhance immunity in response to infection (e.g., acute and chronic) and tumor immunity.

An exemplary PD-L1 antagonist is the anti-PD-L1 antibody atezolizumab. Other antagonistic PD-L1 antibodies are durvalumab and avelumab.

In another embodiment, the anti-PD-L1/PD1 interaction can blocked by antagonist anti-PD-1 antibodies like the antagonistic PD1 antibodies pembrolizumab or nivolumab or an anti-PD1 antibody comprising the variable heavy chain and light chain domainss of PD1-0103-0312.

The term “human PD-L1” refers to the human protein PD-L1 (SEQ ID NO: 13, PD-1 signaling typically). As used herein, “binding to human PD-L1” or “specifically binding to human PD-L1” or “which binds to human PD-L1” or “anti-PD-L1 antibody” or “antagonistic PD-L1” refers to an antibody specifically binding to the human PD-L1 antigen with a binding affinity of KD-value of 1.0×10-8 mol/l or lower, in one embodiment of a KD-value of 1.0×10-9 mol/l or lower. The binding affinity is determined with a standard binding assay, such as surface plasmon resonance technique (BIAcore®, GE-Healthcare Uppsala, Sweden). Thus an “antibody binding to human PD-L1” as used herein refers to an antibody specifically binding to the human PD-L1 antigen with a binding affinity of KD 1.0×10-8 mol/l or lower (in one embodiment 1.0×10-8 mol/l-1.0×10-13 mol/l), in on embodiment of a KD 1.0×10-9 mol/l or lower (in one embodiment 1.0×10-9 mol/l-1.0×10-13 mol/l).

The term “human PD1” refers to the human protein PD1 (SEQ ID NO: 14, PD-1 signaling typically). As used herein, “binding to human PD1” or “specifically binding to human PD1” or “which binds to human PD1” or “anti-PD1 antibody” or “antagonistic PD1” refers to an antibody specifically binding to the human PD1 antigen with a binding affinity of KD-value of 1.0×10-8 mol/l or lower, in one embodiment of a KD-value of 1.0×10-9 mol/l or lower. The binding affinity is determined with a standard binding assay, such as surface plasmon resonance technique (BIAcore®, GE-Healthcare Uppsala, Sweden). Thus an “antibody binding to human PD1” as used herein refers to an antibody specifically binding to the human PD1 antigen with a binding affinity of KD 1.0×10-8 mol/l or lower (in one embodiment 1.0×10-8 mol/l-1.0×10-13 mol/l), in on embodiment of a KD 1.0×10-9 mol/l or lower (in one embodiment 1.0×10-9 mol/l-1.0×10-13 mol/l).

The “variable domain” (variable domain of a light chain (VL), variable domain of a heavy chain (VH) as used herein denotes each of the pair of light and heavy chains which is involved directly in binding the antibody to the antigen. The domains of variable human light and heavy chains have the same general structure and each domain comprises four framework (FR) regions whose sequences are widely conserved, connected by three “hypervariable regions” (or complementarity determining regions, CDRs). The framework regions adopt a β-sheet conformation and the CDRs may form loops connecting the β-sheet structure. The CDRs in each chain are held in their three-dimensional structure by the framework regions and form together with the CDRs from the other chain the antigen binding site. The antibody heavy and light chain CDR3 regions play a particularly important role in the binding specificity/affinity of the antibodies according to the invention and therefore provide a further object of the invention.

The term “constant region” as used within the current applications denotes the sum of the domains of an antibody other than the variable region. The constant region is not involved directly in binding of an antigen, but exhibits various effector functions. Depending on the amino acid sequence of the constant region of their heavy chains, antibodies are divided in the classes: IgA, IgD, IgE, IgG and IgM, and several of these may be further divided into subclasses, such as IgG1, IgG2, IgG3, and IgG4, IgA1 and IgA2. The heavy chain constant regions that correspond to the different classes of antibodies are called α, δ, ε, γ, and μ, respectively. The light chain constant regions which can be found in all five antibody classes are called κ (kappa) and λ (lambda).

The terms “constant region derived from human origin” or “human constant region” as used in the current application denotes a constant heavy chain region of a human antibody of the subclass IgG1, IgG2, IgG3, or IgG4 and/or a constant light chain kappa or lambda region. Such constant regions are well known in the state of the art and e.g. described by Kabat, E. A., et al., Sequences of Proteins of Immunological Interest, 5th ed., Public Health Service, National Institutes of Health, Bethesda, Md. (1991) (see also e.g. Johnson, G., and Wu, T. T., Nucleic Acids Res. 28 (2000) 214-218; Kabat, E. A., et al., Proc. Natl. Acad. Sci. USA 72 (1975) 2785-2788). Within the application for the numbering of positions and mutations the EU numbering system (EU Index) according to Kabat, E. A., et al., Sequences of Proteins of Immunological Interest, 5th ed., Public Health Service, National Institutes of Health, Bethesda, Md. (1991) is used and referred to as “numbering according to EU Index of Kabat”.

In one embodiment the antagonistic anti-PD1 antibody which binds to human PD1 used in the combination therapy described herein is nivolumab or pembrolizumab and is characterized in comprising the following VH and VL sequences as described herein:

TABLE

| | amino acid sequence of the | amino acid sequence of the |
|---|---|---|
| anti-PD-L1 | heavy chain variable | light chain variable domain |
| antibody | domain VH, SEQ ID NO: | VL, SEQ ID NO: |
| nivolumab | 1 | 2 |
| pembrolizumab | 3 | 4 |

In one preferred embodiment of the invention the compound of formula I used in the combination therapy described herein is selected from the following:
6-Amino-9-[(4-chlorophenyl)methyl]-N-ethyl-2 [S(S)-ethylsulfonimidoyl]-N-methyl-8-oxo-purine-7-carboxamide;
6-Amino-9-[(4-chlorophenyl)methyl]-N-ethyl-2-[S(R)-ethylsulfonimidoyl]-N-methyl-8-oxo-purine-7-carboxamide;
6-Amino-9-[(4-bromophenyl)methyl]-N-ethyl-2-(ethylsulfonimidoyl)-N-methyl-8-oxo-purine-7-carboxamide;
6-Amino-9-[(4-bromophenyl)methyl]-N-ethyl-2-[S(S)-(ethylsulfonimidoyl)]-N-methyl-8-oxo-purine-7-carboxamide; or
6-Amino-9-[(4-bromophenyl)methyl]-N-ethyl-2-[S(R)-(ethylsulfonimidoyl)]-N-methyl-8-oxo-purine-7-carboxamide;
   or pharmaceutically acceptable salt, enantiomer or diastereomer thereof, (in one preferred embodiment 6-Amino-9-[(4-chlorophenyl)methyl]-N-ethyl-2 [S(S)-ethylsulfonimidoyl]-N-methyl-8-oxo-purine-7-carboxamide);

and the antagonistic PD1 antibody used in the combination therapy is nivolumab or pembrolizumab In one embodiment the antagonistic anti-PD1 antibody which binds to human PD1 used in the combination therapy described herein is either a mono- or multispecific antagonistic PD1 antibody and comprises the following heavy chain variable domain VH and light chain variable domain VL sequences as described herein:

TABLE

| | amino acid sequence of the | amino acid sequence of the |
|---|---|---|
| anti-PD1 | heavy chain variable | light chain variable domain |
| antibody | domain VH, SEQ ID NO: | VL, SEQ ID NO: |
| PD1-0103- | 5 | 6 |
| 0312 | | |

Preferably such anti-PD1 antibody based on the heavy chain variable domain VH and light chain variable domain VL sequences of PD1-0103-0312 comprises a heavy chain constant region of IgG1 subtype (e.g. SEQ ID NO: 16 or SEQ ID NO: 17, eventually also comprising further mutations, see below the bispecific embodiment) and a human kappa light chain constant region (e.g. SEQ ID NO: 15).

In one embodiment such anti-PD1 antibody based on the heavy chain variable domain VH and light chain variable domain VL sequences of PD1-0103-0312 is e.g. bispecific and i) the bispecific antibody comprises a constant heavy chain region of human IgG1 subclass comprising the mutations L234A, L235A and P329G (numberings according to EU Index of Kabat); and wherein ii)) in the constant heavy chain region a S354C and T366W mutations are comprised in one CH3 domain and Y349C, T366S, L368A and Y407V mutations are comprised the other CH3 domain (numberings according to EU Index of Kabat).

In another preferred embodiment of the invention the compound of formula I used in the combination therapy described herein is selected from the following:
6-Amino-9-[(4-chlorophenyl)methyl]-N-ethyl-2[S(S)-ethylsulfonimidoyl]-N-methyl-8-oxo-purine-7-carboxamide;
6-Amino-9-[(4-chlorophenyl)methyl]-N-ethyl-2-[S(R)-ethylsulfonimidoyl]-N-methyl-8-oxo-purine-7-carboxamide;
6-Amino-9-[(4-bromophenyl)methyl]-N-ethyl-2-(ethylsulfonimidoyl)-N-methyl-8-oxo-purine-7-carboxamide;
6-Amino-9-[(4-bromophenyl)methyl]-N-ethyl-2-[S(S)-(ethylsulfonimidoyl)]-N-methyl-8-oxo-purine-7-carboxamide; or
6-Amino-9-[(4-bromophenyl)methyl]-N-ethyl-2-[S(R)-(ethylsulfonimidoyl)]-N-methyl-8-oxo-purine-7-carboxamide;
   or pharmaceutically acceptable salt, enantiomer or diastereomer thereof, (in one preferred embodiment 6-Amino-9-[(4-chlorophenyl)methyl]-N-ethyl-2[S(S)-ethylsulfonimidoyl]-N-methyl-8-oxo-purine-7-carboxamide);
   and the antagonistic PD1 antibody used in the combination therapy comprises a heavy chain variable domain VH with an amino acid sequence of SEQ ID NO: 5 and a light chain variable domain VL with an amino acid sequence of SEQ ID NO: 6.

In one embodiment the antibody which binds to human PD-L1 used in the combination therapy described herein is atezolizumab or durvalumab or avelumab and is characterized in comprising the following VH and VL sequences as described herein:

TABLE

| | amino acid sequence of the | amino acid sequence of the |
|---|---|---|
| anti-PD-L1 | heavy chain variable | light chain variable domain |
| antibody | domain VH, SEQ ID NO: | VL, SEQ ID NO: |
| atezolizumab | 7 | 8 |
| durvalumab | 9 | 10 |
| avelumab | 11 | 12 |

In another preferred embodiment of the invention the compounds of formula I used in the combination therapy described herein are selected from the following:
6-Amino-9-[(4-chlorophenyl)methyl]-N-ethyl-2[S(S)-ethylsulfonimidoyl]-N-methyl-8-oxo-purine-7-carboxamide;
6-Amino-9-[(4-chlorophenyl)methyl]-N-ethyl-2-[S(R)-ethylsulfonimidoyl]-N-methyl-8-oxo-purine-7-carboxamide;
6-Amino-9-[(4-bromophenyl)methyl]-N-ethyl-2-(ethylsulfonimidoyl)-N-methyl-8-oxo-purine-7-carboxamide;
6-Amino-9-[(4-bromophenyl)methyl]-N-ethyl-2-[S(S)-(ethylsulfonimidoyl)]-N-methyl-8-oxo-purine-7-carboxamide; or
6-Amino-9-[(4-bromophenyl)methyl]-N-ethyl-2-[S(R)-(ethylsulfonimidoyl)]-N-methyl-8-oxo-purine-7-carboxamide;
   or pharmaceutically acceptable salt, enantiomer or diastereomer thereof, (in one preferred embodiment 6-Amino-9-[(4-chlorophenyl)methyl]-N-ethyl-2 [S(S)-ethylsulfonimidoyl]-N-methyl-8-oxo-purine-7-carboxamide);
   and the antagonistic PD-L1 antibody used in the combination therapy is atezolizumab or durvalumab or avelumab (in one preferred embodiment atezolizumab).

Another aspect of the invention is the combined treatment (combination treatment) of a patient suffering from liver cancer with the compound of formula I as described above in combination with an anti-angiogenic agent. The anti-angiogenic agent can be co-administered either with compounds of formula I alone or in addition to the combination therapy of the compounds of formula I with an anti-PD-L1/PD1 axis treatment. Antiangiogenic agents as used herein include (but are not limited to) small molecule tyrosine kinase inhibitors (TKIs) that bind competitively to the intracellular receptor domains for VEGF, PDGF, and other angiogenic growth factors, like e.g. sorafenib (4-{4-[3-(4-Chlor-3-trifluormethylphenyl)ureido]phenoxy}pyridin-2-carbonsäuremethylamid; Nexavar™), regorafenib (4-[4-({[4-Chlor-3-(trifluormethyl)phenyl]carbamoyl}amino)-3-fluorphenoxy]-N-methylpyridin-2-carboxamid-Hydrat; Stivarga™), and sunitinib (N-[2-(Diethylamino)ethyl]-5-[(Z)-(5-fluor-1,2-dihydro-2-oxo-3H-indol-3-yliden)-methyl]-2,4-dimethyl-1H-pyrrol-3-carboxamid; Sutent™), but include also anti-VEGF or anti-VEGF receptor antibodies like e.g. bevacizumab (Avastin™).

In one preferred embodiment of the invention the compound of formula I used in the combination therapy with an anti-angiogenic agent described herein is selected from the following:
6-Amino-9-benzyl-N-methyl-8-oxo-N-propyl-2-(propylsulfonimidoyl)purine-7-carboxamide;
6-Amino-9-benzyl-N-(2-methoxyethyl)-N-methyl-8-oxo-2-(propylsulfonimidoyl)purine-7-carboxamide;
6-Amino-9-benzyl-N-ethyl-8-oxo-N-propyl-2-(propylsulfonimidoyl)purine-7-carboxamide;
6-Amino-9-benzyl-7-[4-(1-piperidyl)piperidine-1-carbonyl]-2-(propylsulfonimidoyl)purin-8-one;
6-Amino-9-benzyl-N-ethyl-N-(2-methoxyethyl)-8-oxo-2-(propylsulfonimidoyl)purine-7-carboxamide;
6-Amino-9-benzyl-N-butyl-N-ethyl-8-oxo-2-(propylsulfonimidoyl)purine-7-carboxamide;
6-Amino-9-benzyl-N-(2-methoxyethyl)-8-oxo-N-propyl-2-(propylsulfonimidoyl)purine-7-carboxamide;
6-Amino-9-benzyl-N,N-bis(2-methoxyethyl)-8-oxo-2-(propylsulfonimidoyl)purine-7-carboxamide;
6-Amino-7-(azetidine-1-carbonyl)-9-benzyl-2-(propylsulfonimidoyl)purin-8-one;
6-Amino-9-benzyl-N-isopropyl-N-methyl-8-oxo-2-(propylsulfonimidoyl)purine-7-carboxamide;
6-Amino-9-benzyl-7-(4-methylpiperazine-1-carbonyl)-2-(propylsulfonimidoyl)purin-8-one;
6-Amino-9-benzyl-N-(3-methoxypropyl)-N-methyl-8-oxo-2-(propylsulfonimidoyl)purine-7-carboxamide;
6-Amino-9-benzyl-N-isobutyl-N-methyl-8-oxo-2-(propylsulfonimidoyl)purine-7-carboxamide;
Ethyl 2-[[6-amino-9-benzyl-8-oxo-2-(propylsulfonimidoyl)purine-7-carbonyl]-methyl-amino]acetate;
Ethyl 3-[[6-amino-9-benzyl-8-oxo-2-(propylsulfonimidoyl)purine-7-carbonyl]-methyl-amino]propanoate;
tert-Butyl 3-[[6-amino-9-benzyl-8-oxo-2-(propylsulfonimidoyl)purine-7-carbonyl]-methyl-amino]propanoate;
Ethyl (2S)-2-[[6-amino-9-benzyl-8-oxo-2-(propylsulfonimidoyl)purine-7-carbonyl]-methyl-amino]propanoate;
tert-Butyl (2S)-2-[[6-amino-9-benzyl-8-oxo-2-(propylsulfonimidoyl)purine-7-carbonyl]-methyl-amino]-4-methyl-pentanoate;
Isopropyl (2S)-2-[[6-amino-9-benzyl-8-oxo-2-(propylsulfonimidoyl)purine-7-carbonyl]-methyl-amino]-4-methyl-pentanoate;
Ethyl (2S)-2-[[6-amino-9-benzyl-8-oxo-2-(propylsulfonimidoyl)purine-7-carbonyl]-methyl-amino]-3-methyl-butanoate;
Ethyl (2S)-2-[[6-amino-9-benzyl-8-oxo-2-(propylsulfonimidoyl)purine-7-carbonyl]-methyl-amino]-4-methyl-pentanoate;
Ethyl (2S)-2-[[6-amino-9-benzyl-8-oxo-2-(propylsulfonimidoyl)purine-7-carbonyl]-methyl-amino]-3-phenyl-propanoate;
Isopropyl (2S)-2-[[6-amino-9-benzyl-8-oxo-2-(propylsulfonimidoyl)purine-7-carbonyl]-methyl-amino]-3-phenyl-propanoate;
tert-Butyl (2S)-2-[[6-amino-9-benzyl-8-oxo-2-(propylsulfonimidoyl)purine-7-carbonyl]-methyl-amino]-3-phenyl-propanoate;
N-[2-[Acetyl(methyl)amino]ethyl]-6-amino-9-benzyl-N-methyl-8-oxo-2-(propylsulfonimidoyl)purine-7-carboxamide;
Methyl N-[2-[[6-amino-9-benzyl-8-oxo-2-(propylsulfonimidoyl)purine-7-carbonyl]-methyl-amino]ethyl]-N-methyl-carbamate;
tert-Butyl N-[2-[[6-amino-9-benzyl-8-oxo-2-(propylsulfonimidoyl)purine-7-carbonyl]-methyl-amino]ethyl]-N-methyl-carbamate;
Ethyl N-[2-[[6-amino-9-benzyl-8-oxo-2-(propylsulfonimidoyl)purine-7-carbonyl]-methyl-amino]ethyl]-N-methyl-carbamate;
2-[[6-Amino-9-benzyl-8-oxo-2-(propylsulfonimidoyl)purine-7-carbonyl]-methyl-amino]ethyl N-butyl-N-methyl-carbamate;
2-[[6-Amino-9-benzyl-8-oxo-2-(propylsulfonimidoyl)purine-7-carbonyl]-methyl-amino]ethyl pyrrolidine-1-carboxylate;
2-[[6-Amino-9-benzyl-8-oxo-2-(propylsulfonimidoyl)purine-7-carbonyl]-methyl-amino]ethyl N-methyl-N-propyl-carbamate;
2-[[6-Amino-9-benzyl-8-oxo-2-(propylsulfonimidoyl)purine-7-carbonyl]-methyl-amino]ethyl N,N-diethylcarbamate;
2-[[6-Amino-9-benzyl-8-oxo-2-(propylsulfonimidoyl)purine-7-carbonyl]-methyl-amino]ethyl ethyl carbonate;
6-Amino-N-butyl-9-[(4-chlorophenyl)methyl]-N-methyl-8-oxo-2-[S(S)-propylsulfonimidoyl]purine-7-carboxamide;
6-amino-N-butyl-9-[(4-chlorophenyl)methyl]-N-methyl-8-oxo-2-[S(S)-propylsulfonimidoyl]purine-7-carboxamide;
6-Amino-9-[(4-chlorophenyl)methyl]-N-ethyl-N-methyl-8-oxo-2-(propylsulfonimidoyl)purine-7-carboxamide;
6-Amino-N-methyl-8-oxo-N-propyl-2 [S(S)-propylsulfonimidoyl]-9-(p-tolylmethyl)purine-7-carboxamide;
6-Amino-N-methyl-8-oxo-N-propyl-2 [S(R)-propylsulfonimidoyl]-9-(p-tolylmethyl)purine-7-carboxamide;
6-Amino-2-[S(S)-propylsulfonimidoyl]-9-(p-tolylmethyl)-7-(pyrrolidine-1-carbonyl)purin-8-one;
6-Amino-2-[S(R)-propylsulfonimidoyl]-9-(p-tolylmethyl)-7-(pyrrolidine-1-carbonyl)purin-8-one;
6-Amino-N-(2-methoxyethyl)-N-methyl-8-oxo-2-[S(S)-propylsulfonimidoyl]-9-(p-tolylmethyl)purine-7-carboxamide;
6-Amino-N-(2-methoxyethyl)-N-methyl-8-oxo-2-[S(R)-propylsulfonimidoyl]-9-(p-tolylmethyl)purine-7-carboxamide;
6-Amino-N-ethyl-N-methyl-8-oxo-2-(propylsulfonimidoyl)-9-(p-tolylmethyl)purine-7-carboxamide;
6-Amino-N-butyl-N-methyl-8-oxo-2-(propylsulfonimidoyl)-9-(p-tolylmethyl)purine-7-carboxamide;
6-Amino-9-[(4-chlorophenyl)methyl]-2-[S(R)-ethylsulfonimidoyl]-N-methyl-8-oxo-N-propyl-purine-7-carboxamide;
6-Amino-9-[(4-chlorophenyl)methyl]-2-[S(S)-ethylsulfonimidoyl]-N-methyl-8-oxo-N-propyl-purine-7-carboxamide;
6-Amino-9-[(4-chlorophenyl)methyl]-N-ethyl-2[S(S)-ethylsulfonimidoyl]-N-methyl-8-oxo-purine-7-carboxamide;
6-Amino-9-[(4-chlorophenyl)methyl]-N-ethyl-2-[S(R)-ethylsulfonimidoyl]-N-methyl-8-oxo-purine-7-carboxamide;
6-Amino-2-[S(S)-ethylsulfonimidoyl]-N-methyl-8-oxo-N-propyl-9-(p-tolylmethyl)purine-7-carboxamide;
6-Amino-2-[S(R)-ethylsulfonimidoyl]-N-methyl-8-oxo-N-propyl-9-(p-tolylmethyl)purine-7-carboxamide;
6-Amino-N-ethyl-2 [S(S)-ethylsulfonimidoyl]-N-methyl-8-oxo-9-(p-tolylmethyl)purine-7-carboxamide;
6-Amino-N-ethyl-2-[S(R)-ethyl sulfonimidoyl]-N-methyl-8-oxo-9-(p-tolylmethyl)purine-7-carboxamide;
6-Amino-2-[S(S)ethylsulfonimidoyl]-9-[(4-fluorophenyl)methyl]-N-methyl-8-oxo-N-propyl-purine-7-carboxamide;
6-Amino-2-[S(R)ethylsulfonimidoyl]-9-[(4-fluorophenyl)methyl]-N-methyl-8-oxo-N-propyl-purine-7-carboxamide;
6-Amino-N-ethyl-2-(ethylsulfonimidoyl)-9-[(4-fluorophenyl)methyl]-N-methyl-8-oxo-purine-7-carboxamide;
6-Amino-N-ethyl-2-[S(S)-(ethylsulfonimidoyl)]-9-[(4-fluorophenyl)methyl]-N-methyl-8-oxo-purine-7-carboxamide;
6-Amino-N-ethyl-2-[S(R)-(ethylsulfonimidoyl)]-9-[(4-fluorophenyl)methyl]-N-methyl-8-oxo-purine-7-carboxamide;
6-Amino-9-[(4-bromophenyl)methyl]-2-(ethylsulfonimidoyl)-N-methyl-8-oxo-N-propyl-purine-7-carboxamide;
6-Amino-2-[S(R)-ethyl sulfonimidoyl]-9-[(4-bromophenyl)methyl]-N-methyl-8-oxo-N-propyl-purine-7-carboxamide;
6-Amino-2-[S(S)-ethyl sulfonimidoyl]-9-[(4-bromophenyl)methyl]-N-methyl-8-oxo-N-propyl-purine-7-carboxamide;
6-Amino-9-[(4-bromophenyl)methyl]-N-ethyl-2-(ethylsulfonimidoyl)-N-methyl-8-oxo-purine-7-carboxamide;
6-Amino-9-[(4-bromophenyl)methyl]-N-ethyl-2-[S(S)-(ethylsulfonimidoyl)]-N-methyl-8-oxo-purine-7-carboxamide; and
6-Amino-9-[(4-bromophenyl)methyl]-N-ethyl-2-[S(R)-(ethylsulfonimidoyl)]-N-methyl-8-oxo-purine-7-carboxamide;
   or pharmaceutically acceptable salt, enantiomer or diastereomer thereof,
   and the anti-angiogenic agent used in the combination therapy is sorafenib, regorafenib, sunitinib or bevacizumab (preferably sorafenib or bevacizumab).

In one preferred embodiment of the invention the compound of formula I used in the combination therapy with an anti-angiogenic agent described herein is selected from the following:
6-Amino-9-[(4-chlorophenyl)methyl]-N-ethyl-2[S(S)-ethylsulfonimidoyl]-N-methyl-8-oxo-purine-7-carboxamide;
6-Amino-9-[(4-chlorophenyl)methyl]-N-ethyl-2-[S(R)-ethylsulfonimidoyl]-N-methyl-8-oxo-purine-7-carboxamide;
6-Amino-9-[(4-bromophenyl)methyl]-N-ethyl-2-(ethylsulfonimidoyl)-N-methyl-8-oxo-purine-7-carboxamide;
6-Amino-9-[(4-bromophenyl)methyl]-N-ethyl-2-[S(S)-(ethylsulfonimidoyl)]-N-methyl-8-oxo-purine-7-carboxamide; or
6-Amino-9-[(4-bromophenyl)methyl]-N-ethyl-2-[S(R)-(ethylsulfonimidoyl)]-N-methyl-8-oxo-purine-7-carboxamide;
   or pharmaceutically acceptable salt, enantiomer or diastereomer thereof, (in one preferred embodiment 6-Amino-9-[(4-chlorophenyl)methyl]-N-ethyl-2[S(S)-ethylsulfonimidoyl]-N-methyl-8-oxo-purine-7-carboxamide);
   and the anti-angiogenic agent used in the combination therapy is sorafenib, regorafenib, sunitinib or bevacizumab (preferably sorafenib or bevacizumab).

In one preferred embodiment of the invention the compound of formula I used in the combination therapy with an antagonistic PD1 or antagonistic PD-L1 antibody and an anti-angiogenic agent described herein is selected from the following:
6-Amino-9-benzyl-N-methyl-8-oxo-N-propyl-2-(propylsulfonimidoyl)purine-7-carboxamide;
6-Amino-9-benzyl-N-(2-methoxyethyl)-N-methyl-8-oxo-2-(propylsulfonimidoyl)purine-7-carboxamide;
6-Amino-9-benzyl-N-ethyl-8-oxo-N-propyl-2-(propylsulfonimidoyl)purine-7-carboxamide;
6-Amino-9-benzyl-7-[4-(1-piperidyl)piperidine-1-carbonyl]-2-(propylsulfonimidoyl)purin-8-one;
6-Amino-9-benzyl-N-ethyl-N-(2-methoxyethyl)-8-oxo-2-(propylsulfonimidoyl)purine-7-carboxamide;
6-Amino-9-benzyl-N-butyl-N-ethyl-8-oxo-2-(propylsulfonimidoyl)purine-7-carboxamide;
6-Amino-9-benzyl-N-(2-methoxyethyl)-8-oxo-N-propyl-2-(propylsulfonimidoyl)purine-7-carboxamide;
6-Amino-9-benzyl-N,N-bis(2-methoxyethyl)-8-oxo-2-(propylsulfonimidoyl)purine-7-carboxamide;
6-Amino-7-(azetidine-1-carbonyl)-9-benzyl-2-(propylsulfonimidoyl)purin-8-one;
6-Amino-9-benzyl-N-isopropyl-N-methyl-8-oxo-2-(propylsulfonimidoyl)purine-7-carboxamide;
6-Amino-9-benzyl-7-(4-methylpiperazine-1-carbonyl)-2-(propylsulfonimidoyl)purin-8-one;
6-Amino-9-benzyl-N-(3-methoxypropyl)-N-methyl-8-oxo-2-(propylsulfonimidoyl)purine-7-carboxamide;
6-Amino-9-benzyl-N-isobutyl-N-methyl-8-oxo-2-(propylsulfonimidoyl)purine-7-carboxamide;
Ethyl 2-[[6-amino-9-benzyl-8-oxo-2-(propylsulfonimidoyl)purine-7-carbonyl]-methyl-amino]acetate;
Ethyl 3-[[6-amino-9-benzyl-8-oxo-2-(propylsulfonimidoyl)purine-7-carbonyl]-methyl-amino]propanoate;
tert-Butyl 3-[[6-amino-9-benzyl-8-oxo-2-(propylsulfonimidoyl)purine-7-carbonyl]-methyl-amino]propanoate;
Ethyl (2S)-2-[[6-amino-9-benzyl-8-oxo-2-(propylsulfonimidoyl)purine-7-carbonyl]-methyl-amino]propanoate;
tert-Butyl (2S)-2-[[6-amino-9-benzyl-8-oxo-2-(propylsulfonimidoyl)purine-7-carbonyl]-methyl-amino]-4-methyl-pentanoate;
Isopropyl (2S)-2-[[6-amino-9-benzyl-8-oxo-2-(propylsulfonimidoyl)purine-7-carbonyl]-methyl-amino]-4-methyl-pentanoate;
Ethyl (2S)-2-[[6-amino-9-benzyl-8-oxo-2-(propylsulfonimidoyl)purine-7-carbonyl]-methyl-amino]-3-methyl-butanoate;
Ethyl (2S)-2-[[6-amino-9-benzyl-8-oxo-2-(propylsulfonimidoyl)purine-7-carbonyl]-methyl-amino]-4-methyl-pentanoate;
Ethyl (2S)-2-[[6-amino-9-benzyl-8-oxo-2-(propylsulfonimidoyl)purine-7-carbonyl]-methyl-amino]-3-phenyl-propanoate;
Isopropyl (2S)-2-[[6-amino-9-benzyl-8-oxo-2-(propylsulfonimidoyl)purine-7-carbonyl]-methyl-amino]-3-phenyl-propanoate;
tert-Butyl (2S)-2-[[6-amino-9-benzyl-8-oxo-2-(propylsulfonimidoyl)purine-7-carbonyl]-methyl-amino]-3-phenyl-propanoate;
N-[2-[Acetyl(methyl)amino]ethyl]-6-amino-9-benzyl-N-methyl-8-oxo-2-(propylsulfonimidoyl)purine-7-carboxamide;
Methyl N-[2-[[6-amino-9-benzyl-8-oxo-2-(propylsulfonimidoyl)purine-7-carbonyl]-methyl-amino]ethyl]-N-methyl-carbamate;
tert-Butyl N-[2-[[6-amino-9-benzyl-8-oxo-2-(propylsulfonimidoyl)purine-7-carbonyl]-methyl-amino]ethyl]-N-methyl-carbamate;
Ethyl N-[2-[[6-amino-9-benzyl-8-oxo-2-(propylsulfonimidoyl)purine-7-carbonyl]-methyl-amino]ethyl]-N-methyl-carbamate;
2-[[6-Amino-9-benzyl-8-oxo-2-(propylsulfonimidoyl)purine-7-carbonyl]-methyl-amino]ethyl N-butyl-N-methyl-carbamate;
2-[[6-Amino-9-benzyl-8-oxo-2-(propylsulfonimidoyl)purine-7-carbonyl]-methyl-amino]ethyl pyrrolidine-1-carboxylate;
2-[[6-Amino-9-benzyl-8-oxo-2-(propylsulfonimidoyl)purine-7-carbonyl]-methyl-amino]ethyl N-methyl-N-propyl-carbamate;
2-[[6-Amino-9-benzyl-8-oxo-2-(propylsulfonimidoyl)purine-7-carbonyl]-methyl-amino]ethyl N,N-diethylcarbamate;
2-[[6-Amino-9-benzyl-8-oxo-2-(propylsulfonimidoyl)purine-7-carbonyl]-methyl-amino]ethyl ethyl carbonate;
6-Amino-N-butyl-9-[(4-chlorophenyl)methyl]-N-methyl-8-oxo-2-[S(S)-propylsulfonimidoyl]purine-7-carboxamide;
6-amino-N-butyl-9-[(4-chlorophenyl)methyl]-N-methyl-8-oxo-2-[S(S)-propylsulfonimidoyl]purine-7-carboxamide;
6-Amino-9-[(4-chlorophenyl)methyl]-N-ethyl-N-methyl-8-oxo-2-(propylsulfonimidoyl)purine-7-carboxamide;
6-Amino-N-methyl-8-oxo-N-propyl-2[S(S)-propylsulfonimidoyl]-9-(p-tolylmethyl)purine-7-carboxamide;
6-Amino-N-methyl-8-oxo-N-propyl-2[S(R)-propylsulfonimidoyl]-9-(p-tolylmethyl)purine-7-carboxamide;
6-Amino-2-[S(S)-propylsulfonimidoyl]-9-(p-tolylmethyl)-7-(pyrrolidine-1-carbonyl)purin-8-one;
6-Amino-2-[S(R)-propylsulfonimidoyl]-9-(p-tolylmethyl)-7-(pyrrolidine-1-carbonyl)purin-8-one;
6-Amino-N-(2-methoxyethyl)-N-methyl-8-oxo-2-[S(S)-propylsulfonimidoyl]-9-(p-tolylmethyl)purine-7-carboxamide;
6-Amino-N-(2-methoxyethyl)-N-methyl-8-oxo-2-[S(R)-propyl sulfonimidoyl]-9-(p-tolylmethyl)purine-7-carboxamide;
6-Amino-N-ethyl-N-methyl-8-oxo-2-(propylsulfonimidoyl)-9-(p-tolylmethyl)purine-7-carboxamide;
6-Amino-N-butyl-N-methyl-8-oxo-2-(propylsulfonimidoyl)-9-(p-tolylmethyl)purine-7-carboxamide;
6-Amino-9-[(4-chlorophenyl)methyl]-2-[S(R)-ethylsulfonimidoyl]-N-methyl-8-oxo-N-propyl-purine-7-carboxamide;
6-Amino-9-[(4-chlorophenyl)methyl]-2-[S(S)-ethylsulfonimidoyl]-N-methyl-8-oxo-N-propyl-purine-7-carboxamide;
6-Amino-9-[(4-chlorophenyl)methyl]-N-ethyl-2 [S(S)-ethylsulfonimidoyl]-N-methyl-8-oxo-purine-7-carboxamide;
6-Amino-9-[(4-chlorophenyl)methyl]-N-ethyl-2-[S(R)-ethylsulfonimidoyl]-N-methyl-8-oxo-purine-7-carboxamide;
6-Amino-2-[S(S)-ethylsulfonimidoyl]-N-methyl-8-oxo-N-propyl-9-(p-tolylmethyl)purine-7-carboxamide;
6-Amino-2-[S(R)-ethylsulfonimidoyl]-N-methyl-8-oxo-N-propyl-9-(p-tolylmethyl)purine-7-carboxamide;
6-Amino-N-ethyl-2 [S(S)-ethylsulfonimidoyl]-N-methyl-8-oxo-9-(p-tolylmethyl)purine-7-carboxamide;
6-Amino-N-ethyl-2-[S(R)-ethyl sulfonimidoyl]-N-methyl-8-oxo-9-(p-tolylmethyl)purine-7-carboxamide;
6-Amino-2-[S(S)ethylsulfonimidoyl]-9-[(4-fluorophenyl)methyl]-N-methyl-8-oxo-N-propyl-purine-7-carboxamide;
6-Amino-2-[S(R)ethylsulfonimidoyl]-9-[(4-fluorophenyl)methyl]-N-methyl-8-oxo-N-propyl-purine-7-carboxamide;
6-Amino-N-ethyl-2-(ethylsulfonimidoyl)-9-[(4-fluorophenyl)methyl]-N-methyl-8-oxo-purine-7-carboxamide;
6-Amino-N-ethyl-2-[S(S)-(ethylsulfonimidoyl)]-9-[(4-fluorophenyl)methyl]-N-methyl-8-oxo-purine-7-carboxamide;
6-Amino-N-ethyl-2-[S(R)-(ethylsulfonimidoyl)]-9-[(4-fluorophenyl)methyl]-N-methyl-8-oxo-purine-7-carboxamide;
6-Amino-9-[(4-bromophenyl)methyl]-2-(ethylsulfonimidoyl)-N-methyl-8-oxo-N-propyl-purine-7-carboxamide;
6-Amino-2-[S(R)-ethylsulfonimidoyl]-9-[(4-bromophenyl)methyl]-N-methyl-8-oxo-N-propyl-purine-7-carboxamide;
6-Amino-2-[S(S)-ethylsulfonimidoyl]-9-[(4-bromophenyl)methyl]-N-methyl-8-oxo-N-propyl-purine-7-carboxamide;
6-Amino-9-[(4-bromophenyl)methyl]-N-ethyl-2-(ethylsulfonimidoyl)-N-methyl-8-oxo-purine-7-carboxamide;
6-Amino-9-[(4-bromophenyl)methyl]-N-ethyl-2-[S(S)-(ethylsulfonimidoyl)]-N-methyl-8-oxo-purine-7-carboxamide; and
6-Amino-9-[(4-bromophenyl)methyl]-N-ethyl-2-[S(R)-(ethylsulfonimidoyl)]-N-methyl-8-oxo-purine-7-carboxamide;
   or pharmaceutically acceptable salt, enantiomer or diastereomer thereof;
   and i) the antagonistic PD1 antibody is nivolumab or pembrolizumab or comprises a heavy chain variable domain VH of SEQ ID NO:5 and a light chain variable domain VL of SEQ ID NO:6;
   ii) the antagonistic PD-L1 antibody is atezolizumab or durvalumab or avelumab (in one preferred embodiment atezolizumab)
   and the anti-angiogenic agent used in the combination therapy is sorafenib, regorafenib, sunitinib or bevacizumab (preferably sorafenib or bevacizumab).

In one preferred embodiment of the invention the compound of formula I used in the combination therapy with an antagonistic PD1 or antagonistic PD-L1 antibody and an anti-angiogenic agent described herein is selected from the following:
6-Amino-9-[(4-chlorophenyl)methyl]-N-ethyl-2[S(S)-ethylsulfonimidoyl]-N-methyl-8-oxo-purine-7-carboxamide;
6-Amino-9-[(4-chlorophenyl)methyl]-N-ethyl-2-[S(R)-ethylsulfonimidoyl]-N-methyl-8-oxo-purine-7-carboxamide;
6-Amino-9-[(4-bromophenyl)methyl]-N-ethyl-2-(ethylsulfonimidoyl)-N-methyl-8-oxo-purine-7-carboxamide;
6-Amino-9-[(4-bromophenyl)methyl]-N-ethyl-2-[S(S)-(ethylsulfonimidoyl)]-N-methyl-8-oxo-purine-7-carboxamide; or
6-Amino-9-[(4-bromophenyl)methyl]-N-ethyl-2-[S(R)-(ethylsulfonimidoyl)]-N-methyl-8-oxo-purine-7-carboxamide;
   or pharmaceutically acceptable salt, enantiomer or diastereomer thereof; (in one preferred embodiment 6-Amino-9-[(4-chlorophenyl)methyl]-N-ethyl-2 [S(S)-ethylsulfonimidoyl]-N-methyl-8-oxo-purine-7-carboxamide);
   and i) the antagonistic PD1 antibody is nivolumab or pembrolizumab or comprises a heavy chain variable domain VH of SEQ ID NO:5 and a light chain variable domain VL of SEQ ID NO:6;
   ii) the antagonistic PD-L1 antibody is atezolizumab or durvalumab or avelumab (in one preferred embodiment atezolizumab)
   and the anti-angiogenic agent used in the combination therapy is sorafenib, regorafenib, sunitinib or bevacizumab (preferably sorafenib or bevacizumab).

In the following specific embodiments of the invention are included:

1. A compound of formula (I),

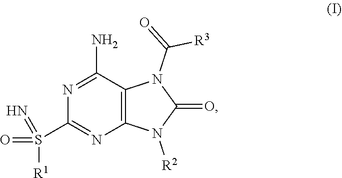

wherein
R¹ is C₁₋₆alkyl;
R² is benzyl, said benzyl being unsubstituted or substituted by one, two or three substituents independently selected from halogen and C₁₋₆alkyl;
R³ is —NR⁴R⁵, wherein
   R⁴ is C₁₋₆-alkyl or C₁₋₆alkoxyC₁₋₆alkyl;
   R⁵ is (C₁₋₆alkyl)₂NCOOC₁₋₆alkyl, C₁₋₆alkoxyC₁₋₆alkyl, C₁₋₆alkoxycarbonyl(C₁₋₆alkyl)aminoC₁₋₆ alkyl, C₁₋₆alkoxycarbonyl(phenyl)C₁₋₆alkyl, C₁₋₆alkoxycarbonylC₁₋₆alkyl, C₁₋₆ alkoxycarbonyloxyC₁₋₆alkyl, C₁₋₆alkyl, C₁₋₆alkylcarbonyl(C₁₋₆alkyl)aminoC₁₋₆alkyl or pyrrolidinylcarbamoyloxyC₁₋₆alkyl; or
   R⁴ and R⁵ together with the nitrogen they are attached to form a heterocyclyl;
      or pharmaceutically acceptable salt, enantiomer or diastereomer thereof, (or a pharmaceutical composition or medicament thereof);
      for use in the treatment or prophylaxis of liver cancer;
      with the proviso that
6-amino-9-benzyl-2-(propylsulfonimidoyl)-7-(pyrrolidine-1-carbonyl)purin-8-one;
6-amino-9-benzyl-7-(piperidine-1-carbonyl)-2-(propylsulfonimidoyl)purin-8-one;
6-amino-9-benzyl-7-(morpholine-4-carbonyl)-2-(propylsulfonimidoyl)purin-8-one;
6-amino-9-benzyl-7-(3,3-dimethylpyrrolidine-1-carbonyl)-2-(propylsulfonimidoyl)purin-8-one;
ethyl 1-[6-amino-9-benzyl-8-oxo-2-(propylsulfonimidoyl)purine-7-carbonyl]pyrrolidine-2-carboxylate;
6-amino-7-(2-azaspiro[3.3]heptane-2-carbonyl)-9-benzyl-2-(propylsulfonimidoyl)purin-8-one;
6-amino-9-benzyl-7-(2-oxa-6-azaspiro[3.3]heptane-6-carbonyl)-2-(propylsulfonimidoyl)purin-8-one;
6-amino-9-benzyl-7-(3,3-difluoropyrrolidine-1-carbonyl)-2-(propylsulfonimidoyl)purin-8-one;
6-amino-9-benzyl-7-(3-fluoro-3-methyl-pyrrolidine-1-carbonyl)-2-(propylsulfonimidoyl)purin-8-one;
   and their enantiomers or diastereomers are excluded.

2. A compound for use according to embodiment 1, wherein
R¹ is C₁₋₆alkyl;
R² is benzyl, said benzyl being unsubstituted or substituted by halogen or C₁₋₆alkyl;
R³ is azetidinyl;
   piperazinyl substituted by C₁₋₆alkyl;
   piperidinyl substituted by piperidinyl;
   pyrrolidinyl; or
   —NR⁴R, wherein
      R⁴ is C₁₋₆-alkyl or C₁₋₆alkoxyC₁₋₆alkyl;
      R⁵ is (C₁₋₆alkyl)₂NCOOC₁₋₆alkyl, C₁₋₆alkoxyC₁₋₆alkyl, C₁₋₆alkoxycarbonyl(C₁₋₆alkyl)aminoC₁₋₆ alkyl, C₁₋₆alkoxycarbonyl(phenyl)C₁₋₆alkyl, C₁₋₆alkoxycarbonylC₁₋₆alkyl, C₁₋₆ alkoxycarbonyloxyC₁₋₆alkyl, C₁₋₆alkyl, C₁₋₆alkylcarbonyl(C₁₋₆alkyl)aminoC₁₋₆alkyl or pyrrolidinylcarbamoyloxyC₁₋₆alkyl.
3. A compound for use according to embodiment 1 or 2, wherein
R¹ is ethyl or propyl;
R² is benzyl, bromobenzyl, chlorobenzyl, fluorobenzyl or methylbenzyl;
R³ is azetidinyl;
   4-methylpiperazinyl;
   piperidinylpiperidinyl;
   pyrrolidinyl; or
   —NR⁴R⁵, wherein
      R⁴ is methyl, ethyl, propyl or methoxyethyl;
      R⁵ is acetyl(methyl)aminoethyl, butyl, butyl(methyl)carbamoyloxyethyl, diethylcarbamoyloxyethyl, ethoxycarbonyl(methyl)aminoethyl, ethoxycarbonylethyl, ethoxycarbonylisobutyl, ethoxycarbonylisopentyl, ethoxycarbonylmethyl, ethoxycarbonyloxyethyl, ethoxycarbonyl(phenyl)ethyl, ethyl, isobutyl, isopropoxycarbonylisopentyl, isopropoxycarbonyl(phenyl)ethyl, isopropyl, methoxycarbonyl(methyl)aminoethyl, methoxyethyl, methoxypropyl, propyl, propyl(methyl)carbamoyloxyethyl, pyrrolidinylcarbamoyloxyethyl, tert-butoxycarbonyl(methyl)aminoethyl, tert-butoxycarbonylethyl, tert-butoxycarbonylisopentyl or tert-butoxycarbonyl(phenyl)ethyl.

4. A compound for use according to embodiment 3, wherein R³ is azetidinyl, 4-methylpiperazinyl, piperidinylpiperidinyl, pyrrolidinyl, acetyl(methyl)aminoethyl(methyl)amino, bis(methoxyethyl)amino, butyl(ethyl)amino, butyl(methyl)amino, butyl(methyl)carbamoyloxyethyl(methyl)amino, diethylcarbamoyloxyethyl(methyl)amino, ethoxycarbonyl(methyl)aminoethyl(methyl)amino, ethoxycarbonylethyl(methyl)amino, ethoxycarbonylisobutyl(methyl)amino, ethoxycarbonylisopentyl(methyl)amino, ethoxycarbonylmethyl(methyl)amino, ethoxycarbonyloxyethyl(methyl)amino, ethoxycarbonyl(phenyl)ethyl(methyl)amino, ethyl(methyl)amino, isobutyl(methyl)amino, isopropoxycarbonylisopentyl(methyl)amino, isopropoxycarbonyl(phenyl)ethyl(methyl)amino, isopropyl(methyl)amino, methoxycarbonyl(methyl)aminoethyl(methyl)amino, methoxyethyl(ethyl)amino, methoxyethyl(methyl)amino, methoxyethyl(propyl)amino, methoxypropyl(methyl)amino, propyl(ethyl)amino, propyl(methyl)amino, propyl(methyl)carbamoyloxyethyl(methyl)amino, pyrrolidinylcarbamoyloxyethyl(methyl)amino, tert-butoxycarbonyl(methyl)aminoethyl(methyl)amino, tert-butoxycarbonylethyl(methyl)amino, tert-butoxycarbonylisopentyl(methyl)amino or tert-butoxycarbonyl(phenyl)ethyl(methyl)amino.

5. A compound for use according to any one of embodiments 1 to 4, wherein R¹ is ethyl.

6. A compound for use according to embodiment 1 or 2, wherein R² is benzyl substituted by halogen or C₁₋₆ alkyl.

7. A compound for use according to any one of embodiments 2 to 6, wherein R² is bromobenzyl, chlorobenzyl, fluorobenzyl or methylbenzyl.

8. A compound for use according to embodiment 7, wherein R² is bromobenzyl, chlorobenzyl or fluorobenzyl.

9. A compound for use according to embodiment 1 or 2, wherein R³ is —NR⁴R⁵, wherein R⁴ is C₁₋₆-alkyl, R⁵ is C₁₋₆alkyl.

10. A compound for use according to embodiment 9, wherein R³ is propyl(methyl)amino or ethyl(methyl)amino.

11. A compound for use according to any one of embodiments 1, 2, 6 and 9, wherein
R¹ is C₁₋₆alkyl;
R² is benzyl, said benzyl being substituted by halogen or C₁₋₆alkyl;
R³ is —NR⁴R⁵, wherein R⁴ is C₁₋₆alkyl, R⁵ is C₁₋₆alkyl.

12. A compound for use according to embodiment 11, wherein
R¹ is ethyl;
R² is methylbenzyl, bromobenzyl, chlorobenzyl or fluorobenzyl;
R³ is propyl(methyl)amino or ethyl(methyl)amino.

13. A compound for use in the treatment or prophylaxis of liver cancer selected from:
6-Amino-9-benzyl-N-methyl-8-oxo-N-propyl-2-(propylsulfonimidoyl)purine-7-carboxamide;
6-Amino-9-benzyl-N-(2-methoxyethyl)-N-methyl-8-oxo-2-(propylsulfonimidoyl)purine-7-carboxamide;
6-Amino-9-benzyl-N-ethyl-8-oxo-N-propyl-2-(propylsulfonimidoyl)purine-7-carboxamide;
6-Amino-9-benzyl-7-[4-(1-piperidyl)piperidine-1-carbonyl]-2-(propylsulfonimidoyl)purin-8-one;
6-Amino-9-benzyl-N-ethyl-N-(2-methoxyethyl)-8-oxo-2-(propylsulfonimidoyl)purine-7-carboxamide;
6-Amino-9-benzyl-N-butyl-N-ethyl-8-oxo-2-(propylsulfonimidoyl)purine-7-carboxamide;
6-Amino-9-benzyl-N-(2-methoxyethyl)-8-oxo-N-propyl-2-(propylsulfonimidoyl)purine-7-carboxamide;
6-Amino-9-benzyl-N,N-bis(2-methoxyethyl)-8-oxo-2-(propylsulfonimidoyl)purine-7-carboxamide;
6-Amino-7-(azetidine-1-carbonyl)-9-benzyl-2-(propylsulfonimidoyl)purin-8-one;
6-Amino-9-benzyl-N-isopropyl-N-methyl-8-oxo-2-(propylsulfonimidoyl)purine-7-carboxamide;
6-Amino-9-benzyl-7-(4-methylpiperazine-1-carbonyl)-2-(propylsulfonimidoyl)purin-8-one;
6-Amino-9-benzyl-N-(3-methoxypropyl)-N-methyl-8-oxo-2-(propylsulfonimidoyl)purine-7-carboxamide;
6-Amino-9-benzyl-N-isobutyl-N-methyl-8-oxo-2-(propylsulfonimidoyl)purine-7-carboxamide;
Ethyl 2-[[6-amino-9-benzyl-8-oxo-2-(propylsulfonimidoyl)purine-7-carbonyl]-methyl-amino]acetate;
Ethyl 3-[[6-amino-9-benzyl-8-oxo-2-(propylsulfonimidoyl)purine-7-carbonyl]-methyl-amino]propanoate;
tert-Butyl 3-[[6-amino-9-benzyl-8-oxo-2-(propylsulfonimidoyl)purine-7-carbonyl]-methyl-amino]propanoate;
Ethyl (2S)-2-[[6-amino-9-benzyl-8-oxo-2-(propylsulfonimidoyl)purine-7-carbonyl]-methyl-amino]propanoate;
tert-Butyl (2S)-2-[[6-amino-9-benzyl-8-oxo-2-(propylsulfonimidoyl)purine-7-carbonyl]-methyl-amino]-4-methyl-pentanoate;
Isopropyl (2S)-2-[[6-amino-9-benzyl-8-oxo-2-(propylsulfonimidoyl)purine-7-carbonyl]-methyl-amino]-4-methyl-pentanoate;
Ethyl (2S)-2-[[6-amino-9-benzyl-8-oxo-2-(propylsulfonimidoyl)purine-7-carbonyl]-methyl-amino]-3-methyl-butanoate;
Ethyl (2S)-2-[[6-amino-9-benzyl-8-oxo-2-(propylsulfonimidoyl)purine-7-carbonyl]-methyl-amino]-4-methyl-pentanoate;
Ethyl (2S)-2-[[6-amino-9-benzyl-8-oxo-2-(propylsulfonimidoyl)purine-7-carbonyl]-methyl-amino]-3-phenyl-propanoate;
Isopropyl (2S)-2-[[6-amino-9-benzyl-8-oxo-2-(propylsulfonimidoyl)purine-7-carbonyl]-methyl-amino]-3-phenyl-propanoate;
tert-Butyl (2S)-2-[[6-amino-9-benzyl-8-oxo-2-(propylsulfonimidoyl)purine-7-carbonyl]-methyl-amino]-3-phenyl-propanoate;
N-[2-[Acetyl(methyl)amino]ethyl]-6-amino-9-benzyl-N-methyl-8-oxo-2-(propylsulfonimidoyl)purine-7-carboxamide;
Methyl N-[2-[[6-amino-9-benzyl-8-oxo-2-(propylsulfonimidoyl)purine-7-carbonyl]-methyl-amino]ethyl]-N-methyl-carbamate;
tert-Butyl N-[2-[[6-amino-9-benzyl-8-oxo-2-(propylsulfonimidoyl)purine-7-carbonyl]-methyl-amino]ethyl]-N-methyl-carbamate;
Ethyl N-[2-[[6-amino-9-benzyl-8-oxo-2-(propylsulfonimidoyl)purine-7-carbonyl]-methyl-amino]ethyl]-N-methyl-carbamate;
2-[[6-Amino-9-benzyl-8-oxo-2-(propylsulfonimidoyl)purine-7-carbonyl]-methyl-amino]ethyl N-butyl-N-methyl-carbamate;
2-[[6-Amino-9-benzyl-8-oxo-2-(propylsulfonimidoyl)purine-7-carbonyl]-methyl-amino]ethyl pyrrolidine-1-carboxylate;
2-[[6-Amino-9-benzyl-8-oxo-2-(propylsulfonimidoyl)purine-7-carbonyl]-methyl-amino]ethyl N-methyl-N-propyl-carbamate;
2-[[6-Amino-9-benzyl-8-oxo-2-(propylsulfonimidoyl)purine-7-carbonyl]-methyl-amino]ethyl N,N-diethylcarbamate;
2-[[6-Amino-9-benzyl-8-oxo-2-(propylsulfonimidoyl)purine-7-carbonyl]-methyl-amino]ethyl ethyl carbonate;
6-Amino-N-butyl-9-[(4-chlorophenyl)methyl]-N-methyl-8-oxo-2-[S(S)-propylsulfonimidoyl]purine-7-carboxamide;
6-amino-N-butyl-9-[(4-chlorophenyl)methyl]-N-methyl-8-oxo-2-[S(S)-propylsulfonimidoyl]purine-7-carboxamide;
6-Amino-9-[(4-chlorophenyl)methyl]-N-ethyl-N-methyl-8-oxo-2-(propylsulfonimidoyl)purine-7-carboxamide;
6-Amino-N-methyl-8-oxo-N-propyl-2[S(S)-propylsulfonimidoyl]-9-(p-tolylmethyl)purine-7-carboxamide;
6-Amino-N-methyl-8-oxo-N-propyl-2[S(R)-propylsulfonimidoyl]-9-(p-tolylmethyl)purine-7-carboxamide;
6-Amino-2-[S(S)-propylsulfonimidoyl]-9-(p-tolylmethyl)-7-(pyrrolidine-1-carbonyl)purin-8-one;
6-Amino-2-[S(R)-propylsulfonimidoyl]-9-(p-tolylmethyl)-7-(pyrrolidine-1-carbonyl)purin-8-one;
6-Amino-N-(2-methoxyethyl)-N-methyl-8-oxo-2-[S(S)-propylsulfonimidoyl]-9-(p-tolylmethyl)purine-7-carboxamide;
6-Amino-N-(2-methoxyethyl)-N-methyl-8-oxo-2-[S(R)-propylsulfonimidoyl]-9-(p-tolylmethyl)purine-7-carboxamide;
6-Amino-N-ethyl-N-methyl-8-oxo-2-(propylsulfonimidoyl)-9-(p-tolylmethyl)purine-7-carboxamide;
6-Amino-N-butyl-N-methyl-8-oxo-2-(propylsulfonimidoyl)-9-(p-tolylmethyl)purine-7-carboxamide;
6-Amino-9-[(4-chlorophenyl)methyl]-2-[S(R)-ethylsulfonimidoyl]-N-methyl-8-oxo-N-propyl-purine-7-carboxamide;
6-Amino-9-[(4-chlorophenyl)methyl]-2-[S(S)-ethylsulfonimidoyl]-N-methyl-8-oxo-N-propyl-purine-7-carboxamide;
6-Amino-9-[(4-chlorophenyl)methyl]-N-ethyl-2 [S(S)-ethylsulfonimidoyl]-N-methyl-8-oxo-purine-7-carboxamide;
6-Amino-9-[(4-chlorophenyl)methyl]-N-ethyl-2-[S(R)-ethylsulfonimidoyl]-N-methyl-8-oxo-purine-7-carboxamide;
6-Amino-2-[S(S)-ethylsulfonimidoyl]-N-methyl-8-oxo-N-propyl-9-(p-tolylmethyl)purine-7-carboxamide;
6-Amino-2-[S(R)-ethyl sulfonimidoyl]-N-methyl-8-oxo-N-propyl-9-(p-tolylmethyl)purine-7-carboxamide;
6-Amino-N-ethyl-2 [S(S)-ethylsulfonimidoyl]-N-methyl-8-oxo-9-(p-tolylmethyl)purine-7-carboxamide;
6-Amino-N-ethyl-2-[S(R)-ethylsulfonimidoyl]-N-methyl-8-oxo-9-(p-tolylmethyl)purine-7-carboxamide;
6-Amino-2-[S(S)ethylsulfonimidoyl]-9-[(4-fluorophenyl)methyl]-N-methyl-8-oxo-N-propyl-purine-7-carboxamide;
6-Amino-2-[S(R)ethylsulfonimidoyl]-9-[(4-fluorophenyl)methyl]-N-methyl-8-oxo-N-propyl-purine-7-carboxamide;
6-Amino-N-ethyl-2-(ethylsulfonimidoyl)-9-[(4-fluorophenyl)methyl]-N-methyl-8-oxo-purine-7-carboxamide;
6-Amino-N-ethyl-2-[S(S)-(ethylsulfonimidoyl)]-9-[(4-fluorophenyl)methyl]-N-methyl-8-oxo-purine-7-carboxamide;
6-Amino-N-ethyl-2-[S(R)-(ethylsulfonimidoyl)]-9-[(4-fluorophenyl)methyl]-N-methyl-8-oxo-purine-7-carboxamide;
6-Amino-9-[(4-bromophenyl)methyl]-2-(ethylsulfonimidoyl)-N-methyl-8-oxo-N-propyl-purine-7-carboxamide;
6-Amino-2-[S(R)-ethylsulfonimidoyl]-9-[(4-bromophenyl)methyl]-N-methyl-8-oxo-N-propyl-purine-7-carboxamide;
6-Amino-2-[S(S)-ethylsulfonimidoyl]-9-[(4-bromophenyl)methyl]-N-methyl-8-oxo-N-propyl-purine-7-carboxamide;
6-Amino-9-[(4-bromophenyl)methyl]-N-ethyl-2-(ethylsulfonimidoyl)-N-methyl-8-oxo-purine-7-carboxamide;
6-Amino-9-[(4-bromophenyl)methyl]-N-ethyl-2-[S(S)-(ethylsulfonimidoyl)]-N-methyl-8-oxo-purine-7-carboxamide; and
6-Amino-9-[(4-bromophenyl)methyl]-N-ethyl-2-[S(R)-(ethylsulfonimidoyl)]-N-methyl-8-oxo-purine-7-carboxamide;
   or pharmaceutically acceptable salt, enantiomer or diastereomer thereof.

14. A compound according to embodiment 13, selected from:
6-Amino-9-[(4-chlorophenyl)methyl]-N-ethyl-2 [S(S)-ethylsulfonimidoyl]-N-methyl-8-oxo-purine-7-carboxamide;
6-Amino-9-[(4-chlorophenyl)methyl]-N-ethyl-2-[S(R)-ethylsulfonimidoyl]-N-methyl-8-oxo-purine-7-carboxamide;
6-Amino-9-[(4-bromophenyl)methyl]-N-ethyl-2-(ethylsulfonimidoyl)-N-methyl-8-oxo-purine-7-carboxamide;
6-Amino-9-[(4-bromophenyl)methyl]-N-ethyl-2-[S(S)-(ethylsulfonimidoyl)]-N-methyl-8-oxo-purine-7-carboxamide; and
6-Amino-9-[(4-bromophenyl)methyl]-N-ethyl-2-[S(R)-(ethylsulfonimidoyl)]-N-methyl-8-oxo-purine-7-carboxamide;
   or pharmaceutically acceptable salt, enantiomer or diastereomer thereof.

15. The compound or pharmaceutically acceptable salt, enantiomer or diastereomer for use according to any one of embodiments 1 to 14, wherein the liver cancer is hepatocellular carcinoma, hepatoma, cholangiocarcinoma, hepatoblastoma, hepatic carcinoma, hepatic angiosarcoma, or metastatic liver cancer.

16. The compound or pharmaceutically acceptable salt, enantiomer or diastereomer for use according to any one of embodiments 1 to 14, wherein the liver cancer is hepatocellular carcinoma.

17. A pharmaceutical composition or medicament comprising a compound in accordance with any one of embodiments 1 to 14 and a therapeutically inert carrier, for use in the treatment or prophylaxis of liver cancer.

18. The use of a compound according to any one of embodiments 1 to 14 for the preparation of a medicament for the treatment or prophylaxis of liver cancer.

19. A method for the treatment or prophylaxis of liver cancer, which method comprises administering a therapeutically effective amount of a compound as defined in any one of embodiments 1 to 14.

20. A compound as defined in any one of embodiments 1 to 14, or a pharmaceutical composition or a medicament comprising such compound for use in
a) the treatment or prophylaxis of liver cancer in combination with an antagonistic PD1 antibody or antagonistic PD-L1 antibody,
or
b) the treatment of a patient suffering from liver cancer in combination with an antagonistic PD1 antibody or antagonistic PD-L1 antibody.

21. A compound as defined in any one of embodiments 1 to 14, or a pharmaceutical composition or a medicament comprising such compound
for use in the treatment or prophylaxis of liver cancer
wherein the treatment is in combination with an antagonistic PD1 antibody or antagonistic PD-L1 antibody.

22. Use of a compound as defined in any one of embodiments 1 to 14;
for the preparation of a medicament for the treatment or prophylaxis of liver cancer
wherein the treatment is in combination with an antagonistic PD1 antibody or antagonistic PD-L1 antibody.

23. The compound, composition, medicament or use, according to any one of embodiments 20 to 22, wherein the treatment is in combination with an antagonistic PD1 antibody.

24. The compound, composition, medicament or use, according to embodiment 23, wherein the antagonistic PD1 antibody is nivolumab or pemprolizumab.

25. The compound, composition, medicament or use, according to embodiment 24, wherein the compound is 6-Amino-9-[(4-chlorophenyl)methyl]-N-ethyl-2[S(S)-ethylsulfonimidoyl]-N-methyl-8-oxo-purine-7-carboxamide

26. The compound, composition, medicament or use, according to embodiment 23, wherein the antagonistic PD1 antibody comprises a heavy chain variable domain VH with an amino acid sequence of SEQ ID NO: 5 and a light chain variable domain VL with an amino acid sequence of SEQ ID NO:6.

27. The compound, composition, medicament or use, according to embodiment 26, wherein the compound is 6-Amino-9-[(4-chlorophenyl)methyl]-N-ethyl-2[S(S)-ethylsulfonimidoyl]-N-methyl-8-oxo-purine-7-carboxamide.

28. The compound, composition, medicament or use, according to any one of embodiments 20 to 22, wherein the treatment is in combination with an antagonistic PD-L1 antibody.

29. The compound, composition, medicament or use, according to embodiment 28,

wherein the antagonistic PD-L1 antibody used in the combination therapy is atezolizumab or durvalumab or avelumab (in one preferred embodiment atezolizumab)

30. The compound, composition, medicament or use, according to embodiment 29, wherein the compound is 6-Amino-9-[(4-chlorophenyl)methyl]-N-ethyl-2[S(S)-ethylsulfonimidoyl]-N-methyl-8-oxo-purine-7-carboxamide.

31. The compound, composition, medicament or use according to any one of embodiments 20 to 30 wherein additionally an anti-angiogenic agent is used in the combination therapy

32. The compound, composition, medicament or use according to any one of embodiments 20 to 30 wherein additionally an anti-angiogenic agent selected from is sorafenib, regorafenib, sunitinib or bevacizumab (in one preferred embodiment the anti-angiogenic agent is sorafenib; in one preferred embodiment the anti-angiogenic agent is bevacizumab) is used in the combination therapy.

33. A compound as defined in any one of embodiments 1 to 14, or a pharmaceutical composition or a medicament comprising such compound for use in
a) the treatment or prophylaxis of liver cancer in combination with an anti-angiogenic agent,
or
b) the treatment of a patient suffering from liver cancer in combination with an anti-angiogenic agent.

34. A compound as defined in any one of embodiments 1 to 14, or a pharmaceutical composition or a medicament comprising such compound
for use in the treatment or prophylaxis of liver cancer
   wherein the treatment is in combination with an anti-angiogenic agent.

35. Use of a compound as defined in any one of embodiments 1 to 14;
for the preparation of a medicament for the treatment or prophylaxis of liver cancer
wherein the treatment is in combination with an anti-angiogenic agent.

36. The compound, composition, medicament or use according to any one of embodiments 33 to 35 wherein the anti-angiogenic agent selected from is sorafenib, regorafenib, sunitinib or bevacizumab (in one preferred embodiment the anti-angiogenic agent is sorafenib; in one preferred embodiment the anti-angiogenic agent is bevacizumab).

37. The compound, composition, medicament or use, according to embodiment 36, wherein the compound is 6-Amino-9-[(4-chlorophenyl)methyl]-N-ethyl-2[S(S)-ethylsulfonimidoyl]-N-methyl-8-oxo-purine-7-carboxamide.

38. The invention as hereinbefore described.

### DESCRIPTION OF THE AMINO ACID SEQUENCES

SEQ ID NO: 1 heavy chain variable domain of anti-PD1 antibody nivolumab
SEQ ID NO: 2 light chain variable domain of anti-PD1 antibody nivolumab
SEQ ID NO: 3 heavy chain variable domain of anti-PD1 antibody pembrolizumab
SEQ ID NO: 4 light chain variable domain of anti-PD1 antibody pembrolizumab
SEQ ID NO: 5 heavy chain variable domain of anti-PD1 antibody PD1-0103-0312
SEQ ID NO: 6 light chain variable domain of anti-PD1 antibody PD1-0103-0312
SEQ ID NO: 7 heavy chain variable domain of anti-PD-L1 antibody atezolizumab
SEQ ID NO: 8 light chain variable domain of anti-PD-L1 antibody atezolizumab
SEQ ID NO: 9 heavy chain variable domain of anti-PD-L1 antibody durvalumab
SEQ ID NO: 10 light chain variable domain of anti-PD-L1 antibody durvalumab
SEQ ID NO: 11 heavy chain variable domain of anti-PD-L1 antibody avelumab
SEQ ID NO: 12 light chain variable domain of anti-PD-L1 antibody avelumab
SEQ ID NO: 13 exemplary human PD-L1
SEQ ID NO: 14 exemplary human PD1
SEQ ID NO: 15 human kappa light chain constant region
SEQ ID NO: 16 human heavy chain constant region derived from IgG1
SEQ ID NO: 17 human heavy chain constant region derived from IgG1 mutated on L234A, L235A, P329G.

### BRIEF DESCRIPTION OF THE FIGURES

FIG. 1: Combination of an active form of the compounds of the present invention (compound 41-A) and Sorafenib results in 2 tumor-free mice in the iAST mouse model of hepatocellular carcinoma.

FIG. 2: Treatment with an active form of the compounds of the present invention (compound 41-A) induces PD-L1 expression on tumor cells in the iAST mouse model of hepatocellular carcinoma.

FIG. 3: Treatment with an active form of the compounds of the present invention (compound 41-A) results in tumor stasis in the transplanted Hep55.1c mouse model of hepatocellular carcinoma.

FIG. 4: Combination of an active form of the compounds of the present invention (compound 41-A) and anti-PD-1 antibodies results in survival benefit in the Hep55.1c mouse model of hepatocellular carcinoma.

FIG. 5: Treatment with an active form of the compounds of the present invention (compound 41c-B) does not induce enhanced tumor cell proliferation in cell lines originating from hepatocellular carcinoma and cholangiocarcinoma.

FIG. 6: Factors released in peripheral blood upon treatment with an active form of the compounds of the present invention (compound 41c-B) result in inhibition of proliferation in tumor cell lines.

FIG. 7: Single crystal X-ray diffraction of Example 41-B.

FIG. 8: Single crystal X-ray diffraction of Example 42-A.

FIG. 9: Single crystal X-ray diffraction of Example 43-B.

### EXAMPLES

The invention will be more fully understood by reference to the following examples. They should not, however, be construed as limiting the scope of the invention.

### ABBREVIATIONS

aq. aqueous
BSA: N, O-bis(trimethylsilyl)acetamide
CDI: N,N′-carbonyl diimidazole
DIEPA: N, N-diethylpropylamine
DBU: 1,8-Diazabicycloundec-7-ene
DPPA: diphenylphosphoryl azide
EC₅₀: the molar concentration of an agonist, which produces 50% of the maximum possible response for that agonist.
EDC: N1-((ethylimino)methylene)-N3N3-dimethylpropane-1,3-diamine
EtOAc or EA: ethyl acetate
HATU: (1-[Bis(dimethylamino)methylene]-1H-1,2,3-triazolo[4,5-b]pyridinium 3-oxid hexafluorophosphate)
hr(s): hour(s)
HPLC: high performance liquid chromatography
HOBt: N-hydroxybenzotriazole
MS (ESI): mass spectroscopy (electron spray ionization)
m-CPBA: 3-chloroperbenzoic acid
MTEB: methyl tert-butyl ether
NMP: N-methylpyrrolidone
obsd. observed
PE: petroleum ether
PMB: p-methoxybenzyl
PPA: polyphosphoric acid
QOD every other day
QW once a week
RT or rt: room temperature
sat. saturated
TFA: trifluoroacetic acid
TEA: triethylamine
V/V volume ratio

### General Experimental Conditions

Intermediates and final compounds were purified by flash chromatography using one of the following instruments: i) Biotage SP1 system and the Quad 12/25 Cartridge module. ii) ISCO combi-flash chromatography instrument. Silica gel Brand and pore size: i) KP-SIL 60 Å, particle size: 40-60 μm; ii) CAS registry NO: Silica Gel: 63231-67-4, particle size: 47-60 micron silica gel; iii) ZCX from Qingdao Haiyang Chemical Co., Ltd, pore: 200-300 or 300-400.

Intermediates and final compounds were purified by preparative HPLC on reversed phase column using X Bridge™ Perp C₁₈ (5 μm, OBD™ 30×100 mm) column or SunFire™ Perp C₁₈ (5 μm, OBD™ 30×100 mm) column.

LC/MS spectra were obtained using a Waters UPLC-SQD Mass. Standard LC/MS conditions were as follows (running time 3 minutes):

Acidic condition: A: 0.1% formic acid and 1% acetonitrile in H₂O; B: 0.1% formic acid in acetonitrile;

Basic condition: A: 0.05% NH₃H₂O in H₂O; B: acetonitrile.

Mass spectra (MS): generally only ions which indicate the parent mass are reported, and unless otherwise stated the mass ion quoted is the positive mass ion (M+H)⁺.

NMR Spectra were obtained using Bruker Avance 400 MHz.

All reactions involving air-sensitive reagents were performed under an argon atmosphere. Reagents were used as received from commercial suppliers without further purification unless otherwise noted.

### PREPARATIVE EXAMPLES

### Preparation of Intermediate

### Intermediate AA

### N-methyl-N-propyl-carbamoyl chloride

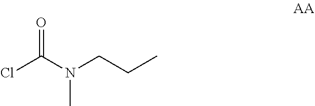

To a mixture of N-methylpropan-1-amine (5 g, 68.4 mmol) and sodium hydrogencarbonate (11.5 g, 137 mmol) in DCM (70 mL) at 0° C. was added bis(trichloromethyl) carbonate (8.11 g, 27.3 mmol) in DCM (30 mL) dropwise. The mixture was stirred at room temperature for 2 hrs and filtered. The filtrate was concentrated in vacuo. The obtained N-methyl-N-propyl-carbamoyl chloride (7.2 g, Intermediate AA) was used for next step without further purification.

### Intermediate AB

### N-(2-Methoxyethyl)-N-methyl-carbamoyl chloride

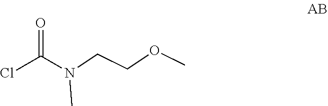

Intermediate AB was prepared in analogy to Intermediate AA by using 2-methoxy-N-methyl-ethanamine instead of N-methylpropan-1-amine. N-(2-Methoxyethyl)-N-methyl-carbamoyl chloride (8 g, Intermediate AB) was obtained and used for next step without further purification.

### Intermediate AC

### N-Ethyl-N-propyl-carbamoyl chloride

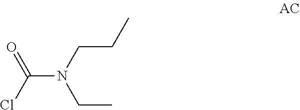

Intermediate AC was prepared in analogy to Intermediate AA by using N-ethylpropan-1-amine instead of N-methylpropan-1-amine. N-Ethyl-N-propyl-carbamoyl chloride (12.6 g, Intermediate AC) was obtained as a yellow oil and used for next step without further purification.

### Intermediate AD

### N-Ethyl-N-(2-methoxyethyl)carbamoyl chloride

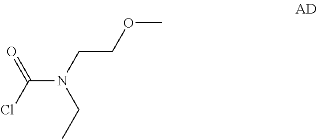

Intermediate AD was prepared in analogy to Intermediate AA by using N-ethyl-2-methoxyethanamine instead of N-methylpropan-1-amine. The crude N-ethyl-N-(2-methoxyethyl)carbamoyl chloride (2.5 g, Intermediate AD) was obtained as a light yellow oil and used for next step without further purification.

### Intermediate AE

### N-Butyl-N-ethyl-carbamoyl chloride

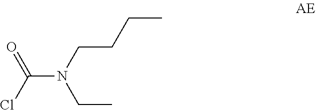

Intermediate AE was prepared in analogy to Intermediate AA by using N-ethylbutan-1-amine (5 g) instead of N-methylpropan-1-amine. The crude N-butyl-N-ethyl-carbamoyl chloride (6.3 g, Intermediate AE) was obtained as a light yellow oil and used for next step without further purification.

### Intermediate AF

### N-(2-Methoxyethyl)-N-propyl-carbamoyl chloride

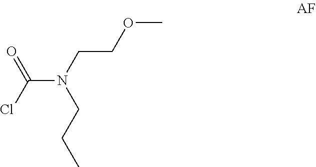

Intermediate AF was prepared in analogy to Intermediate AA by using N-(2-methoxyethyl)propan-1-amine (2 g, 17.1 mmol) instead of N-methylpropan-1-amine. The crude N-(2-methoxyethyl)-N-propyl-carbamoyl chloride (2.5 g, Intermediate AF) was obtained as a light yellow oil and used for next step without further purification.

### Intermediate AG

### N,N-Bis(2-methoxyethyl)carbamoyl chloride

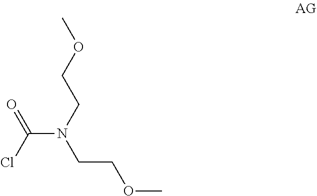

Intermediate AG was prepared in analogy to Intermediate AA by using of bis(2-methoxyethyl)amine (2 g, 15 mmol) instead of N-methylpropan-1-amine. The crude product N,N-bis(2-methoxyethyl)carbamoyl chloride (2.6 g, Intermediate AG) was obtained as a light yellow oil and used for next step without further purification.

Intermediate AH Azetidine-1-carbonyl chloride

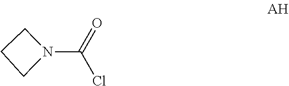

Intermediate AH was prepared in analogy to Intermediate AA by using azetidine hydrochloride (10.7 g, 107 mmol) and sodium bicarbonate (3 equiv.) instead of N-methylpropan-1-amine and sodium bicarbonate (2 equiv.). The crude azetidine-1-carbonyl chloride (1.5 g, Intermediate AH) was obtained as a light yellow oil and used for next step without further purification.

### Intermediate AI

### N-Isopropyl-N-methyl-carbamoyl chloride

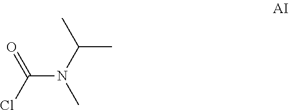

Intermediate AI was prepared in analogy to Intermediate AA by using N-methylpropan-2-amine (5 g, 19.4 mmol) instead of N-methylpropan-1-amine. The crude N-isopropyl-N-methyl-carbamoyl chloride (8.6 g, Intermediate AI) was obtained as a yellow oil and used for next step without further purification.

### Intermediate AL

### N-Isobutyl-N-methyl-carbamoyl chloride

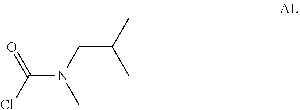

Intermediate AL was prepared in analogy to Intermediate AA by using N-2-dimethylpropan-1-amine (4.8 g) instead of N-methylpropan-1-amine. The crude N-isobutyl-N-methyl-carbamoyl chloride (8.1 g, Intermediate AL) was obtained as a light yellow oil and used for next step without further purification.

### Intermediate AP

### Ethyl 2-[chlorocarbonyl(methyl)amino]acetate

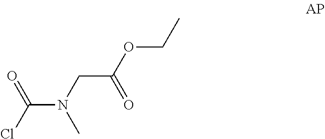

To a solution of triphosgene (728 mg, 2.45 mmol) in DCM (5 mL) was added a solution of ethyl 2-(methylamino)acetate hydrochloride (1.3 g, 8.46 mmol) and pyridine (1 mL) in DCM (5 mL) dropwise at 0° C. The reaction mixture became orange and a yellow precipitate appeared, then it was allowed to warm to room temperature. After stirred for 1 hr, aqueous HCl (0.1N, 25 mL) was added to the reaction mixture, the organic layer was separated, washed with 0.1 N HCl (10 mL) twice, brine (10 mL), dried over Na₂SO₄ and concentrated in vacuo to give the crude ethyl 2-[chlorocarbonyl(methyl)amino]acetate (2.0 g, Intermediate AP) as a light yellow oil and used for next step without further purification.

### Intermediate AR

### tert-Butyl 3-[chlorocarbonyl(methyl)amino]propanoate

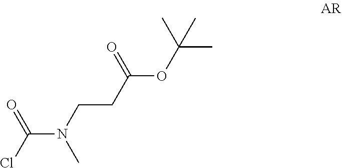

### Step 1: Preparation of tert-butyl 3-(methylamino)propanoate (Compound AR-1)

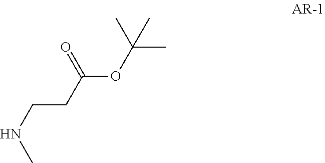

To a solution of tert-butyl acrylate (3 g) in DMF (40 mL) was added methylamine hydrochloride (4.74 g, 70 mmol) and DBU (21.4 g, 140 mmol) at −45° C. Then the reaction temperature was allowed to warm to −10° C. The reaction mixture was stirred at the same temperature for 2.5 hrs. Et₂O (200 mL) was added and the resulting mixture was washed with brine (50 mL) four times. The separated organic layer was dried over Na₂SO₄ and concentrated in vacuo to afford tert-butyl 3-(methylamino)propanoate (3.5 g, Compound AR-1) as a light yellow oil.

### Step 2: Preparation of tert-butyl 3-[chlorocarbonyl(methyl)amino]propanoate (Intermediate AR)

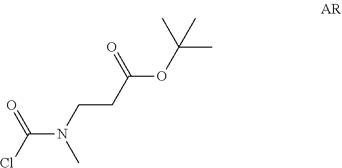

Intermediate AR was prepared in analogy to Intermediate AP by using tert-butyl 3-(methylamino)propanoate (3.4 g, Compound AR-1) instead of ethyl 2-(methylamino)acetate hydrochloride. The crude tert-butyl 3-[chlorocarbonyl(methyl)amino]propanoate (3.5 g, Intermediate AR) was obtained and used for next step without further purification.

### Intermediate AS

### Ethyl (2S)-2-[chlorocarbonyl(methyl)amino]propanoate

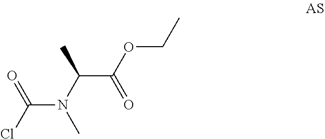

### Step 1: Preparation of ethyl (2S)-2-(methylamino)propanoate hydrochloride (Compound AS-1)

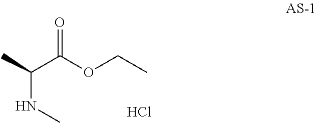

To a solution of (2S)-2-(methylamino)propanoic acid (1 g, 9.70 mmol) in EtOH (10 mL) was added SOCl₂ (1.50 g, 12.61 mmol) dropwise at 0° C. in 0.5 hr. The reaction mixture was stirred at 25° C. for 15.5 hrs, then diluted with EA (20 mL), washed with H₂O (5 mL) and brine (5 mL). The organic layer was dried over Na₂SO₄ and concentrated in vacuo. Ethyl (2S)-2-(methylamino)propanoate hydrochloride (1.8 g, Compound AS-1) was obtained as a yellow oil and used for next step without further purification.

### Step 2: Preparation of ethyl (2S)-2-(methylamino)propanoate (Compound AS-2)

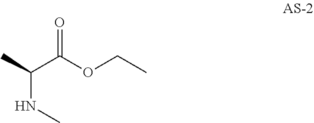

A solution of ethyl (2S)-2-(methylamino)propanoate hydrochloride (1.8 g, Compound AS-1) in EA (10 mL) was adjusted to pH=8 with 10 wt. % aqueous NaHCO₃. The reaction mixture was stirred at room temperature for 0.5 hr. The organic layer was washed with brine (5 mL), dried over Na₂SO₄ and concentrated in vacuo. Ethyl (2S)-2-(methylamino)propanoate (620 mg, Compound AS-2) was obtained as a yellow oil and used for the next step without further purification.

### Step 3: Preparation of ethyl (2S)-2-[chlorocarbonyl(methyl)amino]propanoate (Intermediate AS)

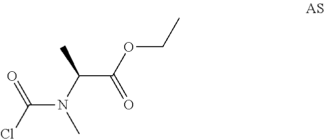

Intermediate AS was prepared in analogy to Intermediate AP by using ethyl (2S)-2-(methylamino)propanoate (260 mg, Compound AS-2) instead of ethyl 2-(methylamino)acetate hydrochloride. The crude ethyl (2S)-2-[chlorocarbonyl(methyl)amino]propanoate (200 mg, Intermediate AS) was obtained as a yellow oil and used for the next step without further purification.

### Intermediate AT

### tert-Butyl (2S)-2-[chlorocarbonyl(methyl)amino]-4-methyl-pentanoate

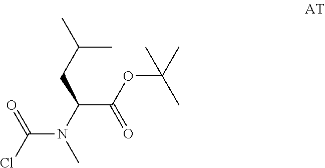 [SMILES: CC(C)C[C@@H](C(=O)OC(C)(C)C)N(C)C(=O)Cl]

### Step 1: Preparation of tert-butyl (2S)-4-methyl-2-(methylamino)pentanoate (Compound AT-1)

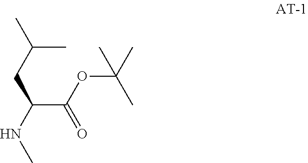

2-Methylpropene (25 g, 446 mmol) was bubbled into DCM (50 mL) at −78° C. Then the 2-methylpropene solution was added to a solution of (S)-4-methyl-2-(methylamino)pentanoic acid hydrochloride (500 mg, 2.75 mmol) and H₂SO₄ (3.68 g, 2 mL, 37.5 mmol) in dioxane (20 mL) at 0° C. The reaction mixture was stirred at room temperature for 18 hrs in a sealed tube. The reaction solution was poured into an ice cold aqueous KOH solution (8.4 g in water (30 mL)) and the resulting mixture was extracted with DCM (50 mL) twice. The combined organic layer was washed with brine (30 mL) twice, dried over Na₂SO₄ and concentrated in vacuo to afford the crude product tert-butyl (2S)-4-methyl-2-(methylamino)pentanoate (Compound AT-1) as a light yellow oil.

### Step 2: Preparation of tert-butyl (2S)-2-[chlorocarbonyl(methyl)amino]-4-methyl-pentanoate (Intermediate AT)

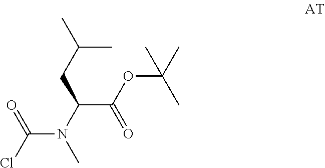 [SMILES: CC(C)C[C@@H](C(=O)OC(C)(C)C)N(C)C(=O)Cl]

Intermediate AT was prepared in analogy to Intermediate AP by using tert-butyl (2S)-4-methyl-2-(methylamino)pentanoate (300 mg, Compound AT-1) instead of ethyl 2-(methylamino)acetate hydrochloride. The crude tert-butyl (2S)-2-[chlorocarbonyl(methyl)amino]-4-methyl-pentanoate (350 mg, Intermediate AT) was obtained as a light yellow oil and used for the next step without further purification.

### Intermediate AU

### Isopropyl (2S)-2-[chlorocarbonyl(methyl)amino]-4-methyl-pentanoate

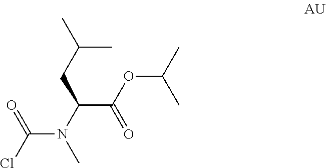 [SMILES: CC(C)C[C@@H](C(=O)OC(C)C)N(C)C(=O)Cl]

### Step 1: Preparation of isopropyl (2S)-4-methyl-2-(methylamino)pentanoate hydrochloride (Compound AU-1)

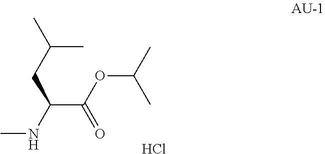

To a solution of (S)-4-methyl-2-(methylamino)pentanoic acid hydrochloride (0.5 g) in i-PrOH (7.8 g, 10 mL) was added thionyl chloride (655 mg, 402 μL) dropwise at room temperature. The resulting mixture was stirred and refluxed for 16 hrs and then concentrated in vacuo. The residue was basified with saturated aqueous NaHCO₃ (30 mL) and extracted with DCM (50 mL). The organic layer was washed with brine, dried over Na₂SO₄ and concentrated in vacuo. The residue was salified with HCl/EtOAc (10 mL, 1 mmol/mL) and concentrated to afford isopropyl (2S)-4-methyl-2-(methylamino)pentanoate hydrochloride (510 mg, Compound AU-1) as a white solid.

### Step 2: Preparation of isopropyl (2S)-2-[chlorocarbonyl(methyl)amino]-4-methyl-pentanoate (Intermediate AU)

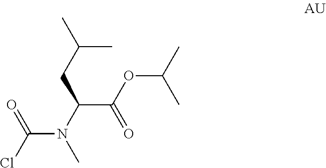 [SMILES: CC(C)C[C@@H](C(=O)OC(C)C)N(C)C(=O)Cl]

Intermediate AU was prepared in analogy to Intermediate AP by using isopropyl (2S)-4-methyl-2-(methylamino)pentanoate hydrochloride (500 mg, Compound AU-1) instead of ethyl 2-(methylamino)acetate hydrochloride. The crude isopropyl (2S)-2-[chlorocarbonyl(methyl)amino]-4-methyl-pentanoate (650 mg, Intermediate AU) was obtained as a light yellow oil and used for the next step without further purification.

### Intermediate AV

### Ethyl (2S)-2-[chlorocarbonyl(methyl)amino]-3-methyl-butanoate

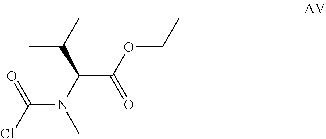

### Step 1: Preparation of ethyl (2S)-3-methyl-2-(methylamino)butanoate hydrochloride (Compound AV-1)

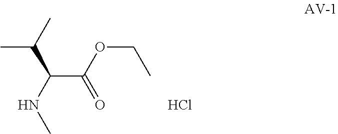

To a solution of (2S)-3-methyl-2-(methylamino)butanoic acid (1.0 g, 7.6 mmol) in EtOH (10 mL) was added thionyl chloride (2.45 g, 21 mmol) dropwise at room temperature. The resulting mixture was stirred and refluxed for 16 hrs and then concentrated in vacuo. The residue was basified with saturated aqueous NaHCO₃ (30 mL) and extracted with DCM (50 mL) twice. The combined organic layer was washed with brine, dried over Na₂SO₄ and concentrated in vacuo. The residue was dissolved in HCl/EtOAc (10 mL, 1 M) and concentrated to afford ethyl (2S)-3-methyl-2-(methylamino)butanoate hydrochloride (1.9 g, Compound AV-1) as a white solid.

### Step 2: Preparation of ethyl (2S)-2-[chlorocarbonyl(methyl)amino]-3-methyl-butanoate (Intermediate AV)

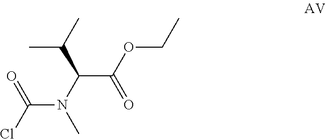

Intermediate AV was prepared in analogy to Intermediate AP by using ethyl (2S)-3-methyl-2-(methylamino)butanoate hydrochloride (500 mg, Compound AV-1) instead of ethyl 2-(methylamino)acetate hydrochloride. The crude ethyl (2S)-2-[chlorocarbonyl(methyl)amino]-3-methyl-butanoate (600 mg, Intermediate AV) was obtained as a light yellow oil and used for the next step without further purification.

### Intermediate AW

### Ethyl (2S)-2-[chlorocarbonyl(methyl)amino]-4-methyl-pentanoate

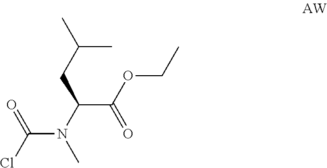

### Step 1: Preparation of ethyl (2S)-4-methyl-2-(methylamino)pentanoate hydrochloride (Compound AW-1)

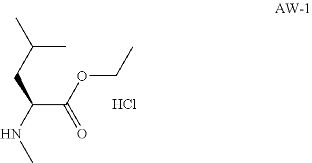

To a solution of (2S)-4-methyl-2-(methylamino)pentanoic acid (1 g, 6.9 mmol) in EtOH (10 mL) was added thionyl chloride (1.07 g, 8.3 mmol) dropwise at room temperature. The resulting mixture was stirred at reflux for 16 hrs and then concentrated in vacuo. The residue was basified with saturated aqueous NaHCO₃ (30 mL) and extracted with DCM (50 mL). The organic layer was washed with brine, dried over Na₂SO₄ and concentrated in vacuo. The residue was salified with HCl/EtOAc (10 mL, 1 mmol/mL) and concentrated to give ethyl (2S)-4-methyl-2-(methylamino)pentanoate hydrochloride (1.8 g, Compound AW-1) as a white solid.

### Step 2: Preparation of ethyl (2S)-2-[chlorocarbonyl(methyl)amino]-4-methyl-pentanoate (Intermediate AW)

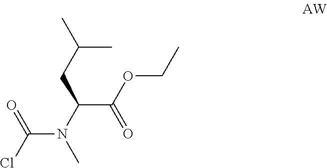

Intermediate AW was prepared in analogy to Intermediate AP by using ethyl (2S)-4-methyl-2-(methylamino)pentanoate hydrochloride (610 mg, AW-1) instead of ethyl 2-(methylamino)acetate hydrochloride. The crude ethyl (2S)-2-[chlorocarbonyl(methyl)amino]-4-methyl-pentanoate (280 mg, Intermediate AW) was obtained as a light yellow oil and used for the next step without further purification.

### Intermediate AX

### Ethyl (2S)-2-[chlorocarbonyl(methyl)amino]-3-phenyl-propanoate

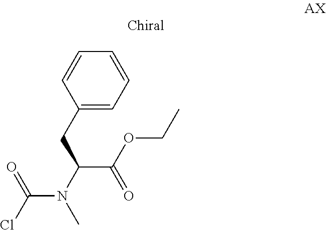 [SMILES: CCOC(=O)[C@H](Cc1ccccc1)N(C)C(=O)Cl]

Intermediate AX was prepared in analogy to Intermediate AP by using (S)-ethyl-2-(methylamino)-3-phenylpropanoate instead of ethyl 2-(methylamino)acetate hydrochloride. The crude ethyl (2S)-2-[chlorocarbonyl(methyl)amino]-3-phenyl-propanoate (200 mg, Intermediate AX) was obtained as a light yellow oil and used for the next step without further purification

### Intermediate AY

### Isopropyl (2S)-2-[chlorocarbonyl(methyl)amino]-3-phenyl-propanoate

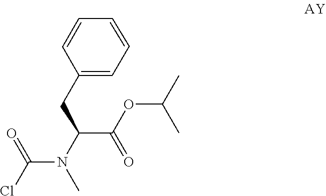 [SMILES: CC(C)OC(=O)[C@H](Cc1ccccc1)N(C)C(=O)Cl]

Intermediate AY was prepared in analogy to Intermediate AP by using isopropyl (2S)-2-(methylamino)-3-phenyl-propanoate (190 mg) instead of ethyl 2-(methylamino)acetate hydrochloride. The crude isopropyl (2S)-2-[chlorocarbonyl(methyl)amino]-3-phenyl-propanoate (220 mg, Intermediate AY) was obtained as light brown oil and used for the next step without further purification.

### Intermediate AZ

### (S)-tert-butyl 2-((chlorocarbonyl)(methyl)amino)-3-phenylpropanoate

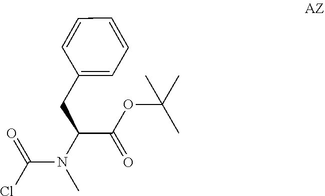 [SMILES: CN(C(=O)Cl)[C@@H](Cc1ccccc1)C(=O)OC(C)(C)C]

### Step 1: Preparation of tert-butyl (2S)-2-(methylamino)-3-phenyl-propanoate (Compound AZ-1)

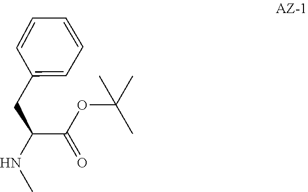 [SMILES: CN[C@@H](Cc1ccccc1)C(=O)OC(C)(C)C]

2-Methylpropene (25 g, 446 mmol) was bubbled into DCM (50 mL) at −78° C. Then the 2-methylpropene solution was added to a solution of (S)-2-(methylamino)-3-phenylpropanoic acid (500 mg) and H₂SO₄ (3.68 g, 2 mL) in dioxane (20 mL) at 0° C. The reaction mixture was stirred at room temperature for 18 hrs in a sealed tube. The reaction mixture was poured into an ice cold aqueous KOH solution (8.4 g in water (30 mL)) and the resulting mixture was extracted with DCM (50 mL) twice. The organic layer was washed with brine (30 mL) 2 times, dried over Na₂SO₄ and concentrated in vacuo to afford tert-butyl (2S)-2-(methylamino)-3-phenyl-propanoate (710 mg, Compound AZ-1) as a light yellow oil.

### Step 2: Preparation of (S)-tert-butyl 2-((chlorocarbonyl)(methyl)amino)-3-phenylpropanoate (Intermediate AZ)

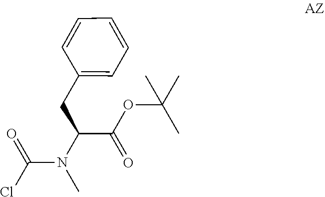 [SMILES: CN(C(=O)Cl)[C@@H](Cc1ccccc1)C(=O)OC(C)(C)C]

Intermediate AZ was prepared in analogy to intermediate AP by using tert-butyl (2S)-2-(methylamino)-3-phenyl-propanoate (Compound AZ-1) instead of ethyl 2-(methylamino)acetate hydrochloride. The crude tert-butyl (2S)-2-[chlorocarbonyl(methyl)amino]-3-phenyl-propanoate (360 mg, Intermediate AZ) was obtained as a light yellow oil and used for next step without further purification

### Intermediate BA

### N-[2-[acetyl(methyl)amino]ethyl]-N-methyl-carbamoyl chloride

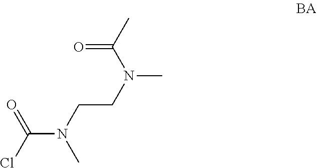

### Step 1: Preparation of tert-butyl N-[2-[acetyl(methyl)amino]ethyl]-N-methyl-carbamate (Compound BA-1)

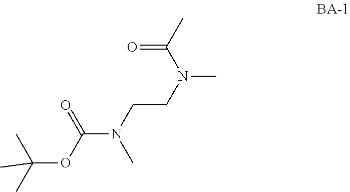 [SMILES: CC(=O)N(C)CCN(C)C(=O)OC(C)(C)C]

To a solution of tert-butyl methyl(2-(methylamino)ethyl)carbamate (1.13 g, 6 mmol) in pyridine (10 mL) was added acetic anhydride (3.06 g, 30 mmol) dropwise at 0° C. Then the solution was stirred at room temperature for 0.5 hr. The solvent was removed in vacuo and the residue was partitioned between EtOAc (50 mL) and saturated aqueous NaHCO₃ (25 mL). The organic layer was separated, washed with brine (20 mL), dried over Na₂SO₄ and concentrated in vacuo to afford tert-butyl N-[2-[acetyl(methyl)amino]ethyl]-N-methyl-carbamate (1.28 g, Compound BA-1) as a yellow oil.

### Step 2: Preparation of N-methyl-N-(2-(methylamino)ethyl)acetamide hydrochloride (Compound BA-2)

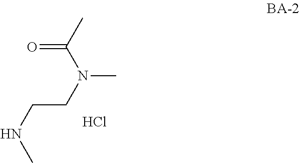

A mixture of tert-butyl N-[2-[acetyl(methyl)amino]ethyl]-N-methyl-carbamate (1.1 g, Compound BA-1) in HCl/EtOAc (10 mL, 1N HCl in EtOAc) was stirred at room temperature for 2 hrs, then the mixture was filtered. The collected solid was washed with EtOAc (5 mL) three times and dried in vacuo to afford the crude N-methyl-N-(2-(methylamino)ethyl)acetamide hydrochloride (460 mg, Compound BA-2) as a white solid.

### Step 3: Preparation of N-[2-[acetyl(methyl)amino]ethyl]-N-methyl-carbamoyl chloride (Intermediate BA)

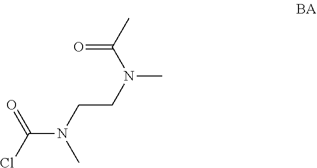

Intermediate BA was prepared in analogy to Intermediate AP by using N-methyl-N-(2-(methylamino)ethyl)acetamide hydrochloride (200 mg, Compound BA-2) instead of ethyl 2-(methylamino)acetate hydrochloride The crude N-[2-[acetyl(methyl)amino]ethyl]-N-methyl-carbamoyl chloride (300 mg, Intermediate BA) was obtained and used for next step without further purification.

### Intermediate BB

### Methyl N-[2-[chlorocarbonyl(methyl)amino]ethyl]-N-methyl-carbamate

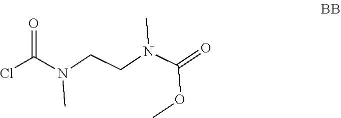

### Step 1: Preparation of methyl N-methyl-N-[2-(methylamino)ethyl]carbamate (Compound BB-1)

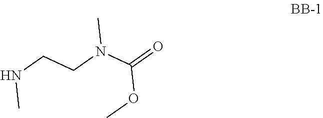

To a solution of N,N-dimethylethane-1,2-diamine (10 g) in THF (40 mL) was added methyl chloroformate (1.92 g) dropwise at −70° C. in 1 hr. The mixture was stirred at 25° C. for 15 hrs and then filtered and washed with water and brine. The organic layer was dried and concentrated to afford a yellow residue, which was purified by column chromatography to afford methyl N-methyl-N-[2-(methylamino)ethyl]carbamate (2 g, Compound BB-1) as a colorless oil.

### Step 2: Preparation of methyl N-[2-[chlorocarbonyl(methyl)amino]ethyl]-N-methyl-carbamate (Intermediate BB)

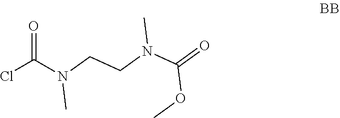

Intermediate BB was prepared in analogy to Intermediate AP by using methyl N-methyl-N-[2-(methylamino)ethyl]carbamate (2.0 g, Compound BB-1) instead of ethyl 2-(methylamino)acetate hydrochloride. The crude methyl N-[2-[chlorocarbonyl(methyl)amino]ethyl]-N-methyl-carbamate (2.2 g, Intermediate BB) was obtained and used for next step without further purification.

### Intermediate BC

### tert-Butyl N-[2-[chlorocarbonyl(methyl)amino]ethyl]-N-methyl-carbamate

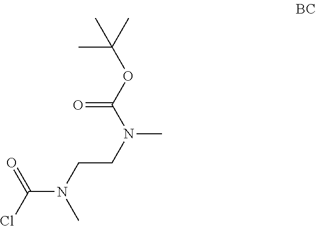 [SMILES: CN(CCN(C)C(=O)OC(C)(C)C)C(=O)Cl]

### Step 1: Preparation of tert-butyl N-methyl-N-[2-(methylamino)ethyl]carbamate (Compound BC-1)

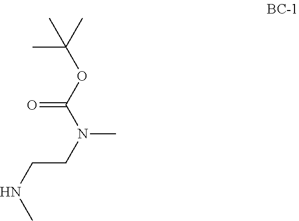

To a solution of N,N′-dimethylethane-1,2-diamine (40.4 g) in DCM (300 mL) was added a solution of Boc₂O (10 g, 10.6 mL, 45.8 mmol) in DCM (100 mL) dropwise at 0° C. over 1 hr. The reaction mixture was stirred at room temperature for 18 hrs. The organic layer was washed with saturated aqueous NaHCO₃ (50 mL), brine (50 mL), dried over Na₂SO₄ and concentrated in vacuo. The residue was purified by column chromatography to afford tert-butyl N-methyl-N-[2-(methylamino)ethyl]carbamate (6.8 g, Compound BC-1) as a yellow oil. ¹H NMR (400 MHz, CDCl₃) δ ppm: 3.34 (br. s., 2H), 2.89 (s, 3H), 2.74 (t, J=6.7 Hz, 2H), 2.46 (s, 3H), 1.47 (s, 9H).

### Step 2: Preparation of tert-butyl N-[2-[chlorocarbonyl(methyl)amino]ethyl]-N-methyl-carbamate (Intermediate BC)

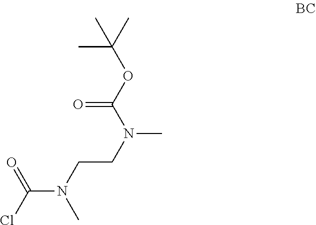 [SMILES: CN(CCN(C)C(=O)OC(C)(C)C)C(=O)Cl]

Intermediate BC was prepared in analogy to Intermediate AP by using tert-butyl N-methyl-N-[2-(methylamino)ethyl]carbamate (1.15 g, Compound BC-1) instead of ethyl 2-(methylamino)acetate hydrochloride. The crude tert-butyl N-[2-[chlorocarbonyl(methyl)amino]ethyl]-N-methyl-carbamate (1.3 g, Intermediate BC) was obtained and used for the next step without further purification.

### Intermediate BD

### Ethyl N-[2-[chlorocarbonyl(methyl)amino]ethyl]-N-methyl-carbamate

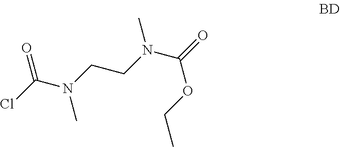

### Step 1: Preparation of ethyl N-methyl-N-[2-(methylamino)ethyl]carbamate (Compound BD-1)

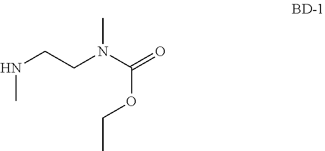

To a solution of N,N′-dimethylethane-1,2-diamine (10 g) in DCM (40 mL) was added ethyl chloroformate (2.58 g) dropwise at −70° C. in 1 hr. The reaction mixture was stirred at 25° C. for 15 hrs and then filtered and washed with water and brine. The organic layer was dried and concentrated in vacuo. The yellow residue was purified by column chromatography to afford ethyl N-methyl-N-[2-(methylamino)ethyl]carbamate (2 g, Compound BD-1) as a colorless oil.

### Step 2: Preparation of ethyl N-[2-[chlorocarbonyl(methyl)amino]ethyl]-N-methyl-carbamate (Intermediate BD)

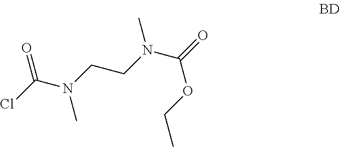

Intermediate BD was prepared in analogy to Intermediate AA by using ethyl N-methyl-N-[2-(methylamino)ethyl]carbamate (Compound BD-1) instead of ethyl 2-(methylamino)acetate hydrochloride. The crude ethyl N-[2-[chlorocarbonyl(methyl)amino]ethyl]-N-methyl-carbamate (2.2 g, Intermediate BD) was obtained and used for the next step without further purification.

### Intermediate BE

### 2-[Chlorocarbonyl(methyl)amino]ethyl N-butyl-N-methyl-carbamate

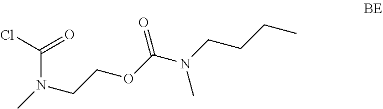 [SMILES: CCCCN(C)C(=O)OCCN(C)C(=O)Cl]

### Step 1: Preparation of tert-butyl N-(2-hydroxyethyl)-N-methyl-carbamate (Compound BE-1)

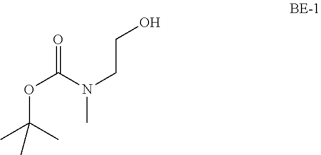

To a solution of 2-(methylamino)ethanol (10 g, 133.14 mmol) in DCM (10 mL) was added Boc₂O (34.87 g, 159.77 mmol) at 25° C. The mixture was stirred at 25° C. for 16 hrs and then concentrated. The residue was purified by column chromatography to afford tert-butyl N-(2-hydroxyethyl)-N-methyl-carbamate (20 g, Compound BE-1) as a colorless oil.

### Step 2: Preparation of 2-[tert-butoxycarbonyl(methyl)amino]ethyl N-butyl-N-methyl-carbamate (Compound BE-2)

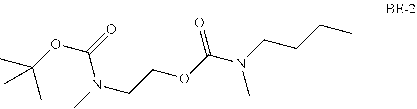 [SMILES: CCCCN(C)C(=O)OCCN(C)C(=O)OC(C)(C)C]

To a solution of tert-butyl N-(2-hydroxyethyl)-N-methyl-carbamate (880 mg, Compound BE-1) and Et₃N (1 g, 10.08 mmol) in DCM (10 mL) was added N-butyl-N-methyl-carbamoyl chloride (903 mg, 7.04 mmol) dropwise at −10° C. in 1 hr. The reaction mixture was stirred at 25° C. for 15 hrs and then filtered and washed with water and brine. The organic layer was dried and concentrated to afford 2-[tert-butoxycarbonyl(methyl)amino]ethyl N-butyl-N-methyl-carbamate (2 g, Compound BE-2) as a colorless oil.

### Step 3: Preparation of 2-(methylamino)ethyl N-butyl-N-methyl-carbamate hydrochloride (Compound BE-3)

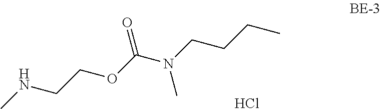

To a solution of 2-[tert-butoxycarbonyl(methyl)amino]ethyl N-butyl-N-methyl-carbamate (1 g, Compound BE-2) was added HCl/EA (40 mL, 1M). The reaction mixture was stirred at 0° C. for 0.5 hr and warmed to 25° C. and stirred for another 15.5 hrs. The reaction mixture was concentrated to afford 2-(methylamino)ethyl-N-butyl-N-methyl-carbamate hydrochloride (400 mg, Compound BE-3) as a colorless oil.

### Step 4: Preparation of 2-[chlorocarbonyl(methyl)amino]ethyl N-butyl-N-methyl-carbamate (Intermediate BE)

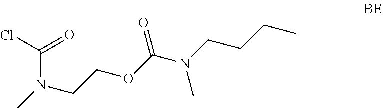 [SMILES: CCCCN(C)C(=O)OCCN(C)C(=O)Cl]

Intermediate BE was prepared in analogy to Intermediate AP by using 2-(methylamino)ethyl N-butyl-N-methyl-carbamate hydrochloride (374 mg, Compound BE-3) instead of ethyl 2-(methylamino)acetate hydrochloride. The crude 2-[chlorocarbonyl(methyl)amino]ethyl N-butyl-N-methyl-carbamate (330 mg, Intermediate BE) was obtained and used for next step without further purification.

### Intermediate BF

### 2-[Chlorocarbonyl(methyl)amino]ethyl pyrrolidine-1-carboxylate

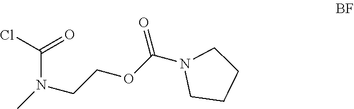

### Step 1: Preparation of tert-butyl N-(2-hydroxyethyl)-N-methyl-carbamate (Compound BF-1)

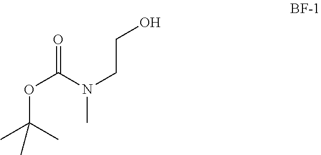

To a solution of 2-(methylamino)ethanol (10 g, 133.14 mmol) in DCM (10 mL) was added Boc₂O (34.87 g, 159.77 mmol) at 25° C. The mixture was stirred at 25° C. for 16 hrs. The reaction mixture was concentrated to give the residue, which was purified by column chromatography to afford tert-butyl N-(2-hydroxyethyl)-N-methyl-carbamate (20 g, Compound BF-1) as a colorless oil.

### Step 2: Preparation of 2-[tert-butoxycarbonyl(methyl)amino]ethyl pyrrolidine-1-carboxylate (Compound BF-2)

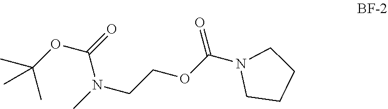 [SMILES: CN(CCOC(=O)N1CCCC1)C(=O)OC(C)(C)C]

To a solution of tert-butyl N-(2-hydroxyethyl)-N-methyl-carbamate (300 mg, 1.71 mmol, Compound BF-1) and Et₃N (578 mg, 5.71 mmol) in DCM (5 mL) was added pyrrolidine-1-carbonyl chloride (458 mg, 3.4 mmol) dropwise at 0° C. for 0.5 hr and then stirred at 25° C. for 15.5 hrs. After filtration, the filtrate was washed with water and brine. The organic layer was dried and concentrated to afford the 2-[tert-butoxycarbonyl(methyl)amino]ethyl pyrrolidine-1-carboxylate (335 mg, Compound BF-2) as a colorless oil.

### Step 3: Preparation of 2-(methylamino)ethyl pyrrolidine-1-carboxylate hydrochloride (Compound BF-3)

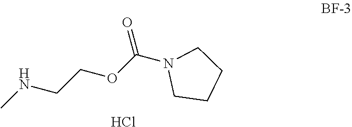

2-[tert-butoxycarbonyl(methyl)amino]ethyl pyrrolidine-1-carboxylate (335 mg, Compound BF-2) was added to HCl in EA (12.3 mL, 1M) and the mixture was stirred at 0° C. for 0.5 hr and then at 25° C. for another 15.5 hrs. The reaction mixture was concentrated to afford 2-(methylamino)ethyl pyrrolidine-1-carboxylate hydrochloride (300 mg, Compound BF-3) as a colorless oil.

### Step 4: Preparation of 2-[chlorocarbonyl(methyl)amino]ethyl pyrrolidine-1-carboxylate (Intermediate BF)

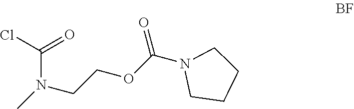

Intermediate BF was prepared in analogy to Intermediate AP by using the 2-(methylamino)ethyl pyrrolidine-1-carboxylate hydrochloride (299 mg, Compound BF-3) instead of ethyl 2-(methylamino)acetate hydrochloride. The crude 2-[chlorocarbonyl(methyl)amino]ethyl pyrrolidine-1-carboxylate (230 mg, Intermediate BF) was obtained and used for next step without further purification.

### Intermediate BG

### 2-[Chlorocarbonyl(methyl)amino]ethyl N-methyl-N-propyl-carbamate

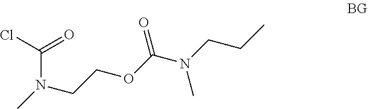

### Step 1: Preparation of tert-butyl N-(2-hydroxyethyl)-N-methyl-carbamate (Compound BG-1)

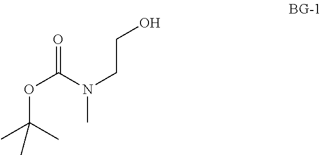

To a solution of 2-(methylamino)ethanol (10 g, 133.14 mmol) in DCM (10 mL) was added Boc₂O (34.87 g, 159.77 mmol) at 25° C. The reaction mixture was stirred at 25° C. for 16 hrs, then concentrated to give the residue, which was purified by column chromatography to afford tert-butyl N-(2-hydroxyethyl)-N-methyl-carbamate (20 g, Compound BG-1) as a colorless oil.

### Step 2: Preparation of tert-butyl-N-methyl-N-[2-[methyl(propyl)carbamoyl]oxyethyl] carbamate (Compound BG-2)

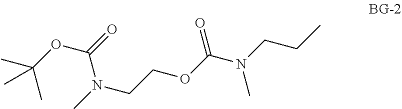 [SMILES: CCCN(C)C(=O)OCCN(C)C(=O)OC(C)(C)C]

To a solution of tert-butyl N-(2-hydroxyethyl)-N-methyl-carbamate (265 mg, Compound BG-1) and Et₃N (1 mL, 5.71 mmol) in DCM (5 mL) was added N-methyl-N-propyl-carbamoyl chloride (410 mg, 1.83 mmol) dropwise at 0° C. for 0.5 hr. The reaction mixture was stirred at 25° C. for 15.5 hrs and then filtered and the filtrate was washed with water and brine. The organic layer was dried and concentrated to afford tert-butyl N-methyl-N-[2-[methyl(propyl)carbamoyl]oxyethyl]carbamate (380 mg, Compound BG-2) as a colorless oil.

### Step 3: Preparation of 2-(methylamino)ethyl N-methyl-N-propyl-carbamate hydrochloride (Compound BG-3)

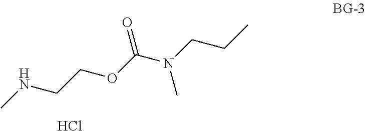

tert-butyl N-methyl-N-[2-[methyl(propyl)carbamoyl]oxyethyl]carbamate (380 mg, Compound BG-2) was added to HCl in EA (13.7 mL, 1M). The mixture was stirred at 0° C. for 0.5 hr. Then the mixture was stirred at 25° C. for another 15.5 hrs and concentrated to afford 2-(methylamino)ethyl N-methyl-N-propyl-carbamate hydrochloride (300 mg, Compound BG-3) as a colorless oil.

### Step 4: Preparation of 2-[chlorocarbonyl(methyl)amino]ethyl N-methyl-N-propyl-carbamate (Intermediate BG)

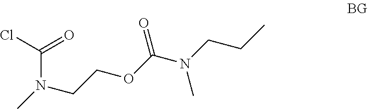

Intermediate BG was prepared in analogy to Intermediate AP by using 2-(methylamino)ethyl N-methyl-N-propyl-carbamate hydrochloride (330 mg, Compound BG-3) instead of ethyl 2-(methylamino)acetate hydrochloride. The 2-[chlorocarbonyl(methyl)amino]ethyl-N-methyl-N-propyl-carbamate (300 mg, Intermediate BG) was obtained and used for next step without further purification.

### Intermediate BH

### 2-[Chlorocarbonyl(methyl)amino]ethyl N,N-diethylcarbamate

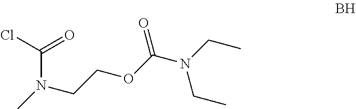

### Step 1: Preparation of tert-butyl N-(2-hydroxyethyl)-N-methyl-carbamate (Compound BH-1)

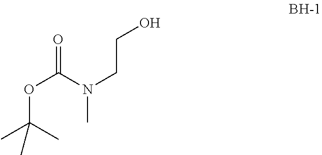

To a solution of 2-(methylamino)ethanol (10 g, 133.14 mmol) in DCM (10 mL) was added Boc₂O (34.87 g, 159.77 mmol) at 25° C. The mixture was stirred at 25° C. for 16 hrs and then concentrated, the residue was purified by column chromatography to afford tert-butyl N-(2-hydroxyethyl)-N-methyl-carbamate (20 g, Compound BH-1) as a colorless oil.

### Step 2: Preparation of 2-[tert-butoxycarbonyl(methyl)amino]ethyl-N,N-diethylcarbamate (Compound BH-2)

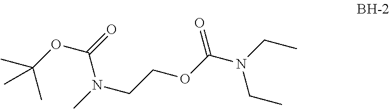 [SMILES: CCN(CC)C(=O)OCCN(C)C(=O)OC(C)(C)C]

To a solution of tert-butyl N-(2-hydroxyethyl)-N-methyl-carbamate (200 mg, 1.14 mmol, Compound BH-1) and Et₃N (578 mg, 5.71 mmol) in DCM (5 mL) was added N,N-diethylcarbamoyl chloride (248 mg, 1.83 mmol) dropwise at 0° C. for 0.5 hr and stirred at 25° C. for 15.5 hrs. After filtration, the filtrate was washed with water and brine. The organic layer was dried and concentrated to afford the 2-[tert-butoxycarbonyl(methyl)amino]ethyl N,N-diethylcarbamate (313 mg, Compound BH-2) as a colorless oil.

### Step 3: Preparation of 2-(methylamino)ethyl N,N-diethylcarbamate hydrochloride (Compound BH-3)

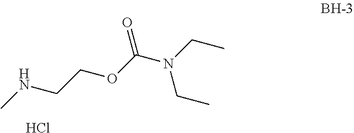

2-[tert-butoxycarbonyl(methyl)amino]ethyl N,N-diethylcarbamate (436 mg, 1.77 mmol, Compound BH-2) was added to HCl in EA (17 mL, 1M). The mixture was stirred at 0° C. for 0.5 hr. Then the mixture was stirred at 25° C. for another 15.5 hrs and concentrated to afford 2-(methylamino)ethyl N,N-diethylcarbamate hydrochloride (230 mg, Compound BH-3) as a colorless oil.

### Step 4: Preparation of 2-[chlorocarbonyl(methyl)amino]ethyl N,N-diethylcarbamate (Intermediate BH)

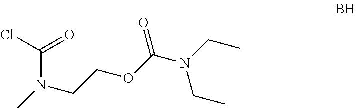

Intermediate BH was prepared in analogy to Intermediate AP by using 2-(methylamino)ethyl N,N-diethylcarbamate hydrochloride (274 mg, Compound BH-3) instead of ethyl 2-(methylamino)acetate hydrochloride. The crude 2-[chlorocarbonyl(methyl)amino]ethyl N,N-diethylcarbamate (250 mg, Intermediate BH) was obtained and used for next step without further purification.

### Intermediate BI

### 2-[Chlorocarbonyl(methyl)amino]ethyl ethyl carbonate

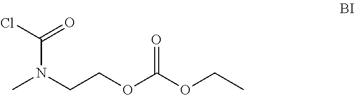

### Step 1: Preparation of tert-butyl N-(2-hydroxyethyl)-N-methyl-carbamate (Compound BI-1)

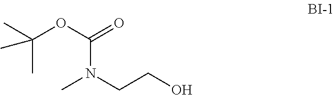

To a solution of 2-(methylamino)ethanol (1 g, 13.31 mmol) in DCM (10 mL) was added Boc₂O (3.49 g, 15.98 mmol) at 25° C. The reaction mixture was stirred at 25° C. for 16 hrs, then concentrated to give the crude product, which was purified by column chromatography to afford tert-butyl N-(2-hydroxyethyl)-N-methyl-carbamate (1.6 g, Compound BI-1) as a colorless oil.

### Step 2: Preparation of 2-[tert-butoxycarbonyl(methyl)amino]ethyl methyl carbonate (Compound BI-2)

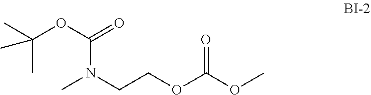 [SMILES: COC(=O)OCCN(C)C(=O)OC(C)(C)C]

To a solution of tert-butyl N-(2-hydroxyethyl)-N-methyl-carbamate (1 g, Compound BI-1), DMAP (0.1 g) and pyridine (1.15 g, 11.41 mmol) in EA (20 mL) was added methyl chloroformate (1.21 g, 11.15 mmol) dropwise at −10° C. The mixture was stirred at −10° C. for 1 hr. The reaction mixture was filtered and the filtrate was washed with 5% citric acid and brine. The organic layer was dried and concentrated to afford 2-[tert-butoxycarbonyl(methyl)amino]ethyl methyl carbonate (1.22 g, Compound BI-2) as a colorless oil.

### Step 3: Preparation of ethyl 2-(methylamino)ethyl carbonate hydrochloride (Compound BI-3)

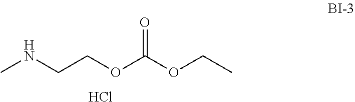

2-[tert-butoxycarbonyl(methyl)amino]ethyl methyl carbonate (1.22 g, 4.94 mmol, Compound BI-2) was added to HCl in EA (10 mL, 40 mmol) and the mixture was stirred at 0° C. for 0.5 hr and at 25° C. for another 15.5 hrs. The reaction mixture was concentrated to afford ethyl 2-(methylamino)ethyl carbonate hydrochloride (1.06 g, Compound BI-3).

### Step 4: Preparation of 2-[chlorocarbonyl(methyl)amino]ethyl ethyl carbonate (Intermediate BI)

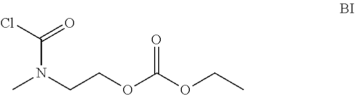

Intermediate BI was prepared in analogy to Intermediate AP by using ethyl 2-(methylamino)ethyl carbonate hydrochloride (150 mg, Intermediate BI-3) instead of ethyl 2-(methylamino)acetate hydrochloride. The crude 2-[chlorocarbonyl(methyl)amino]ethyl ethyl carbonate (145 mg, Intermediate BI) was obtained and used for next step without further purification.

### PREPARATIVE EXAMPLES

### Example 1

### 6-Amino-9-benzyl-N-methyl-8-oxo-N-propyl-2-(propylsulfonimidoyl)purine-7-carboxamide

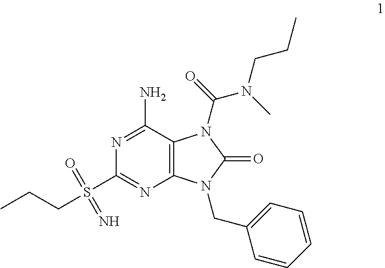 [SMILES: CCCN(C)C(=O)n1c(=O)n(Cc2ccccc2)c2nc(S(=N)(=O)CCC)nc(N)c21]

### Method A

### Step 1: Preparation of 4-amino-3-benzyl-2-oxo-1H-imidazole-5-carbonitrile (Compound 1a)

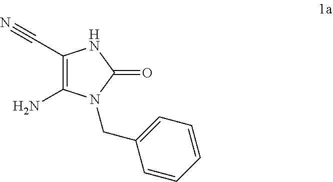 [SMILES: N#Cc1[nH]c(=O)n(Cc2ccccc2)c1N]

To a solution of aminomalononitrile p-toluenesulfonate (25 g, 98.5 mmol, TCI, Catalog number: A1119-25G) in dry THF (100 mL) was added benzyl isocyanate (13.2 g, 98.5 mmol) and TEA (10.2 g, 79.0 mmol) at RT. After stirred at RT for 24 hrs, the reaction was concentrated in vacuo and the residue was partitioned between EtOAc (500 mL) and water (250 mL). The separated organic layer was washed with brine (50 mL) twice, and extracted with sodium hydroxide solution (50 mL, 1N) twice. The combined sodium hydroxide solution layer was neutralized with 10 wt. % sodium hydrogen sulfate solution and extracted with EtOAc. The separated organic layer was washed with brine, dried over Na₂SO₄, filtered and concentrated in vacuo. The residue was triturated in 2-isopropoxypropane and then the suspension was filtered to give 4-amino-3-benzyl-2-oxo-1H-imidazole-5-carbonitrile (15 g, Compound 1a) as a yellow solid. The product was used for the next step without further purification. MS obsd. (ESI⁺) [(M+H)⁺]: 215.

### Step 2: Preparation of 6-amino-9-benzyl-2-sulfanyl-7H-purin-8-one (Compound 1b)

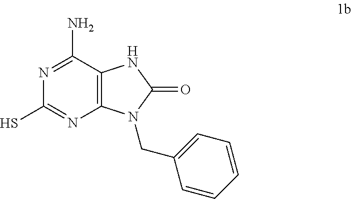 [SMILES: Nc1nc(S)nc2c1[nH]c(=O)n2Cc1ccccc1]

To a solution of 4-amino-3-benzyl-2-oxo-1H-imidazole-5-carbonitrile (15.0 g, 70.0 mmol, Compound 1a) in THF (700 mL) was added benzoylisothiocyanate (28.6 g, 175.1 mmol, TCI, Catalog number: A11596-100G) dropwise. After stirred at RT for 12 hrs, the reaction mixture was concentrated in vacuo. The residue was triturated in diethyl ether (100 mL) and the resulting precipitate was collected by filtration.

To a solution of the obtained precipitate in THF (700 mL) was added sodium hydroxide (70 mL, 2 N).

The mixture was refluxed for 50 hrs, and then acidified to pH=3 with 10 wt. % aqueous sodium hydrogen sulfate solution. The resulting precipitate was collected by filtration to give a crude 6-amino-9-benzyl-2-sulfanyl-7H-purin-8-one (8.1 g, Compound 1b) as a yellow solid. The product was used for the next step without further purification. MS obsd. (ESI⁺) [(M+H)⁺]: 274.

### Step 3: Preparation of 6-amino-9-benzyl-2-(2-propylsulfanyl)-7H-purin-8-one (Compound 1c)

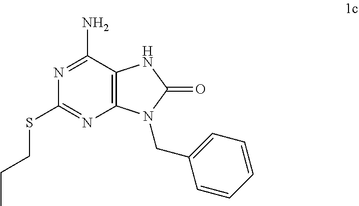 [SMILES: CCCSc1nc(N)c2[nH]c(=O)n(Cc3ccccc3)c2n1]

To a solution of 6-amino-9-benzyl-2-sulfanyl-7H-purin-8-one (5.46 g, 20.0 mmol, Compound 1b) in DMF was added potassium carbonate (2.76 g, 20.0 mmol). And then 1-bromopropane (2.44 g, 20.0 mmol, TCI, Catalog number: B0638-500G) in DMF (5.0 mL) was slowly added to previous solution. After stirred at RT for 12 hrs, the reaction mixture was poured into water (200 mL), then acidified with 10 wt. % aqueous sodium hydrogen sulfate solution and extracted with EtOAc (100 mL) twice. The organic layer was washed with brine, dried over Na₂SO₄ and concentrated in vacuo to give the crude product, which was purified by flash chromatography on silica gel to give 6-amino-9-benzyl-2-(2-propylsulfanyl)-7H-purin-8-one (4.8 g, Compound 1c) as a white solid. MS obsd. (ESI⁺) [(M+H)⁺]: 316.

### Step 4: Preparation of 6-amino-9-benzyl-2-propylsulfinyl-7H-purin-8-one (Compound 1d)

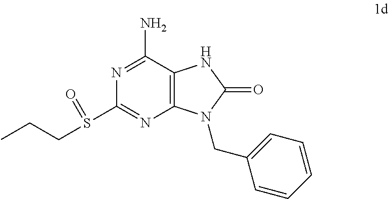 [SMILES: CCCS(=O)c1nc(N)c2[nH]c(=O)n(Cc3ccccc3)c2n1]

To a suspension of compound 6-amino-9-benzyl-2-(2-propylsulfanyl)-7H-purin-8-one (2.7 g, 8.7 mmol, Compound 1c) in DCM/MeOH (500 mL, V/V=1:1) was added 3-chloroperbenzoic acid (2.15 g, 8.7 mmol, 70% purity, Aldrich, Catalog number: 273031-100G). After reaction mixture was stirred for 2 hrs, the volume of reaction mixture was reduced in vacuo to about 50 mL. The resulting precipitate was collected by filtration, washed with methanol and dried to give 6-amino-9-benzyl-2-propylsulfinyl-7H-purin-8-one (1.0 g, Compound 1d) as a white solid. The product was used for the next step without further purification. MS obsd. (ESI⁺) [(M+H)⁺]: 332.

### Step 5: Preparation of 6-amino-9-benzyl-2-(propylsulfonimidoyl)-7H-purin-8-one (Compound 1e)

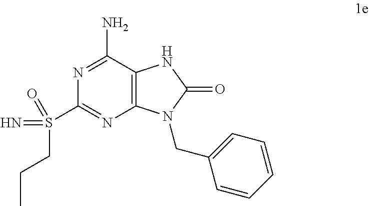 [SMILES: CCCS(=N)(=O)c1nc(N)c2[nH]c(=O)n(Cc3ccccc3)c2n1]

To a solution of 6-amino-9-benzyl-2-propylsulfinyl-7H-purin-8-one (1.52 g, 4.6 mmol, Compound 1d) in Eaton's reagent (40 mL, phosphorus pentoxide, 7.5 wt. % in methanesulphonic acid, Aldrich, Catalog number: 380814-100ML) was added sodium azide (360 mg, 5.5 mmol) at 50° C. After being stirred at this temperature for 30 minutes, the reaction mixture was cooled to RT and poured into sat. aqueous sodium bicarbonate solution. The reaction mixture was extracted with n-BuOH (100 mL) twice, and the organic phase was concentrated in vacuo. The residue was submitted for purification by prep-HPLC to give 6-amino-9-benzyl-2-(propylsulfonimidoyl)-7H-purin-8-one (1.2 g, Compound 1e) as a white solid. ¹H NMR (400 MHz, DMSO-d₆) δ ppm: 10.65 (br. s., 1H), 7.26-7.37 (m, 5H), 6.98 (br. s., 2H), 4.97 (s, 2H), 4.02 (s, 1H), 3.33 (t, J=7.53 Hz, 2H), 1.55-1.74 (m, 2H), 0.92 (t, J=7.53 Hz, 3H). MS obsd. (ESI⁺) [(M+H)⁺]: 347.

Separation of compound 1e by chiral HPLC afforded Compound 1e-A (slower eluting, 500 mg) and Compound 1e-B (faster eluting, 490 mg) as white solid. (Separation condition: methanol 5%-40% (0.05% DEA)/CO₂ on ChiralPak AS-3 column.)

Compound 1e-A: ¹H NMR (DMSO-d₆, 400 MHz) δ ppm: 10.56 (s, 1H), 7.21-7.46 (m, 5H), 7.03 (s, 2H), 4.96 (s, 2H), 4.04 (s, 1H), 3.25-3.33 (m, 2H), 1.59-1.67 (m, 2H), 0.92 (t, J=7.4 Hz, 3H).

Compound 1e-B: ¹H NMR (DMSO-d₆, 400 MHz) δ ppm: 10.57 (s, 1H), 7.23-7.39 (m, 5H), 6.97 (s, 2H), 4.96 (s, 2H), 4.05 (s, 1H), 3.31-3.30 (m, 2H), 1.49-1.74 (m, 2H), 0.91 (t, J=7.4 Hz, 3H).

### Step 6: Preparation of 6-amino-9-benzyl-N-methyl-8-oxo-N-propyl-2-(propylsulfonimidoyl)purine-7-carboxamide (Example 1)

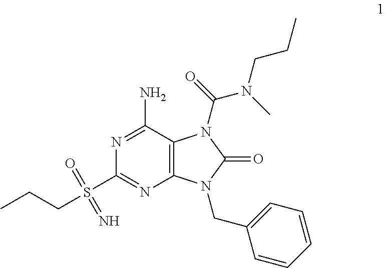 [SMILES: CCCN(C)C(=O)n1c(=O)n(Cc2ccccc2)c2nc(S(=N)(=O)CCC)nc(N)c21]

To a solution of 6-amino-9-benzyl-2-(propylsulfonimidoyl)-7H-purin-8-one (300 mg, Compound 1e), pyridine (329 mg, 4.2 mmol) and DIPEA (538 mg, 4.2 mmol) in NMP (5 mL) was added N-methyl-N-propyl-carbamoyl chloride (564 mg, 4.2 mmol, Intermediate AA) at RT. The mixture was stirred at RT for 10 hrs. The reaction mixture was concentrated and the residue was purified by prep-HPLC to give 6-amino-9-benzyl-N-methyl-8-oxo-N-propyl-2-(propylsulfonimidoyl)purine-7-carboxamide (108 mg, Example 1) as a white solid. ¹H NMR (400 MHz, DMSO-d₆) δ ppm: 7.45-7.24 (m, 5H), 6.89 (s, 2H), 5.01 (s, 2H), 4.17 (s, 1H), 3.44-3.34 (m, 2H), 3.36-3.34 (m, 2H), 3.10-3.00 (m, 3H), 1.74-1.52 (m, 4H), 1.01-0.72 (m, 6H). MS obsd. (ESI⁺) [(M+H)⁺]: 446.

Separation of compound of Example 1 by chiral HPLC afforded Example 1-A (slower eluting, 50 mg) and Example 1-B (faster eluting, 40 mg) as white solid with isopropanol 5%-40% (0.05% DEA)/CO₂ on ChiralPak AD-3 column.

### Example 1-A

¹H NMR (400 MHz, DMSO-d₆) δ ppm: 7.44-7.24 (m, 5H), 6.89 (s, 2H), 5.01 (s, 2H), 4.17 (s, 1H), 3.44-3.37 (m, 2H), 3.37-3.35 (m, 2H), 3.10-3.00 (m, 3H), 1.74-1.52 (m, 4H), 1.00-0.72 (m, 6H). MS obsd. (ESI⁺) [(M+H)⁺]: 446.

### Example 1-B

¹H NMR (400 MHz, DMSO-d₆) δ ppm: 7.45-7.26 (m, 5H), 6.88 (s, 2H), 5.01 (s, 2H), 4.15 (s, 1H), 3.44-3.36 (m, 2H), 3.34 (s, 2H), 3.10-3.01 (m, 3H), 1.77-1.52 (m, 4H), 1.02-0.67 (m, 6H). MS obsd. (ESI⁺) [(M+H)⁺]: 446.

### Method B: Alternative Method to Prepare 6-amino-9-benzyl-2-(propylsulfonimidoyl)-7H-purin-8-one (Compound 1e)

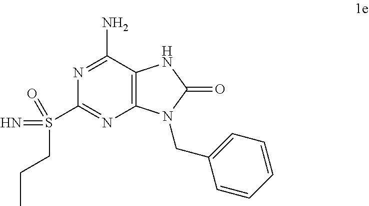 [SMILES: CCCS(=N)(=O)c1nc(N)c2[nH]c(=O)n(Cc3ccccc3)c2n1]

### Step 1: Preparation of N-benzyl-6-chloro-5-nitro-2-propylsulfanyl-pyrimidin-4-amine (Compound 1f)

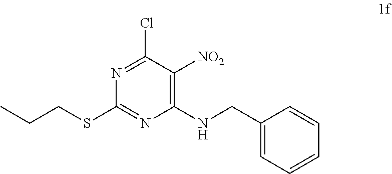 [SMILES: CCCSc1nc(Cl)c([N+](=O)[O-])c(NCc2ccccc2)n1]

To a solution of 4,6-dichloro-5-nitro-2-propylsulfanylpyrimidine (150.0 g, 559.5 mmol) and DIPEA (108.5 g, 839.2 mmol) in THF (1.5 L) was added phenylmethanamine (60.0 g, 559.5 mmol) in THF (200 mL) slowly at −78° C. After addition, the mixture was warmed to 25° C., and stirred at this temperature for 16 hrs. The resulting mixture was diluted with EA (1 L), washed with water (400 mL) three times and brine (500 mL). The separated organic phase was dried over Na₂SO₄, filtered and concentrated in vacuo to give N-benzyl-6-chloro-5-nitro-2-propylsulfanyl-pyrimidin-4-amine (180.0 g, Compound 1f) as a yellow solid and used for next step without further purification. MS obsd. (ESI⁺) [(M+H)⁺]: 339.1.

### Step 2: Preparation of N4-benzyl-6-chloro-2-propylsulfanyl-pyrimidine-4,5-diamine (Compound 1g)

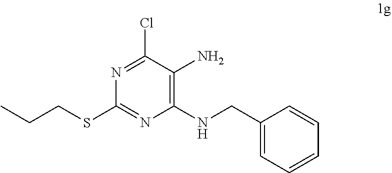 [SMILES: CCCSc1nc(Cl)c(N)c(NCc2ccccc2)n1]

To a solution of N-benzyl-6-chloro-5-nitro-2-propylsulfanyl-pyrimidin-4-amine (180 g, Compound 1f) and HOAc (319 g, 5.31 mol) in THF (3.0 L) was added Zn (174 g, 2.66 mol) slowly at 25° C. After the addition, the mixture was stirred at 25° C. for 16 hrs. The reaction was filtered and the filtrate was basified with saturated aq. NaHCO₃ (800 mL), extracted with EA (400 mL) three times, dried over Na₂SO₄ and concentrated in vacuo. The residue was purified by silica gel chromatography to give N4-benzyl-6-chloro-2-propylsulfanyl-pyrimidine-4,5-diamine (125 g, Compound 1g) as a brown solid. MS obsd. (ESI⁺) [(M+H)⁺]: 309.1.

### Step 3: Preparation of 9-benzyl-6-chloro-2-propylsulfanyl-7H-purin-8-one (Compound 1h)

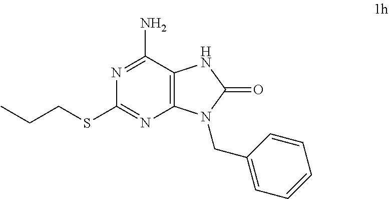 [SMILES: CCCSc1nc(N)c2[nH]c(=O)n(Cc3ccccc3)c2n1]

To a solution of N-benzyl-6-chloro-2-(propylsulfanyl)pyrimidine-4,5-diamine (72.0 g, 233.1 mmol, Compound 1g) and CDI (75.2 g, 233.1 mmol) in THF (800 mL) was stirred at 80° C. for 16 hrs. The resulting mixture was diluted with EA (400 mL), washed with water (200 mL) twice and brine (200 mL). The separated organic layer was dried over Na₂SO₄, concentrated in vacuo. The residue was washed with MTBE (200 mL) to give 9-benzyl-6-chloro-2-propylsulfanyl-7H-purin-8-one (58.0 g, Compound 1h) as a white solid and was used in next step without further purification. MS obsd. (ESI⁺) [(M+H)⁺]: 335.1.

### Step 4: Preparation of 9-benzyl-6-[(4-methoxyphenyl)methylamino]-2-propylsulfanyl-7H-purin-8-one (Compound 1i)

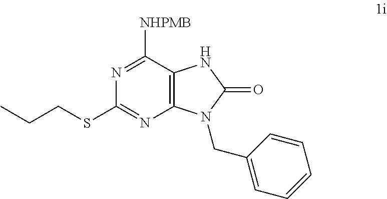

A solution of 9-benzyl-6-chloro-2-propylsulfanyl-7H-purin-8-one (58.0 g, Compound 1h) and PMBNH₂ (54.7 g, 398.42 mmol) in n-BuOH (600 mL) was stirred at 120° C. for 20 hrs. The reaction was concentrated and the residue was washed with MTBE (400 mL) to give 9-benzyl-6-[(4-methoxyphenyl)methylamino]-2-propylsulfanyl-7H-purin-8-one (75 g, Compound 1i) as a white solid and was used in next step without further purification. MS obsd. (ESI⁺) [(M+H)⁺]: 436.2.

### Step 5: Preparation of 6-amino-9-benzyl-2-propylsulfanyl-7H-purin-8-one (Compound 1c)

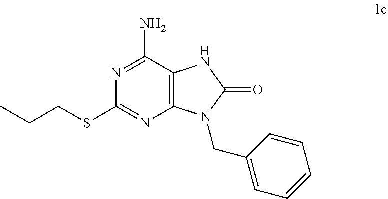 [SMILES: CCCSc1nc(N)c2[nH]c(=O)n(Cc3ccccc3)c2n1]

9-Benzyl-6-[(4-methoxyphenyl)methylamino]-2-propylsulfanyl-7H-purin-8-one (87.0 g, Compound ii) in TFA (200 mL) was stirred at 80° C. for 16 hrs. The resulting reaction mixture was concentrated, basified with saturated aq. NaHCO₃ (600 mL). The resulting precipitate was collected by filtration and washed with (PE/DCM=2:1, 400 mL) to give 6-amino-9-benzyl-2-propylsulfanyl-7H-purin-8-one (38.0 g, Compound 1c) as a white solid. MS obsd. (ESI⁺) [(M+H)⁺]: 316.1.

### Step 6: Preparation of 6-amino-9-benzyl-2-propylsulfinyl-7H-purin-8-one (Compound 1d)

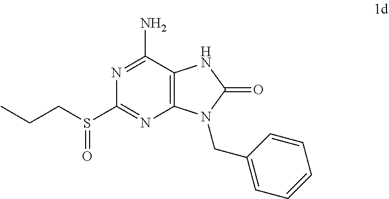 [SMILES: CCCS(=O)c1nc(N)c2[nH]c(=O)n(Cc3ccccc3)c2n1]

To a solution of m-CPBA (22.98 g, 113.2 mmol) in THF (50 mL) was added dropwise to a suspension of 6-amino-9-benzyl-2-propylsulfanyl-7H-purin-8-one (35.0 g, compound 1c) in THF (200 mL) at 0° C. After the addition, the reaction mixture was stirred at 25° C. for 0.5 hr. The mixture was filtered and washed with MeCN (400 mL), MTBE (500 mL) to give 6-amino-9-benzyl-2-propylsulfinyl-7H-purin-8-one (35.1 g, Compound 1d) as a white solid, which was used for the next step without further purification. MS obsd. (ESI⁺) [(M+H)⁺]: 332.1.

### Step 7: Preparation of 6-amino-9-benzyl-2-(propylsulfonimidoyl)-7H-purin-8-one (Compound 1e)

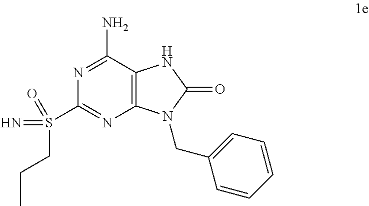 [SMILES: CCCS(=N)(=O)c1nc(N)c2[nH]c(=O)n(Cc3ccccc3)c2n1]

To a solution of 6-amino-9-benzyl-2-propylsulfinyl-7H-purin-8-one (34.0 g, Compound 1d) in Eaton's reagent (170.0 mL, 7.5 wt. % in methanesulphonic acid) was added NaN₃ (15.34 g, 253.97 mmol) at 60° C. slowly. Then the mixture was stirred at 60° C. for 30 mins. The resulting reaction mixture was cooled to 25° C., poured into ice cold NH₃—H₂O (500 mL, 1 mol/L), extracted with n-BuOH (100 mL) four times and concentrated in vacuo. The residue was purified by prep-HPLC to give 6-amino-9-benzyl-2-(propylsulfonimidoyl)-7H-purin-8-one (10 g, Compound 1e). ¹H NMR (400 MHz, DMSO-d₆) & ppm: 10.65 (br. s., 1H), 7.26-7.37 (m, 5H), 6.98 (br. s., 2H), 4.97 (s, 2H), 4.02 (s, 1H), 3.33 (t, J=7.53 Hz, 2H), 1.55-1.74 (m, 2H), 0.92 (t, J=7.53 Hz, 3H). MS obsd. (ESI⁺) [(M+H)⁺]: 347.

### Example 2

### 6-Amino-9-benzyl-N-(2-methoxyethyl)-N-methyl-8-oxo-2-(propylsulfonimidoyl)purine-7-carboxamide

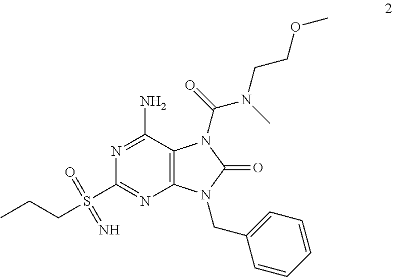 [SMILES: CCCS(=N)(=O)c1nc(N)c2c(n1)n(Cc1ccccc1)c(=O)n2C(=O)N(C)CCOC]

The title compound was prepared in analogy to Example 1, Method A, Step 6 by using N-(2-methoxyethyl)-N-methyl-carbamoyl chloride (Intermediate AB) instead of N-methyl-N-propyl-carbamoyl chloride (Intermediate AA). 6-Amino-9-benzyl-N-(2-methoxyethyl)-N-methyl-8-oxo-2-(propylsulfonimidoyl)purine-7-carboxamide (120 mg, Example 2) was obtained as a white solid. ¹H NMR (400 MHz, DMSO-d₆) δ ppm: 7.27-7.39 (m, 5H), 6.89 (br. s., 1H), 6.78 (br. s., 1H), 5.00 (s, 2H), 4.16 (br. d, J=4 Hz, 1H), 3.62 (br. dd, J=4, 12 Hz, 2H), 3.28-3.42 (m, 6H), 3.12 (d, J=12 Hz, 3H), 3.05 (s, 1H), 1.58-1.72 (m, 2H), 0.93 (t, J=8 Hz, 3H). MS obsd. (ESI⁺) [(M+H)⁺]: 462.

Separation of compound of Example 2 by chiral HPLC afforded Example 2-A (faster eluting, 33 mg) and Example 2-B (slower eluting, 46 mg) as white solid with methanol 5%-40% (0.05% DEA)/CO₂ on ChiralPak OJ-3 column.

### Example 2-A

¹H NMR (400 MHz, DMSO-d₆) δ ppm: 7.27-7.39 (m, 5H), 6.89 (br. s., 1H), 6.78 (br. s., 1H), 5.00 (s, 2H), 4.16 (br. d, J=4 Hz, 1H), 3.62 (br. dd, J=4, 12 Hz, 2H), 3.28-3.42 (m, 6H), 3.12 (d, J=12 Hz, 3H), 3.05 (s, 1H), 1.58-1.72 (m, 2H), 0.93 (t, J=8 Hz, 3H). MS obsd. (ESI⁺) [(M+H)⁺]: 462.

### Example 2-B

¹H NMR (400 MHz, DMSO-d₆) δ ppm: 7.27-7.39 (m, 5H), 6.89 (br. s., 1H), 6.78 (br. s., 1H), 5.00 (s, 2H), 4.16 (br. d, J=4 Hz, 1H), 3.62 (br. dd, J=4, 12 Hz, 2H), 3.28-3.42 (m, 6H), 3.12 (d, J=12 Hz, 3H), 3.05 (s, 1H), 1.58-1.72 (m, 2H), 0.93 (t, J=8 Hz, 3H). MS obsd. (ESI⁺) [(M+H)⁺]: 462.

### Example 3

### 6-Amino-9-benzyl-N-ethyl-8-oxo-N-propyl-2-(propylsulfonimidoyl)purine-7-carboxamide

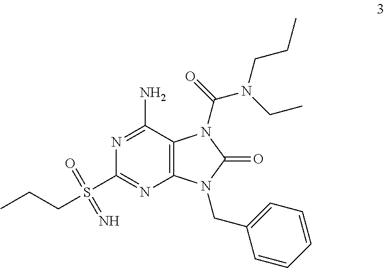 [SMILES: CCCN(CC)C(=O)n1c(=O)n(Cc2ccccc2)c2nc(S(=N)(=O)CCC)nc(N)c21]

The title compound was prepared in analogy to Example 1, Method A, Step 6 by using N-ethyl-N-propyl-carbamoyl chloride (Intermediate AC) instead of N-methyl-N-propyl-carbamoyl chloride (Intermediate AA). 6-Amino-9-benzyl-N-ethyl-8-oxo-N-propyl-2-(propylsulfonimidoyl)purine-7-carboxamide (51 mg, Example 3) was obtained as a white solid. ¹H NMR (400 MHz, DMSO-d₆) δ ppm: 7.27-7.39 (m, 5H), 6.85 (br. s., 2H), 4.99 (s, 2H), 4.20 (br. d, J=8.0 Hz, 1H), 3.13-3.54 (m, 4H), 1.46-1.72 (m, 4H), 1.30-1.39 (m, 1H), 1.00-1.26 (m, 6H), 0.81-0.95 (m, 5H), 0.73 (t, J=8 Hz, 1H). MS obsd. (ESI⁺) [(M+H)⁺]: 474.

### Example 4

### 6-Amino-9-benzyl-7-[4-(1-piperidyl)piperidine-1-carbonyl]-2-(propylsulfonimidoyl)purin-8-one

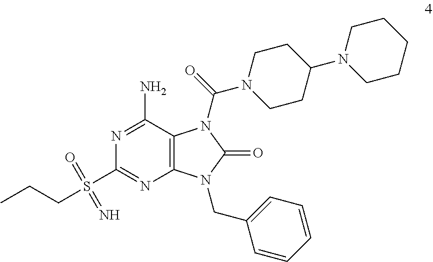 [SMILES: CCCS(=N)(=O)c1nc(N)c2c(n1)n(Cc1ccccc1)c(=O)n2C(=O)N1CCC(N2CCCCC2)CC1]

The title compound was prepared in analogy to Example 1, Method A, Step 6 by using (1,4′-bipiperidine)-1′-carbonyl chloride instead of N-methyl-N-propyl-carbamoyl chloride (Intermediate AA). 6-Amino-9-benzyl-7-[4-(1-piperidyl)piperidine-1-carbonyl]-2-(propylsulfonimidoyl)purin-8-one (55 mg, Example 4) was obtained as a white powder. ¹H NMR (400 MHz, DMSO-d₆) δ ppm: 7.39-7.27 (m, 5H), 6.97 (br. s., 2H), 4.99 (s, 2H), 4.20 (br. s., 2H), 3.85 (d, J=12.5 Hz, 1H), 3.43-3.15 (m, 3H), 2.96 (t, J=12.3 Hz, 2H), 2.56 (m, 4H), 1.83 (m, 1H), 1.79-1.54 (m, 4H), 1.50 (br. s., 4H), 1.45-1.33 (m, 3H), 0.93 (t, J=7.4 Hz, 3H). MS obsd. (ESI⁺) [(M+H)⁺]: 541.2.

### Example 5

### 6-Amino-9-benzyl-N-ethyl-N-(2-methoxyethyl)-8-oxo-2-(propylsulfonimidoyl)purine-7-carboxamide

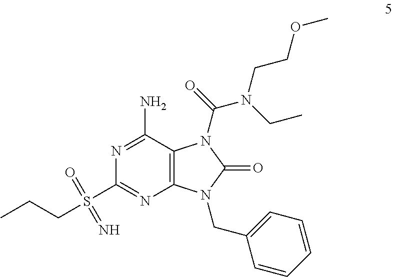 [SMILES: CCCS(=N)(=O)c1nc(N)c2c(n1)n(Cc1ccccc1)c(=O)n2C(=O)N(CC)CCOC]

The title compound was prepared in analogy to Example 1, Method A, Step 6 by using N-ethyl-N-(2-methoxyethyl)carbamoyl chloride (Intermediate AD) instead of N-methyl-N-propyl-carbamoyl chloride (Intermediate AA). 6-Amino-9-benzyl-N-ethyl-N-(2-methoxyethyl)-8-oxo-2-(propylsulfonimidoyl)purine-7-carboxamide (34 mg, Example 5) was obtained as a white powder. ¹H NMR (400 MHz, DMSO-d₆) δ ppm: 7.39-7.28 (m, 5H), 6.89 (br. s., 1H), 6.74 (br. s., 1H), 4.99 (s, 2H), 4.17 (d, J=8.1 Hz, 1H), 3.67 (br. s., 2H), 3.63-3.51 (m, 2H), 3.50-3.34 (m, 4H), 3.29 (s, 1H), 3.11 (s, 2H), 1.73-1.59 (m, 2H), 1.23-1.07 (m, 3H), 0.93 (t, J=7.5 Hz, 3H). MS obsd. (ESI⁺) [(M+H)⁺]: 476.3.

### Example 6

### 6-Amino-9-benzyl-N-butyl-N-ethyl-8-oxo-2-(propylsulfonimidoyl)purine-7-carboxamide

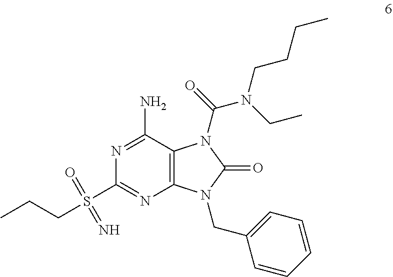 [SMILES: CCCCN(CC)C(=O)n1c(=O)n(Cc2ccccc2)c2nc(S(=N)(=O)CCC)nc(N)c21]

The title compound was prepared in analogy to Example 1, Method A, Step 6 by using N-butyl-N-ethyl-carbamoyl chloride (Intermediate AE) instead of N-methyl-N-propyl-carbamoyl chloride (Intermediate AA). 6-Amino-9-benzyl-N-butyl-N-ethyl-8-oxo-2-(propylsulfonimidoyl)purine-7-carboxamide (51 mg, Example 6) was obtained as a white solid. ¹H NMR (400 MHz, DMSO-d₆) δ ppm: 7.27-7.39 (m, 5H), 6.85 (br. s., 2H), 4.99 (s, 2H), 4.20 (br. d, J=8.0 Hz, 1H), 3.13-3.54 (m, 4H), 1.46-1.72 (m, 4H), 1.30-1.39 (m, 1H), 1.00-1.26 (m, 6H), 0.81-0.95 (m, 5H), 0.73 (t, J=8 Hz, 1H). MS obsd. (ESI⁺) [(M+H)⁺]: 474.

### Example 7

### 6-Amino-9-benzyl-N-(2-methoxyethyl)-8-oxo-N-propyl-2-(propylsulfonimidoyl)purine-7-carboxamide

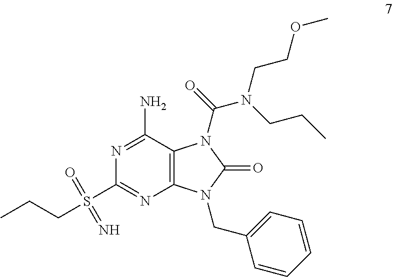 [SMILES: CCCN(CCOC)C(=O)n1c(=O)n(Cc2ccccc2)c2nc(S(=N)(=O)CCC)nc(N)c21]

The title compound was prepared in analogy to Example 1, Method A, Step 6 by using N-ethyl-N-(2-methoxyethyl)carbamoyl chloride (Intermediate AF) instead of N-methyl-N-propyl-carbamoyl chloride (Intermediate AA). 6-amino-9-benzyl-N-(2-methoxyethyl)-8-oxo-N-propyl-2-(propylsulfonimidoyl)purine-7-carboxamide (35 mg, Example 7) was obtained as a white powder. ¹H NMR (400 MHz, DMSO-d₆) δ ppm: 7.40-7.28 (m, 5H), 6.89 (br. s., 1H), 6.75 (br. s., 1H), 5.00 (d, J=5.5 Hz, 2H), 4.24-4.16 (m, 1H), 3.77 (br. s., 1H), 3.67 (br. s., 1H), 3.62-3.53 (m, 1H), 3.42-3.27 (m, 5H), 3.23-3.02 (m, 3H), 1.66-1.38 (m, 4H), 0.96-0.70 (m, 6H). MS obsd. (ESI⁺) [(M+H)⁺]: 490.5.

### Example 8

### 6-Amino-9-benzyl-N,N-bis(2-methoxyethyl)-8-oxo-2-(propylsulfonimidoyl)purine-7-carboxamide

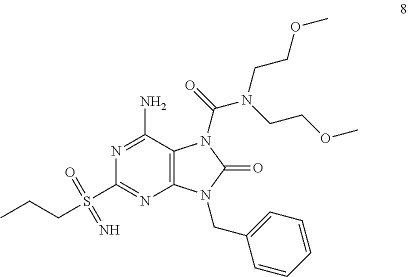 [SMILES: CCCS(=N)(=O)c1nc(N)c2c(n1)n(Cc1ccccc1)c(=O)n2C(=O)N(CCOC)CCOC]

The title compound was prepared in analogy to Example 1, Method A, Step 6 by using bis(2-methoxyethyl)carbamic chloride (Intermediate AG) instead of N-methyl-N-propyl-carbamoyl chloride (Intermediate AA). 6-Amino-9-benzyl-N,N-bis(2-methoxyethyl)-8-oxo-2-(propylsulfonimidoyl)purine-7-carboxamide (35 mg, Example 8) was obtained as a white powder. ¹H NMR (400 MHz, DMSO-d₆) δ ppm: 7.40-7.28 (m, 5H), 6.83 (br. s., 2H), 4.99 (s, 2H), 3.71 (br. s., 3H), 3.52-3.27 (m, 11H), 3.09 (s, 3H), 1.73-1.59 (m, 2H), 0.93 (t, J=7.5 Hz, 3H). MS obsd. (ESI⁺) [(M+H)⁺]: 506.

### Example 9

### 6-Amino-7-(azetidine-1-carbonyl)-9-benzyl-2-(propylsulfonimidoyl)purin-8-one

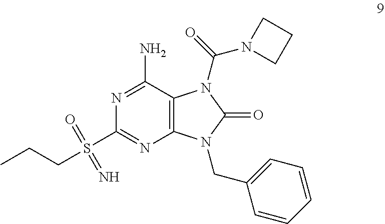 [SMILES: CCCS(=N)(=O)c1nc(N)c2c(n1)n(Cc1ccccc1)c(=O)n2C(=O)N1CCC1]

The title compound was prepared in analogy to Example 1, Method A, Step 6 by using azetidine-1-carbonyl chloride (Intermediate AH) instead of N-methyl-N-propyl-carbamoyl chloride (Intermediate AA). 6-Amino-7-(azetidine-1-carbonyl)-9-benzyl-2-(propylsulfonimidoyl)purin-8-one (120 mg, Example 9) was obtained as a white powder. ¹HNMR (400 MHz, DMSO-d₆) δ ppm: 7.02-7.43 (m, 7H), 4.99 (s, 2H), 4.31 (t, J=7.65 Hz, 2H), 4.08-4.23 (m, 3H), 3.34-3.41 (m, 2H), 2.28 (m, 2H), 1.56-1.73 (m, 2H), 0.93 (t, J=7.40 Hz, 3H). MS obsd. (ESI⁺) [(M+H)⁺]: 430.

### Example 10

### 6-Amino-9-benzyl-N-isopropyl-N-methyl-8-oxo-2-(propylsulfonimidoyl)purine-7-carboxamide

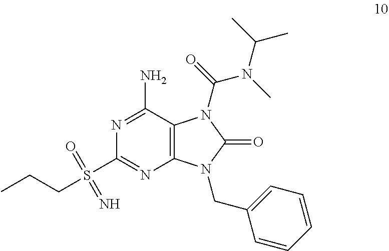 [SMILES: CCCS(=N)(=O)c1nc(N)c2c(n1)n(Cc1ccccc1)c(=O)n2C(=O)N(C)C(C)C]

The title compound was prepared in analogy to Example 1, Method A, Step 6 by using N-isopropyl-N-methyl-carbamoyl chloride (Intermediate AI) instead of N-methyl-N-propyl-carbamoyl chloride (Intermediate AA). 6-Amino-9-benzyl-N-isopropyl-N-methyl-8-oxo-2-(propylsulfonimidoyl)purine-7-carboxamide (97 mg, Example 10) was obtained as a white solid. ¹H NMR (400 MHz, DMSO-d₆) δ ppm: 7.27-7.39 (m, 5H), 6.87 (br. s., 2H), 4.99 (s, 2H), 4.38-4.45 (m, 1H), 4.09-4.21 (m, 1H), 3.29-3.43 (m, 2H), 2.89-2.95 (m, 3H), 1.58-1.73 (m, 2H), 1.21 (br d, J=8 Hz, 6H), 0.93 (t, J=8 Hz, 3H). MS obsd. (ESI⁺) [(M+H)⁺]: 446.

### Example 11

### 6-Amino-9-benzyl-7-(4-methylpiperazine-1-carbonyl)-2-(propylsulfonimidoyl)purin-8-one

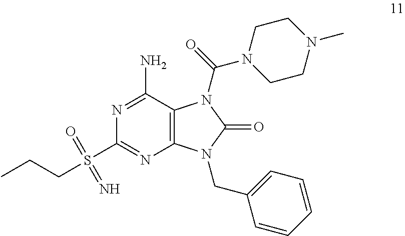 [SMILES: CCCS(=N)(=O)c1nc(N)c2c(n1)n(Cc1ccccc1)c(=O)n2C(=O)N1CCN(C)CC1]

The title compound was prepared in analogy to Example 1, Method A, Step 6 by using 4-methylpiperazine-1-carbonyl chloride instead of N-methyl-N-propyl-carbamoyl chloride (Intermediate AA). 6-Amino-9-benzyl-7-(4-methylpiperazine-1-carbonyl)-2-(propylsulfonimidoyl)purin-8-one (59.5 mg, Example 11) was obtained as a yellow solid. ¹H NMR (400 MHz, DMSO-d₆) δ ppm: 7.39-7.31 (m, 5H), 6.99 (s, 2H), 4.98 (s, 2H), 4.18 (s, 1H), 3.58-3.49 (m, 6H), 2.42 (m, 4H), 2.22 (s, 3H), 1.66-1.61 (m, 2H), 0.95-0.91 (t, J=7.2 Hz, 3H). MS obsd. (ESI⁺) [(M+H)⁺]: 473.

### Example 12

### 6-Amino-9-benzyl-N-(3-methoxypropyl)-N-methyl-8-oxo-2-(propylsulfonimidoyl)purine-7-carboxamide

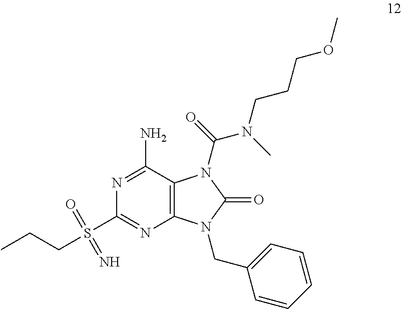 [SMILES: CCCS(=N)(=O)c1nc(N)c2c(n1)n(Cc1ccccc1)c(=O)n2C(=O)N(C)CCCOC]

The title compound was prepared in analogy to Example 1, Method A, Step 6 by using N-(3-methoxypropyl)-N-methyl-carbamoyl chloride instead of N-methyl-N-propyl-carbamoyl chloride (Intermediate AA). 6-Amino-9-benzyl-N-(3-methoxypropyl)-N-methyl-8-oxo-2-(propylsulfonimidoyl)purine-7-carboxamide (92.2 mg, Example 12) was obtained as a white solid. ¹H NMR (400 MHz, DMSO-d₆) δ ppm: 7.23-7.45 (m, 5H), 6.94 (s., 2H), 4.93-5.08 (m, 2H), 4.19 (s, 1H), 3.30-3.62 (m, 6H), 3.25 (s, 3H), 3.02-3.10 (m, 3H), 1.74-1.90 (m, 2H), 1.55-1.77 (m, 2H), 0.98-0.82 (m, 3H). MS obsd. (ESI⁺) [(M+H)⁺]: 476.3.

### Example 13

### 6-Amino-9-benzyl-N-isobutyl-N-methyl-8-oxo-2-(propylsulfonimidoyl)purine-7-carboxamide

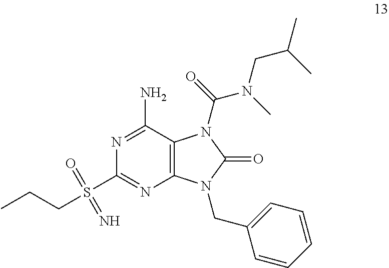 [SMILES: CCCS(=N)(=O)c1nc(N)c2c(n1)n(Cc1ccccc1)c(=O)n2C(=O)N(C)CC(C)C]

The title compound was prepared in analogy to Example 1, Method A, Step 6 by using N-isobutyl-N-methyl-carbamoyl chloride (Intermediate AL) instead of N-methyl-N-propyl-carbamoyl chloride (Intermediate AA). 6-Amino-9-benzyl-N-isobutyl-N-methyl-8-oxo-2-(propylsulfonimidoyl)purine-7-carboxamide (64 mg, Example 13) was obtained as a white solid. ¹H NMR (400 MHz, DMSO-d₆) δ ppm: 7.27-7.40 (m, 5H), 6.89 (br. s., 2H), 5.00 (s, 2H), 4.16 (br. s., 1H), 3.25-3.44 (m, 4H), 3.07 (s, 2H), 3.03 (s, 1H), 1.87-2.09 (m, 1H), 1.57-1.74 (m, 2H), 0.75-0.99 (m, 9H). MS obsd. (ESI⁺) [(M+H)⁺]: 460.

### Example 14

### Ethyl 2-[[6-amino-9-benzyl-8-oxo-2-(propylsulfonimidoyl)purine-7-carbonyl]-methyl-amino]acetate

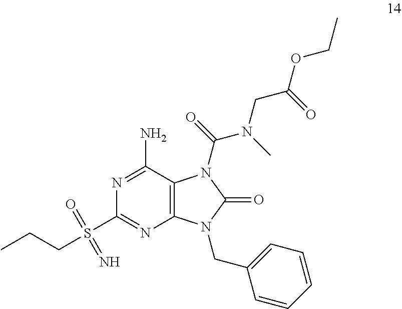 [SMILES: CCCS(=N)(=O)c1nc(N)c2c(n1)n(Cc1ccccc1)c(=O)n2C(=O)N(C)CC(=O)OCC]

The title compound was prepared in analogy to Example 1, Method A, Step 6 by using ethyl 2-((chlorocarbonyl)(methyl)amino)acetate (Intermediate AP) instead of N-methyl-N-propyl-carbamoyl chloride (Intermediate AA). Ethyl 2-[[6-amino-9-benzyl-8-oxo-2-(propylsulfonimidoyl)purine-7-carbonyl]-methyl-amino]acetate (38 mg, Example 14) was obtained as a light yellow powder. ¹H NMR (400 MHz, DMSO-d₆) δ ppm: 7.41-7.27 (m, 5H), 6.82 (br. s., 1H), 5.04-4.95 (m, 2H), 4.35 (br. s., 1H), 4.28 (br. s., 1H), 4.23-4.16 (m, 2H), 4.08 (q, J=7.2 Hz, 1H), 3.43-3.28 (m, 3H), 3.15 (s, 2H), 3.08 (s, 1H), 1.71-1.58 (m, 2H), 1.24 (t, J=7.0 Hz, 2H), 1.12 (t, J=7.0 Hz, 1H), 0.93 (t, J=7.4 Hz, 3H). MS obsd. (ESI⁺) [(M+H)⁺]: 490.

### Example 15

### Ethyl 3-[[6-amino-9-benzyl-8-oxo-2-(propylsulfonimidoyl)purine-7-carbonyl]-methyl-amino]propanoate

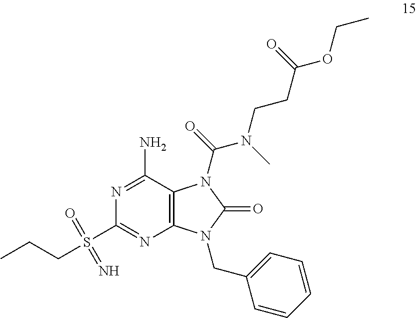 [SMILES: CCCS(=N)(=O)c1nc(N)c2c(n1)n(Cc1ccccc1)c(=O)n2C(=O)N(C)CCC(=O)OCC]

The title compound was prepared in analogy to Example 1, Method A, Step 6 by using ethyl 3-((chlorocarbonyl)(methyl)amino)propanoate instead of N-methyl-N-propyl-carbamoyl chloride (Intermediate AA). Ethyl 3-[[6-amino-9-benzyl-8-oxo-2-(propylsulfonimidoyl)purine-7-carbonyl]-methyl-amino]propanoate (35 mg, Example 15) was obtained as a white powder. ¹H NMR (400 MHz, DMSO-d₆) δ ppm: 7.43-7.26 (m, 5H), 6.93 (br. s., 2H), 4.99 (s, 2H), 4.16 (s, 1H), 4.08 (q, J=7.1 Hz, 1H), 3.99 (d, J=7.0 Hz, 1H), 3.67 (br. s., 2H), 3.40-3.29 (m, 2H), 3.08 (s, 2H), 2.99 (s, 1H), 2.71 (t, J=6.4 Hz, 2H), 1.74-1.56 (m, 2H), 1.27-1.05 (m, 3H), 0.93 (t, J=7.5 Hz, 3H). MS obsd. (ESI⁺) [(M+H)⁺]: 504.

### Example 16

### tert-Butyl 3-[[6-amino-9-benzyl-8-oxo-2-(propylsulfonimidoyl)purine-7-carbonyl]-methyl-amino]propanoate

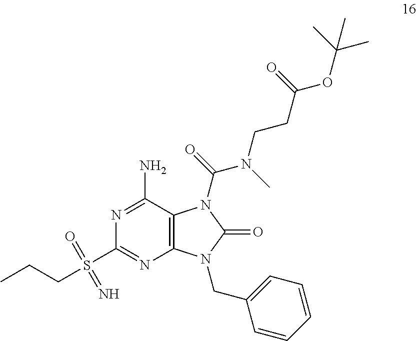 [SMILES: CCCS(=N)(=O)c1nc(N)c2c(n1)n(Cc1ccccc1)c(=O)n2C(=O)N(C)CCC(=O)OC(C)(C)C]

The title compound was prepared in analogy to Example 1, Method A, Step 6 by using tert-butyl 3-[chlorocarbonyl(methyl)amino]propanoate (Intermediate AR) instead of N-methyl-N-propyl-carbamoyl chloride (Intermediate AA). tert-Butyl 3-[[6-amino-9-benzyl-8-oxo-2-(propylsulfonimidoyl)purine-7-carbonyl]-methyl-amino]propanoate (60 mg, Example 16) was obtained as a white powder. ¹H NMR (400 MHz, DMSO-d₆) δ ppm: 7.41-7.27 (m, 5H), 6.93 (br. s., 2H), 4.99 (s, 2H), 4.15 (s, 1H), 3.64 (br. s., 2H), 3.51-3.33 (m, 2H), 3.08 (s, 2H), 2.98 (s, 1H), 2.62 (t, J=6.9 Hz, 2H), 1.71-1.57 (m, 2H), 1.41 (s, 6H), 1.34 (s, 3H), 0.93 (t, J=7.4 Hz, 3H). MS obsd. (ESI⁺) [(M+H)⁺]: 532.

### Example 17

### Ethyl (2S)-2-[[6-amino-9-benzyl-8-oxo-2-(propylsulfonimidoyl)purine-7-carbonyl]-methyl-amino]propanoate

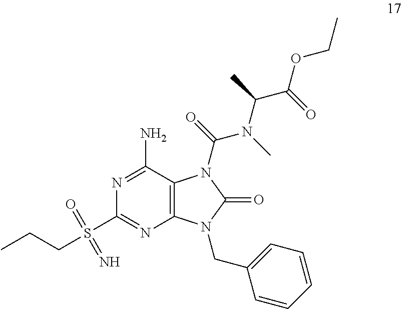 [SMILES: CCCS(=N)(=O)c1nc(N)c2c(n1)n(Cc1ccccc1)c(=O)n2C(=O)N(C)[C@@H](C)C(=O)OCC]

The title compound was prepared in analogy to Example 1, Method A, Step 6 by using ethyl (2S)-2-[chlorocarbonyl(methyl)amino]propanoate (Intermediate AS) instead of N-methyl-N-propyl-carbamoyl chloride (Intermediate AA). Ethyl (2S)-2-[[6-amino-9-benzyl-8-oxo-2-(propylsulfonimidoyl)purine-7-carbonyl]-methyl-amino]propanoate (34.1 mg, Example 17) was obtained as a yellow solid. ¹H NMR (300 MHz, DMSO-d₆) δ ppm: 7.22-7.49 (m, 5H), 6.78 (br. s., 2H), 4.93-5.08 (m, 2H), 4.75 (br. s., 1H), 3.96-4.29 (m, 3H), 3.30-3.46 (m, 2H), 3.09 (s, 2H), 2.93 (br. s., 1H), 1.55-1.77 (m, 2H), 1.48 (d, J=7.16 Hz, 3H), 1.09-1.29 (m, 3H), 0.94 (t, J=7.44 Hz, 3H). MS obsd. (ESI⁺) [(M+H)⁺]: 504.2.

### Example 18

### tert-Butyl (2S)-2-[[6-amino-9-benzyl-8-oxo-2-(propylsulfonimidoyl)purine-7-carbonyl]-methyl-amino]-4-methyl-pentanoate

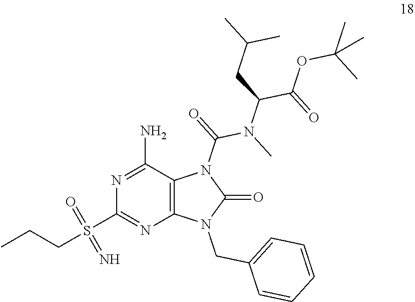 [SMILES: CCCS(=N)(=O)c1nc(N)c2c(n1)n(Cc1ccccc1)c(=O)n2C(=O)N(C)[C@@H](CC(C)C)C(=O)OC(C)(C)C]

The title compound was prepared in analogy to Example 1, Method A, Step 6 by using tert-butyl (2S)-2-[chlorocarbonyl(methyl)amino]-4-methyl-pentanoate (Intermediate AT) instead of N-methyl-N-propyl-carbamoyl chloride (Intermediate AA). tert-Butyl (2S)-2-[[6-amino-9-benzyl-8-oxo-2-(propylsulfonimidoyl)purine-7-carbonyl]-methyl-amino]-4-methyl-pentanoate (22 mg, Example 18) was obtained as a white solid. ¹H NMR (400 MHz, DMSO-d₆) δ ppm: 7.42-7.27 (m, 5H), 6.78 (br. s., 2H), 5.05-4.96 (m, 2H), 4.78 (br. s., 1H), 4.33 (br. s., 1H), 3.51-3.37 (m, 2H), 3.01 (s, 3H), 1.75-1.54 (m, 4H), 1.44 (s, 8H), 1.33-1.11 (m, 2H), 0.99-0.82 (m, 9H). MS obsd. (ESI⁺) [(M+H)⁺]: 574.3.

### Example 19

### Isopropyl (2S)-2-[[6-amino-9-benzyl-8-oxo-2-(propylsulfonimidoyl)purine-7-carbonyl]-methyl-amino]-4-methyl-pentanoate

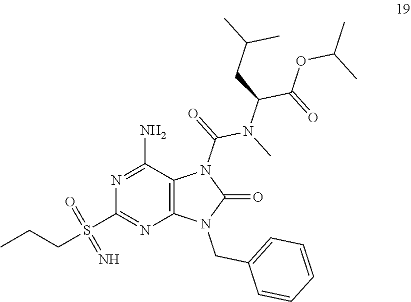 [SMILES: CCCS(=N)(=O)c1nc(N)c2c(n1)n(Cc1ccccc1)c(=O)n2C(=O)N(C)[C@@H](CC(C)C)C(=O)OC(C)C]

The title compound was prepared in analogy to Example 1, Method A, Step 6 by using isopropyl (2S)-2-[chlorocarbonyl(methyl)amino]-4-methyl-pentanoate (Intermediate AU) instead of N-methyl-N-propyl-carbamoyl chloride (Intermediate AA). Isopropyl (2S)-2-[[6-amino-9-benzyl-8-oxo-2-(propylsulfonimidoyl)purine-7-carbonyl]-methyl-amino]-4-methyl-pentanoate (43 mg, Example 19) was obtained as a white powder. ¹H NMR (400 MHz, DMSO-d₆) δ ppm: 7.43-7.27 (m, 5H), 6.75 (br. s., 2H), 5.05-4.94 (m, 3H), 4.88 (br. s., 1H), 4.19 (br. s., 1H), 3.43-3.34 (m, 2H), 3.01 (s, 3H), 1.91 (br. s., 1H), 1.77-1.56 (m, 4H), 1.25-1.16 (m, 6H), 0.99-0.83 (m, 9H). MS obsd. (ESI⁺) [(M+H)⁺]: 560.3.

### Example 20

### Ethyl (2S)-2-[[6-amino-9-benzyl-8-oxo-2-(propylsulfonimidoyl)purine-7-carbonyl]-methyl-amino]-3-methyl-butanoate

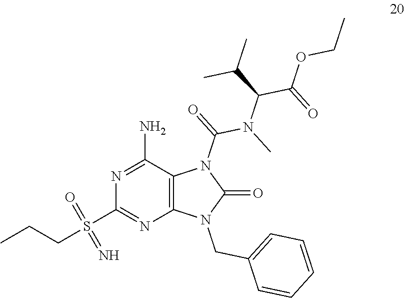 [SMILES: CCCS(=N)(=O)c1nc(N)c2c(n1)n(Cc1ccccc1)c(=O)n2C(=O)N(C)[C@H](C(=O)OCC)C(C)C]

The title compound was prepared in analogy to Example 1, Method A, Step 6 by using ethyl (2S)-2-[chlorocarbonyl(methyl)amino]-3-methyl-butanoate (Intermediate AV) instead of N-methyl-N-propyl-carbamoyl chloride (Intermediate AA). Ethyl (2S)-2-[[6-amino-9-benzyl-8-oxo-2-(propylsulfonimidoyl)purine-7-carbonyl]-methyl-amino]-3-methyl-butanoate (51.5 mg, Example 20) was obtained as a white powder. ¹H NMR (400 MHz, DMSO-d₆) δ ppm: 7.23-7.51 (m, 5H), 6.76 (br. s., 2H), 5.01 (br. s., 2H), 4.42 (br. s., 1H), 3.97-4.26 (m, 3H), 3.34-3.45 (m, 2H), 3.12 (br. s., 3H), 2.24 (br. s., 1H), 1.65 (br. s., 2H), 1.13-1.29 (m, 3H), 0.88-1.10 (m, 9H). MS obsd. (ESI⁺) [M+H⁺]: 532.2.

### Example 21

### Ethyl (2S)-2-[[6-amino-9-benzyl-8-oxo-2-(propylsulfonimidoyl)purine-7-carbonyl]-methyl-amino]-4-methyl-pentanoate

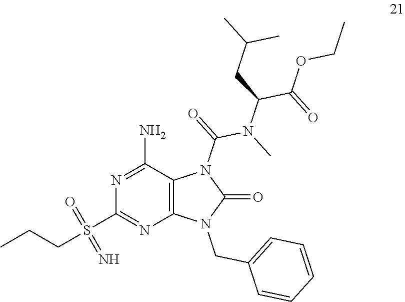 [SMILES: CCCS(=N)(=O)c1nc(N)c2c(n1)n(Cc1ccccc1)c(=O)n2C(=O)N(C)[C@@H](CC(C)C)C(=O)OCC]

The title compound was prepared in analogy to Example 1, Method A, Step 6 by using ethyl (2S)-2-[chlorocarbonyl(methyl)amino]-4-methyl-pentanoate (Intermediate AW) instead of N-methyl-N-propyl-carbamoyl chloride (Intermediate AA). Ethyl (2S)-2-[[6-amino-9-benzyl-8-oxo-2-(propylsulfonimidoyl)purine-7-carbonyl]-methyl-amino]-4-methyl-pentanoate (17.3 mg, Example 21) was obtained as a white powder. ¹H NMR (400 MHz, DMSO-d₆) δ ppm: 7.26-7.45 (m, 5H), 6.73 (br. s., 2H), 4.91-5.09 (m, 3H), 4.06-4.25 (m, 3H), 3.34-3.45 (m, 2H), 3.04 (br. s., 3H), 1.93 (br. s., 1H), 1.54-1.78 (m, 4H), 1.22 (t, J=7.09 Hz, 3H), 0.77-1.01 (m, 9H). MS obsd. (ESI⁺) [(M+H)⁺]: 546.3.

### Example 22

### Ethyl (2S)-2-[[6-amino-9-benzyl-8-oxo-2-(propylsulfonimidoyl)purine-7-carbonyl]-methyl-amino]-3-phenyl-propanoate

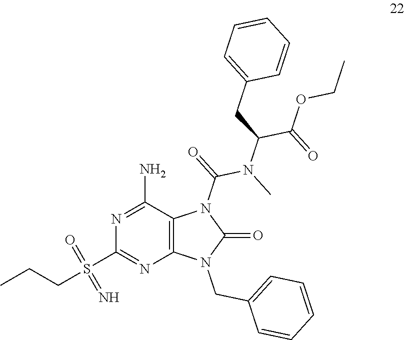 [SMILES: CCCS(=N)(=O)c1nc(N)c2c(n1)n(Cc1ccccc1)c(=O)n2C(=O)N(C)[C@@H](Cc1ccccc1)C(=O)OCC]

The title compound was prepared in analogy to Example 1, Method A, Step 6 by using ethyl (2S)-2-[chlorocarbonyl(methyl)amino]-3-phenyl-propanoate (Intermediate AX) instead of N-methyl-N-propyl-carbamoyl chloride (Intermediate AA). Ethyl (2S)-2-[[6-amino-9-benzyl-8-oxo-2-(propylsulfonimidoyl)purine-7-carbonyl]-methyl-amino]-3-phenyl-propanoate (30 mg, Example 22) was obtained as a white powder. ¹H NMR (400 MHz, DMSO-d₆) δ ppm: 7.42-7.16 (m, 10H), 4.97 (s, 3H), 4.19 (q, J=7.1 Hz, 2H), 3.35-3.15 (m, 6H), 3.10-2.90 (m, 3H), 1.71-1.46 (m, 2H), 1.28-1.18 (m, 4H), 0.97-0.85 (m, 3H). MS obsd. (ESI⁺) [(M+H)⁺]: 580.

### Example 23

### Isopropyl (2S)-2-[[6-amino-9-benzyl-8-oxo-2-(propylsulfonimidoyl)purine-7-carbonyl]-methyl-aminol]-3-phenyl-propanoate

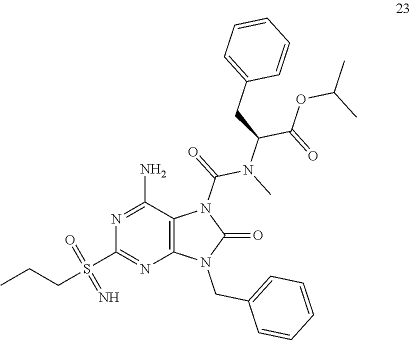 [SMILES: CCCS(=N)(=O)c1nc(N)c2c(n1)n(Cc1ccccc1)c(=O)n2C(=O)N(C)[C@@H](Cc1ccccc1)C(=O)OC(C)C]

The title compound was prepared in analogy to Example 1, Method A, Step 6 by using isopropyl (2S)-2-[chlorocarbonyl(methyl)amino]-3-phenyl-propanoate (Intermediate AY) instead of N-methyl-N-propyl-carbamoyl chloride (Intermediate AA). Isopropyl (2S)-2-[[6-amino-9-benzyl-8-oxo-2-(propylsulfonimidoyl)purine-7-carbonyl]-methyl-amino]-3-phenyl-propanoate (22 mg, Example 23) was obtained as a white powder. ¹H NMR (400 MHz, DMSO-d₆) δ ppm: 7.35-7.01 (m, 10H), 5.02-4.89 (m, 3H), 3.37-3.17 (m, 3H), 3.02-3.09 (m, 3H), 3.10-2.90 (m, 3H), 1.66-1.62 (m, 2H), 1.22-1.11 (m, 8H), 0.92 (t, J=7.4 Hz, 3H). MS obsd. (ESI⁺) [(M+H)⁺]: 594.

### Example 24

### tert-Butyl (2S)-2-[[6-amino-9-benzyl-8-oxo-2-(propylsulfonimidoyl)purine-7-carbonyl]-methyl-amino]-3-phenyl-propanoate

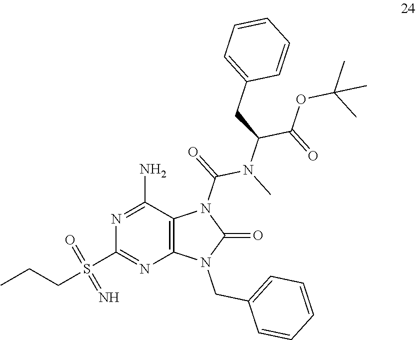 [SMILES: CCCS(=N)(=O)c1nc(N)c2c(n1)n(Cc1ccccc1)c(=O)n2C(=O)N(C)[C@@H](Cc1ccccc1)C(=O)OC(C)(C)C]

The title compound was prepared in analogy to Example 1, Method A, Step 6 by using tert-butyl (2S)-2-[chlorocarbonyl(methyl)amino]-3-phenyl-propanoate (Intermediate AZ) instead of N-methyl-N-propyl-carbamoyl chloride (Intermediate AA). tert-Butyl (2S)-2-[[6-amino-9-benzyl-8-oxo-2-(propylsulfonimidoyl)purine-7-carbonyl]-methyl-amino]-3-phenyl-propanoate (34 mg, Example 24) was obtained as a white powder. ¹H NMR (400 MHz, DMSO-d₆) δ ppm: 7.42-7.16 (m, 10H), 5.03-4.90 (m, 3H), 3.68-3.24 (m, 5H), 3.24-3.09 (m, 2H), 3.01 (s, 3H), 1.68-1.57 (m, 2H), 1.43 (s, 9H), 0.99-0.85 (m, 3H). MS obsd. (ESI⁺) [(M+H)⁺]: 608.3.

### Example 25

### N-[2-[Acetyl(methyl)amino]ethyl]-6-amino-9-benzyl-N-methyl-8-oxo-2-(propylsulfonimidoyl)purine-7-carboxamide

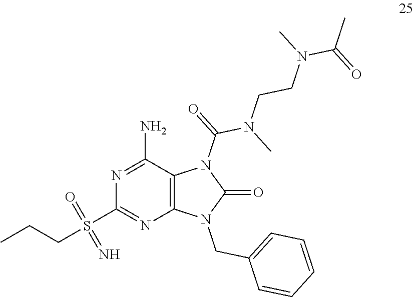 [SMILES: CCCS(=N)(=O)c1nc(N)c2c(n1)n(Cc1ccccc1)c(=O)n2C(=O)N(C)CCN(C)C(C)=O]

The title compound was prepared in analogy to Example 1, Method A, Step 6 by using N-[2-[acetyl(methyl)amino]ethyl]-N-methyl-carbamoyl chloride (Intermediate BA) instead of N-methyl-N-propyl-carbamoyl chloride (Intermediate AA). N-[2-[Acetyl(methyl)amino]ethyl]-6-amino-9-benzyl-N-methyl-8-oxo-2-(propylsulfonimidoyl)purine-7-carboxamide (26.1 mg, Example 25) was obtained as a white powder. H NMR (400 MHz, DMSO-d₆) δ ppm: 7.43-7.27 (m, 5H), 7.02 (br, 2H), 5.04-4.97 (m, 2H), 4.19-4.13 (m, 1H), 3.57 (d, J=5.5 Hz, 2H), 3.49-3.34 (m, 2H), 3.14 (s, 1H), 3.12-3.02 (m, 4H), 2.86 (d, J=7.5 Hz, 2H), 2.69-2.64 (m, 1H), 2.05 (s, 1H), 1.99 (s, 1H), 1.91-1.83 (m, 1H), 1.70-1.59 (m, 2H), 0.97-0.90 (m, 3H). MS obsd. (ESI⁺) [(M+H)⁺]: 503.2.

### Example 26

### Methyl N-[2-[[6-amino-9-benzyl-8-oxo-2-(propylsulfonimidoyl)purine-7-carbonyl]-methyl-amino]ethyl]-N-methyl-carbamate

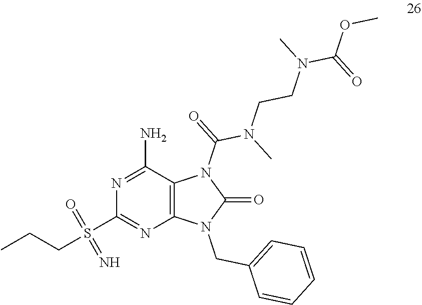 [SMILES: CCCS(=N)(=O)c1nc(N)c2c(n1)n(Cc1ccccc1)c(=O)n2C(=O)N(C)CCN(C)C(=O)OC]

The title compound was prepared in analogy to Example 1, Method A, Step 6 by using methyl N-[2-[chlorocarbonyl(methyl)amino]ethyl]-N-methyl-carbamate (Intermediate BB) instead of N-methyl-N-propyl-carbamoyl chloride (Intermediate AA). Methyl N-[2-[[6-amino-9-benzyl-8-oxo-2-(propylsulfonimidoyl)purine-7-carbonyl]-methyl-amino]ethyl]-N-methyl-carbamate (65 mg, Example 26) was obtained as a yellow solid. ¹H NMR (400 MHz, CDCl₃) δ ppm: 7.29-7.49 (m, 5H), 5.63-5.92 (m, 2H), 5.03-5.17 (m, 2H), 3.43-3.69 (m, 8H), 3.13-3.27 (m, 3H), 2.96-3.05 (m, 2H), 2.72 (br. s., 1H), 1.05 (t, J=7.40 Hz, 3H), 1.87 (dd, J=14.12, 6.96 Hz, 2H). MS obsd. (ESI⁺) [(M+H)⁺]: 519.2.

### Example 27

### tert-Butyl N-[2-[[6-amino-9-benzyl-8-oxo-2-(propylsulfonimidoyl)purine-7-carbonyl]-methyl-amino]ethyl]-N-methyl-carbamate

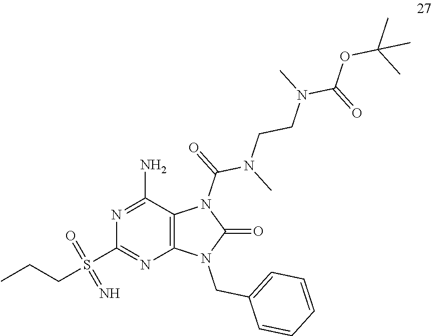 [SMILES: CCCS(=N)(=O)c1nc(N)c2c(n1)n(Cc1ccccc1)c(=O)n2C(=O)N(C)CCN(C)C(=O)OC(C)(C)C]

The title compound was prepared in analogy to Example 1, Method A, Step 6 by using tert-butyl N-[2-[chlorocarbonyl(methyl)amino]ethyl]-N-methyl-carbamate (Intermediate BC) instead of N-methyl-N-propyl-carbamoyl chloride (Intermediate AA). tert-Butyl N-[2-[[6-amino-9-benzyl-8-oxo-2-(propylsulfonimidoyl)purine-7-carbonyl]-methyl-amino]ethyl]-N-methyl-carbamate (32 mg, Example 27) was obtained as a white powder. ¹H NMR (400 MHz, DMSO-d₆) δ ppm: 7.43-7.26 (m, 5H), 6.89 (br. s., 2H), 4.99 (d, J=5.0 Hz, 2H), 4.16 (s, 1H), 3.55 (br. s., 2H), 3.48-3.34 (m, 2H), 3.10 (s, 2H), 3.07 (s, 1H), 2.86 (d, J=12.8 Hz, 2H), 2.74 (d, J=9.5 Hz, 1H), 2.70-2.60 (m, 1H), 1.72-1.54 (m, 2H), 1.39 (s, 6H), 1.23 (s, 2H), 1.13 (s, 2H), 0.93 (t, J=7.4 Hz, 3H). MS obsd. (ESI⁺) [(M+H)⁺]: 562.

### Example 28

### Ethyl N-[2-[[6-amino-9-benzyl-8-oxo-2-(propylsulfonimidoyl)purine-7-carbonyl]-methyl-amino]ethyl]-N-methyl-carbamate

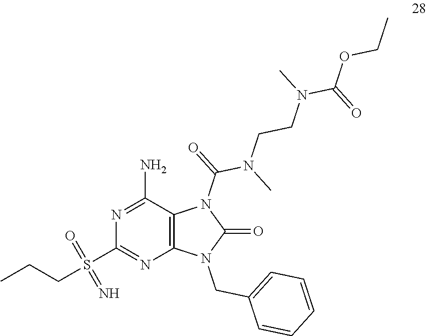 [SMILES: CCCS(=N)(=O)c1nc(N)c2c(n1)n(Cc1ccccc1)c(=O)n2C(=O)N(C)CCN(C)C(=O)OCC]

The title compound was prepared in analogy to Example 1, Method A, Step 6 by using ethyl N-[2-[chlorocarbonyl(methyl)amino]ethyl]-N-methyl-carbamate (Intermediate BD) instead of N-methyl-N-propyl-carbamoyl chloride (Intermediate AA). Ethyl N-[2-[[6-amino-9-benzyl-8-oxo-2-(propylsulfonimidoyl)purine-7-carbonyl]-methyl-amino]ethyl]-N-methyl-carbamate (87 mg, Example 28) was obtained as a yellow solid. ¹H NMR (400 MHz, CDCl₃) δ ppm: 7.29-7.53 (m, 5H), 5.65-5.90 (m, 2H), 5.02-5.14 (m, 2H), 3.38-4.21 (m, 9H), 3.14-3.26 (m, 3H), 3.00 (br. s., 2H), 2.73 (s, 1H), 1.76-1.99 (m, 2H), 1.22-1.31 (m, 3H), 1.05 (s, 3H). MS obsd. (ESI⁺) [(M+H)⁺]: 533.2.

### Example 29

### 2-[[6-Amino-9-benzyl-8-oxo-2-(propylsulfonimidoyl)purine-7-carbonyl]-methyl-amino]ethyl N-butyl-N-methyl-carbamate

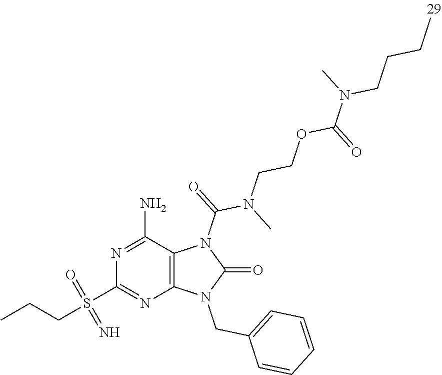 [SMILES: CCCCN(C)C(=O)OCCN(C)C(=O)n1c(=O)n(Cc2ccccc2)c2nc(S(=N)(=O)CCC)nc(N)c21]

The title compound was prepared in analogy to Example 1, Method A, Step 6 by using 2-[chlorocarbonyl(methyl)amino]ethyl N-butyl-N-methyl-carbamate (Intermediate BE) instead of N-methyl-N-propyl-carbamoyl chloride (Intermediate AA). 2-[[6-Amino-9-benzyl-8-oxo-2-(propylsulfonimidoyl)purine-7-carbonyl]-methyl-amino]ethyl N-butyl-N-methyl-carbamate (19 mg, Compound 29) was obtained as yellow solid. ¹H NMR (400 MHz, DMSO-d₆) δ ppm: 7.25-7.48 (m, 5H), 6.96 (br. s., 2H), 4.99 (s, 2H), 4.06-4.36 (m, 3H), 3.59-3.83 (m, 1H), 3.33-3.49 (m, 3H), 3.07-3.21 (m, 4H), 2.79 (s, 2H), 1.65 (br. s., 2H), 1.05-1.47 (m, 6H), 0.93 (t, J=7.40 Hz, 3H), 0.70-0.87 (m, 3H). MS obsd. (ESI⁺) [(M+H)⁺]: 561.2.

### Example 30

### 2-[[6-Amino-9-benzyl-8-oxo-2-(propylsulfonimidoyl)purine-7-carbonyl]-methyl-amino]ethyl pyrrolidine-1-carboxylate

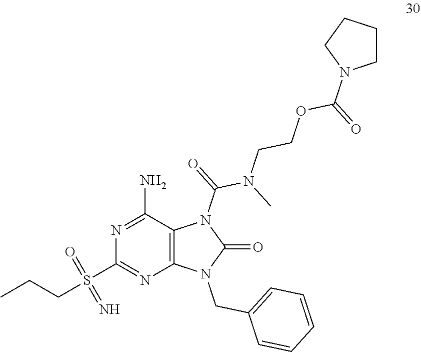 [SMILES: CCCS(=N)(=O)c1nc(N)c2c(n1)n(Cc1ccccc1)c(=O)n2C(=O)N(C)CCOC(=O)N1CCCC1]

The title compound was prepared in analogy to Example 1, Method A, Step 6 by using 2-[chlorocarbonyl(methyl)amino]ethyl pyrrolidine-1-carboxylate (Intermediate BF) instead of N-methyl-N-propyl-carbamoyl chloride (Intermediate AA). 2-[[6-Amino-9-benzyl-8-oxo-2-(propylsulfonimidoyl)purine-7-carbonyl]-methyl-amino]ethyl pyrrolidine-1-carboxylate (10.0 mg, Example 30) was obtained as a yellow solid. ¹H NMR (400 MHz, DMSO-d₆) δ ppm: 7.26-7.41 (m, 5H), 6.96 (br.s., 2H), 4.99 (s, 2H), 4.01-4.35 (m, 4H), 3.29-3.47 (m, 3H), 3.23 (br. s., 3H), 3.03-3.17 (m, 4H), 1.52-1.84 (m, 6H), 0.90-0.96 (m, 3H). MS obsd. (ESI⁺) [(M+H)⁺]: 545.2.

### Example 31

### 2-[[6-Amino-9-benzyl-8-oxo-2-(propylsulfonimidoyl)purine-7-carbonyl]-methyl-amino]ethyl N-methyl-N-propyl-carbamate

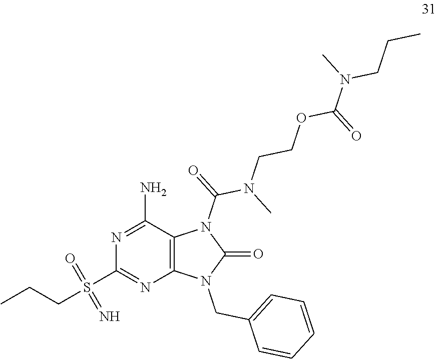 [SMILES: CCCN(C)C(=O)OCCN(C)C(=O)n1c(=O)n(Cc2ccccc2)c2nc(S(=N)(=O)CCC)nc(N)c21]

The title compound was prepared in analogy to Example 1, Method A, Step 6 by using 2-[chlorocarbonyl(methyl)amino]ethyl N-methyl-N-propyl-carbamate (Intermediate BG) instead of N-methyl-N-propyl-carbamoyl chloride (Intermediate AA). 2-[[6-Amino-9-benzyl-8-oxo-2-(propylsulfonimidoyl)purine-7-carbonyl]-methyl-amino]ethyl N-methyl-N-propyl-carbamate (3.7 mg, Example 31) was obtained as a yellow solid. ¹H NMR (400 MHz, CD₃OD) δ ppm: 7.22-7.48 (m, 5H), 5.09-5.22 (m, 4H), 4.55 (s, 2H), 3.38-3.57 (m, 4H), 3.13 (s, 3H), 1.61-1.85 (m, 4H), 1.22-1.41 (m, 3H), 0.88-1.13 (m, 6H). MS obsd. (ESI⁺) [(M+H)⁺]: 547.2.

### Example 32

### 2-[[6-Amino-9-benzyl-8-oxo-2-(propylsulfonimidoyl)purine-7-carbonyl]-methyl-amino]ethyl N,N-diethylcarbamate

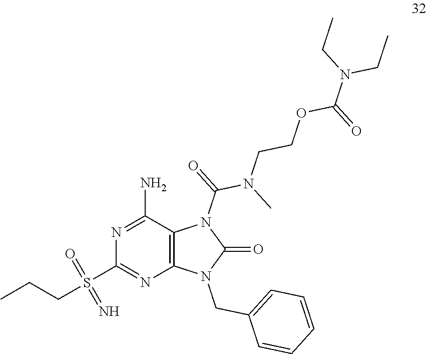 [SMILES: CCCS(=N)(=O)c1nc(N)c2c(n1)n(Cc1ccccc1)c(=O)n2C(=O)N(C)CCOC(=O)N(CC)CC]

The title compound was prepared in analogy to Example 1, Method A, Step 6 by using 2-[chlorocarbonyl(methyl)amino]ethyl N,N-diethylcarbamate (Intermediate BH) instead of N-methyl-N-propyl-carbamoyl chloride (Intermediate AA). 2-[[6-Amino-9-benzyl-8-oxo-2-(propylsulfonimidoyl)purine-7-carbonyl]-methyl-amino]ethyl N,N-diethylcarbamate (21.7 mg, Example 32) was obtained as yellow solid. ¹H NMR (400 MHz, DMSO-d₆) δ ppm: 7.25-7.41 (m, 5H), 6.96 (br. s., 2H), 4.99 (s, 2H), 4.08-4.36 (m, 3H), 3.70 (br, 1H), 3.33-3.46 (m, 3H), 3.01-3.24 (m, 7H), 1.55-1.74 (m, 2H), 0.86-1.05 (m, 9H). MS obsd. (ESI⁺) [(M+H)⁺]: 547.2.

### Example 33

### 2-[[6-Amino-9-benzyl-8-oxo-2-(propylsulfonimidoyl)purine-7-carbonyl]-methyl-amino]ethyl ethyl carbonate

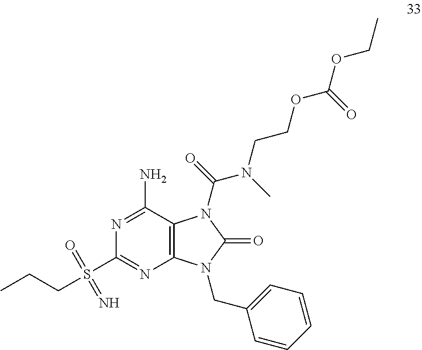 [SMILES: CCCS(=N)(=O)c1nc(N)c2c(n1)n(Cc1ccccc1)c(=O)n2C(=O)N(C)CCOC(=O)OCC]

The title compound was prepared in analogy to Example 1, Method A, Step 6 by using 2-[chlorocarbonyl(methyl)amino]ethyl ethyl carbonate (Intermediate BI) instead of N-methyl-N-propyl-carbamoyl chloride (Intermediate AA). 2-[[6-Amino-9-benzyl-8-oxo-2-(propylsulfonimidoyl)purine-7-carbonyl]-methyl-amino]ethyl ethyl carbonate (46 mg, Example 33) was obtained as yellow solid. ¹H NMR (400 MHz, DMSO-d₆) δ ppm: 0.82-0.99 (m, 3H), 1.02-1.28 (m, 3H), 1.56-1.76 (m, 2H), 3.05-3.18 (m, 3H), 3.35-3.48 (m, 3H), 3.73 (t, J=5.08 Hz, 2H), 4.08-4.27 (m, 3H), 4.37 (br. s., 1H), 5.00 (s, 2H), 6.76-7.11 (m, 2H), 7.22-7.45 (m, 5H). MS obsd. (ESI⁺) [(M+H)⁺]: 520.

### Example 34-A and Example 34-B

### 6-Amino-N-butyl-9-[(4-chlorophenyl)methyl]-N-methyl-8-oxo-2-[S(S)-propylsulfonimidoyl]purine-7-carboxamide and 6-amino-N-butyl-9-[(4-chlorophenyl)methyl]-N-methyl-8-oxo-2-[S(S)-propylsulfonimidoyl]purine-7-carboxamide

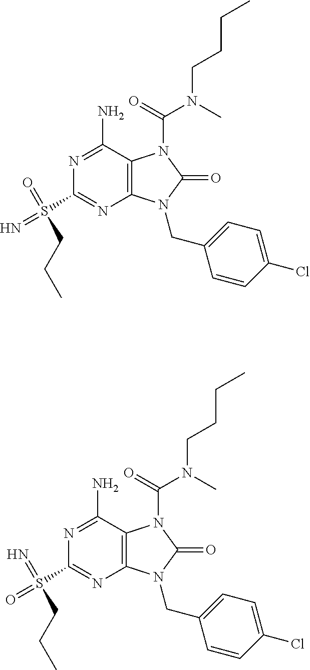

### Step 1: Preparation of 4-amino-3-[(4-chlorophenyl)methyl]-2-oxo-1H-imidazole-5-carbonitrile (Compound 34a)

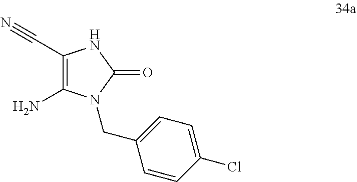 [SMILES: N#Cc1[nH]c(=O)n(Cc2ccc(Cl)cc2)c1N]

Compound 34a was prepared in analogy to Example 1, Method A, Step 1 by using 4-chlorobenzyl isocyanate instead of benzyl isocyanate. 4-Amino-3-[(4-chlorophenyl)methyl]-2-oxo-1H-imidazole-5-carbonitrile (8.0 g, Compound 34a) was obtained as a yellow solid. MS obsd. (ESI⁺) [(M+H)⁺]: 249.

### Step 2: Preparation of 6-amino-9-[(4-chlorophenyl)methyl]-2-sulfanyl-7H-purin-8-one (Compound 34b)

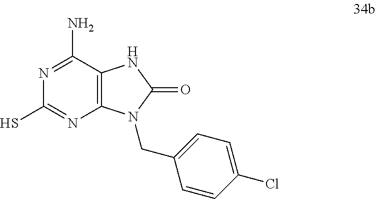 [SMILES: Nc1nc(S)nc2c1[nH]c(=O)n2Cc1ccc(Cl)cc1]

Compound 34b was prepared in analogy to Example 1, Method A, Step 2 by using 4-Amino-3-[(4-chlorophenyl)methyl]-2-oxo-1H-imidazole-5-carbonitrile (Compound 34a) instead of 4-amino-3-phenylmethyl-2-oxo-1H-imidazole-5-carbonitrile (Compound 1a). 6-Amino-9-[(4-chlorophenyl)methyl]-2-sulfanyl-7H-purin-8-one (6.4 g, Compound 34b) was obtained as a yellow solid and was used for the next step without further purification. MS obsd. (ESI⁺) [(M+H)⁺]: 308.

### Step 3: Preparation of 6-amino-9-[(4-chlorophenyl)methyl]-2-propylsulfanyl-7H-purin-8-one (Compound 34c)

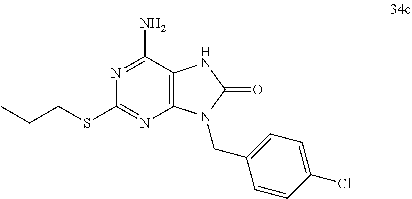 [SMILES: CCCSc1nc(N)c2[nH]c(=O)n(Cc3ccc(Cl)cc3)c2n1]

Compound 34c was prepared in analogy to Example 1, Method A, Step 3 by using 6-amino-9-[(4-chlorophenyl)methyl]-2-sulfanyl-7H-purin-8-one (Compound 34b) instead of 6-amino-9-phenylmethyl-2-sulfanyl-7H-purin-8-one (Compound 1b). 6-Amino-9-[(4-chlorophenyl)methyl]-2-propylsulfanyl-7H-purin-8-one (800 mg, Compound 34c) was obtained as a white solid. MS obsd. (ESI⁺) [(M+H)⁺]: 350.

### Step 4: Preparation of 6-amino-9-[(4-chlorophenyl)methyl]-2-propylsulfinyl-7H-purin-8-one (Compound 34d)

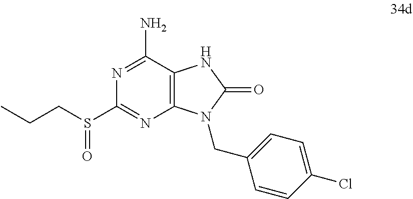 [SMILES: CCCS(=O)c1nc(N)c2[nH]c(=O)n(Cc3ccc(Cl)cc3)c2n1]

Compound 34d was prepared in analogy to Example 1, Method A, Step 4 by using 6-amino-9-[(4-chlorophenyl)methyl]-2-propylsulfanyl-7H-purin-8-one (Compound 34c) instead of 6-amino-9-benzyl-2-propylsulfanyl-7H-purin-8-one (Compound 1c). 6-Amino-9-[(4-chlorophenyl)methyl]-2-propylsulfinyl-7H-purin-8-one (150 mg, Compound 34d) was obtained as a white solid. MS obsd. (ESI⁺) [(M+H)⁺]: 366.

### Step 5: Preparation of 6-amino-9-[(4-chlorophenyl)methyl]-2-(propylsulfonimidoyl)-7H-purin-8-one (compound 34e), 6-amino-9-[(4-chlorophenyl)methyl]-2-[S(S)-propylsulfonimidoyl)-7H-purin-8-one and 6-amino-9-[(4-chlorophenyl)methyl]-2-[S(S)-propylsulfonimidoyl)-7H-purin-8-one (Compound 34e-A and Compound 34e-B)

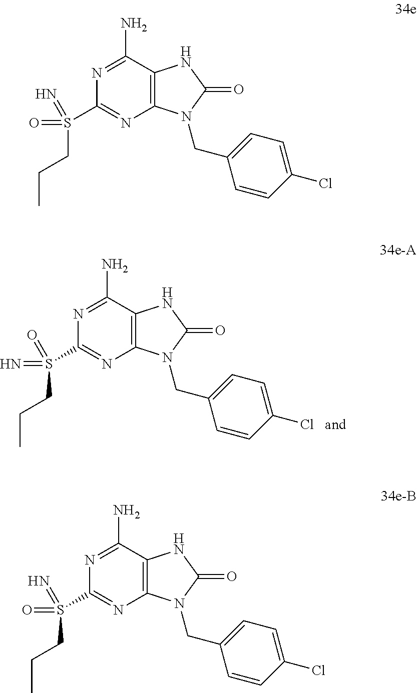 [SMILES: CCCS(=N)(=O)c1nc(N)c2[nH]c(=O)n(Cc3ccc(Cl)cc3)c2n1 ; CCC[S@@](=N)(=O)c1nc(N)c2[nH]c(=O)n(Cc3ccc(Cl)cc3)c2n1 ; "and" ; CCC[S@](=N)(=O)c1nc(N)c2[nH]c(=O)n(Cc3ccc(Cl)cc3)c2n1]

Compound 34e was prepared in analogy to Example 1, Method A, Step 5 by using 6-amino-9-[(4-chlorophenyl)methyl]-2-propylsulfinyl-7H-purin-8-one (Compound 34d) instead of 6-amino-9-benzyl-2-(2-propylsulfinyl)-7H-purin-8-one (Compound 1d). 6-Amino-9-[(4-chlorophenyl)methyl]-2-(propylsulfonimidoyl)-7H-purin-8-one (250 mg, compound 34e) was obtained as a white solid. ¹H NMR (400 MHz, DMSO-d₆) δ ppm: 10.60 (br. s, 1H), 7.32-7.42 (m, 4H), 6.98 (br. s, 2H), 4.96 (s, 2H), 4.03 (s, 1H), 3.25-3.41 (m, 2H), 1.56-1.68 (m, 2H), 0.91 (t, J=8 Hz, 3H). MS obsd. (ESI⁺) [(M⁺H)⁺]: 381.

Separation of compound of Compound 34e by chiral HPLC afforded Compound 34e-A (faster eluting, 110 mg) and Compound 34e-B (slower eluting, 100 mg) as white solid with methanol 5%-40% (0.05% DEA)/CO₂ on ChiralPak OJ-3 column.

Compound 34e-A: ¹H NMR (400 MHz, DMSO-d₆) δ ppm: 10.63 (br. s, 1H), 7.33-7.42 (m, 4H), 6.99 (br. s, 2H), 4.96 (s, 2H), 4.05 (br. s, 1H), 3.26-3.39 (m, 2H), 1.53-1.69 (m, 2H), 0.91 (t, J=7.4 Hz, 3H). MS obsd. (ESI⁺) [(M+H)⁺]: 381.

Compound 34e-B: ¹H NMR (400 MHz, DMSO-d₆) δ ppm: 10.63 (br. s, 1H), 7.33-7.42 (m, 4H), 6.99 (br. s, 2H), 4.96 (s, 2H), 4.05 (br. s, 1H), 3.26-3.40 (m, 2H), 1.54-1.69 (m, 2H), 0.91 (t, J=7.5 Hz, 3H). MS obsd. (ESI⁺) [(M+H)⁺]: 381.

### Step 6: 6-Amino-N-butyl-9-[(4-chlorophenyl)methyl]-N-methyl-8-oxo-2-[S(S)-propylsulfonimidoyl]purine-7-carboxamide and 6-amino-N-butyl-9-[(4-chlorophenyl)methyl]-N-methyl-8-oxo-2-[S(S)-propylsulfonimidoyl]purine-7-carboxamide (Example 34-A and Example 34-B)

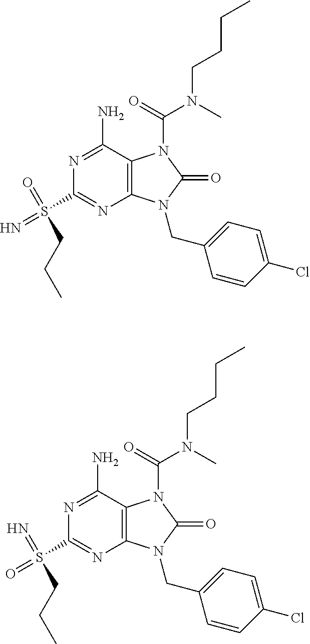 [SMILES: CCCCN(C)C(=O)n1c(=O)n(Cc2ccc(Cl)cc2)c2nc([S@](=N)(=O)CCC)nc(N)c21 ; CCCCN(C)C(=O)n1c(=O)n(Cc2ccc(Cl)cc2)c2nc([S@@](=N)(=O)CCC)nc(N)c21]

Example 34-A was prepared in analogy to Example 1, Method A, Step 6 by using Compound 34e-A and N-butyl-N-methyl-carbamoyl chloride instead of 6-amino-9-benzyl-2-(propylsulfonimidoyl)-7H-purin-8-one (Compound 1e) and N-methyl-N-propyl-carbamoyl chloride (Intermediate AA).

### Example 34-A (160 mg)

¹H NMR (400 MHz, DMSO-d₆) δ ppm: 7.37-7.45 (m, 4H), 6.91 (br. s., 2H), 4.99 (s, 2H), 4.17 (s, 1H), 3.28-3.40 (m, 4H), 3.05 (s, 2H), 3.02 (s, 1H), 1.49-1.70 (m, 4H), 1.15-1.37 (m, 2H), 0.89-0.94 (m, 5H), 0.76 (t, J=8 Hz, 1H). MS obsd. (ESI⁺) [(M+H)⁺]: 494.

### Example 34-B

(167 mg) was prepared in analogy to Example 34-A by using Compound 34e-B instead of Compound 34e-A.

### Example 34-B

¹H NMR (400 MHz, DMSO-d₆) δ ppm: 7.36-7.45 (m, 4H), 6.91 (br. s., 2H), 4.99 (s, 2H), 4.17 (s, 1H), 3.28-3.41 (m, 4H), 3.05 (s, 2H), 3.02 (s, 1H), 1.50-1.71 (m, 4H), 1.15-1.37 (m, 2H), 0.89-0.94 (m, 5H), 0.76 (t, J=7.4 Hz, 1H). MS obsd. (ESI⁺) [(M+H)⁺]: 494.

### Example 35

### 6-Amino-9-[(4-chlorophenyl)methyl]-N-ethyl-N-methyl-8-oxo-2-(propylsulfonimidoyl)purine-7-carboxamide

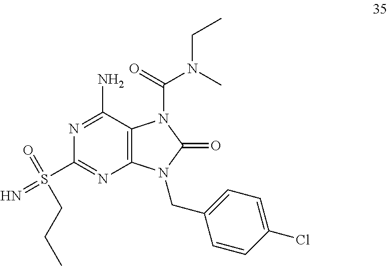 [SMILES: CCCS(=N)(=O)c1nc(N)c2c(n1)n(Cc1ccc(Cl)cc1)c(=O)n2C(=O)N(C)CC]

The title compound was prepared in analogy to Example 1, Method A, Step 6 by using 6-amino-9-[(4-chlorophenyl)methyl]-2-(propylsulfonimidoyl)-7H-purin-8-one (Compound 34e) and N-ethyl-N-methyl-carbamoyl chloride instead of 6-amino-9-benzyl-2-(propylsulfonimidoyl)-7H-purin-8-one (Compound 1e) and N-methyl-N-propyl-carbamoyl chloride (Intermediate AA). 6-Amino-9-[(4-chlorophenyl)methyl]-N-ethyl-N-methyl-8-oxo-2-(propylsulfonimidoyl)purine-7-carboxamide (60 mg, Example 35) was obtained as a white solid. ¹H NMR (400 MHz, DMSO-d₆) δ ppm: 7.40 (s, 4H), 6.91 (br s, 2H), 4.99 (s, 2H), 4.16 (s, 1H), 3.34-3.44 (m, 4H), 3.05 (s, 2H), 3.01 (s, 1H), 1.58-1.67 (m, 2H), 1.18 (t, J=8.0 Hz, 3H), 0.92 (t, J=8.0 Hz, 3H). MS obsd. (ESI⁺) [(M+H)⁺]: 466.

### Example 36-A and Example 36-B

### 6-Amino-N-methyl-8-oxo-N-propyl-2[S(S)-propylsulfonimidoyl]-9-(p-tolylmethyl)purine-7-carboxamide and 6-amino-N-methyl-8-oxo-N-propyl-2 [S(R)-propylsulfonimidoyl]-9-(p-tolylmethyl)purine-7-carboxamide

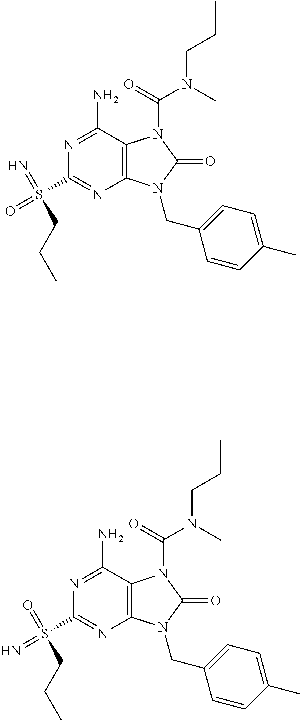 [SMILES: CCCN(C)C(=O)n1c(=O)n(Cc2ccc(C)cc2)c2nc([S@@](=N)(=O)CCC)nc(N)c21 ; CCCN(C)C(=O)n1c(=O)n(Cc2ccc(C)cc2)c2nc([S@](=N)(=O)CCC)nc(N)c21]

### Step 1: Preparation of 6-chloro-5-nitro-2-propylsulfanyl-N-(p-tolylmethyl)pyrimidin-4-amine (Compound 36a)

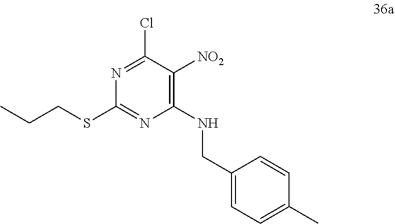 [SMILES: CCCSc1nc(Cl)c([N+](=O)[O-])c(NCc2ccc(C)cc2)n1]

Compound 36a was prepared in analogy to Example 1, Method B, Step 1 by using p-tolylmethylamine instead of phenylmethanamine. 6-Chloro-5-nitro-2-propylsulfanyl-N-(p-tolylmethyl)pyrimidin-4-amine (3.9 g, Compound 36a) was obtained as a white solid. MS obsd. (ESI⁺) [(M+H)⁺]: 353.

### Step 2: Preparation of 6-Chloro-2-Propylsulfanyl-N4-(P-Tolylmethyl)Pyrimidine-4,5-Diamine (Compound 36B)

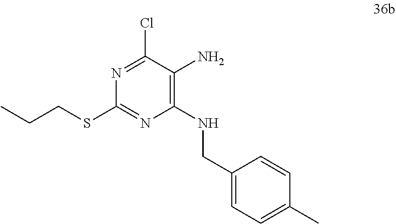 [SMILES: CCCSc1nc(Cl)c(N)c(NCc2ccc(C)cc2)n1]

Compound 36b was prepared in analogy to Example 1, Method B, Step 2 by using 6-chloro-5-nitro-2-propylsulfanyl-N-(p-tolylmethyl)pyrimidin-4-amine (Compound 36a) instead of N-benzyl-6-chloro-5-nitro-2-propylsulfanyl-pyrimidin-4-amine (Compound 1f). 6-Chloro-2-propylsulfanyl-N4-(p-tolylmethyl)pyrimidine-4,5-diamine (2.2 g, Compound 36b) was obtained as a white solid. MS obsd. (ESI⁺) [(M+H)⁺]: 323.

### Step 3: Preparation of 6-Chloro-2-Propylsulfanyl-9-(P-Tolylmethyl)-7H-Purin-8-One (Compound 36C)

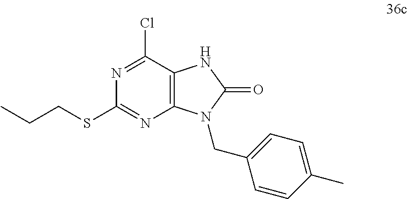 [SMILES: CCCSc1nc(Cl)c2[nH]c(=O)n(Cc3ccc(C)cc3)c2n1]

Compound 36c was prepared in analogy to Example 1, Method B, Step 3 by using 6-chloro-2-propylsulfanyl-N4-(p-tolylmethyl)pyrimidine-4,5-diamine (Compound 36b) instead of N-benzyl-6-chloro-2-(propylsulfanyl)pyrimidine-4,5-diamine (Compound 1g). 6-Chloro-2-propylsulfanyl-9-(p-tolylmethyl)-7H-purin-8-one (2.2 g, Compound 36c) was obtained as a white solid. MS obsd. (ESI⁺) [(M+H)⁺]: 349.

### Step 4: Preparation of 6-[(4-Methoxyphenyl)Methylamino]-2-Propylsulfanyl-9-(P-Tolylmethyl)-7H-Purin-8-One (Compound 36D)

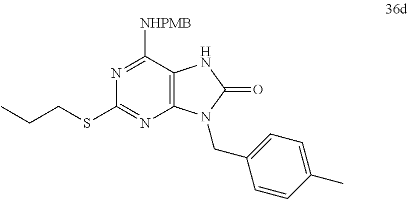

Compound 36d was prepared in analogy to Example 1, Method B, Step 4, by using 6-chloro-2-propylsulfanyl-9-(p-tolylmethyl)-7H-purin-8-one (Compound 36c) instead of 9-benzyl-6-chloro-2-propylsulfanyl-7H-purin-8-one (Compound 1h). 6-[(4-methoxyphenyl)methylamino]-2-propylsulfanyl-9-(p-tolylmethyl)-7H-purin-8-one (2.0 g, Compound 36d) was obtained as a white solid. MS obsd. (ESI⁺) [(M+H)⁺]: 450.

### Step 5: Preparation of 6-Amino-2-Propylsulfanyl-9-(P-Tolylmethyl)-7H-Purin-8-One (Compound 36E)

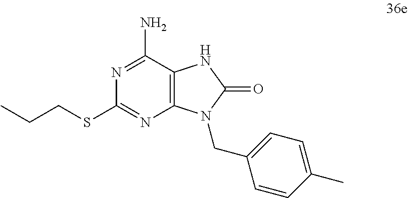 [SMILES: CCCSc1nc(N)c2[nH]c(=O)n(Cc3ccc(C)cc3)c2n1]

Compound 36e was prepared in analogy to Example 1, Method B, Step 5 by using 6-[(4-methoxyphenyl)methylamino]-2-propylsulfanyl-9-(p-tolylmethyl)-7H-purin-8-one (Compound 36d) instead of 6-amino-9-benzyl-2-propylsulfanyl-7H-purin-8-one (Compound 1i). 6-amino-2-propylsulfanyl-9-(p-tolylmethyl)-7H-purin-8-one (1.0 g, Compound 36e) was obtained as a white solid. MS obsd. (ESI⁺) [(M+H)⁺]: 330.

### Step 6: Preparation of 6-Amino-2-Propylsulfinyl-9-(P-Tolylmethyl)-7H-Purin-8-One (Compound 36F)

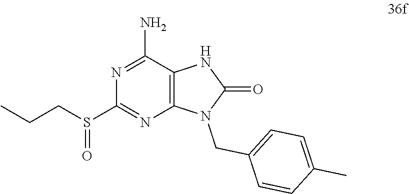 [SMILES: CCCS(=O)c1nc(N)c2[nH]c(=O)n(Cc3ccc(C)cc3)c2n1]

Compound 36f was prepared in analogy to Example 1, Method B, Step 6 by using 6-amino-2-propylsulfanyl-9-(p-tolylmethyl)-7H-purin-8-one (Compound 36e) instead of 6-amino-9-benzyl-2-(2-propylsulfanyl)-7H-purin-8-one (Compound 1c). 6-amino-2-propylsulfinyl-9-(p-tolylmethyl)-7H-purin-8-one (220 mg, Compound 36f) was obtained as a white solid MS obsd. (ESI⁺) [(M+H)⁺]: 345.

### Step 7: Preparation of 6-Amino-2-(Propylsulfonimidoyl)-9-(P-Tolylmethyl)-7H-Purin-8-One (Compound 36G)

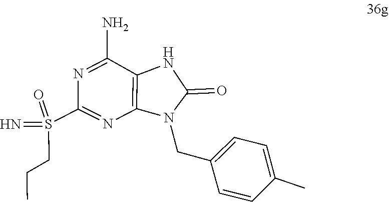 [SMILES: CCCS(=N)(=O)c1nc(N)c2[nH]c(=O)n(Cc3ccc(C)cc3)c2n1]

Compound 36g was prepared in analogy to Example 1, Method B, Step 7 by using 6-amino-2-propylsulfinyl-9-(p-tolylmethyl)-7H-purin-8-one (Compound 36f) instead of 6-amino-9-benzyl-2-propylsulfinyl-7H-purin-8-one (Compound 1d). 6-Amino-2-(propylsulfonimidoyl)-9-(p-tolylmethyl)-7H-purin-8-one (127 mg, Compound 36g) was obtained as a white solid. ¹H NMR (400 MHz, DMSO-d₆) δ ppm: 10.67 (br. s., 1H), 7.23 (d, J=8.0 Hz, 2H), 7.13 (d, J=8.0 Hz, 2H), 6.98 (br. s., 2H), 4.91 (s, 2H), 4.05 (s, 1H), 3.34-3.27 (m, 2H), 2.26 (s, 3H), 1.67-1.62 (m, 2H), 0.92 (t, J=8.0 Hz, 3H). MS obsd. (ESI⁺) [(M+H)⁺]: 361.

Separation of compound 36g by chiral HPLC afforded compound 36g-A (faster eluting, 50 mg) and compound 36g-B (slower eluting, 49 mg) as white solid with 30% isopropanol (0.05% DEA)/CO₂ on ChiralPak AD-3 column.

Compound 36g-A: ¹H NMR: (400 MHz, DMSO-d₆) δ ppm: 10.51 (s, 1H), 7.22 (d, J=8.0 Hz, 2H), 7.12 (d, J=8.0 Hz, 2H), 7.00 (s, 2H), 4.91 (s, 2H), 4.03 (s, 1H), 3.35-3.31 (m, 2H), 2.26 (s, 3H), 1.70-1.58 (m, 2H), 0.93 (t, J=7.40 Hz, 3H). MS obsd. (ESI⁺) [(M+H)⁺]: 361.

Compound 36g-B: ¹H NMR: (400 MHz, DMSO-d₆) δ ppm: 10.54 (s, 1H), 7.23 (d, J=8.0 Hz, 2H), 7.13 (d, J=8.0 Hz, 2H), 6.97 (s, 2H), 4.91 (s, 2H), 4.04 (s, 1H), 3.34-3.30 (m, 2H), 2.26 (s, 3H), 1.72-1.57 (m, 2H), 0.93 (t, J=7.40 Hz, 3H). MS obsd. (ESI⁺) [(M+H)⁺]: 361.

### Step 8: Preparation of 6-Amino-N-methyl-8-oxo-N-propyl-2[S(S)-propylsulfonimidoyl]-9-(p-tolylmethyl)purine-7-carboxamide and 6-amino-N-methyl-8-oxo-N-propyl-2[S(R)-propylsulfonimidoyl]-9-(p-tolylmethyl)purine-7-carboxamide (Example 36-A and Example 36-B)

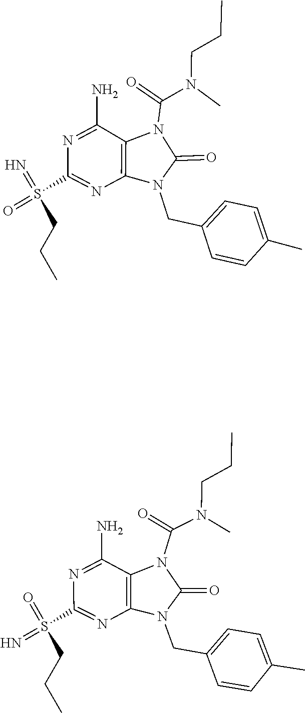 [SMILES: CCCN(C)C(=O)n1c(=O)n(Cc2ccc(C)cc2)c2nc([S@@](=N)(=O)CCC)nc(N)c21 ; CCCN(C)C(=O)n1c(=O)n(Cc2ccc(C)cc2)c2nc([S@](=N)(=O)CCC)nc(N)c21]

Example 36-A was prepared in analogy to Example 1, Method A, Step 6 by using Compound 36g-A instead of 6-amino-9-benzyl-2-(propylsulfonimidoyl)-7H-purin-8-one (Compound 1e). Example 36-A (108 mg) was obtained as a white solid. ¹H NMR (400 MHz, DMSO-d₆) δ ppm: 7.27 (d, J=8 Hz, 2H), 7.14 (d, J=8 Hz, 2H), 6.87 (br. s., 2H), 4.95 (s, 2H), 4.15 (s, 1H), 3.33-3.57 (m, 4H), 3.05 (s, 2H), 3.02 (s, 1H), 2.26 (s, 3H), 1.52-1.73 (m, 4H), 0.75-0.97 (m, 6H). MS obsd. (ESI⁺) [(M+H)⁺]: 460.

Example 36-B was prepared in analogy to Example 1, Method A, Step 6 by using Compound 36g-B instead of 6-amino-9-benzyl-2-(propylsulfonimidoyl)-7H-purin-8-one (compound 1e). Example 36-B (125 mg): ¹H NMR (400 MHz, DMSO-d₆) δ ppm: 7.27 (d, J=8 Hz, 2H), 7.14 (d, J=8 Hz, 2H), 6.87 (br. s., 2H), 4.95 (s, 2H), 4.15 (s, 1H), 3.33-3.57 (m, 4H), 3.05 (s, 2H), 3.02 (s, 1H), 2.26 (s, 3H), 1.52-1.73 (m, 4H), 0.75-0.97 (m, 5H). MS obsd. (ESI⁺) [(M+H)⁺]: 460.

### Example 37-A and Example 37-B

### 6-Amino-2-[S(S)-propylsulfonimidoyl]-9-(p-tolylmethyl)-7-(pyrrolidine-1-carbonyl)purin-8-one and 6-amino-2-[S(R)-propylsulfonimidoyl]-9-(p-tolylmethyl)-7-(pyrrolidine-1-carbonyl)purin-8-one

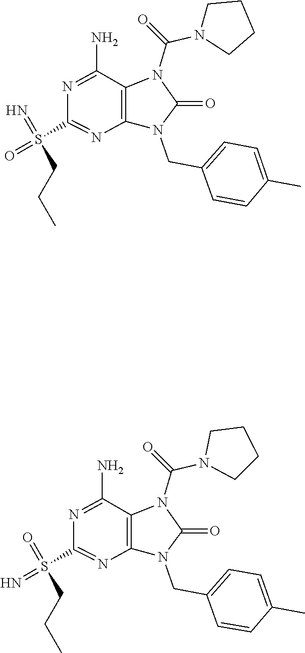 [SMILES: CCC[S@](=N)(=O)c1nc(N)c2c(n1)n(Cc1ccc(C)cc1)c(=O)n2C(=O)N1CCCC1 ; CCC[S@@](=N)(=O)c1nc(N)c2c(n1)n(Cc1ccc(C)cc1)c(=O)n2C(=O)N1CCCC1]

Example 37-A was prepared in analogy to Example 1, Method A, Step 6 by using Compound 36g-A and pyrrolidine-1-carbonyl chloride instead of 6-amino-9-benzyl-2-(propylsulfonimidoyl)-7H-purin-8-one (Compound 1e) and N-methyl-N-propyl-carbamoyl chloride (Intermediate AA).

Example 37-A (390 mg) was obtained as a white solid. ¹H NMR (400 MHz, DMSO-d₆) δ ppm: 7.31-7.11 (m, 4H), 7.04 (s, 2H), 4.95 (s, 2H), 4.15 (s, 1H), 3.65-3.47 (m, 4H), 3.37 (m, 2H), 2.27 (s, 3H), 1.97-1.81 (m, 4H), 1.71-1.59 (m, 2H), 0.94 (t, J=7.4 Hz, 3H). MS obsd. (ESI⁺) [(M+H)⁺]: 458.2.

Example 37-B (125 mg) was prepared in analogy to Example 37-A by using Compound 36g-B instead of Compound 36g-A. ¹H NMR (400 MHz, DMSO-d₆) δ ppm: 7.28-7.14 (m, 4H), 7.04 (s, 2H), 4.95 (s, 2H), 4.15 (s, 1H), 3.65-3.47 (m, 4H), 3.37 (m, 2H), 2.27 (s, 3H), 1.93-1.84 (m, 4H), 1.65-1.60 (m, 2H), 0.95 (t, J=7.4 Hz, 3H). MS obsd. (ESI⁺) [(M+H)⁺]: 458.3.

### Example 38-A and Example 38-B

### 6-Amino-N-(2-methoxyethyl)-N-methyl-8-oxo-2-[S(S)-propylsulfonimidoyl]-9-(p-tolylmethyl)purine-7-carboxamide and 6-amino-N-(2-methoxyethyl)-N-methyl-8-oxo-2-[S(R)-propylsulfonimidoyl]-9-(p-tolylmethyl)purine-7-carboxamide

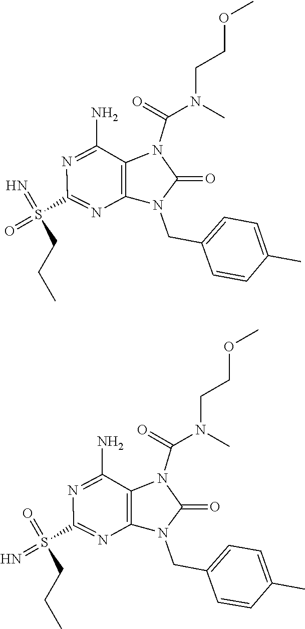 [SMILES: CCC[S@](=N)(=O)c1nc(N)c2c(n1)n(Cc1ccc(C)cc1)c(=O)n2C(=O)N(C)CCOC ; CCC[S@@](=N)(=O)c1nc(N)c2c(n1)n(Cc1ccc(C)cc1)c(=O)n2C(=O)N(C)CCOC]

Example 38-A was prepared in analogy to Example 1, Method A, Step 6 by using Compound 36g-A and N-(2-methoxyethyl)-N-methyl-carbamoyl chloride (Intermediate AB) instead of 6-amino-9-benzyl-2-(propylsulfonimidoyl)-7H-purin-8-one (Compound 1e) and N-methyl-N-propyl-carbamoyl chloride (Intermediate AA).

Example 38-A (57.8 mg) was obtained as a white solid. ¹H NMR (400 MHz, DMSO-d₆) δ ppm: 7.26 (d, J=7.6 Hz, 2H), 7.14 (d, J=7.6 Hz, 2H), 6.89-6.78 (m, 2H), 4.95 (s, 2H), 4.18 (s, 1H), 3.62-3.58 (m, 2H), 3.43-3.37 (m, 2H), 3.30-3.10 (m, 3H), 3.09-3.08 (m, 3H), 3.08-3.05 (m, 2H), 2.27 (s, 3H), 1.77-1.54 (m, 2H), 0.95 (t, J=7.4 Hz, 3H). MS obsd. (ESI⁺) [(M+H)⁺]: 476.3.

Example 38-B (46.6 mg) was prepared in analogy to Example 38-A by using Compound 36g-B instead of Compound 36g-A. ¹H NMR (400 MHz, DMSO-d₆) δ ppm: 7.26 (d, J=7.6 Hz, 2H), 7.14 (d, J=7.6 Hz, 2H), 6.89-6.78 (m, 2H), 4.95 (s, 2H), 4.18 (s, 1H), 3.62-3.58 (m, 2H), 3.43-3.37 (m, 2H), 3.30-3.10 (m, 3H), 3.09-3.08 (m, 3H), 3.08-3.05 (m, 2H), 2.27 (s, 3H), 1.77-1.54 (m, 2H), 0.95 (t, J=7.4 Hz, 3H). MS obsd. (ESI⁺) [(M+H)⁺]: 476.3.

### Example 39

### 6-Amino-N-ethyl-N-methyl-8-oxo-2-(propylsulfonimidoyl)-9-(p-tolylmethyl)purine-7-carboxamide

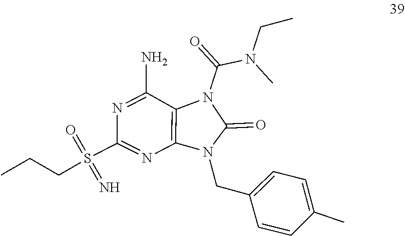 [SMILES: CCCS(=N)(=O)c1nc(N)c2c(n1)n(Cc1ccc(C)cc1)c(=O)n2C(=O)N(C)CC]

The title compound was prepared in analogy to Example 1, Method A, Step 6 by using N-ethyl-N-methyl-carbamoyl chloride and 6-amino-2-(propylsulfonimidoyl)-9-(p-tolylmethyl)-7H-purin-8-one (Compound 36g) instead of N-methyl-N-propyl-carbamoyl chloride (Intermediate AA) and 6-amino-9-benzyl-2-(propylsulfonimidoyl)-7H-purin-8-one (Compound 1e). 6-Amino-N-ethyl-N-methyl-8-oxo-2-(propylsulfonimidoyl)-9-(p-tolylmethyl)purine-7-carboxamide (141.8 mg, Example 39) was obtained as a light yellow solid. ¹H NMR (400 MHz, DMSO-d₆) δ ppm: 7.26 (d, J=7.9 Hz, 2H), 7.15 (d, J=7.9 Hz, 2H), 6.89 (s, 2H), 4.95 (s, 2H), 4.24-4.07 (m, 1H), 3.52-3.35 (m, 4H), 3.10-2.95 (m, 3H), 2.26 (s, 3H), 1.77-1.55 (m, 2H), 1.24-1.10 (m, 3H), 0.95 (t, J=7.4 Hz, 3H). MS obsd. (ESI⁺) [(M+H)⁺]: 446.1.

### Example 40

### 6-Amino-N-butyl-N-methyl-8-oxo-2-(propylsulfonimidoyl)-9-(p-tolylmethyl)purine-7-carboxamide

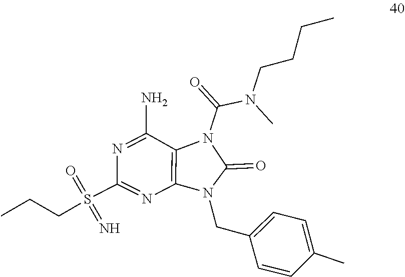 [SMILES: CCCCN(C)C(=O)n1c(=O)n(Cc2ccc(C)cc2)c2nc(S(=N)(=O)CCC)nc(N)c21]

The title compound was prepared in analogy to Example 1, Method A, Step 6 by using 6-amino-2-(propylsulfonimidoyl)-9-(p-tolylmethyl)-7H-purin-8-one (Compound 36g) and N-butyl-N-methyl-carbamoyl chloride instead of 6-amino-9-benzyl-2-(propylsulfonimidoyl)-7H-purin-8-one (Compound 1e) and N-methyl-N-propyl-carbamoyl chloride (Intermediate AA). 6-Amino-N-butyl-N-methyl-8-oxo-2-(propylsulfonimidoyl)-9-(p-tolylmethyl)purine-7-carboxamide (32 mg, Example 40) was obtained as a white solid. ¹H NMR (400 MHz, DMSO-d₆) δ ppm: 7.28-7.14 (m, 4H), 6.88 (s, 2H), 4.95 (s, 2H), 4.16 (s, 1H), 3.41-3.36 (m, 2H), 3.10-2.99 (m, 3H), 2.53-2.51 (m, 2H), 2.27 (s, 3H), 1.71-1.63 (m, 2H), 1.62-1.51 (m, 2H), 1.42-1.26 (m, 2H), 0.97-0.74 (m, 6H). MS obsd. (ESI⁺) [(M+H)⁺]: 474.3

### Example 41-A and Example 41-B

### 6-Amino-9-[(4-chlorophenyl)methyl]-2-[S(R)-ethylsulfonimidoyl]-N-methyl-8-oxo-N-propyl-purine-7-carboxamide (Example 41-A) and 6-Amino-9-[(4-chlorophenyl)methyl]-2-[S(S)-ethylsulfonimidoyl]-N-methyl-8-oxo-N-propyl-purine-7-carboxamide (Example 41-B)

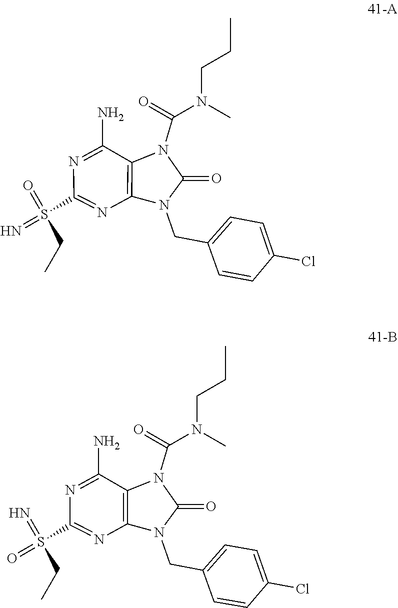 [SMILES: CCCN(C)C(=O)n1c(=O)n(Cc2ccc(Cl)cc2)c2nc([S@](=N)(=O)CC)nc(N)c21 ; CCCN(C)C(=O)n1c(=O)n(Cc2ccc(Cl)cc2)c2nc([S@@](=N)(=O)CC)nc(N)c21]

### Step 1: Preparation of 6-amino-9-[(4-chlorophenyl)methyl]-2-ethylsulfanyl-7H-purin-8-one (Compound 41a)

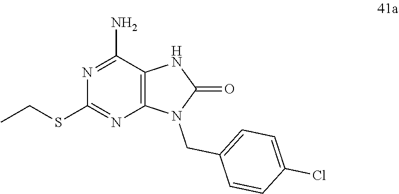 [SMILES: CCSc1nc(N)c2[nH]c(=O)n(Cc3ccc(Cl)cc3)c2n1]

Compound 41a was prepared in analogy to Example 1, Method A, Step 3 by using iodoethane and 6-amino-9-[(4-chlorophenyl)methyl]-2-sulfanyl-7H-purin-8-one (Compound 34b) instead of bromopropane and 6-amino-9-phenylmethyl-2-sulfanyl-7H-purin-8-one (Compound 1b). 6-Amino-9-[(4-chlorophenyl)methyl]-2-ethylsulfanyl-7H-purin-8-one (2.5 g, Compound 41a) was obtained as a white solid. MS obsd. (ESI⁺) [(M+H)⁺]: 336.

### Step 2: Preparation of 6-amino-9-(4-chlorobenzyl)-2-ethylsulfinyl-7H-purin-8-one (Compound 41b)

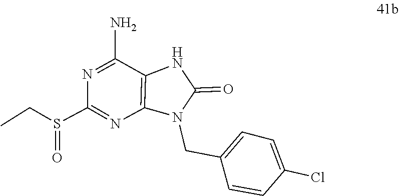 [SMILES: CCS(=O)c1nc(N)c2[nH]c(=O)n(Cc3ccc(Cl)cc3)c2n1]

Compound 41b was prepared in analogy to Example 1, Method A, Step 4 by using 6-amino-9-[(4-chlorophenyl)methyl]-2-ethylsulfanyl-7H-purin-8-one (Compound 41a) instead of 6-amino-9-benzyl-2-propylsulfanyl-7H-purin-8-one (Compound 1c). 6-Amino-9-(4-chlorobenzyl)-2-ethylsulfinyl-7H-purin-8-one (1.94 g, Compound 41b) was obtained as a white solid. MS obsd. (ESI⁺) [(M+H)⁺]: 352.

### Step 3: Preparation of 6-amino-9-[(4-chlorophenyl)methyl]-2-(ethylsulfonimidoyl)-7H-purin-8-one (Compound 41c)

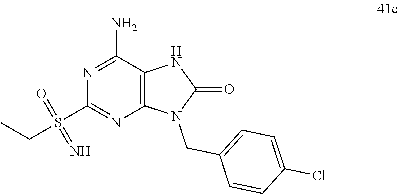 [SMILES: CCS(=N)(=O)c1nc(N)c2[nH]c(=O)n(Cc3ccc(Cl)cc3)c2n1]

Compound 41c was prepared in analogy to Example 1, Method A, Step 5 by using 6-amino-9-(4-chlorobenzyl)-2-ethylsulfinyl-7H-purin-8-one (Compound 41b) instead of 6-amino-9-benzyl-2-(2-methylsulfinyl)-7H-purin-8-one (Compound 1d). 6-Amino-9-[(4-chlorophenyl)methyl]-2-(ethylsulfonimidoyl)-7H-purin-8-one (217 mg, Example 41c) was obtained as a white solid. ¹H NMR (400 MHz, DMSO-d₆) δ ppm: 10.61 (s, 1H), 7.42-7.35 (m, 4H), 6.98 (s, 2H), 4.96 (s, 2H), 4.05 (s, 1H), 3.42-3.37 (m, 2H), 1.16 (t, J=7.4 Hz, 3H). MS obsd. (ESI⁺) [(M+H)⁺]: 367.0.

Separation of compound of Compound 41c by chiral HPLC afforded Compound 41c-A (faster eluting, 31.8 mg) and Compound 41c-B (slower eluting, 10 mg) as white solid with methanol 5%-40% (0.05% DEA)/CO₂ on ChiralPak IC-3 column.

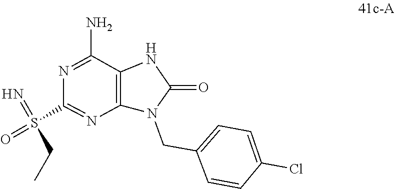 [SMILES: CC[S@](=N)(=O)c1nc(N)c2[nH]c(=O)n(Cc3ccc(Cl)cc3)c2n1]

Compound 41c-A: ¹H NMR (400 MHz, DMSO-d₆) δ ppm: 10.76 (s, 1H), 7.45-7.33 (m, 4H), 7.01 (s, 2H), 4.96 (s, 2H), 4.03 (s, 1H), 3.40-3.34 (m, 2H), 1.17 (t, J=7.4 Hz, 3H). MS obsd. (ESI⁺) [(M+H)⁺]: 367.0.

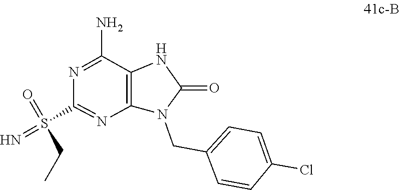 [SMILES: CC[S@@](=N)(=O)c1nc(N)c2[nH]c(=O)n(Cc3ccc(Cl)cc3)c2n1]

Compound 41c-B: 1H NMR (400 MHz, DMSO-d₆) δ ppm: 10.70 (s, 1H), 7.46-7.28 (m, 4H), 7.01 (s, 2H), 4.96 (s, 2H), 4.03 (s, 1H), 3.44-3.36 (m, 2H), 1.17 (t, J=7.4 Hz, 3H). MS obsd. (ESI⁺) [(M+H)⁺]: 367.0.

### Step 4: 6-Amino-9-[(4-chlorophenyl)methyl]-2-[S(R)-ethylsulfonimidoyl]-N-methyl-8-oxo-N-propyl-purine-7-carboxamide (Example 41-A) and 6-amino-9-[(4-chlorophenyl)methyl]-2-[S(S)-ethylsulfonimidoyl]-N-methyl-8-oxo-N-propyl-purine-7-carboxamide (Example 41-B)

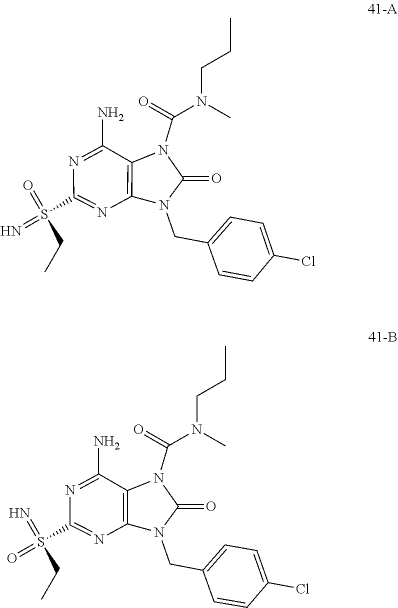 [SMILES: CCCN(C)C(=O)n1c(=O)n(Cc2ccc(Cl)cc2)c2nc([S@](=N)(=O)CC)nc(N)c21 ; CCCN(C)C(=O)n1c(=O)n(Cc2ccc(Cl)cc2)c2nc([S@@](=N)(=O)CC)nc(N)c21]

Example 41-A was prepared in analogy to Example 1, Method A, Step 6 by using Compound 41β-B instead of 6-amino-9-benzyl-2-(propylsulfonimidoyl)-7H-purin-8-one (Compound 1e). 6-Amino-9-[(4-chlorophenyl)methyl]-2-[S(R)-ethylsulfonimidoyl]-N-methyl-8-oxo-N-propyl-purine-7-carboxamide (Example 41-A, 78 mg) was obtained as a white solid. ¹H NMR (400 MHz, DMSO-d₆) δ ppm: 7.43-7.41 (m, 4H), 6.90 (s, 2H), 5.00 (s, 2H), 4.19 (s, 1H), 3.46-3.39 (m, 2H), 3.39-3.38 (m, 2H), 3.09-2.99 (m, 3H), 1.69-1.52 (m, 2H), 1.19 (t, J=7.28 Hz, 3H), 0.95-0.66 (m, 3H). MS obsd. (ESI⁺) [(M+H)⁺]: 466.1.

Example 41-B (125 mg) was prepared in analogy to Example 1, Method A, Step 6 by using Compound 41ϵ-A instead of 6-amino-9-benzyl-2-(propylsulfonimidoyl)-7H-purin-8-one (Compound 1e). 6-Amino-9-[(4-chlorophenyl)methyl]-2-[S(S)-ethylsulfonimidoyl]-N-methyl-8-oxo-N-propyl-purine-7-carboxamide (Example 41-B, 38 mg) was obtained as a white solid. ¹H NMR (400 MHz, DMSO-d₆) δ ppm: 7.43-7.41 (m, 4H), 6.90 (s, 2H), 5.00 (s, 2H), 4.20 (s, 1H), 3.46-3.41 (m, 2H), 3.40-3.39 (m, 2H), 3.10-3.00 (m, 3H), 1.69-1.50 (m, 2H), 1.24-1.12 (m, 3H), 0.93-0.73 (m, 3H). (MS obsd. (ESI⁺) [(M+H)+]: 466.2.

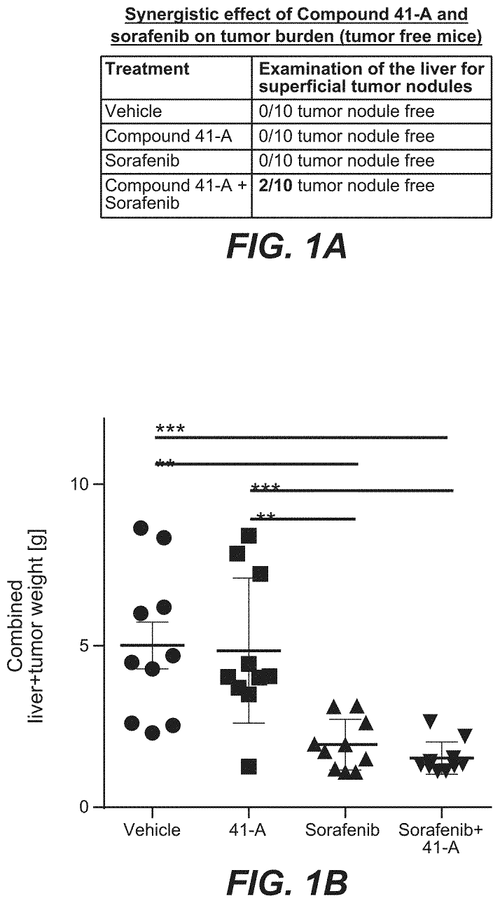

The stereochemistry of Example 41-B was determined by single crystal X-ray diffraction shown in FIG. 1.

### Example 42-A and Example 42-B

### 6-Amino-9-[(4-chlorophenyl)methyl]-N-ethyl-2 [S(S)-ethylsulfonimidoyl]-N-methyl-8-oxo-purine-7-carboxamide (Example 42-A) and 6-Amino-9-[(4-chlorophenyl)methyl]-N-ethyl-2-[S(R)-ethylsulfonimidoyl]-N-methyl-8-oxo-purine-7-carboxamide (Example 42-B)

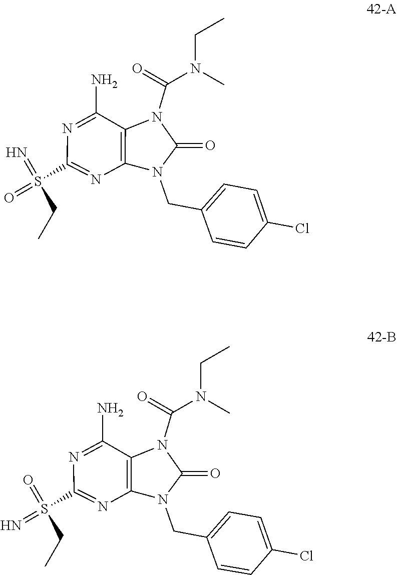 [SMILES: CCN(C)C(=O)n1c(=O)n(Cc2ccc(Cl)cc2)c2nc([S@@](=N)(=O)CC)nc(N)c21 ; CCN(C)C(=O)n1c(=O)n(Cc2ccc(Cl)cc2)c2nc([S@](=N)(=O)CC)nc(N)c21]

Example 42-A was prepared in analogy to Example 1, Method A, step 6 by using Compound 41α-A and N-ethyl-N-methyl-carbamoyl chloride instead of 6-amino-9-benzyl-2-(propylsulfonimidoyl)-7H-purin-8-one (Compound 1e) and N-methyl-N-propyl-carbamoyl chloride (Intermediate AA). 6-Amino-9-[(4-chlorophenyl)methyl]-N-ethyl-2[S(S)-ethylsulfonimidoyl]-N-methyl-8-oxo-purine-7-carboxamide (Example 42-A, 40 mg) was obtained as a white solid. ¹H NMR (400 MHz, DMSO-d₆) δ ppm: 7.43-7.41 (m, 4H), 6.90 (s, 2H), 4.99 (s, 2H), 4.18 (s, 1H), 3.48-3.40 (m, 2H), 3.39 (s, 2H), 3.05-3.01 (m, 3H), 1.20-1.14 (m, 6H). MS obsd. (ESI⁺) [(M+H)⁺]: 452.2.

Example 42-B was prepared in analogy to Example 1, Method A, Step 6 by using Compound 41β-B and N-ethyl-N-methyl-carbamoyl chloride instead of 6-amino-9-benzyl-2-(propylsulfonimidoyl)-7H-purin-8-one (Compound 1e) and N-methyl-N-propyl-carbamoyl chloride (Intermediate AA). 6-Amino-9-[(4-chlorophenyl)methyl]-N-ethyl-2-[S(R)-ethylsulfonimidoyl]-N-methyl-8-oxo-purine-7-carboxamide (Example 42-B, 38 mg) was obtained as a white solid. ¹H NMR (400 MHz, DMSO-d₆) δ ppm: 7.43-7.41 (m, 4H), 6.91 (s, 2H), 4.98 (s, 2H), 4.19 (s, 1H), 3.48-3.40 (m, 2H), 3.39 (s, 2H), 3.09-2.97 (m, 3H), 1.23-1.11 (m, 6H). MS obsd. (ESI⁺) [(M+H)⁺]: 452.2.

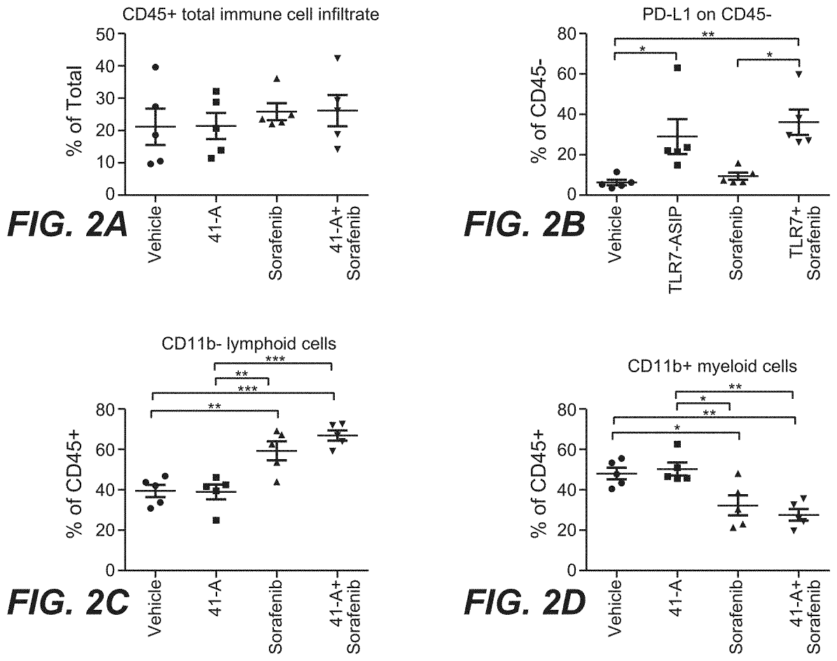

The stereochemistry of Example 42-A was determined by single crystal X-ray diffraction shown in FIG. 2.

### Example 43-A and Example 43-B

### 6-Amino-2-[S(R)-ethylsulfonimidoyl]-N-methyl-8-oxo-N-propyl-9-(p-tolylmethyl)purine-7-carb oxamide (Example 43-A) and 6-Amino-2-[S(S)-ethylsulfonimidoyl]-N-methyl-8-oxo-N-propyl-9-(p-tolylmethyl)purine-7-carboxamide (Example 43-B)

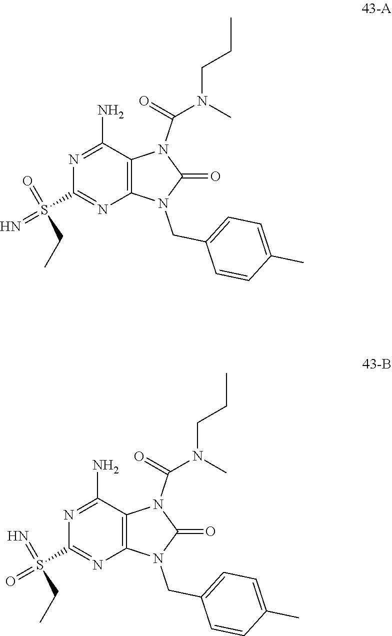 [SMILES: CCCN(C)C(=O)n1c(=O)n(Cc2ccc(C)cc2)c2nc([S@](=N)(=O)CC)nc(N)c21 ; CCCN(C)C(=O)n1c(=O)n(Cc2ccc(C)cc2)c2nc([S@@](=N)(=O)CC)nc(N)c21]

### Step 1: Preparation of 4-amino-2-oxo-3-(p-tolylmethyl)-1H-imidazole-5-carbonitrile (Compound 43a)

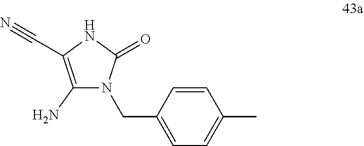 [SMILES: Cc1ccc(Cn2c(N)c(C#N)[nH]c2=O)cc1]

Compound 43a was prepared in analogy to Example 1, Method A, Step 1 by using 4-methylbenzyl isocyanate instead of benzyl isocyanate. 4-Amino-2-oxo-3-(p-tolylmethyl)-1H-imidazole-5-carbonitrile (26.6 g, Compound 43a) was obtained as a grey solid and used directly for next step without further purification. MS obsd. (ESI⁺) [(M+H)⁺]: 229.2.

### Step 2: Preparation of 6-amino-9-(p-tolylmethyl)-2-sulfanyl-7H-purin-8-one (Compound 43b)

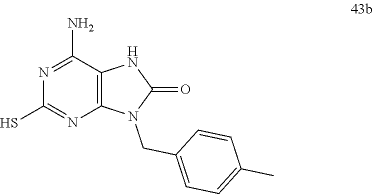 [SMILES: Cc1ccc(Cn2c(=O)[nH]c3c(N)nc(S)nc32)cc1]

Compound 43b was prepared in analogy to Example 1, Method A, Step 2 by using of 4-amino-2-oxo-3-(p-tolylmethyl)-1H-imidazole-5-carbonitrile (compound 43a) instead of 4-amino-3-benzyl-2-oxo-1H-imidazole-5-carbonitrile (Compound 1a). 6-Amino-9-(p-tolylmethyl)-2-sulfanyl-7H-purin-8-one (20.0 g, Compound 43b) was obtained as a yellow solid. MS obsd. (ESI⁺) [(M+H)⁺]: 288.

### Step 3: Preparation of 6-amino-2-ethylsulfanyl-9-(p-tolylmethyl)-7H-purin-8-one (Compound 43c)

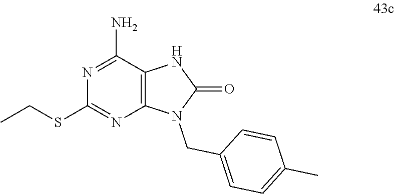 [SMILES: CCSc1nc(N)c2[nH]c(=O)n(Cc3ccc(C)cc3)c2n1]

Compound 43c was prepared in analogy to Example 1, Method A, Step 3 by using 6-amino-9-(p-tolylmethyl)-2-sulfanyl-7H-purin-8-one (Compound 43b) and iodoethane instead of 6-amino-9-benzyl-2-sulfanyl-7H-purin-8-one (Compound 1b) and bromopropane. 6-Amino-2-ethylsulfanyl-9-(p-tolylmethyl)-7H-purin-8-one (13 g, Compound 43c) was obtained as a yellow solid. MS obsd. (ESI⁺) [(M+H)⁺]: 316.

### Step 4: Preparation of 6-amino-2-ethylsulfinyl-9-(p-tolylmethyl)-7H-purin-8-one (Compound 43d)

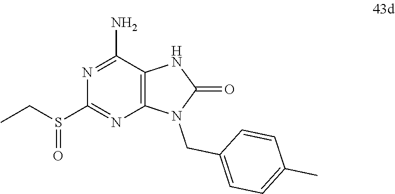 [SMILES: CCS(=O)c1nc(N)c2[nH]c(=O)n(Cc3ccc(C)cc3)c2n1]

Compound 43d was prepared in analogy to Example 1, Method A, Step 4 by using 6-amino-2-ethylsulfanyl-9-(p-tolylmethyl)-7H-purin-8-one (Compound 43c) instead of 6-amino-9-benzyl-2-methylsulfanyl-7H-purin-8-one (Compound 1c). 6-Amino-2-ethylsulfinyl-9-(p-tolylmethyl)-7H-purin-8-one 6 (3.5 g, Compound 43d) was obtained as a yellow solid. MS obsd. (ESI⁺) [(M+H)⁺]: 332.

### Step 5: Preparation of 6-amino-2-(ethylsulfonimidoyl)-9-(p-tolylmethyl)-7H-purin-8-one (Compound 43e)

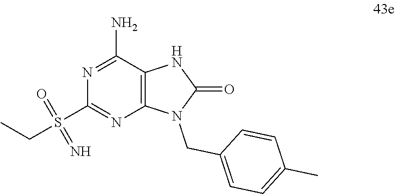 [SMILES: CCS(=N)(=O)c1nc(N)c2[nH]c(=O)n(Cc3ccc(C)cc3)c2n1]

Compound 43e was prepared in analogy to Example 1, Method A, Step 5 by using 6-amino-2-ethylsulfinyl-9-(p-tolylmethyl)-7H-purin-8-one (Compound 43d) instead of 6-amino-9-benzyl-2-methylsulfinyl-7H-purin-8-one (Compound 1d). 6-Amino-2-(ethylsulfonimidoyl)-9-(p-tolylmethyl)-7H-purin-8-one (530 mg, Compound 43e) was obtained as a yellow solid. ¹H NMR (400 MHz, DMSO-d₆) δ ppm: 10.53 (s, 1H), 7.24 (d, J=8.03 Hz, 2H), 7.13 (d, J=8.03 Hz, 2H), 6.94 (br. s., 2H), 4.91 (s, 2H), 4.03 (s, 1H), 3.36-3.41 (m, 2H), 2.26 (s, 3H), 1.18 (t, J=7.28 Hz, 3H). MS obsd. (ESI⁺) [(M+H)⁺]: 347.

Separation of compound of Compound 43e by chiral HPLC afforded Compound 43e-A (faster eluting, 56.8 mg) and Compound 43e-B (slower eluting, 56.7 mg) as white solid with methanol 5%-40% (0.05% DEA)/CO₂ on ChiralPak AD-3 column.

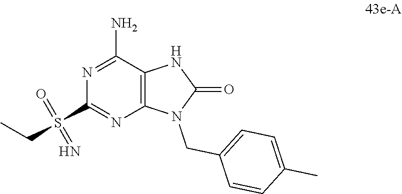 [SMILES: CC[S@@](=N)(=O)c1nc(N)c2[nH]c(=O)n(Cc3ccc(C)cc3)c2n1]

Compound 43e-A: ¹H NMR (400 MHz, DMSO-d₆) δ ppm: 10.52 (br. s., 1H), 7.23 (d, J=8.0 Hz, 2H), 7.13 (d, J=7.9 Hz, 2H), 6.94 (br. s., 2H), 4.90 (s, 2H), 4.03 (s, 1H), 3.42-3.33 (m, 2H), 2.25 (s, 3H), 1.17 (t, J=7.3 Hz, 3H). MS obsd. (ESI⁺) [(M+H)⁺]: 347.

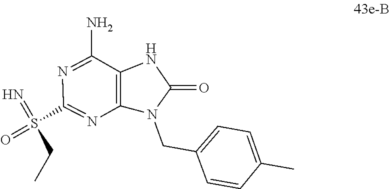 [SMILES: CC[S@](=N)(=O)c1nc(N)c2[nH]c(=O)n(Cc3ccc(C)cc3)c2n1]

Compound 43e-B: ¹H NMR (400 MHz, DMSO-d₆) δ ppm: 10.56 (br. s., 1H), 7.23 (d, J=8.0 Hz, 2H), 7.13 (d, J=8.0 Hz, 2H), 6.95 (br. s., 2H), 4.90 (s, 2H) 4.03 (s, 1H), 3.44-3.29 (m, 2H), 2.25 (s, 3H), 1.17 (t, J=7.3 Hz, 3H). MS obsd. (ESI⁺) [(M+H)⁺]: 347.

### Step 6: Preparation of 6-Amino-2-[S(R)-ethylsulfonimidoyl]-N-methyl-8-oxo-N-propyl-9-(p-tolylmethyl)purine-7-carboxamide (Example 43-A) and 6-Amino-2-[S(S)-ethylsulfonimidoyl]-N-methyl-8-oxo-N-propyl-9-(p-tolylmethyl)purine-7-carboxamide (Example 43-B)

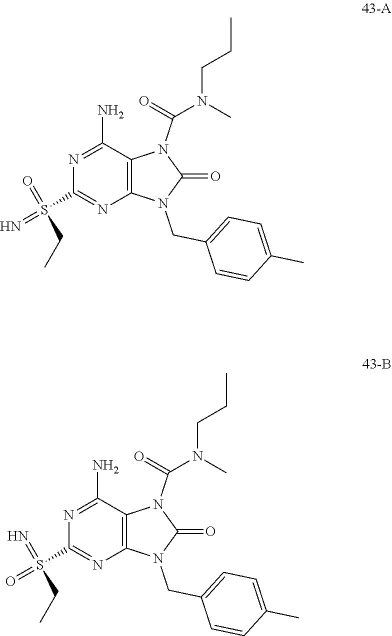 [SMILES: CCCN(C)C(=O)n1c(=O)n(Cc2ccc(C)cc2)c2nc([S@](=N)(=O)CC)nc(N)c21 ; CCCN(C)C(=O)n1c(=O)n(Cc2ccc(C)cc2)c2nc([S@@](=N)(=O)CC)nc(N)c21]

Example 43-A was prepared in analogy to Example 1, Method A, Step 6 by using Compound 43e-A instead of 6-amino-9-benzyl-2-(propylsulfonimidoyl)-7H-purin-8-one (Compound 1e). 6-Amino-2-[S(R)-ethylsulfonimidoyl]-N-methyl-8-oxo-N-propyl-9-(p-tolylmethyl)purine-7-carboxamide (Example 43-A, 58.1 mg, faster eluting, isopropanol from 5% to 40% (0.05% DEA)/CO₂ on ChiralPak AD-3 column) was obtained as a white solid. ¹H NMR (400 MHz, DMSO-d₆) δ ppm: 7.28 (d, J=7.8 Hz, 2H), 7.15 (d, J=7.8 Hz, 2H), 6.88 (br. s., 2H), 5.03-4.87 (m, 2H), 4.19 (s, 1H), 3.61-3.36 (m, 4H), 3.11-2.96 (m, 3H), 2.26 (s, 3H), 1.72-1.45 (m, 2H), 1.20 (t, J=7.2 Hz, 3H), 0.97-0.65 (m, 3H). MS obsd. (ESI⁺) [(M+H)⁺]: 446.

Example 43-B was prepared in analogy to Example 1, Method A, Step 6 by using Compound 43e-B instead of 6-amino-9-benzyl-2-(propylsulfonimidoyl)-7H-purin-8-one (Compound 1e). 6-Amino-2-[S(S)-ethylsulfonimidoyl]-N-methyl-8-oxo-N-propyl-9-(p-tolylmethyl)purine-7-carboxamide (Example 43-B, 40.1 mg, slower eluting, isopropanol from 5% to 40% (0.05% DEA)/CO₂ on ChiralPak AD-3 column) was obtained as a white solid: ¹H NMR (400 MHz, DMSO-d₆) δ ppm: 7.28 (d, J=7.5 Hz, 2H), 7.15 (d, J=7.5 Hz, 2H), 6.89 (br. s., 2H), 5.03-4.86 (m, 2H), 4.19 (s, 1H), 3.49-3.37 (m, 4H), 3.08-3.00 (m, 3H), 2.27 (s, 3H), 1.70-1.48 (m, 2H), 1.20 (t, J=7.2 Hz, 3H), 0.95-0.71 (m, 3H). MS obsd. (ESI⁺) [(M+H)⁺]: 446.3.

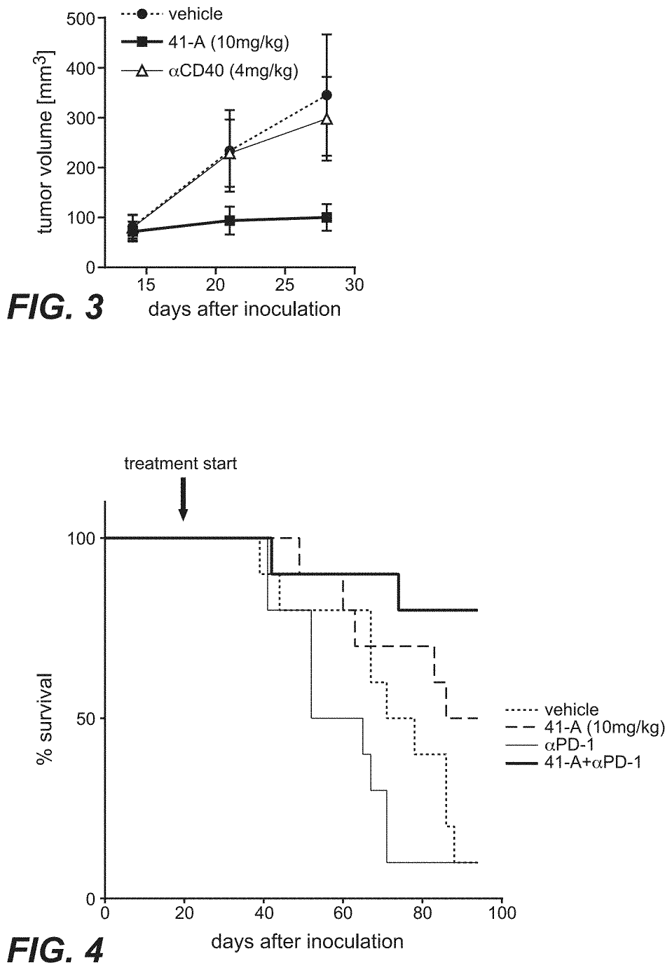

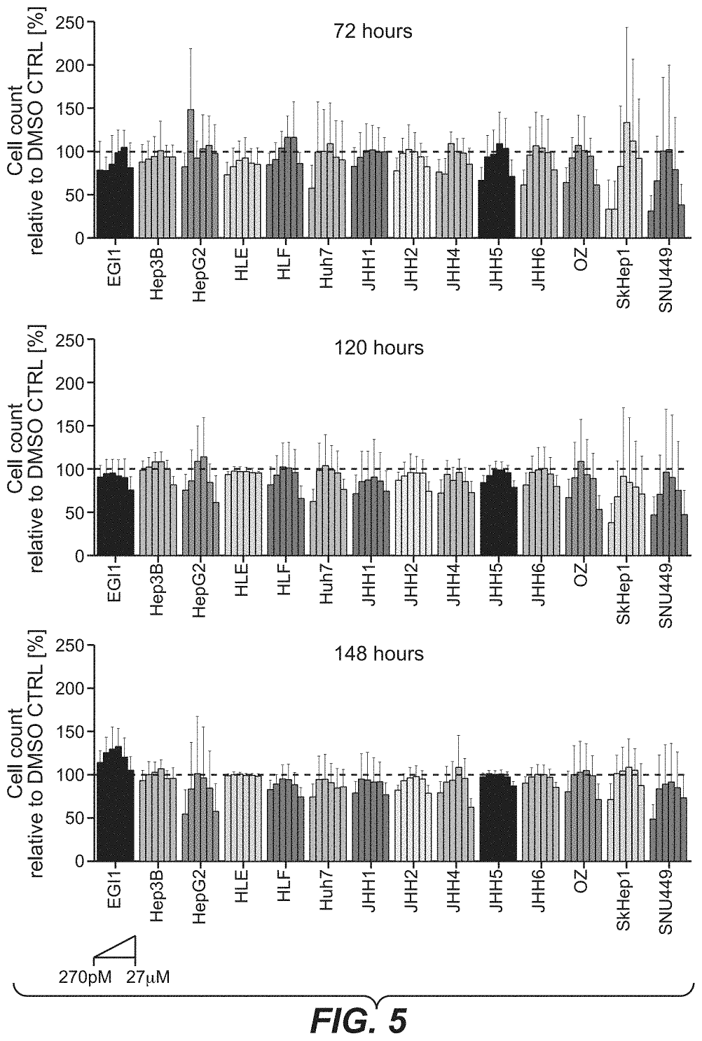

The stereochemistry of Example 43-B was determined by single crystal X-ray diffraction shown in FIG. 3.

### Example 44-A and Example 44-B

### 6-Amino-N-ethyl-2 [S(S)-ethylsulfonimidoyl]-N-methyl-8-oxo-9-(p-tolylmethyl)purine-7-carboxamide (Example 44-A) and 6-Amino-N-ethyl-2-[S(R)-ethylsulfonimidoyl]-N-methyl-8-oxo-9-(p-tolylmethyl)purine-7-carboxamide (Example 44-B)

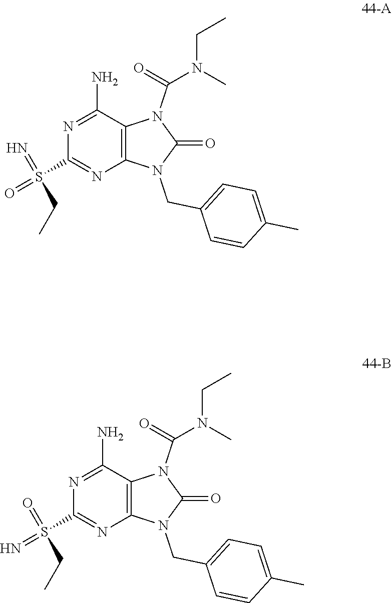 [SMILES: CCN(C)C(=O)n1c(=O)n(Cc2ccc(C)cc2)c2nc([S@@](=N)(=O)CC)nc(N)c21 ; CCN(C)C(=O)n1c(=O)n(Cc2ccc(C)cc2)c2nc([S@](=N)(=O)CC)nc(N)c21]

Example 44-A was prepared in analogy to Example 1, Method A, Step 6 by using Compound 43e-B and N-ethyl-N-methyl-carbamoyl chloride instead of 6-amino-9-benzyl-2-(propylsulfonimidoyl)-7H-purin-8-one (Compound 1e) and N-methyl-N-propyl-carbamoyl chloride (Intermediate AA). 6-Amino-N-ethyl-2[S(S)-ethylsulfonimidoyl]-N-methyl-8-oxo-9-(p-tolylmethyl)purine-7-carboxamide (Example 44-A, 73.1 mg) was obtained as a white solid. ¹H NMR (400 MHz, DMSO-d₆) δ ppm: 7.28 (d, J=7.8 Hz, 2H), 7.15 (d, J=7.8 Hz, 2H), 6.90 (br. s., 2H), 4.95 (s, 2H), 4.19 (br. s., 1H), 3.48-3.39 (m, 4H), 3.06-3.00 (m, 3H), 2.27 (s, 3H), 1.29-1.04 (m, 6H). MS obsd. (ESI⁺) [(M+H)⁺]: 432.

Example 44-B was prepared in analogy to Example 1, Method A, Step 6 by using Compound 43e-A and N-ethyl-N-methyl-carbamoyl chloride instead of 6-amino-9-benzyl-2-(propylsulfonimidoyl)-7H-purin-8-one (Compound 1e) and N-methyl-N-propyl-carbamoyl chloride (Intermediate AA). 6-Amino-N-ethyl-2-[S(R)-ethylsulfonimidoyl]-N-methyl-8-oxo-9-(p-tolylmethyl)purine-7-carboxamide (Example 44-B, 46.7 mg) was obtained as a white solid: ¹H NMR (400 MHz, DMSO-d₆) δ ppm: 7.28 (d, J=7.9 Hz, 2H), 7.15 (d, J=7.9 Hz, 2H), 6.90 (br. s., 2H), 4.95 (s, 2H), 4.19 (br. s., 1H), 3.50-3.39 (m, 4H), 3.10-2.96 (m, 3H), 2.27 (s, 3H), 1.27-1.10 (m, 6H). MS obsd. (ESI⁺) [(M+H)⁺]: 432.

### Example 45-A and Example 45-B

### 6-Amino-2-[S(R)ethylsulfonimidoyl]-9-[(4-fluorophenyl)methyl]-N-methyl-8-oxo-N-propyl-purine-7-carboxamide and 6-Amino-2-[S(S)ethylsulfonimidoyl]-9-[(4-fluorophenyl)methyl]-N-methyl-8-oxo-N-propyl-purine-7-carboxamide

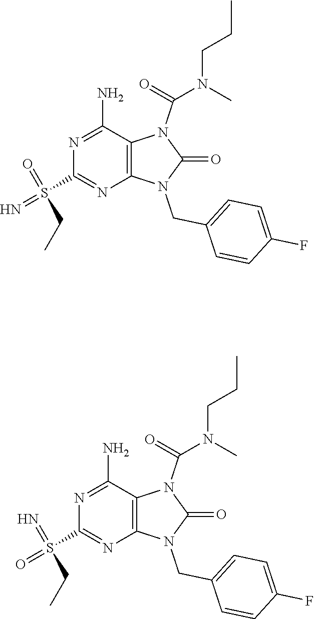 [SMILES: CCCN(C)C(=O)n1c(=O)n(Cc2ccc(F)cc2)c2nc([S@](=N)(=O)CC)nc(N)c21 ; CCCN(C)C(=O)n1c(=O)n(Cc2ccc(F)cc2)c2nc([S@@](=N)(=O)CC)nc(N)c21]

### Step 1: Preparation of 4-amino-3-[(4-fluorophenyl)methyl]-2-oxo-1H-imidazole-5-carbonitrile (Compound 45a)

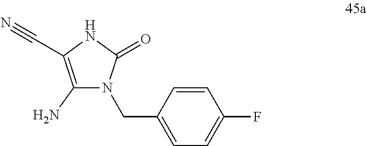 [SMILES: N#Cc1[nH]c(=O)n(Cc2ccc(F)cc2)c1N]

Compound 45a was prepared in analogy to Example 1, Method A, Step 1 by using 4-fluorobenzyl isocyanate instead of benzyl isocyanate. 4-Amino-3-[(4-fluorophenyl)methyl]-2-oxo-1H-imidazole-5-carbonitrile (48 g, Compound 45a) was obtained as a light yellow solid and was used directly for next step without further purification. MS obsd. (ESI⁺) [(M+H)⁺]: 233.

### Step 2: Preparation of 6-amino-9-[(4-fluorophenyl)methyl]-2-sulfanyl-7H-purin-8-one (Compound 45b)

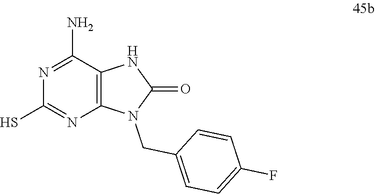 [SMILES: Nc1nc(S)nc2c1[nH]c(=O)n2Cc1ccc(F)cc1]

Compound 45b was prepared in analogy to Example 1, Method A, Step 2 by using of 4-amino-3-[(4-fluorophenyl)methyl]-2-oxo-1H-imidazole-5-carbonitrile (Compound 45a) instead of 4-amino-3-phenylmethyl-2-oxo-1H-imidazole-5-carbonitrile (Compound 1a). 6-Amino-9-[(4-fluorophenyl)methyl]-2-sulfanyl-7H-purin-8-one (32.0 g, Compound 45b) was obtained as a yellow solid. MS obsd. (ESI⁺) [(M+H)⁺]: 292.

### Step 3: Preparation of 6-amino-2-ethylsulfanyl-9-[(4-fluorophenyl)methyl]-7H-purin-8-one (Compound 45c)

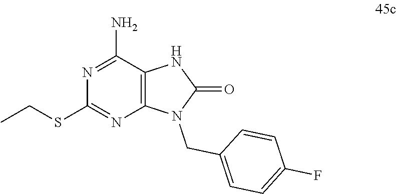 [SMILES: CCSc1nc(N)c2[nH]c(=O)n(Cc3ccc(F)cc3)c2n1]

Compound 45c was prepared in analogy to Example 1, Method A, Step 3 by using 6-amino-9-[(4-fluorophenyl)methyl]-2-sulfanyl-7H-purin-8-one (Compound 45b) and iodoethane instead of 6-amino-9-benzyl-2-sulfanyl-7H-purin-8-one (Compound 1b) and bromopropane. 6-Amino-2-ethylsulfanyl-9-[(4-fluorophenyl)methyl]-7H-purin-8-one (5.6 g, Compound 45c) was obtained as a yellow solid. MS obsd. (ESI⁺) [(M+H)⁺]: 320.

### Step 5: Preparation of 6-amino-2-ethylsulfinyl-9-[(4-fluorophenyl)methyl]-7H-purin-8-one (Compound 45d)

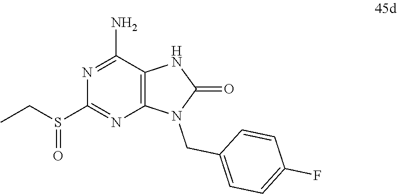 [SMILES: CCS(=O)c1nc(N)c2[nH]c(=O)n(Cc3ccc(F)cc3)c2n1]

Compound 45d was prepared in analogy to Example 1, Method A, Step 4 by using 6-amino-2-ethylsulfanyl-9-[(4-fluorophenyl)methyl]-7H-purin-8-one (Compound 45c) instead of 6-amino-9-benzyl-2-propylsulfanyl-7H-purin-8-one (Compound 1c). 6-Amino-2-ethylsulfinyl-9-[(4-fluorophenyl)methyl]-7H-purin-8-one (4.8 g, Compound 45d) was obtained as a yellow solid. MS obsd. (ESI⁺) [(M+H)⁺]: 332.

### Step 6: Preparation of 6-amino-2-(ethylsulfonimidoyl)-9-[(4-fluorophenyl)methyl]-7H-purin-8-one (Compound 45e)

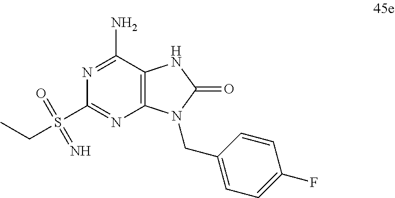 [SMILES: CCS(=N)(=O)c1nc(N)c2[nH]c(=O)n(Cc3ccc(F)cc3)c2n1]

Compound 45e was prepared in analogy to Example 1, Method A, Step 5 by using 6-amino-2-ethylsulfinyl-9-[(4-fluorophenyl)methyl]-7H-purin-8-one (Compound 45d) instead of 6-amino-9-benzyl-2-propylsulfinyl-7H-purin-8-one (Compound 1d). 6-Amino-2-(ethylsulfonimidoyl)-9-[(4-fluorophenyl)methyl]-7H-purin-8-one (2.9 g, Compound 45e) was obtained as a yellow solid. ¹H NMR (400 MHz, DMSO-d₆) δ ppm: 10.57 (br. s., 1H), 7.40 (dd, J=8.5, 5.5 Hz, 2H), 7.16 (t, J=8.9 Hz, 2H), 6.97 (br. s., 2H), 4.94 (s, 2H), 4.07 (s, 1H), 3.43-3.36 (m, 2H), 1.17 (t, J=7.4 Hz, 3H). MS obsd. (ESI⁺) [(M+H)⁺]: 351.

Separation of compound of Compound 45e by chiral HPLC afforded Compound 45e-A (faster eluting, 85.4 mg) and Compound 45e-B (slower eluting, 36.4 mg) as white solid with methanol 5%-40% (0.05% DEA)/CO₂ on ChiralPak AD-3 column.

Compound 45e-A: ¹H NMR (400 MHz, DMSO-d₆) ppm: 10.53 (br. s., 1H), 7.41 (dd, J=8.5, 5.5 Hz, 2H), 7.17 (t, J=8.9 Hz, 2H), 6.98 (br. s., 2H), 4.95 (s, 2H), 4.07 (s, 1H), 3.45-3.36 (m, 2H), 1.17 (t, J=7.3 Hz, 3H). MS obsd. (ESI⁺) [(M+H)⁺]: 351.

Compound 45e-B: ¹H NMR (400 MHz, DMSO-d₆) δ ppm: 10.53 (br. s., 1H), 7.41 (dd, J=8.5, 5.5 Hz, 2H), 7.17 (t, J=8.9 Hz, 2H), 6.98 (br. s., 2H), 4.95 (s, 2H), 4.07 (s, 1H), 3.44-3.37 (m, 2H) 1.17 (t, J=7.3 Hz, 3H). MS obsd. (ESI⁺) [(M+H)⁺]: 351.

### Step 7: Preparation of 6-amino-2-(ethylsulfonimidoyl)-9-[(4-fluorophenyl)methyl]-N-methyl-8-oxo-N-propyl-purine-7-carboxamide (Example 45), 6-Amino-2-[S(R)ethylsulfonimidoyl]-9-[(4-fluorophenyl)methyl]-N-methyl-8-oxo-N-propyl-purine-7-carboxamide and 6-Amino-2-[S(S)ethylsulfonimidoyl]-9-[(4-fluorophenyl)methyl]-N-methyl-8-oxo-N-propyl-purine-7-carboxamide (Example 45-A and Example 45-B)

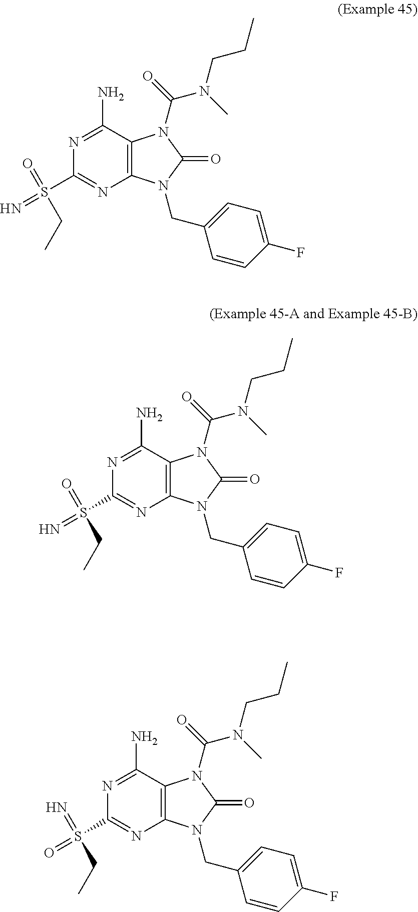 [SMILES: CCCN(C)C(=O)n1c(=O)n(Cc2ccc(F)cc2)c2nc(S(=N)(=O)CC)nc(N)c21 ; CCCN(C)C(=O)n1c(=O)n(Cc2ccc(F)cc2)c2nc([S@](=N)(=O)CC)nc(N)c21 ; CCCN(C)C(=O)n1c(=O)n(Cc2ccc(F)cc2)c2nc([S@@](=N)(=O)CC)nc(N)c21]

Example 45 was prepared in analogy to Example 1, Method A, Step 6 by using 6-amino-2-(ethylsulfonimidoyl)-9-[(4-fluorophenyl)methyl]-7H-purin-8-one (Compound 45e) instead of 6-amino-9-benzyl-2-(propylsulfonimidoyl)-7H-purin-8-one (Compound 1e). 6-Amino-2-(ethylsulfonimidoyl)-9-[(4-fluorophenyl)methyl]-N-methyl-8-oxo-N-propyl-purine-7-carboxamide (162.4 mg, Example 45) was obtained as a white solid.

Separation of compound of Example 45 by chiral HPLC afforded Example 45-A (faster eluting, 85.3 mg) and Example 45-B (slower eluting, 52 mg) as white solid with methanol 5%-40% (0.05% DEA)/CO₂ on ChiralPak AD-3 column

Example 45-A: ¹H NMR (400 MHz, DMSO-d₆) δ ppm: 7.53-7.38 (m, 2H), 7.18 (t, J=8.9 Hz, 2H), 6.90 (br. s., 2H), 4.99 (s, 2H), 4.21 (s, 1H), 3.48-3.37 (m, 4H), 3.10-3.01 (m, 3H), 1.69-1.49 (m, 2H), 1.25-1.14 (m, 3H), 0.94-0.72 (m, 3H). MS obsd. (ESI⁺) [(M+H)⁺]: 450.

Example 45-B: ¹H NMR (400 MHz, DMSO-d₆) δ ppm: 7.54-7.38 (m, 2H), 7.18 (t, J=8.9 Hz, 2H), 7.01-6.72 (m, 2H), 4.99 (s, 2H), 4.21 (s, 1H), 3.46-3.38 (m, 4H), 3.10-3.01 (m, 3H), 1.76-1.50 (m, 2H), 1.25-1.16 (m, 3H), 0.99-0.69 (m, 3H). MS obsd. (ESI⁺) [(M+H)⁺]: 450.

### Example 46-A and Example 46-B

### 6-Amino-N-ethyl-2-(ethylsulfonimidoyl)-9-[(4-fluorophenyl)methyl]-N-methyl-8-oxo-purine-7-carboxamide (Example 46), 6-amino-N-ethyl-2-[S(S)-(ethylsulfonimidoyl)]-9-[(4-fluorophenyl)methyl]-N-methyl-8-oxo-purine-7-carboxamide and 6-amino-N-ethyl-2-[S(R)-(ethylsulfonimidoyl)]-9-[(4-fluorophenyl)methyl]-N-methyl-8-oxo-purine-7-carboxamide (Example 46-A and Example 46-B)

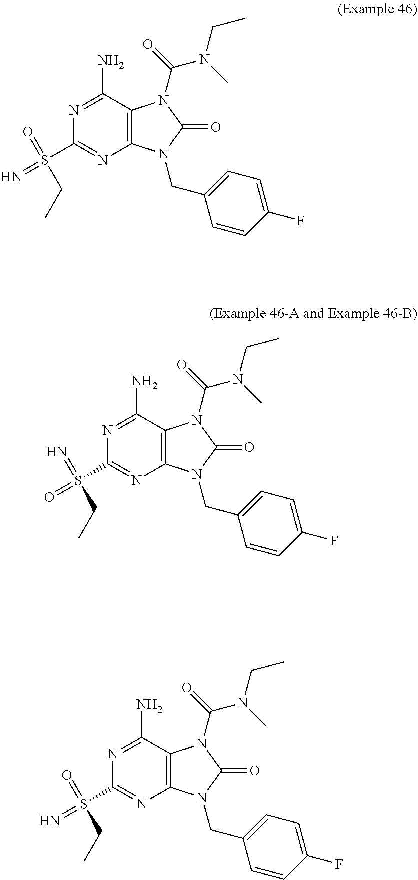 [SMILES: CCN(C)C(=O)n1c(=O)n(Cc2ccc(F)cc2)c2nc(S(=N)(=O)CC)nc(N)c21 ; CCN(C)C(=O)n1c(=O)n(Cc2ccc(F)cc2)c2nc([S@@](=N)(=O)CC)nc(N)c21 ; CCN(C)C(=O)n1c(=O)n(Cc2ccc(F)cc2)c2nc([S@](=N)(=O)CC)nc(N)c21]

Example 46 was prepared in analogy to Example 1, Method A, Step 6 by using 6-amino-2-(ethylsulfonimidoyl)-9-[(4-fluorophenyl)methyl]-7H-purin-8-one (Compound 45e) and N-ethyl-N-methyl carbamoyl chloride instead of 6-amino-9-benzyl-2-(propylsulfonimidoyl)-7H-purin-8-one (Compound 1e) and N-methyl-N-propyl-carbamoyl chloride (Intermediate AA). 6-Amino-N-ethyl-2-(ethylsulfonimidoyl)-9-[(4-fluorophenyl)methyl]-N-methyl-8-oxo-purine-7-carboxamide (51 mg, Example 46) was obtained as a white solid. ¹H NMR (400 MHz, DMSO-d₆) δ ppm: 7.46-7.43 (m, 2H), 7.20-7.15 (m, 2H), 6.90 (br. s., 2H), 4.98 (s, 2H), 4.18 (s, 1H), 3.47-3.32 (m, 4H), 3.05-3.01 (m, 3H), 1.21-1.14 (m, 6H). MS obsd. (ESI⁺) [(M+H)⁺]: 436.

Separation of compound of Example 46 by chiral HPLC afforded Example 46-A (faster eluting, 72 mg) and Example 46-B (slower eluting, 45 mg) as white solid with methanol 5%-40% (0.05% DEA)/CO₂ on ChiralPak AD-3 column

Example 46-A: ¹H NMR (400 MHz, DMSO-d₆) δ ppm: 7.46-7.43 (m, 2H), 7.20-7.16 (m, 2H), 6.90 (br. s., 2H), 4.98 (s, 2H), 4.18 (s, 1H), 3.47-3.32 (m, 4H), 3.05-3.01 (m, 3H), 1.21-1.14 (m, 6H). MS obsd. (ESI⁺) [(M+H)⁺]: 436.

Example 46-B: ¹H NMR (400 MHz, DMSO-d₆) δ ppm: 7.46-7.43 (m, 2H), 7.20-7.14 (m, 2H), 6.92 (br. s., 2H), 4.98 (s, 2H), 4.20 (br. s., 1H), 3.47-3.32 (m, 4H), 3.05-3.01 (m, 3H), 1.23-1.19 (m, 6H). MS obsd. (ESI⁺) [(M+H)⁺]: 436.

### Example 47-A and Example 47-B

### 6-Amino-9-[(4-bromophenyl)methyl]-2-(ethylsulfonimidoyl)-N-methyl-8-oxo-N-propyl-purine-7-carboxamide (Example 47), 6-amino-2-[S(R)-ethylsulfonimidoyl]-9-[(4-bromophenyl)methyl]-N-methyl-8-oxo-N-propyl-purine-7-carboxamide and 6-amino-2-[S(S)-ethylsulfonimidoyl]-9-[(4-bromophenyl)methyl]-N-methyl-8-oxo-N-propyl-purine-7-carboxamide

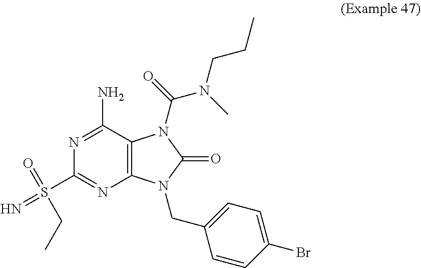 [SMILES: CCCN(C)C(=O)n1c(=O)n(Cc2ccc(Br)cc2)c2nc(S(=N)(=O)CC)nc(N)c21]

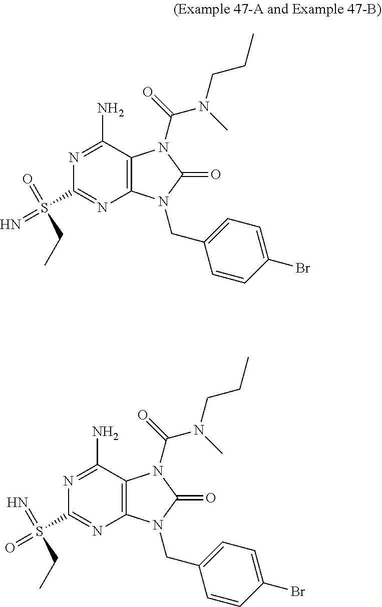 [SMILES: CCCN(C)C(=O)n1c(=O)n(Cc2ccc(Br)cc2)c2nc([S@](=N)(=O)CC)nc(N)c21 ; CCCN(C)C(=O)n1c(=O)n(Cc2ccc(Br)cc2)c2nc([S@@](=N)(=O)CC)nc(N)c21]

### Step 1: Preparation of 4-amino-3-[(4-bromophenyl)methyl]-2-oxo-1H-imidazole-5-carbonitrile (Compound 47a)

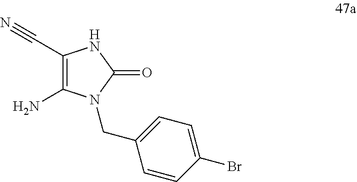 [SMILES: N#Cc1[nH]c(=O)n(Cc2ccc(Br)cc2)c1N]

Compound 47a was prepared in analogy to Example 1, Method A, Step 1 by using 4-bromobenzyl isocyanate instead of benzyl isocyanate. 4-Amino-3-[(4-bromophenyl)methyl]-2-oxo-1H-imidazole-5-carbonitrile (500 mg, Compound 47a) was obtained as a light yellow solid and was used directly for next step without further purification. ¹H NMR (400 MHz, DMSO-d₆) δ ppm: 9.94 (S, 1H), 7.55-7.53 (d, J=8.0 Hz, 2H), 7.20-7.18 (d, J=8.0 Hz, 2H), 6.52 (br. s., 2H), 4.74 (s, 2H). MS obsd. (ESI⁺) [(M+H)⁺]: 293.

### Step 2: Preparation of 6-amino-9-[(4-bromophenyl)methyl]-2-sulfanyl-7H-purin-8-one (Compound 47b)

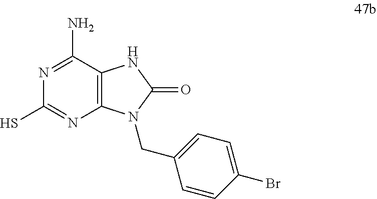 [SMILES: Nc1nc(S)nc2c1[nH]c(=O)n2Cc1ccc(Br)cc1]

Compound 47b was prepared in analogy to Example 1, Method A, Step 2 by using of 4-amino-3-[(4-bromophenyl)methyl]-2-oxo-1H-imidazole-5-carbonitrile (Compound 47a) instead of 4-amino-3-phenylmethyl-2-oxo-1H-imidazole-5-carbonitrile (Compound 1a). 6-Amino-9-[(4-bromophenyl)methyl]-2-sulfanyl-7H-purin-8-one (300 mg, Compound 47b) was obtained as a yellow solid. MS obsd. (ESI⁺) [(M+H)⁺]: 352.

### Step 3: Preparation of 6-amino-2-ethylsulfanyl-9-[(4-bromophenyl)methyl]-7H-purin-8-one (Compound 47c)

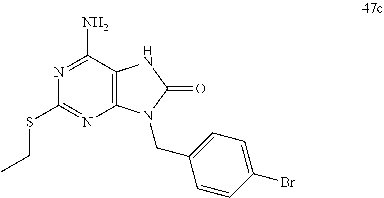 [SMILES: CCSc1nc(N)c2[nH]c(=O)n(Cc3ccc(Br)cc3)c2n1]

Compound 47c was prepared in analogy to Example 1, Method A, Step 3 by using 6-amino-9-[(4-bromophenyl)methyl]-2-sulfanyl-7H-purin-8-one (Compound 45b) and iodoethane instead of 6-amino-9-benzyl-2-sulfanyl-7H-purin-8-one (Compound 1b) and bromopropane. 6-Amino-2-ethylsulfanyl-9-[(4-bromophenyl)methyl]-7H-purin-8-one (5.6 g, Compound 47c) was obtained as a yellow solid. MS obsd. (ESI⁺) [(M+H)⁺]: 380.

### Step 4: Preparation of 6-amino-9-[(4-bromophenyl)methyl]-2-ethylsulfinyl-7h-purin-8-one (compound 47d)

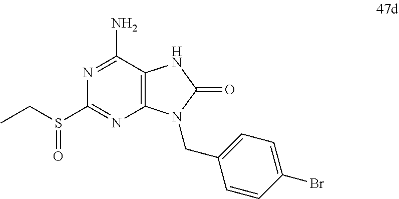 [SMILES: CCS(=O)c1nc(N)c2[nH]c(=O)n(Cc3ccc(Br)cc3)c2n1]

Compound 47d was prepared in analogy to Example 1, Method B, Step 6 by using 6-amino-9-[(4-bromophenyl)methyl]-2-ethylsulfanyl-7H-purin-8-one (Compound 47c) instead of 6-amino-9-benzyl-2-(2-propylsulfanyl)-7H-purin-8-one (Compound 1c). 6-Amino-9-[(4-bromophenyl)methyl]-2-ethylsulfinyl-7H-purin-8-one (3.2 g, Compound 47d) was obtained as a white solid. MS obsd. (ESI⁺) [(M+H)⁺]: 396.

### Step 5: Preparation of 6-amino-9-[(4-bromophenyl)methyl]-2-(ethylsulfonimidoyl)-7h-purin-8-one (Compound 47e)

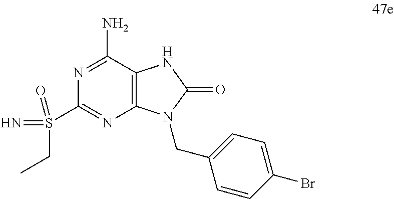 [SMILES: CCS(=N)(=O)c1nc(N)c2[nH]c(=O)n(Cc3ccc(Br)cc3)c2n1]

Compound 47e was prepared in analogy to Example 1, Method B, Step 7 by using 6-amino-9-[(4-bromophenyl)methyl]-2-ethylsulfinyl-7H-purin-8-one (Compound 47d) instead of 6-amino-9-benzyl-2-propylsulfinyl-7H-purin-8-one (Compound 1d). 6-Amino-9-[(4-bromophenyl)methyl]-2-(ethylsulfonimidoyl)-7H-purin-8-one (4.0 g, Compound 47e) was obtained as a white solid. MS obsd. (ESI⁺) [(M+H)⁺]: 411.

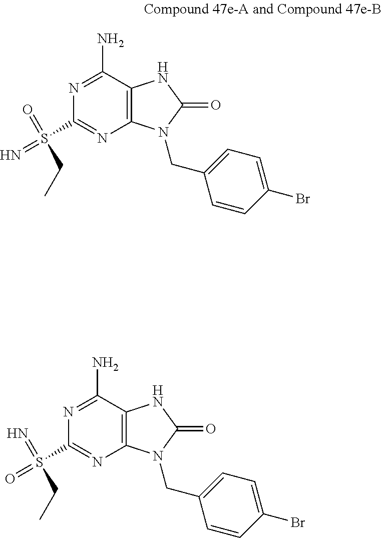 [SMILES: CC[S@@](=N)(=O)c1nc(N)c2[nH]c(=O)n(Cc3ccc(Br)cc3)c2n1 ; CC[S@](=N)(=O)c1nc(N)c2[nH]c(=O)n(Cc3ccc(Br)cc3)c2n1]

Separation of compound of Compound 47e by chiral HPLC afforded Compound 47e-A (faster eluting, 112 mg) and Compound 47e-B (slower eluting, 99 mg) as white solid with methanol 5%-40% (0.05% DEA)/CO₂ on ChiralPak AD-3 column.

Compound 47e-A: ¹H NMR (400 MHz, DMSO-d₆) δ ppm: 10.58 (br. s., 1H), 7.52-7.54 (d, J=8.0, 2H), 7.31-7.29 (t, J=8.0 Hz, 2H), 6.54 (br. s., 2H), 4.93 (s, 2H), 4.05 (s, 1H), 3.42-3.31 (m, 2H), 1.15 (t, J=7.3 Hz, 3H). MS obsd. (ESI⁺) [(M+H)⁺]: 411.

Compound 47e-B: ¹H NMR (400 MHz, DMSO-d₆) δ ppm: 10.58 (br. s., 1H), 7.54-7.52 (d, J=8.0, 2H), 7.31-7.29 (t, J=8.0 Hz, 2H), 6.98 (br. s., 2H), 4.93 (s, 2H), 4.06 (s, 1H), 3.40-3.37 (m, 2H), 1.15 (t, J=7.3 Hz, 3H). MS obsd. (ESI⁺) [(M+H)⁺]: 411.

### Step 6: Preparation of 6-amino-9-[(4-bromophenyl)methyl]-2-(ethylsulfonimidoyl)-N-methyl-8-oxo-N-propyl-purine-7-carboxamide (Example 47), 6-amino-9-[(4-bromophenyl)methyl]-2-[S(R)-ethylsulfonimidoyl]-N-methyl-8-oxo-N-propyl-purine-7-carboxamide and 6-amino-9-[(4-bromophenyl)methyl]-2-[S(S)-ethylsulfonimidoyl]-N-methyl-8-oxo-N-propyl-purine-7-carboxamide (Example 47-A and Example 47-B)

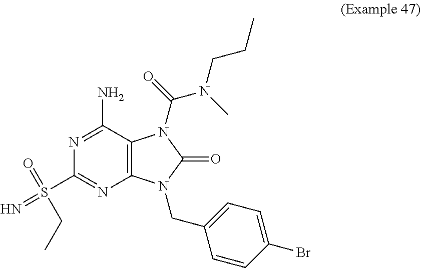 [SMILES: CCCN(C)C(=O)n1c(=O)n(Cc2ccc(Br)cc2)c2nc(S(=N)(=O)CC)nc(N)c21]

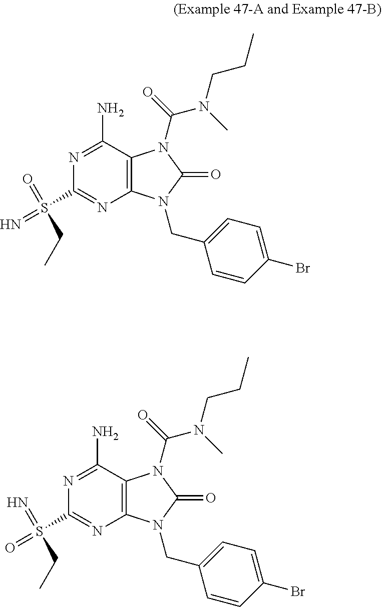 [SMILES: CCCN(C)C(=O)n1c(=O)n(Cc2ccc(Br)cc2)c2nc([S@](=N)(=O)CC)nc(N)c21 ; CCCN(C)C(=O)n1c(=O)n(Cc2ccc(Br)cc2)c2nc([S@@](=N)(=O)CC)nc(N)c21]

Example 47 was prepared in analogy to Example 1, Method A, Step 6 by using 6-amino-9-[(4-bromophenyl)methyl]-2-(ethylsulfonimidoyl)-7H-purin-8-one (Compound 47e) instead of 6-amino-9-benzyl-2-(propylsulfonimidoyl)-7H-purin-8-one (Compound 1e). 6-Amino-9-[(4-bromophenyl)methyl]-2-(ethylsulfonimidoyl)-N-methyl-8-oxo-N-propyl-purine-7-carboxamide (570 mg, Example 47) was obtained as a white solid. ¹H NMR (400 MHz, DMSO-d₆) δ ppm: 7.56-7.53 (m, 2H), 7.36-7.34 (m, 2H), 6.92 (br. s., 2H), 4.97 (s, 2H), 4.18 (s, 1H), 3.45-3.38 (m, 4H), 3.05-3.02 (m, 3H), 1.65-1.56 (m, 2H), 1.19 (t, J=8.0 Hz, 3H), 0.93-0.75 (m, 3H). MS obsd. (ESI⁺) [(M+H)⁺]: 510.

Separation of compound of Example 47 by chiral HPLC afforded Example 47-A (faster eluting, 260 mg) and Example 47-B (slower eluting, 266 mg) as white solid with methanol 5%-40% (0.05% DEA)/CO₂ on ChiralPak AD-3 column

Example 47-A: ¹H NMR (400 MHz, DMSO-d₆) δ ppm: 7.56-7.54 (d, J=8.0 Hz, 2H), 7.36-7.33 (d, J=8.0 Hz, 2H), 6.90 (br. s., 2H), 4.97 (s, 2H), 4.21 (s, 1H), 3.46-3.41 (m, 4H), 3.05-3.02 (m, 3H), 1.65-1.54 (m, 2H), 1.24-1.16 (m, 3H), 0.93-0.75 (m, 3H). MS obsd. (ESI⁺) [(M+H)⁺]: 510.

Example 47-B: ¹H NMR (400 MHz, DMSO-d₆) δ ppm: 7.54-7.53 (d, J=8.0 Hz, 2H), 7.36-7.33 (d, J=8.0 Hz, 2H), 6.90 (br. s., 2H), 4.97 (s, 2H), 4.21 (s, 1H), 3.46-3.41 (m, 4H), 3.06-3.02 (m, 3H), 1.65-1.54 (m, 2H), 1.20-1.16 (m, 3H), 0.93-0.75 (m, 3H). MS obsd. (ESI⁺) [(M+H)⁺]: 510.

### Example 48-A and Example 48-B

### 6-Amino-9-[(4-bromophenyl)methyl]-N-ethyl-2-(ethylsulfonimidoyl)-N-methyl-8-oxo-purine-7-carboxamide (Example 48), 6-amino-9-[(4-bromophenyl)methyl]-N-ethyl-2-[S(S)-(ethylsulfonimidoyl)]-N-methyl-8-oxo-purine-7-carboxamide and 6-amino-9-[(4-bromophenyl)methyl]-N-ethyl-2-[S(R)-(ethylsulfonimidoyl)]-N-methyl-8-oxo-purine-7-carboxamide (Example 48-A and Example 48-B)

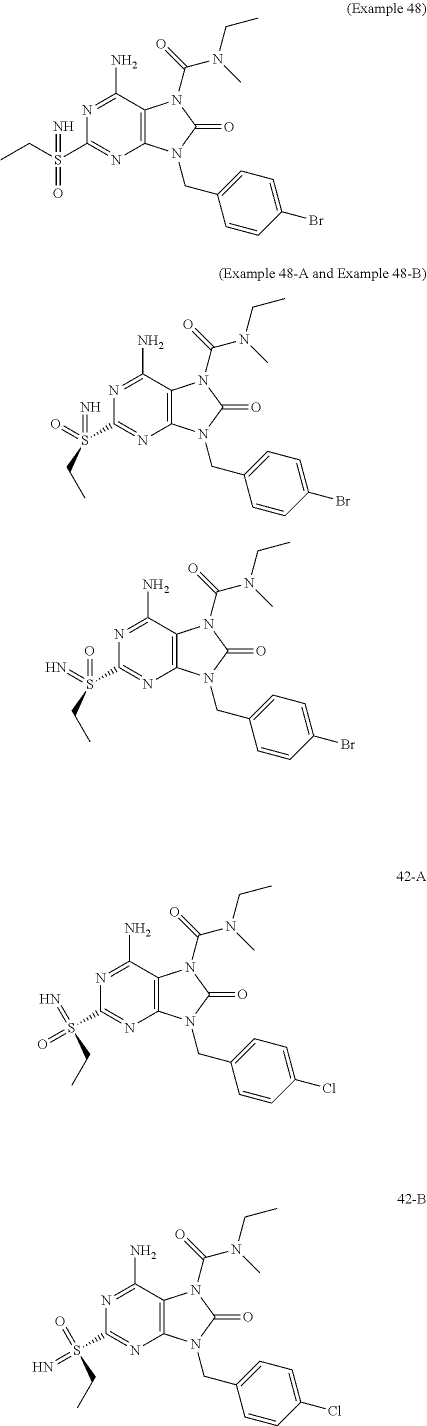

Example 48 was prepared in analogy to Example 1, Method A, Step 6 by using 6-amino-9-[(4-bromophenyl)methyl]-2-(ethylsulfonimidoyl)-7H-purin-8-one (Compound 47e) and N-ethyl-N-methyl-carbamoyl chloride instead of 6-amino-9-benzyl-2-(propylsulfonimidoyl)-7H-purin-8-one (Compound 1e) and N-methyl-N-propyl-carbamoyl chloride (Intermediate AA). 6-Amino-9-[(4-bromophenyl)methyl]-2-(ethylsulfonimidoyl)-N-methyl-8-oxo-N-propyl-purine-7-carboxamide (469 mg, Example 48) was obtained as a white solid. ¹H NMR (400 MHz, DMSO-d₆) δ ppm: 7.56-7.54 (d, J=8.0 Hz, 2H), 7.36-7.34 (d, J=8.0 Hz, 2H), 6.98 (br. s., 2H), 4.97 (s, 2H), 3.53-3.46 (m, 4H), 3.05-3.01 (m, 3H), 1.22-1.16 (m, 6H). MS obsd. (ESI⁺) [(M+H)⁺]: 496.

Separation of compound of Example 48 by chiral HPLC afforded Example 48-A (faster eluting, 198 mg) and Example 48-B (slower eluting, 202 mg) as white solid with methanol 5%-40% (0.05% DEA)/CO₂ on ChiralPak AD-3 column.

Example 48-A: ¹H NMR (400 MHz, DMSO-d) δ ppm: 7.56-7.54 (d, J=8.0 Hz, 2H), 7.36-7.34 (d, J=8.0 Hz, 2H), 6.92 (br. s., 2H), 4.97 (s, 2H), 4.19-4.18 (m, 1H), 3.46-3.41 (m, 4H), 3.05-3.01 (m, 3H), 1.20-1.14 (m, 6H). MS obsd. (ESI⁺) [(M+H)⁺]: 496.

Example 48-B: ¹H NMR (400 MHz, DMSO-d₆) δ ppm: 7.56-7.54 (d, J=8.0 Hz, 2H), 7.36-7.34 (d, J=8.0 Hz, 2H), 6.92 (br. s., 2H), 4.97 (s, 2H), 4.24 (br. s., 1H), 3.58-3.41 (m, 4H), 3.05-3.01 (m, 3H), 1.26-1.01 (m, 6H). MS obsd. (ESI⁺) [(M+H)⁺]: 496.

### Example 49

### Activity of Compounds and Examples in HEK293-hTLR-7 Assay

HEK293-Blue-hTLR-7 cells assay:

A stable HEK293-Blue-hTLR-7 cell line was purchased from InvivoGen (Cat. #: hkb-htlr7, San Diego, Calif., USA). These cells were designed for studying the stimulation of human TLR7 by monitoring the activation of NF-κB. A SEAP (secreted embryonic alkaline phosphatase) reporter gene was placed under the control of the IFN-β minimal promoter fused to five NF-κB and AP-1-binding sites. The SEAP was induced by activating NF-κB and AP-1 via stimulating HEK-Blue hTLR7 cells with TLR7 ligands. Therefore the reporter expression was regulated by the NF-κB promoter upon stimulation of human TLR7 for 20 hrs. The cell culture supernatant SEAP reporter activity was determined using QUANTI-Blue™ kit (Cat. #: rep-qbl, Invivogen, San Diego, Ca, USA) at a wavelength of 640 nm, a detection medium that turns purple or blue in the presence of alkaline phosphatase.

HEK293-Blue-hTLR7 cells were incubated at a density of 250,000-450,000 cells/mL in a volume of 180 μL in a 96-well plate in Dulbecco's Modified Eagle's medium (DMEM) containing 4.5 g/L glucose, 50 U/mL penicillin, 50 mg/mL streptomycin, 100 mg/mL Normocin, 2 mM L-glutamine, 10% (V/V) heat-inactivated fetal bovine serum for 24 hrs. Then the HEK293-Blue-hTLR-7 cells were incubated with addition of 20 μL test compound in a serial dilution in the presence of final DMSO at 1% and perform incubation under 37° C. in a CO₂ incubator for 20 hrs. Then 20 μL of the supernatant from each well was incubated with 180 μL Quanti-blue substrate solution at 37° C. for 2 hrs and the absorbance was read at 620-655 nm using a spectrophotometer. The signalling pathway that TLR7 activation leads to downstream NF-κB activation has been widely accepted, and therefore similar reporter assay was also widely used for evaluating TLR7 agonist (Tsuneyasu Kaisho and Takashi Tanaka, Trends in Immunology, Volume 29, Issue 7, July 2008, Pages 329.sci; Hiroaki Hemmi et al, Nature Immunology 3, 196-200 (2002)).

The Compounds and Examples of the present invention were tested in HEK293-hTLR-7 assay for their TLR7 agonism activity as described herein and results are listed in Table 1. The Examples of prodrugs were found to have EC₅₀ of about 2.1 μM to about 1000 μM, the Compounds of active forms were found to have EC₅₀ less than 0.2 μM. The calculated ratio of EC_{50(prodrug)}/EC_{50(active form)} were within the range from 32 to about 7600.

TABLE 1
Activity of Examples and Compounds of present
invention in HEK293-hTLR-7 assay

| | | | HEK293- | |
|---|---|---|---|---|
| | HEK293- | | hTLR-7 EC₅₀ | Ratio |
| | hTLR-7 EC₅₀ | Corresponding | (Active form, | (EC_{50(prodrug)}/ |
| Prodrug | (Prodrug, μM) | Active Form | μM) | EC_{50(active form)}) |
| Example 1 | 50.4 | Compound 1e | 0.065 | 775.4 |
| Example 1-A | 42.5 | Compound 1e-A | 0.067 | 634.3 |
| Example 1-B | 27 | Compound 1e-B | 0.086 | 314.0 |
| Example 2 | 32 | Compound 1e | 0.065 | 372.1 |
| Example 2-A | 3.7 | Compound 1e-B | 0.086 | 43.0 |
| Example 2-B | 4.4 | Compound 1e-A | 0.067 | 65.7 |
| Example 3 | 15.1 | Compound 1e | 0.065 | 232.3 |
| Example 4 | 23 | Compound 1e | 0.065 | 353.8 |
| Example 5 | 41 | Compound 1e | 0.065 | 630.8 |
| Example 6 | 82.3 | Compound 1e | 0.065 | 1266.2 |
| Example 7 | 19.9 | Compound 1e | 0.065 | 306.2 |
| Example 8 | 2.1 | Compound 1e | 0.065 | 32.3 |
| Example 9 | 19.2 | Compound 1e | 0.065 | 295.4 |
| Example 10 | 68.5 | Compound 1e | 0.065 | 1053.8 |
| Example 11 | 5.6 | Compound 1e | 0.065 | 86.2 |
| Example 12 | 43.9 | Compound 1e | 0.065 | 675.4 |
| Example 13 | 67 | Compound 1e | 0.065 | 1030.8 |
| Example 14 | 2.4 | Compound 1e | 0.065 | 36.9 |
| Example 15 | 494 | Compound 1e | 0.065 | 7600 |
| Example 16 | 32.1 | Compound 1e | 0.065 | 493.8 |
| Example 25 | 24.2 | Compound 1e | 0.065 | 372.3 |
| Example 26 | 13.4 | Compound 1e | 0.065 | 206.2 |
| Example 27 | 31.7 | Compound 1e | 0.065 | 487.7 |
| Example 28 | 6.9 | Compound 1e | 0.065 | 106.2 |
| Example 29 | 48.8 | Compound 1e | 0.065 | 750.8 |
| Example 32 | 22.5 | Compound 1e | 0.065 | 346.2 |
| Example 34-A | 6.0 | Compound 34e-A | 0.014 | 428.6 |
| Example 34-B | 6.36 | Compound 34e-B | 0.011 | 578.2 |
| Example 36-A | 31.8 | Compound 36g-A | 0.019 | 1673.7 |
| Example 37-A | 26.6 | Compound 36g-A | 0.019 | 1400 |
| Example 37-B | 47.4 | Compound 36g-B | 0.022 | 2154.5 |
| Example 38-A | 26.2 | Compound 36g-A | 0.019 | 1378.9 |
| Example 38-B | 19.5 | Compound 36g-B | 0.022 | 886.4 |
| Example 39 | 4.3 | Compound 36g | 0.027 | 159.3 |
| Example 40 | 52.8 | Compound 36g | 0.027 | 1955.6 |
| Example 41 | 36 | Compound 41c | 0.053 | 679.2 |
| Example 41-A | 44.1 | Compound 41c-B | 0.085 | 518.8 |
| Example 41-B | 32.1 | Compound 41c-A | 0.071 | 452.1 |
| Example 42-A | 40.5 | Compound 41c-A | 0.071 | 570.4 |
| Example 42-B | 49.2 | Compound 41c-B | 0.085 | 578.8 |
| Example 43-A | 110 | Compound 43e-A | 0.11 | 1000 |
| Example 43-B | 78.4 | Compound 43e-B | 0.035 | 2240 |
| Example 44-A | 65.4 | Compound 43e-B | 0.035 | 1868.6 |
| Example 44-B | 96.7 | Compound 43e-A | 0.11 | 879.1 |
| Example 45-A | 153 | Compound 45e-B or | 0.26 or 0.39 | 588 or 392 |
| | | Compound 45e-A | | |
| Example 45-B | >1000 | Compound 45e-B or | 0.26 or 0.39 | >3846 or >2564 |
| | | Compound 45e-A | | |
| Example 46-A | 45.5 | Compound 45e-A or | 0.26 or 0.39 | 175 or 116.7 |
| | | Compound 45e-B | | |
| Example 46-B | 45.7 | Compound 45e-B or | 0.26 or 0.39 | 175.7 or 117.2 |
| | | Compound 45e-A | | |
| Example 47-A | 10.9 | Compound 47e-A or | 0.021 or 0.025 | 519.0 or 436 |
| | | Compound 47e-B | | |
| Example 47-B | 13.1 | Compound 47e-A or | 0.021 or 0.025 | 623.8 or 524 |
| | | Compound 47e-B | | |
| Example 48-A | 18.3 | Compound 47e-A or | 0.021 or 0.025 | 871.4 or 732 |
| | | Compound 47e-B | | |
| Example 48-B | 20.8 | Compound 47e-A or | 0.021 or 0.025 | 990.5 or 832 |
| | | Compound 47e-B | | |

### Example 50

### Metabolism of Prodrugs of Compound of Formula (I)

A study was undertaken to evaluate the metabolic conversion of prodrugs, compound of formula (I), to its corresponding active form. The compounds of formula (I), if served as prodrugs, can be metabolized to the active compound or other compounds of the invention in the body. Human liver microsomes are often used to assess the degree of metabolic conversion of prodrugs in the body of animal or human.

### Materials

NADPH cofactor system including 3-Nicotinamide adenine dinucleotide phosphate (NADP), isocitric acid and isocitric dehydrogenase were purchased from Sigma-Aldrich Co. (St. Louis, Mo., USA). Human liver microsomes (Cat No. 452117, Lot No. 38290) were obtained from Corning (Woburn, Mass., USA). Mouse liver microsomes (Cat No. M1000, Lot No. 1310028) were obtained from Xenotech.

### Working Solution of the Compounds and Other Solution

Compounds were dissolved in DMSO to make 10 mM stock solutions. 10 μL of the stock solution was diluted with acetonitrile (990 μL) to get a 100 μM working solution.

### Incubation

Microsomes were preincubated with test compound for 10 min at 37° C. in 100 mM potassium phosphate buffer with pH 7.4. The reactions were initiated by adding NADPH regenerating system to give a final incubation volume of 200 μL and shaken in a water bath at 37° C. Incubation mixtures consisted of liver microsomes (0.5 mg microsomal protein/mL), substrates (1.0 μM), and NADP (1 mM), isocitric dehydrogenase (1 unit/mL), isocitric acid (6 mM).

### Preparation of Samples for Analysis

At 30 min, reaction was quenched by adding 600 L cold acetonitrile (including 100 ng/mL tolbutamide and 100 ng/mL labetalol as internal standard). The samples were centrifuged at 4000 rpm for 20 minutes and the resultant supernatants were subjected to LC-MS/MS analysis.

The samples for calibration curve were prepared as followed. Dispense 100 μL/well liver microsomes and 98 μL/well NADPH regenerating system solution to 96-well plate. Add 600 μL quenching solution first, and then followed by 2 μL Standard curve and QC working solution.

### Bioanalysis

The compounds were quantified on an API4000 LC-MC/MC instrument in the ESI-Positive MRM mode.

A study was undertaken to evaluate the metabolic conversion of prodrugs (1 μM), Example 1, Example 1-A, Example 1-B, Example 2, Example 2-A, Example 2-B, Example 3, Example 4, Example 5, Example 6, Example 7, Example 8, Example 9, Example 10, Example 11, Example 12, Example 13, Example 14, Example 15, Example 16, Example 17, Example 21, Example 22, Example 23, Example 25, Example 26, Example 27, Example 28Example 29, Example 30, Example 31, Example 32, Example 33, Example 34-A, Example 34-B, Example 36-A, Example 36-B, Example 37-A, Example 37-B, Example 38-A, Example 38-B, Example 39, Example 40, Example 41, Example 41-A, Example 41-B, Example 42, Example 42-A, Example 42-B, Example 43, Example 43-A, Example 43-B, Example 44, Example 44-A, Example 44-B and Example 45-A, Example 46-A, Example 46-B, Example 47-A, Example 47-B, Example 48-A, Example 48-B to the corresponding active forms, Compound 1e, Compound 1e-A, Compound 1e-B, Compound 34e-A, Compound 34e-B, Compound 36g-A, Compound 36g-B, Compound 36g, Compound 41c, Compound 41c-B, Compound 41c-A, Compound 43e, Compound 43e-A, Compound 43e-B, Compound 45e-A, Compound 45e-B, Compound 47e-A, and Compound 47e-B in the presence of human liver microsomes. Results were summarized and shown in Table 2.

TABLE 2
Metabolic conversion of prodrugs in human liver microsomes

| | | Metabolized product |
|---|---|---|
| | Corresponding | concentration |
| | Metabolized Product | in human liver |
| Example No. | (active form) | microsomes (μM) |
| Example 1 | Compound 1e | 0.0214 |
| Example 1- [symbol] | Compound 1e-A | 0.018 |
| Example 1- [symbol] | Compound 1e-B | 0.022 |
| Example 2 | Compound 1e | 0.028 |
| Example 2- [symbol] | Compound 1e-B | 0.036 |
| Example 2- [symbol] | Compound 1e-A | 0.029 |
| Example 3 | Compound 1e | 0.12 |
| Example 5 | Compound 1e | 0.078 |
| Example 6 | Compound 1e | 0.074 |
| Example 7 | Compound 1e | 0.15 |
| Example 8 | Compound 1e | 0.043 |
| Example 9 | Compound 1e | 0.002 |
| Example 10 | Compound 1e | 0.005 |
| Example 11 | Compound 1e | 0.001 |
| Example 12 | Compound 1e | 0.018 |
| Example 13 | Compound 1e | 0.04 |
| Example 14 | Compound 1e | 0.026 |
| Example 15 | Compound 1e | 0.002 |
| Example 16 | Compound 1e | 0.024 |
| Example 17 | Compound 1e | 0.075 |
| Example 21 | Compound 1e | 0.48 |
| Example 22 | Compound 1e | 0.42 |
| Example 23 | Compound 1e | 0.42 |
| Example 25 | Compound 1e | 0.018 |
| Example 26 | Compound 1e | 0.042 |
| Example 27 | Compound 1e | 0.11 |
| Example 28 | Compound 1e | 0.084 |
| Example 29 | Compound 1e | 0.009 |
| Example 31 | Compound 1e | 0.005 |
| Example 32 | Compound 1e | 0.013 |
| Example 33 | Compound 1e | 0.59 |
| Example 34-A | Compound 34e-A | 0.2 |
| Example 34-B | Compound 34e- [symbol] | 0.088 |
| Example 36-A | Compound 36g- [symbol] | 0.02 |
| Example 36-B | Compound 36g- [symbol] | 0.019 |
| Example 37-A | Compound 36g- [symbol] | 0.004 |
| Example 37-B | Compound 36g- [symbol] | 0.002 |
| Example 38-A | Compound 36g- [symbol] | 0.026 |
| Example 38-B | Compound 36g- [symbol] | 0.034 |
| Example 40 | Compound 36g | 0.032 |
| Example 41-A | Compound 41c- [symbol] | 0.38 |
| Example 41-B | Compound 41c- [symbol] | 0.36 |
| Example 42-A | Compound 41c- [symbol] | 0.14 |
| Example 42-B | Compound 41c- [symbol] | 0.004 |
| Example 43-A | Compound 43e- [symbol] | 0.014 |
| Example 43-B | Compound 43e-B | 0.016 |
| Example 44-A | Compound 43e- [symbol] | 0.002 |
| Example 44-B | Compound 43e- [symbol] | 0.002 |
| Example 45-A | Compound 45e-B [symbol] | 0.41 |
| Example 46-A | Compound 45e-A [symbol] | 0.039 |
| Example 46-B | Compound 45e-B [symbol] | 0.18 |
| Example 47-A | Compound 47e-A [symbol] | 0.36 |
| Example 47-B | Compound 47e-B [symbol] | 0.41 |
| Example 48-A | Compound 47e-A [symbol] | 0.11 |
| Example 48-B | Compound 47e-B [symbol] | 0.053 |

[symbol]

indicates data missing or illegible when filed

### Example 51

### In Vivo Combined Efficacy (Tumor Free Mice) of an Active Form of Compounds of the Present Invention (Compound 41-A) and Sorafenib in a Highly Aggressive Model of Hepatocellular Carcinoma

In iAST mice tumorigenesis was initiated by intravenous injection of 5×10⁸ IFU adenovirus expressing Cre recombinase (Ad-CMV-iCre vector in vivo application, Vector Biolabs) into transgenic mice expressing the hepatocyte-specific albumin promoter, a loxP-flanked stop cassette, and the SV40 large T-antigen (Runge A, at al., Cancer Res. 74 (2014) 4157-69). The Cre recombinase excises the stop cassette in transduced cells and leads to a transient viral hepatitis and resulting in multinodular tumorigenesis within 8 weeks. Female mice were treated with either vehicle (7.5% Gelatine/0.22% NaCl for Sorafenib; or 2% Klucel® Hydroxypropylcellulose LF (Asland), 0.5% D-α-Tocopherol polyethylene glycol 1000 succinate (TPGS, Sigma), 0.09% Methylparaben (Sigma), 0.01% Propylparabens (Sigma) in water for 41-A), or 90 mg/kg in Sorafenib (Nexavar R, Bayer HealthCare) daily or were treated with compound 41-A (10 mg/kg) once a week by oral gavage. Treatment using vehicle or Sorafenib started on week 7.5 upon adenovirus administration and 3 days prior to administration compound 41-A. Animals were sacrificed on day 12 after treatment start and total liver and tumor weights were determined. Per group n=10 were analyzed by One-way ANOVA and Tukey correction shown as individual dots with means±SEM using GraphPad Prism software version 6. Although Sorafenib was highly effective in monotherapy, the combination with an active form of the compounds of the present invention (compound 41-A) resulted even in 2/10 tumor-free mice by superficial examination of the livers in this highly aggressive model of hepatocellular carcinoma.

Synergistic effect of Compound 41-A and sorafenib
on tumor burden (tumor free mice)

| | Examination of the liver for |
|---|---|
| Treatment | superficial tumor nodules |
| Vehicle | 0/10 tumor nodule free |
| Compound 41-A | 0/10 tumor nodule free |
| Sorafenib | 0/10 tumor nodule free |
| Compound 41-A + | 2/10 tumor nodule free |
| Sorafenib | |

### Example 52

Treatment with an Active Form of the Compounds of the Present Invention (Compound 41-A) Induces PD-L1 Expression on Tumor Cells in Hepatocellular Carcinoma.

Tumors from iAST mice were treated as described in FIG. 1. Animals were sacrificed on day 12 after treatment start and tumors analyzed by flow cytometry. For flow cytometry, tumors were excised and single cell suspensions obtained by mechanical processing and enzymatic digestion (DNAse 0.01%, Collagenase IV 1 mg/ml). Staining procedures started with Fc receptor blocking using 2.4G2 antibody clone (1:200 dilution, BD Bioscience), and the following antibodies (clones) were used to analyzed leukocyte infiltrate: CD45-FITC (30-F11, BioLegend) and CD11b-BUV737 (M1/70, BD Bioscience). Samples were acquired using a LSR Fortessa machine (BD Bioscience) and analyzed by FlowJo version 10 (Treestar). Data are shown of n=5 per group, analyzed by One-way ANOVA and Tukey correction shown as individual dots with means±SEM using GraphPad Prism software version 6. Although the absolute immune cell infiltrate in iAST tumor did not change by any treatments described (FIG. 2A), significant changes were observed in the overall lymphoid and myeloid composition of the tumors (FIGS. 2 C and D). Here, the changes were clearly driven by Sorafenib, which was previously shown to act also on immune cells (Martin del Campo, et al, J Immunol. 195 (2015) 1995-2005). 41-A treatment however induced PD-L1 expression on tumor cells in monotherapy as well as in combination with Sorafenib (FIG. 2 B).

### Example 53

Treatment with an Active Form of the Compounds of the Present Invention (Compound 41-A) in the Transplanted Hep55.1c Mouse Model of Hepatocellular Carcinoma

Female C57BL/6N mice (Jackson Laboratories) were injected intra hepatically with 5×10⁵ Hep55.1c tumor cell line together with Matrigel (Matrigel Basement Membrane Matrix, Corning Cat #354234) in a total volume of 20 l (10 μl cell suspension plus 10 μl Martigel). Tumor volume was monitored weekly using μCT (TomoScope Synergy Twin, CT Imaging GmbH) upon a single intravenous administration of contrasting agent Exitron 6000 (Viscovert). Imaging data were reconstructed by TomoScope software and analyzed using Osirix software. Once tumors reached 80 mm³, mice were treated weekly with either 10 mg/kg 41-A compound or vehicle (2% Klucel® Hydroxypropylcellulose LF (Asland), 0.5% D-α-Tocopherol polyethylene glycol 1000 succinate (TPGS, Sigma), 0.09% Methylparaben (Sigma), 0.01% Propylparabens (Sigma) in water) per oral gavage. To compare to another agonistic, immune stimulating agent, a single dose of anti-CD40 antibody (4 mg/kg; clone FGK.45, BioXCell) was given. Data depicted are means±SEM for a minimum of n=9 animals per group.

Weekly administration of compound 41-A resulted in inhibition of tumor growth in Hep55.1c tumor bearing mice when compared to vehicle treatment. As previously published, a single dose of anti-CD40 antibody can lead to tumor eradication in subcutaneous MC38 tumors and it has been shown that the anti-CD40 antibody has an inflammatory effect in the liver (Hoves S, et al, J Exp Med, DOI: 10.1084/jem.20171440; Published Feb. 7, 2018). However, no beneficial treatment effect was observed in Hep55.1c tumor bearing mice with anti-CD40 antibody.

### Example 54

### Combination of an Active Form of the Compounds of the Present Invention (Compound 41-A) and Anti-PD-1 Antibodies Hep55.1c Mouse Model of Hepatocellular Carcinoma.

Female C57BL/6N mice (Jackson Laboratories) were injected intra hepatically with 5×10⁵ Hep55.1c tumor cell line together with Matrigel (Matrigel Basement Membrane Matrix, Corning Cat #354234) in a total volume of 20 μl (10 μl cell suspension plus 10 μl Martigel). After 3 weeks, animals were sacrificed and tumors excised from the liver. Excised tumors were cut into 1×1 mm³ pieces and implanted into the liver of female C57BL/6N mice. Scout animals were sacrificed to determine the time point of treatment start at about 80 mm³ tumor volume. Mice were treated with either 41-A or vehicle (2% Klucel® Hydroxypropylcellulose LF (Asland), 0.5% D-α-Tocopherol polyethylene glycol 1000 succinate (TPGS, Sigma), 0.09% methylparaben (Sigma), 0.01% propylparaben (Sigma) in water) per oral gavage, or intra peritoneal administration of 250 μg anti-PD-1 antibody (clone RPM1-14, BioXCell), or a combination of 41-1 plus anti-PD-1. 41-A was given weekly, while anti-PD-1 antibody treatment started one day after 41-A treatment and was continued every three to four days for 8 doses in total. Treatment with both agents was stopped after the last anti-PD-1 administration. Monotherapy with 41-A resulted in longer survival of mice (5/10) compared to vehicle control (1/10). Combined treatment of 41-A and anti-PD-1 enhanced survival of mice even significantly to 8/10 being alive on day 94 after tumor fragment transplantation.

### Example 55

Treatment with an Active Form of the Compounds of the Present Invention (Compound 41c-B) does not Induce Enhanced Tumor Cell Proliferation in Cell Lines Originating from Hepatocellular Carcinoma and Cholangiocarcinoma

Cell lines derived from hepatocellular carcinoma and cholangiocarcinoma (EGI1 and OZ) were maintained and tested in the following media: Huh7 and EGI1 were cultured in DMEM 4.5 g/L glucose (Gibco, Cat #31966-021), 10% FCS (GIBCO, Cat #10500-064 Lot 07G3690K), 2 mM L-glutamine (Thermo Fischer, Cat #25030081), 1 mM sodium pyruvate (GIBCO Cat #11360-039). Hep3B and HepG2 were cultivated in Eagles MEM+Earle's BSS (PAN, Cat # P04-08510), 10% FCS, 2 mM L-glutamine, 0.1 mM NEAA (PAN Cat # P08-32100) and 1 mM sodium pyruvate. JHH1, JHH5, JHH6 and OZ were cultivated in Williams'E (PAN Cat # P04-29050), 10% FCS and 2 mM L-glutamine. JHH2 was cultivated using Williams'E, 10% FCS and 2 mM L-glutamine. HLE was cultivated in DMEM 4.5 g/L glucose, 10% FCS and 2 mM L-glutamine. HLF was cultivated in DMEM 4.5 g/L glucose, 5% FCS, 0.1 mM NEAA and 2 mM L-glutamine. JHH4 was cultivated in Eagles MEM+Earle's BSS, 10% FCS and 2 mM L-glutamine. SkHep1 was cultivated in Eagles MEM+Earle's BSS, 10% FCS, 2 mM L-glutamine, 0.1 mM NEAA and 1 mM sodium pyruvate. SNU449 was cultivated using RPMI 1640 (PAN Cat # P04-18047) 10% FCS and 2 mM L-glutamine. Cells were seeded in the respective media overnight at a density of 5,000 cells per well in 96 well flat clear bottom black polystyrene TC-treated microplates (Corning, Cat #3904). The next day, logarithmic dilutions of 41c-B starting from 27 μM down to 270 pM were added and incubated for 72, 120 and 148 hours, respectively.

Tumor cell counts were determined using Perkin Elmer Operetta Imaging System and Harmony Software by counting nuclei stained for 20 minutes in full media using Hoechst33342 dye (2 μg/ml, Sigma Cat # B2261). Data shown are means+SD from triplicate wells based on the analysis of 9 images per well relative to the DMSO control.

None of the cell lines tested showed a significant increase in proliferation upon treatment with 41c-B directly at the depicted time points.

### Example 56

Treatment of Tumor Cells with an Active Form of the Compounds of the Present Invention (Compound 41c-B) in the Presence of Peripheral Blood Results in Inhibition of Proliferation of Tumor Cells.

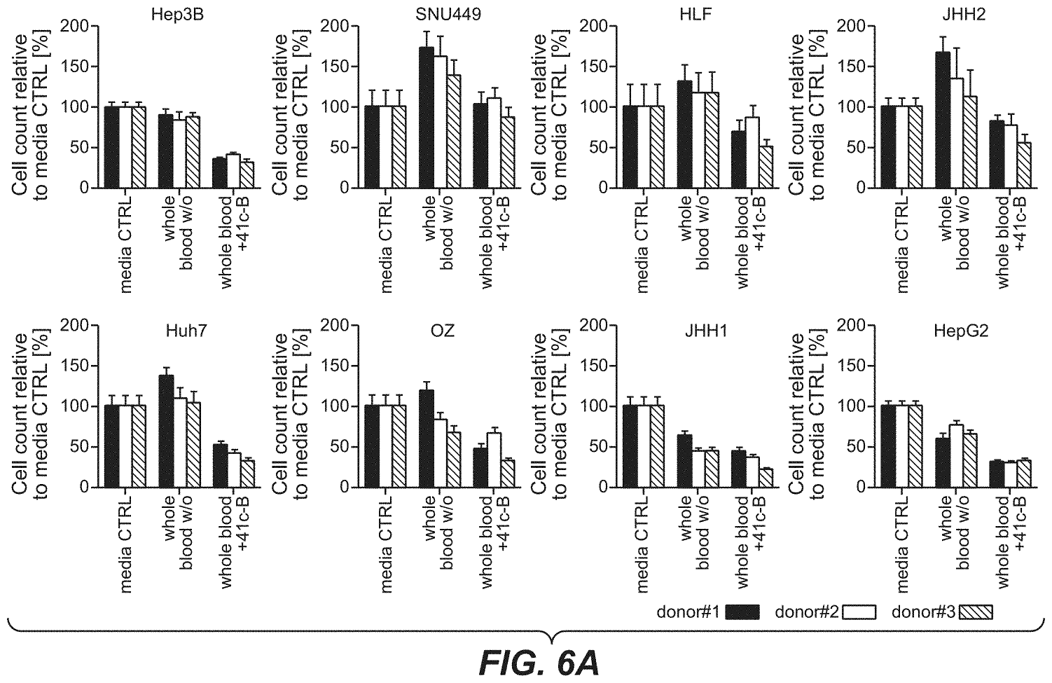

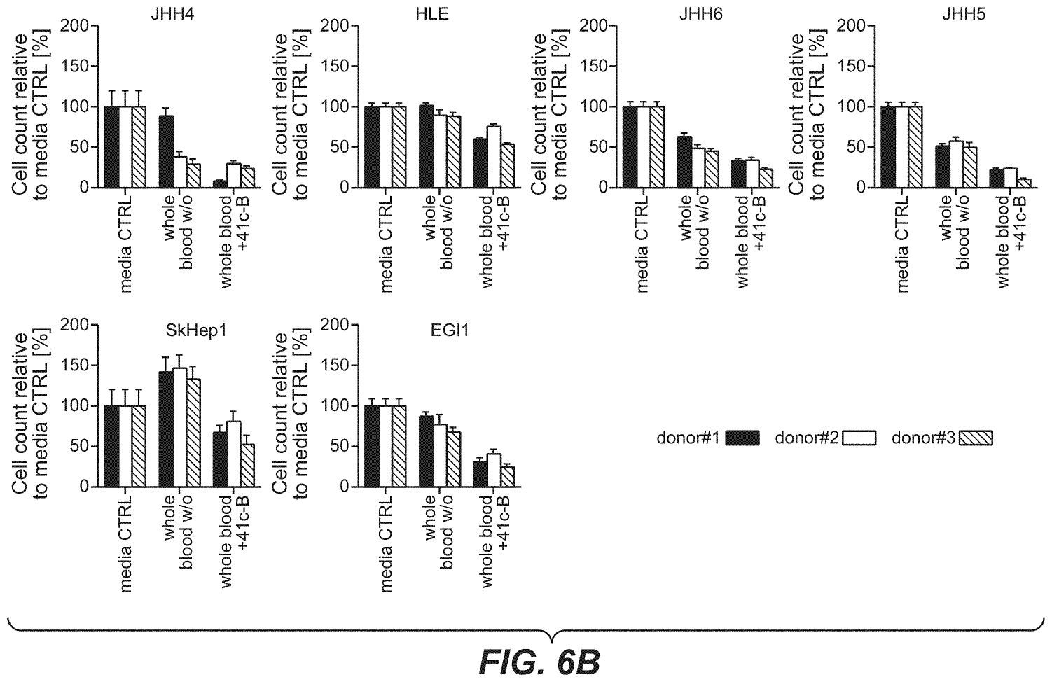

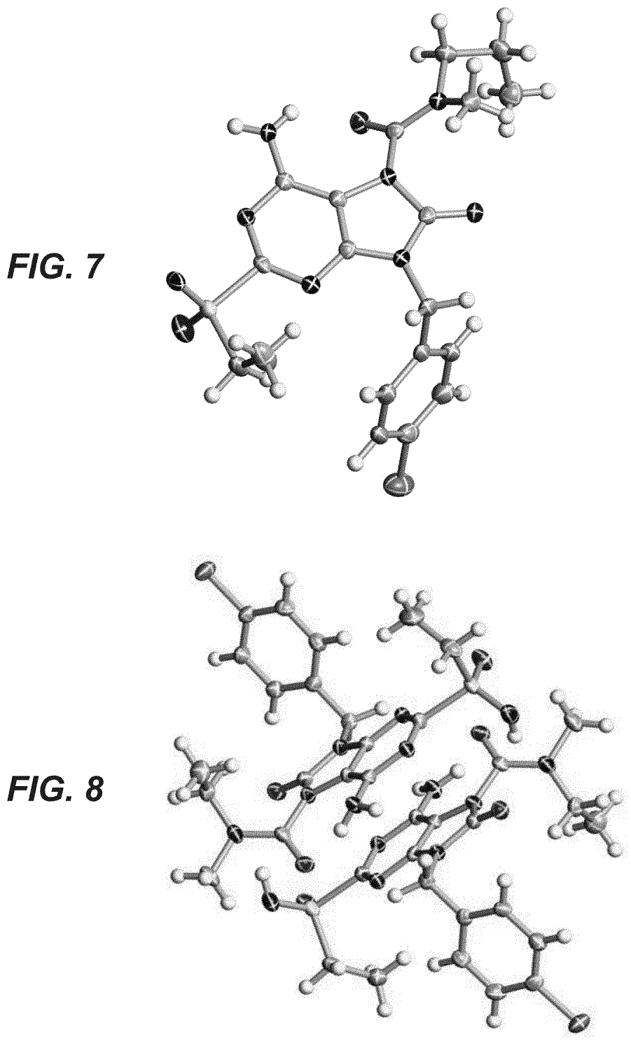

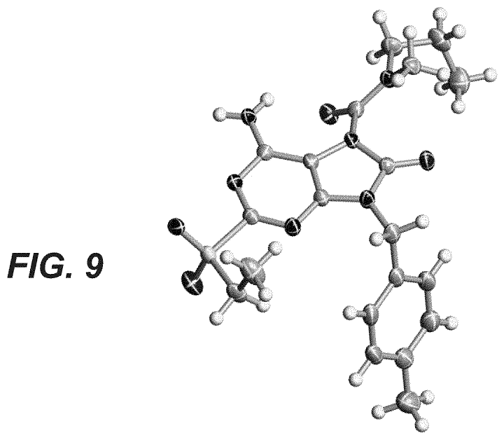

Heparinized whole blood of 3 different donors was diluted 1:1 in RPMI media (PAN Cat. # P04-18047) plus 10% FCS (GIBCO Cat #10500-064, lot 07G3690K) and incubated at 37° C. and 5% CO₂ for 24 hrs with 2.7 μM compound 41c-B. Supernatant was harvested and centrifuged at 600×g for 8 minutes to remove residual leukocytes, platelets and erythrocytes. Supernatants were stored at −80° C. until further use and thawed gently at room temperature prior to addition to the cell lines. Cell lines Huh7, JHH2, HLE, HLF, JHH4, Hep3B, HepG2, JHH1, EGI1, JHH5, JHH6, OZ, SkHep1, SNU449 were seeded in 100 μl of the respective media (as described in FIG. 5) overnight at a density of 5,000 cells per well 96 well flat clear bottom black polystyrene TC-treated microplates (Corning, Cat #3904). The next day, 100 μl of the whole blood supernatants were added to the cell lines. As controls, supernatant of whole blood without addition of 41c-B compound (“whole blood w/o”) or plain RPMI media plus FCS (“media CTRL”) was added. Cell lines were incubated for 72 hours. Tumor cell counts were determined using Perkin Elmer Operetta Imaging System and Harmony Software by counting nuclei stained for 20 minutes in full media using Hoechst33342 (2 μg/ml, Sigma Cat # B2261) and viability was assessed by additional detection of Propidium Iodine (PI, 1 μg/ml, Sigma Cat # P4864). Data shown are means+SD from triplicate wells based on the analysis of 9 images per well.

For some cell lines (SNU449, JHH2 and SkHep) the addition of supernatant of non-stimulated whole blood induced proliferation above the media control level, while others responded with reduced proliferation (OZ, JHH1, HepG2, JHH4, JHH6, JHH5 and EGI1). However, treatment with supernatants derived from whole blood incubated with 41c-B resulted in reduced cell counts in all cases tested compared to the respective “whole blood w/o” controls. The reduced cell counts were mainly attributed to a stop in proliferation, and only the cell lines JHH2, JHH4, JHH6, Hep3B and EGI1 did undergo cell death as determined by considerable PI positivity (data not shown).

### Example 57

### Single Dose PK Study in Male Wister-Han Rats

The single dose PK in Male Wister-Han Rats was performed to assess pharmacokinetic properties of tested compounds. Two groups of animals were dosed via Gavage (POE) of the respective compound. Blood samples (approximately 20 μL) were collected via Jugular vein or an alternate site at 15 min, 30 min, 1H, 2 h, 4 h, 7 h and 24 h post-dose groups. Blood samples were placed into tubes containing EDTA-K2 anticoagulant and centrifuged at 5000 rpm for 6 min at 4° C. to separate plasma from the samples. After centrifugation, the resulting plasma was transferred to clean tubes for bioanalysis of both prodrug and active form on LC/MS/MS. In the groups that prodrug were dosed, the concentration of prodrugs in the plasma samples was under the detection limit. The “tested compound” in Table 8 was used as the internal standard for testing the metabolite (active form) of “dose compound” in vivo. The pharmacokinetic parameters were calculated using non-compartmental module of WinNonlin® Professional 6.2. The peak concentration (Cₘₐₓ) was recorded directly from experimental observations. The area under the plasma concentration-time curve (AUC₀₋ₜ) was calculated using the linear trapezoidal rule up to the last detectable concentration.

Cₘₐₓ and AUC₀₋ₗₐₛₜ are two critical PK parameters related to the in vivo efficacy of the tested compound. Compounds with higher Cₘₐₓ and AUC₀₋ₗₐₛₜ will lead to the better in vivo efficacy. Results of PK parameters following oral administration of active forms and competitor compounds are given in Table 7. The PK parameters of prodrugs are tabulated in Table 8.

Following oral administration of prodrugs, the active forms were observed in plasma and therefore tested. The exemplified prodrugs of present invention (Example 41-B, 42-A, 42-B, 43-A, 45-A and 45-B) surprisingly showed much improved Cₘₐₓ (5-175 folds increase) and AUC₀₋ₗₐₛₜ (2.5-56 folds increase) comparing with reference compounds (GS9620, S-2 and S-3) and compounds mentioned in present invention (Compound 41c-A, 41c-B and 43e-A) which are all active forms. The results clearly demonstrated the unexpected superiority of prodrugs over active forms on PK parameters which led to better in vivo efficacy.

TABLE 7
The mean plasma concentration and PK parameters
of active forms after 5 mg/kg oral dosing

| | Dose compound | | | |
|---|---|---|---|---|
| | | | | Compound |
| | GS9620 | S-2 | S-3 | 41c-A |
| Time (h) | Mean plasma concentration (nM) | | | |
| 0.25 | 56.3 | 9.49 | 8.89 | 16.75 |
| 0.5 | 33.2 | 16.74 | 9.99 | 27.48 |
| 1 | 83.4 | 19.33 | 10.16 | 32.33 |
| 2 | 136 | 24.89 | 8.40 | 27.34 |
| 4 | 16.7 | 47.55 | 11.54 | 27.38 |
| 8* | 9.49 | 52.72 | 8.17 | 18.02 |
| 24 | ND | 4.90 | ND | 5.60 |
| Cₘₐₓ (nM) | 164 | 52.72 | 11.54 | 32.33 |
| AUC₀₋ₗₐₛₜ | 316 | 748 | 95 | 242.5 |
| (nM · h) | | | | |
| | Dose compound | | | |
| | Compound | Compound | Compound | Compound |
| | 41c-B | 43e-A | 45e-A | 45e-B |
| Time (h) | Mean plasma concentration (nM) | | | |
| 0.25 | 3.41 | 12.60 | 64.6 | 42.8 |
| 0.5 | 0.75 | 15.22 | 80.0 | 52.2 |
| 1 | 2.04 | 13.01 | 58.1 | 37.6 |
| 2 | 5.46 | 11.98 | 42.5 | 24.2 |
| 4 | 2.52 | 8.20 | 77.8 | 53.9 |
| 8* | 1.21 | 6.31 | 34.6 | 29 |
| 24 | ND | ND | 8.6 | 5.7 |
| Cₘₐₓ (nM) | 5.46 | 15.22 | 80.0 | 53.9 |
| AUC₀₋ₗₐₛₜ | 55.8 | 77 | 767 | 568 |
| (nM · h) | | | | |

*7 hrs for Compound 41-cA, Compound 41c-B and Compound 43e-A

TABLE 8
PK Parameters of prodrugs after 5 mg/kg oral dosing

| Dose | Tested | Cₘₐₓ | AUC₀₋ₗₐₛₜ |
|---|---|---|---|
| compound | compound | (nM) | (nM · h) |
| Example 41-B | Compound 41c-A | 1315 | 3658 |
| Example 42-A | Compound 41c-A | 1742 | 4867 |
| Example 42-B | Compound 41c-B | 956 | 3148 |
| Example 43-A | Compound 43e-A | 77 | 229 |
| Example 45-A | Compound 45e-B | 922 | 1914 |
| Example 45-B | Compound 45e-A | 1436 | 2619 |

### Example 58

### LYSA Solubility Study

LYSA study is used to determine the aqueous solubility of tested compounds. Samples were prepared in duplicate from 10 mM DMSO stock solution. After evaporation of DMSO with a centrifugal vacuum evaporator, the compounds were dissolved in 0.05 M phosphate buffer (pH 6.5), stirred for one hour and shaken for two hours. After one night, the solutions were filtered using a microtiter filter plate. Then the filtrate and its 1/10 dilution were analyzed by HPLC-UV. In addition, a four-point calibration curve was prepared from the 10 mM stock solutions and used for the solubility determination of the compounds. The results were in μg/mL. In case the percentage of sample measured in solution after evaporation divided by the calculated maximum of sample amount was bigger than 80%, the solubility was reported as bigger than this value.

Results of LYSA were shown in Table 9. It was clear that the solubility of active forms were surprisingly improved by 10 to over 200 folds when converted to various prodrugs.

TABLE 9
Solubility data of particular compounds

| | LYSA of | Corresponding | LYSA of Active |
|---|---|---|---|
| | Prodrugs | Active | Forms |
| Prodrugs | (μg/mL) | Forms | (μg/mL) |
| Example 1 | 290 | Compound 1e | 21 |
| Example 1-A | 315 | Compound 1e-A | 56 |
| Example 1-B | 200 | Compound 1e-B | 50 |
| Example 2 | 615 | Compound 1e | 21 |
| Example 2-A | >600 | Compound 1e-B | 50 |
| Example 2-B | >590 | Compound 1e-A | 56 |
| Example 3 | 240 | Compound 1e | 21 |
| Example 4 | 695 | Compound 1e | 21 |
| Example 5 | >595 | Compound 1e | 21 |
| Example 6 | 140 | Compound 1e | 21 |
| Example 7 | 615 | Compound 1e | 21 |
| Example 8 | 620 | Compound 1e | 21 |
| Example 9 | >520 | Compound 1e | 21 |
| Example 10 | 120 | Compound 1e | 21 |
| Example 11 | >618 | Compound 1e | 21 |
| Example 12 | 120 | Compound 1e | 21 |
| Example 13 | 155 | Compound 1e | 21 |
| Example 14 | 225 | Compound 1e | 21 |
| Example 15 | 405 | Compound 1e | 21 |
| Example 16 | 205 | Compound 1e | 21 |
| Example 17 | 190 | Compound 1e | 21 |
| Example 25 | >670 | Compound 1e | 21 |
| Example 26 | >690 | Compound 1e | 21 |
| Example 27 | >380 | Compound 1e | 21 |
| Example 28 | 695 | Compound 1e | 21 |
| Example 29 | 395 | Compound 1e | 21 |
| Example 32 | 125 | Compound 1e | 21 |
| Example 36-A | 168 | Compound 36g-A | 6 |
| Example 36-B | 209 | Compound 36g-B | 11 |
| Example 41-A | 260 | Compound 41c-B | 5 |
| Example 41-B | 250 | Compound 41c-A | 1 |
| Example 42-A | 225 | Compound 41c-A | 1 |
| Example 42-B | 335 | Compound 41c-B | 5 |
| Example 43-A | 203 | Compound 43e-A | 13 |
| Example 43-B | 170 | Compound 43e-B | 13 |
| Example 45 | 172 | Compound 45e | 152 |
| Example 45-A | >560 | Compound 45e-A or | 90 or 115 |
| | | Compound 45e-B | |
| Example 45-B | 420 | Compound 45e-B Or | 115 or 90 |
| | | Compound 45e-A | |
| Example 46-A | 205 | Compound 45e-A Or | 90 or 115 |
| | | Compound 45e-B | |
| Example 46-B | >580 | Compound 45e-B Or | 115 or 90 |
| | | Compound 45e-A | |
| Example 47-A | 154 | Compound 47e-A or | <1.0 or <1.0 |
| | | Compound 47e-B | |
| Example 47-B | 128 | Compound 47e-B or | <1.0 or <1.0 |
| | | Compound 47e-A | |
| Example 48-A | 305 | Compound 47e-A or | <1.0 or <1.0 |
| | | Compound 47e-B | |
| Example 48-B | 275 | Compound 47e-B or | <1.0 or <1.0 |
| | | Compound 47e-A | |

### Example 59

### Portal Vein Study

The objective of this study was to understand whether prodrug remains unchanged as it was absorbed through the intestine into the portal circulation and demonstrate the primary site of conversion.

### Surgical Procedure for Portal Vein Cannulation (PVC) and Carotid Artery Cannulation (CAC)

Surgery was performed under pentobarbital/isoflurane anesthesia. Briefly, after disinfecting the abdominal area with betadine and 70% isopropyl alcohol, a small abdominal mid-line incision was made. The cecum was pulled out and mesenteric vein was identified and isolated for about 5 mm vessel. A loose ligature was placed proximally and distal end of the vein was ligated. Make a small incision (just enough to allow the insertion of the catheter) on isolated vein and insert the PU catheter towards liver for appropriate length. The catheter was secured in place by tying the loose ligature around the cannulated vessel. The cecum was replaced into abdominal cavity. A hole was made in the right abdominal wall to make the end of catheter pass freely. The catheter was secured by suture on the abdominal wall. The abdominal muscle incision was closed with suture. A small incision was made in the scapular area to serve as the exit site of the catheter. The catheter was subcutaneously tunneled and exteriorized through the scapular incision. A fixed suture was placed in the scapular region. The patency of the catheter was checked and then exteriorized from the subcutaneous space to the dorsal neck region. After gently wiping the area, the abdominal cavity was sutured.

The left carotid artery was then cannulated by inserting a PE50 catheter. Both the exteriorized catheters were tied firmly on the dorsal neck region and fixed. The animals was then allowed to recover in its cage and used for study at least 3 days after surgery. All catheters were flushed once daily with heparinized saline to maintain patency.

### Oral PK Study in PVC/CAC Dual Cannulated Rat

Animals were fasted overnight (n=3) and administered vial oral gavage (10 mg/kg, 10 mL/kg). Blood samples (60 μL) were collected simultaneously from the portal and carotid artery catheters at 0.083, 0.25, 0.5, 1, 2, 4, 7, 24 h. All blood samples will be transferred into microcentrifuge tubes containing 2 μL of K₂EDTA (0.5M) as anti-coagulant and placed on wet ice. Then blood samples will be processed for plasma by centrifugation at approximately 4° C., 3000 g within half an hour of collection. Plasma samples will be stored in polypropylene tubes, quick frozen over dry ice and kept at −70±10° C. until LC/MS/MS analysis.

Pharmacokinetic parameters (mean±SD, n=3) of prodrugs and active forms in portal and carotid samples following oral administration of prodrugs (10 mg/kg) in portal vein cannulated rat were detected and analyzed. The test results of Example 1-B, 41-A, 41-B, 42-A and 43-A were summarized below.

TABLE 10
Pharmacokinetic parameters of Example 41-A and its corresponding
active form Compound 41c-B in portal and carotid samples
following oral administration of Example 41-A (10 mg/kg)
in portal vein cannulated rat

| | Prodrug | | | |
|---|---|---|---|---|
| | Example 41-A | | | |
| | Corresponding Active Form | | | |
| | Compound 41c-B | | | |
| | Portal sampling | | Carotid sampling | |
| PK parameter | prodrug | active form | prodrug | active form |
| Tₘₐₓ (h) | 0.14 | 0.4 | 0.19 | 0.42 |
| Cₘₐₓ (nM) | 9703 | 2223 | 210 | 2185 |
| AUC₀₋₂ (nM · h) | 2188 | 2246 | 114 | 2108 |

| | | |
|---|---|---|
| AUC_{active}/AUCₜₒₜₐₗ | 51% | 95% |

TABLE 11
Pharmacokinetic parameters of Example 43-A and its corresponding
active form Compound 43e-A in portal and carotid samples
following oral administration of Example 43-A (10 mg/kg)
in portal vein cannulated rat

| | Prodrug | | | |
|---|---|---|---|---|
| | Example 43-A | | | |
| | Corresponding Active Form | | | |
| | Compound 43e-A | | | |
| | Portal sampling | | Carotid sampling | |
| PK parameter | prodrug | active form | prodrug | active form |
| Tₘₐₓ (h) | 0.28 | 0.33 | 0.22 | 0.28 |
| Cₘₐₓ (nM) | 4110 | 818 | 191 | 691 |
| AUC₀₋₂ (nM · h) | 2067 | 679 | 124 | 564 |

| | | |
|---|---|---|
| AUC_{active}/AUCₜₒₜₐₗ | 25% | 82% |

TABLE 12
Pharmacokinetic parameters of Example 1-B and its corresponding
active form Compound 1e-A in portal and systemic samples
following oral administration of Example 1-B (10 mg/kg)
in portal vein cannulated rat

| | Prodrug | | | |
|---|---|---|---|---|
| | Example 1-B | | | |
| | Corresponding Active Form | | | |
| | Compound 1e-A | | | |
| | Portal sampling | | Carotid sampling | |
| PK parameter | prodrug | active form | prodrug | active form |
| Tₘₐₓ (h) | 0.083 | 0.25 | 0.083 | 0.5 |
| Cₘₐₓ (nM) | 670 | 192 | 70 | 174 |
| AUC₀₋₂ (nM · h) | 266 | 164 | 40 | 184 |

| | | |
|---|---|---|
| AUC_{active}/AUCₜₒₜₐₗ | 38% | 82% |

TABLE 13
Pharmacokinetic parameters of Example 42-A and its corresponding
active form Compound 41c-A in portal and carotid samples
following oral administration of Example 42-A (10 mg/kg)
in portal vein cannulated rat

| | Prodrug | | | |
|---|---|---|---|---|
| | Example 42-A | | | |
| | Corresponding Active Form | | | |
| | Compound 41c-A | | | |
| | Portal sampling | | Carotid sampling | |
| PK parameter | prodrug | active form | prodrug | active form |
| Tₘₐₓ (h) | 0.19 | 0.42 | 0.22 | 0.36 |
| Cₘₐₓ (nM) | 8917 | 3162 | 286 | 3326 |
| AUC₀₋₂ (nM · h) | 3461 | 3199 | 286 | 3326 |

| | | |
|---|---|---|
| AUC_{active}/AUCₜₒₜₐₗ | 48% | 96% |

TABLE 14
Pharmacokinetic parameters of Example 41-B and its corresponding
active form Compound 41c-A in portal and carotid samples
following oral administration of Example 41-B (10 mg/kg)
in portal vein cannulated rat

| | Prodrug | | | |
|---|---|---|---|---|
| | Example 41-B | | | |
| | Corresponding Active Form | | | |
| | Compound 41c-A | | | |
| | Portal sampling | | Carotid sampling | |
| PK parameter | prodrug | active form | prodrug | active form |
| Tₘₐₓ (h) | 0.19 | 0.5 | 0.25 | 0.5 |
| Cₘₐₓ (nM) | 7068 | 3315 | 29.6 | 3432 |
| AUC₀₋₂ (nM · h) | 1444 | 3211 | 22.5 | 3301 |

| | | |
|---|---|---|
| AUC_{active}/AUCₜₒₜₐₗ | 69% | 99% |

Based on the above results, it was concluded that the primary site of conversion of prodrug was liver rather than intestine, because AUC_{active}/AUCₜₒₜₐₗ was higher in sampling from carotid artery compared to
AUC_{active}/AUCₜₒₜₐₗ in sampling from portal vein.

We claim:

1. A method for the treatment or prophylaxis of liver cancer comprising administering to a patient in need thereof, a therapeutically effective amount of a compound of formula (I), or a pharmaceutically acceptable salt, enantiomer or diastereomer thereof, 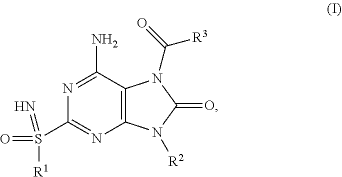
wherein:
R¹ is C₁₋₆alkyl;
R² is benzyl, said benzyl being unsubstituted or substituted by one, two or three substituents independently selected from halogen and C₁₋₆alkyl;
R³ is —NR⁴R⁵, wherein
R⁴ is C₁₋₆-alkyl or C₁₋₆alkoxyC₁₋₆alkyl;
R⁵ is (C₁₋₆alkyl)₂NCOOC₁₋₆alkyl, C₁₋₆alkoxyC₁₋₆alkyl, C₁₋₆alkoxycarbonyl(C₁₋₆alkyl)aminoC₁₋₆ alkyl, C₁₋₆alkoxycarbonyl(phenyl)C₁₋₆alkyl, C₁₋₆alkoxycarbonylC₁₋₆alkyl, C₁₋₆ alkoxycarbonyloxyC₁₋₆alkyl, C₁₋₆alkyl, C₁₋₆alkylcarbonyl(C₁₋₆alkyl)aminoC₁₋₆alkyl or pyrrolidinylcarbamoyloxyC₁₋₆alkyl; or
R⁴ and R⁵ together with the nitrogen they are attached to form a heterocyclyl;
with the proviso that the compound is not:
6-amino-9-benzyl-2-(propylsulfonimidoyl)-7-(pyrrolidine-1-carbonyl)purin-8-one;
6-amino-9-benzyl-7-(piperidine-1-carbonyl)-2-(propylsulfonimidoyl)purin-8-one;
6-amino-9-benzyl-7-(morpholine-4-carbonyl)-2-(propylsulfonimidoyl)purin-8-one;
6-amino-9-benzyl-7-(3,3-dimethylpyrrolidine-1-carbonyl)-2-(propylsulfonimidoyl)purin-8-one;
ethyl 1-[6-amino-9-benzyl-8-oxo-2-(propylsulfonimidoyl)purine-7-carbonyl]pyrrolidine-2-carboxylate;
6-amino-7-(2-azaspiro[3.3]heptane-2-carbonyl)-9-benzyl-2-(propylsulfonimidoyl)purin-8-one;
6-amino-9-benzyl-7-(2-oxa-6-azaspiro[3.3]heptane-6-carbonyl)-2-(propylsulfonimidoyl)purin-8-one;
6-amino-9-benzyl-7-(3,3-difluoropyrrolidine-1-carbonyl)-2-(propylsulfonimidoyl)purin-8-one; or
6-amino-9-benzyl-7-(3-fluoro-3-methyl-pyrrolidine-1-carbonyl)-2-(propylsulfonimidoyl)purin-8-one;
or an enantiomer or diastereomer thereof.

2. The method according to claim 1, wherein
R¹ is C₁₋₆ alkyl;
R² is benzyl, said benzyl being unsubstituted or substituted by halogen or C₁₋₆alkyl;
R³ is azetidinyl;
piperazinyl substituted by C₁₋₆ alkyl;
piperidinyl substituted by piperidinyl;
pyrrolidinyl; or
—NR⁴R⁵, wherein
R⁴ is C₁₋₆ alkyl or C₁₋₆ alkoxyC₁₋₆ alkyl;
R⁵ is (C₁₋₆alkyl)₂NCOOC₁₋₆alkyl, C₁₋₆ alkoxyC₁₋₆ alkyl, C₁₋₆ alkoxycarbonyl(C₁₋₆ alkyl)aminoC₁₋₆ alkyl, C₁₋₆ alkoxycarbonyl(phenyl)C₁₋₆ alkyl, C₁₋₆ alkoxycarbonylC₁₋₆ alkyl, C₁₋₆ alkoxycarbonyloxyC₁₋₆ alkyl, C₁₋₆ alkyl, C₁₋₆ alkylcarbonyl(C₁₋₆alkyl)aminoC₁₋₆ alkyl or pyrrolidinylcarbamoyloxyC₁₋₆ alkyl.

3. The method according to claim 1 wherein:
R¹ is ethyl or propyl;
R² is benzyl, bromobenzyl, chlorobenzyl, fluorobenzyl or methylbenzyl;
R³ is azetidinyl;
4-methylpiperazinyl;
piperidinylpiperidinyl;
pyrrolidinyl; or
—NR⁴R⁵, wherein
R⁴ is methyl, ethyl, propyl or methoxyethyl;
R⁵ is acetyl(methyl)aminoethyl, butyl, butyl(methyl)carbamoyloxyethyl, diethylcarbamoyloxyethyl, ethoxycarbonyl(methyl)aminoethyl, ethoxycarbonylethyl, ethoxycarbonylisobutyl, ethoxycarbonylisopentyl, ethoxycarbonylmethyl, ethoxycarbonyloxyethyl, ethoxycarbonyl(phenyl)ethyl, ethyl, isobutyl, isopropoxycarbonylisopentyl, isopropoxycarbonyl(phenyl)ethyl, isopropyl, methoxycarbonyl(methyl)aminoethyl, methoxyethyl, methoxypropyl, propyl, propyl(methyl)carbamoyloxyethyl, pyrrolidinylcarbamoyloxyethyl, tert-butoxycarbonyl(methyl)aminoethyl, tert-butoxycarbonylethyl, tert-butoxycarbonylisopentyl or tert-butoxycarbonyl(phenyl)ethyl.

4. The method according to claim 3 wherein R³ is azetidinyl, 4-methylpiperazinyl, piperidinylpiperidinyl, pyrrolidinyl, acetyl(methyl)aminoethyl(methyl)amino, bis(methoxyethyl)amino, butyl(ethyl)amino, butyl(methyl)amino, butyl(methyl)carbamoyloxyethyl(methyl)amino, diethylcarbamoyloxyethyl(methyl)amino, ethoxycarbonyl(methyl)aminoethyl(methyl)amino, ethoxycarbonylethyl(methyl)amino, ethoxycarbonylisobutyl(methyl)amino, ethoxycarbonylisopentyl(methyl)amino, ethoxycarbonylmethyl(methyl)amino, ethoxycarbonyloxyethyl(methyl)amino, ethoxycarbonyl(phenyl)ethyl(methyl)amino, ethyl(methyl)amino, isobutyl(methyl)amino, isopropoxycarbonylisopentyl(methyl)amino, isopropoxycarbonyl(phenyl)ethyl(methyl)amino, isopropyl(methyl)amino, methoxycarbonyl(methyl)aminoethyl(methyl)amino, methoxyethyl(ethyl)amino, methoxyethyl(methyl)amino, methoxyethyl(propyl)amino, methoxypropyl(methyl)amino, propyl(ethyl)amino, propyl(methyl)amino, propyl(methyl)carbamoyloxyethyl(methyl)amino, pyrrolidinylcarbamoyloxyethyl(methyl)amino, tert-butoxycarbonyl(methyl)aminoethyl(methyl)amino, tert-butoxycarbonylethyl(methyl)amino, tert-butoxycarbonylisopentyl(methyl)amino or tert-butoxycarbonyl(phenyl)ethyl(methyl)amino.

5. The method according to claim 1 wherein R¹ is ethyl.

6. The method according to claim 1 wherein R² is benzyl substituted by halogen or C₁₋₆ alkyl.

7. The method according to claim 2 wherein R² is bromobenzyl, chlorobenzyl, fluorobenzyl or methylbenzyl.

8. The method according to claim 7 wherein R² is bromobenzyl, chlorobenzyl or fluorobenzyl.

9. The method according to claim 1 wherein R³ is —NR⁴R⁵, R⁴ is C₁₋₆ alkyl and R⁵ is C₁₋₆ alkyl.

10. The method according to claim 9 wherein R³ is propyl(methyl)amino or ethyl(methyl)amino.

11. The method according to claim 1 wherein:
R¹ is C₁₋₆alkyl;
R² is benzyl, said benzyl being substituted by halogen or C₁₋₆alkyl;
R³ is —NR⁴R⁵, wherein R⁴ is C₁₋₆alkyl, R⁵ is C₁₋₆alkyl.

12. The method according to claim 11 wherein:
R¹ is ethyl;
R² is methylbenzyl, bromobenzyl, chlorobenzyl or fluorobenzyl;
R³ is propyl(methyl)amino or ethyl(methyl)amino.

13. The method according to claim 1 wherein the compound is selected from the group consisting of:
6-Amino-9-benzyl-N-methyl-8-oxo-N-propyl-2-(propylsulfonimidoyl)purine-7-carboxamide;
6-Amino-9-benzyl-N-(2-methoxyethyl)-N-methyl-8-oxo-2-(propylsulfonimidoyl)purine-7-carboxamide;
6-Amino-9-benzyl-N-ethyl-8-oxo-N-propyl-2-(propylsulfonimidoyl)purine-7-carboxamide;
6-Amino-9-benzyl-7-[4-(1-piperidyl)piperidine-1-carbonyl]-2-(propylsulfonimidoyl)purin-8-one;
6-Amino-9-benzyl-N-ethyl-N-(2-methoxyethyl)-8-oxo-2-(propylsulfonimidoyl)purine-7-carboxamide;
6-Amino-9-benzyl-N-butyl-N-ethyl-8-oxo-2-(propylsulfonimidoyl)purine-7-carboxamide;
6-Amino-9-benzyl-N-(2-methoxyethyl)-8-oxo-N-propyl-2-(propylsulfonimidoyl)purine-7-carboxamide;
6-Amino-9-benzyl-N,N-bis(2-methoxyethyl)-8-oxo-2-(propylsulfonimidoyl)purine-7-carboxamide;
6-Amino-7-(azetidine-1-carbonyl)-9-benzyl-2-(propylsulfonimidoyl)purin-8-one;
6-Amino-9-benzyl-N-isopropyl-N-methyl-8-oxo-2-(propylsulfonimidoyl)purine-7-carboxamide;
6-Amino-9-benzyl-7-(4-methylpiperazine-1-carbonyl)-2-(propylsulfonimidoyl)purin-8-one;
6-Amino-9-benzyl-N-(3-methoxypropyl)-N-methyl-8-oxo-2-(propylsulfonimidoyl)purine-7-carboxamide;
6-Amino-9-benzyl-N-isobutyl-N-methyl-8-oxo-2-(propylsulfonimidoyl)purine-7-carboxamide;
Ethyl 2-[[6-amino-9-benzyl-8-oxo-2-(propylsulfonimidoyl)purine-7-carbonyl]-methyl-amino]acetate;
Ethyl 3-[[6-amino-9-benzyl-8-oxo-2-(propylsulfonimidoyl)purine-7-carbonyl]-methyl-amino]propanoate;
tert-Butyl 3-[[6-amino-9-benzyl-8-oxo-2-(propylsulfonimidoyl)purine-7-carbonyl]-methyl-amino]propanoate;
Ethyl (2S)-2-[[6-amino-9-benzyl-8-oxo-2-(propylsulfonimidoyl)purine-7-carbonyl]-methyl-amino]propanoate;
tert-Butyl (2S)-2-[[6-amino-9-benzyl-8-oxo-2-(propylsulfonimidoyl)purine-7-carbonyl]-methyl-amino]-4-methyl-pentanoate;
Isopropyl (2S)-2-[[6-amino-9-benzyl-8-oxo-2-(propylsulfonimidoyl)purine-7-carbonyl]-methyl-amino]-4-methyl-pentanoate;
Ethyl (2S)-2-[[6-amino-9-benzyl-8-oxo-2-(propylsulfonimidoyl)purine-7-carbonyl]-methyl-amino]-3-methyl-butanoate;
Ethyl (2S)-2-[[6-amino-9-benzyl-8-oxo-2-(propylsulfonimidoyl)purine-7-carbonyl]-methyl-amino]-4-methyl-pentanoate;
Ethyl (2S)-2-[[6-amino-9-benzyl-8-oxo-2-(propylsulfonimidoyl)purine-7-carbonyl]-methyl-amino]-3-phenyl-propanoate;
Isopropyl (2S)-2-[[6-amino-9-benzyl-8-oxo-2-(propylsulfonimidoyl)purine-7-carbonyl]-methyl-amino]-3-phenyl-propanoate;
tert-Butyl (2S)-2-[[6-amino-9-benzyl-8-oxo-2-(propylsulfonimidoyl)purine-7-carbonyl]-methyl-amino]-3-phenyl-propanoate;
N-[2-[Acetyl(methyl)amino]ethyl]-6-amino-9-benzyl-N-methyl-8-oxo-2-(propylsulfonimidoyl)purine-7-carboxamide;
Methyl N-[2-[[6-amino-9-benzyl-8-oxo-2-(propylsulfonimidoyl)purine-7-carbonyl]-methyl-amino]ethyl]-N-methyl-carbamate;
tert-Butyl N-[2-[[6-amino-9-benzyl-8-oxo-2-(propylsulfonimidoyl)purine-7-carbonyl]-methyl-amino]ethyl]-N-methyl-carbamate;
Ethyl N-[2-[[6-amino-9-benzyl-8-oxo-2-(propylsulfonimidoyl)purine-7-carbonyl]-methyl-amino]ethyl]-N-methyl-carbamate;
2-[[6-Amino-9-benzyl-8-oxo-2-(propylsulfonimidoyl)purine-7-carbonyl]-methyl-amino]ethyl N-butyl-N-methyl-carbamate;
2-[[6-Amino-9-benzyl-8-oxo-2-(propylsulfonimidoyl)purine-7-carbonyl]-methyl-amino]ethyl pyrrolidine-1-carboxylate;
2-[[6-Amino-9-benzyl-8-oxo-2-(propylsulfonimidoyl)purine-7-carbonyl]-methyl-amino]ethyl N-methyl-N-propyl-carbamate;
2-[[6-Amino-9-benzyl-8-oxo-2-(propylsulfonimidoyl)purine-7-carbonyl]-methyl-amino]ethyl N,N-diethylcarbamate;
2-[[6-Amino-9-benzyl-8-oxo-2-(propylsulfonimidoyl)purine-7-carbonyl]-methyl-amino]ethyl ethyl carbonate;
6-Amino-N-butyl-9-[(4-chlorophenyl)methyl]-N-methyl-8-oxo-2-[S(S)-propylsulfonimidoyl]purine-7-carboxamide;
6-amino-N-butyl-9-[(4-chlorophenyl)methyl]-N-methyl-8-oxo-2-[S(S)-propylsulfonimidoyl]purine-7-carboxamide;
6-Amino-9-[(4-chlorophenyl)methyl]-N-ethyl-N-methyl-8-oxo-2-(propylsulfonimidoyl)purine-7-carboxamide;
6-Amino-N-methyl-8-oxo-N-propyl-2[S(S)-propylsulfonimidoyl]-9-(p-tolylmethyl)purine-7-carboxamide;
6-Amino-N-methyl-8-oxo-N-propyl-2[S(R)-propylsulfonimidoyl]-9-(p-tolylmethyl)purine-7-carboxamide;
6-Amino-2-[S(S)-propylsulfonimidoyl]-9-(p-tolylmethyl)-7-(pyrrolidine-1-carbonyl)purin-8-one;
6-Amino-2-[S(R)-propylsulfonimidoyl]-9-(p-tolylmethyl)-7-(pyrrolidine-1-carbonyl)purin-8-one;
6-Amino-N-(2-methoxyethyl)-N-methyl-8-oxo-2-[S(S)-propylsulfonimidoyl]-9-(p-tolylmethyl)purine-7-carboxamide;
6-Amino-N-(2-methoxyethyl)-N-methyl-8-oxo-2-[S(R)-propylsulfonimidoyl]-9-(p-tolylmethyl)purine-7-carboxamide;
6-Amino-N-ethyl-N-methyl-8-oxo-2-(propylsulfonimidoyl)-9-(p-tolylmethyl)purine-7-carboxamide;
6-Amino-N-butyl-N-methyl-8-oxo-2-(propylsulfonimidoyl)-9-(p-tolylmethyl)purine-7-carboxamide;
6-Amino-9-[(4-chlorophenyl)methyl]-2-[S(R)-ethylsulfonimidoyl]-N-methyl-8-oxo-N-propyl-purine-7-carboxamide;
6-Amino-9-[(4-chlorophenyl)methyl]-2-[S(S)-ethylsulfonimidoyl]-N-methyl-8-oxo-N-propyl-purine-7-carboxamide;
6-Amino-9-[(4-chlorophenyl)methyl]-N-ethyl-2 [S(S)-ethylsulfonimidoyl]-N-methyl-8-oxo-purine-7-carboxamide;
6-Amino-9-[(4-chlorophenyl)methyl]-N-ethyl-2-[S(R)-ethylsulfonimidoyl]-N-methyl-8-oxo-purine-7-carboxamide;
6-Amino-2-[S(S)-ethylsulfonimidoyl]-N-methyl-8-oxo-N-propyl-9-(p-tolylmethyl)purine-7-carboxamide;
6-Amino-2-[S(R)-ethylsulfonimidoyl]-N-methyl-8-oxo-N-propyl-9-(p-tolylmethyl)purine-7-carboxamide;
6-Amino-N-ethyl-2 [S(S)-ethylsulfonimidoyl]-N-methyl-8-oxo-9-(p-tolylmethyl)purine-7-carboxamide;
6-Amino-N-ethyl-2-[S(R)-ethylsulfonimidoyl]-N-methyl-8-oxo-9-(p-tolylmethyl)purine-7-carboxamide;
6-Amino-2-[S(S)ethylsulfonimidoyl]-9-[(4-fluorophenyl)methyl]-N-methyl-8-oxo-N-propyl-purine-7-carboxamide;
6-Amino-2-[S(R)ethylsulfonimidoyl]-9-[(4-fluorophenyl)methyl]-N-methyl-8-oxo-N-propyl-purine-7-carboxamide;
6-Amino-N-ethyl-2-(ethylsulfonimidoyl)-9-[(4-fluorophenyl)methyl]-N-methyl-8-oxo-purine-7-carboxamide;
6-Amino-N-ethyl-2-[S(S)-(ethylsulfonimidoyl)]-9-[(4-fluorophenyl)methyl]-N-methyl-8-oxo-purine-7-carboxamide;
6-Amino-N-ethyl-2-[S(R)-(ethylsulfonimidoyl)]-9-[(4-fluorophenyl)methyl]-N-methyl-8-oxo-purine-7-carboxamide;
6-Amino-9-[(4-bromophenyl)methyl]-2-(ethylsulfonimidoyl)-N-methyl-8-oxo-N-propyl-purine-7-carboxamide;
6-Amino-2-[S(R)-ethylsulfonimidoyl]-9-[(4-bromophenyl)methyl]-N-methyl-8-oxo-N-propyl-purine-7-carboxamide;
6-Amino-2-[S(S)-ethylsulfonimidoyl]-9-[(4-bromophenyl)methyl]-N-methyl-8-oxo-N-propyl-purine-7-carboxamide;
6-Amino-9-[(4-bromophenyl)methyl]-N-ethyl-2-(ethylsulfonimidoyl)-N-methyl-8-oxo-purine-7-carboxamide;
6-Amino-9-[(4-bromophenyl)methyl]-N-ethyl-2-[S(S)-(ethylsulfonimidoyl)]-N-methyl-8-oxo-purine-7-carboxamide; and,
6-Amino-9-[(4-bromophenyl)methyl]-N-ethyl-2-[S(R)-(ethylsulfonimidoyl)]-N-methyl-8-oxo-purine-7-carboxamide; or,
a pharmaceutically acceptable salt, enantiomer or diastereomer thereof.

14. The method according to claim 13 wherein the compound is selected from the group consisting of:
6-Amino-9-[(4-chlorophenyl)methyl]-N-ethyl-2[S(S)-ethylsulfonimidoyl]-N-methyl-8-oxo-purine-7-carboxamide;
6-Amino-9-[(4-chlorophenyl)methyl]-N-ethyl-2-[S(R)-ethylsulfonimidoyl]-N-methyl-8-oxo-purine-7-carboxamide;
6-Amino-9-[(4-bromophenyl)methyl]-N-ethyl-2-(ethylsulfonimidoyl)-N-methyl-8-oxo-purine-7-carboxamide;
6-Amino-9-[(4-bromophenyl)methyl]-N-ethyl-2-[S(S)-(ethylsulfonimidoyl)]-N-methyl-8-oxo-purine-7-carboxamide; and,
6-Amino-9-[(4-bromophenyl)methyl]-N-ethyl-2-[S(R)-(ethylsulfonimidoyl)]-N-methyl-8-oxo-purine-7-carboxamide; or,
a pharmaceutically acceptable salt, enantiomer or diastereomer thereof.

15. The method of claim 1 wherein the liver cancer is hepatocellular carcinoma, hepatoma, cholangiocarcinoma, hepatoblastoma, hepatic carcinoma, hepatic angiosarcoma, or metastatic liver cancer.

16. The method of claim 14 wherein the liver cancer is hepatocellular carcinoma.

17. The method of claim 1 comprising administering a composition comprising a compound of claim 1 and at least one therapeutically inert carrier.

18. The method for the treatment of liver cancer comprising administering to a patient in need thereof a therapeutically effective amount of a compound according to claim 1, or a pharmaceutically acceptable salt enantiomer or diastereomer thereof, in combination with an antagonistic PD1 antibody or antagonistic PD-L1 antibody.

19. The method of claim 18 wherein the antagonistic PD1 antibody is nivolumab or pemprolizumab.

20. The method of claim 19, wherein the compound according to claim 1 is 6-amino-9-[(4-chlorophenyl)methyl]-N-ethyl-2[S(S)-ethylsulfonimidoyl]-N-methyl-8-oxo-purine-7-carboxamide

21. The method according to claim 18, wherein the antagonistic PD1 antibody comprises a heavy chain variable domain VH with an amino acid sequence of SEQ ID NO: 5 and a light chain variable domain VL with an amino acid sequence of SEQ ID NO:6.

22. The method according to claim 21, wherein the compound according to claim 1 is 6-Amino-9-[(4-chlorophenyl)methyl]-N-ethyl-2[S(S)-ethylsulfonimidoyl]-N-methyl-8-oxo-purine-7-carboxamide.

23. The method according to claim 18 wherein the treatment comprises a compound according to claim 1 in combination with an antagonistic PD-L1 antibody.

24. The method according to claim 23, wherein the antagonistic PD-L1 antibody used in the combination therapy is atezolizumab or durvalumab or avelumab.

25. The method according to claim 24 wherein the antagonistic PD-L1 antibody is atezolizumab.

26. The method according to claim 25, wherein the compound is 6-amino-9-[(4-chlorophenyl)methyl]-N-ethyl-2[S(S)-ethylsulfonimidoyl]-N-methyl-8-oxo-purine-7-carboxamide.

27. The method for the treatment of liver cancer according to claim 18 which method further comprises treatment with an anti-angiogenic agent in the combination therapy

28. The method of claim 27 wherein the anti-angiogenic agent is selected from is sorafenib, regorafenib, sunitinib or bevacizumab is used in the combination therapy.

29. The method of claim 28 wherein the anti-angiogenic compound is sorafenib.

30. The method of claim 28 wherein the anti-angiogenic compound is bevacizumab.

31. The method according to claim 28 wherein the compound is 6-amino-9-[(4-chlorophenyl)methyl]-N-ethyl-2[S(S)-ethylsulfonimidoyl]-N-methyl-8-oxo-purine-7-carboxamide.


SEQUENCE LISTING (17 sequences)

SEQ ID NO: 1 - protein, 113 aa, Artificial [sequence omitted]

SEQ ID NO: 2 - protein, 107 aa, Artificial [sequence omitted]

SEQ ID NO: 3 - protein, 120 aa, Artificial [sequence omitted]

SEQ ID NO: 4 - protein, 111 aa, Artificial [sequence omitted]

SEQ ID NO: 5 - protein, 120 aa, Artificial
  humanized variant -heavy chain variable domain VH of PD1-0103_01
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSS

SEQ ID NO: 6 - protein, 111 aa, Artificial
  humanized variant -light chain variable domain VL of PD1-0103_01
DIVMTQSPDSLAVSLGERATINCKASESVDTSDNSFIHWYQQKPGQSPKLLIYRSSTLESGVPDRFSGSGSGTDFTLTISSLQAEDVAVYYCQQNYDVPWTFGQGTKVEIK

SEQ ID NO: 7 - protein, 118 aa, Artificial [sequence omitted]

SEQ ID NO: 8 - protein, 108 aa, Artificial
  light chain variable domain of anti-PD-L1 antibody atezolizumab
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKR

SEQ ID NO: 9 - protein, 121 aa, Artificial
  heavy chain variable domain of anti-PD-L1 antibody durvalumab
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSS

SEQ ID NO: 10 - protein, 108 aa, Artificial
  light chain variable domain of anti-PD-L1 antibody durvalumab
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIK

SEQ ID NO: 11 - protein, 120 aa, Artificial
  heavy chain of anti-PD-L1 antibody avelumab
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSS

SEQ ID NO: 12 - protein, 110 aa, Artificial
  light chain of anti-PD-L1 antibody avelumab
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVL

SEQ ID NO: 13 - protein, 290 aa, homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 14 - protein, 268 aa, Homo Sapiens
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 15 - protein, 107 aa, Homo sapiens [sequence omitted]

SEQ ID NO: 16 - protein, 328 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 17 - protein, 328 aa, homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP